(19) United States
(12) Patent Application Publication
(10) Pub. No.: US 2004/0048265 A1
(43) Pub. Date: Mar. 11, 2004

(54) Obesity associated biallelic marker maps

(76) Inventors: Daniel Cohen, Paris (FR); Marta Blumenfeld, Paris (FR); Ilya Chumakov, Vaux-le Penil (FR); Hadi Abderrahim, Charenton le Pont (FR); Bernard Bihain, Cancale (FR)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/333,429

(22) PCT Filed: Jun. 28, 2001
(86) PCT No.: PCT/IB01/01477

(51) Int. Cl. C12Q 1/68

(52) U.S. Cl. 435/6

(57) ABSTRACT

The present invention relates to genomic maps comprising biallelic markers, new biallelic markers, and methods of using biallelic markers. Primers hybridizing to regions flanking these biallelic markers are also provided. This invention provides polynucleotides and methods suitable for genotyping a nucleic acid containing sample for one or more biallelic markers of the invention. Further, the invention provides a number of methods utilizing the biallelic markers of the invention including methods to detect a statistical correlation between a biallelic marker allele and a phenotype and/or between a biallelic marker haplotype and a phenotype.

### FIELD OF THE INVENTION

The present invention relates to genomic maps comprising biallelic markers, new biallelic markers, and methods of using biallelic markers.

### BACKGROUND OF THE INVENTION

Recent advances in genetic engineering and bioinformatics have enabled the manipulation and characterization of large portions of the human genome. While efforts to obtain the full sequence of the human genome are rapidly progressing, there are many practical uses for genetic information which can be implemented with partial knowledge of the sequence of the human genome.

As the full sequence of the human genome is assembled, the partial sequence information available can be used to identify genes responsible for detectable human traits, such as genes associated with human diseases, and to develop diagnostic tests capable of identifying individuals who express a detectable trait as the result of a specific genotype or individuals whose genotype places them at risk of developing a detectable trait at a subsequent time. Each of these applications for partial genomic sequence information is based upon the assembly of genetic and physical maps which order the known genomic sequences along the human chromosomes.

The present invention relates to an ordered set of human genomic sequences comprising single nucleotide polymorphisms, as well as the use of these polymorphisms as a high resolution map of the human genome, methods of identifying genes associated with detectable human traits, and diagnostics for identifying individuals who carry a gene which causes them to express a detectable trait or which places them at risk of expressing a detectable trait in the future.

Advantages of the Biallelic Markers of the Present Invention

The map-related biallelic markers of the present invention offer a number of important advantages over other genetic markers such as RFLP (Restriction fragment length polymorphism), VNTR (Variable Number of Tandem Repeats) markers and earlier STS-(sequence tagged sites) derived markers.

The first generation of markers, were RFLPs, which are variations that modify the length of a restriction fragment. But methods used to identify and to type RFLPs are relatively wasteful of materials, effort, and time. Since they are biallelic markers (they present only two alleles, the restriction site being either present or absent), their maximum heterozygosity is 0.5. The theoretical number of RFLPs distributed along the entire human genome is more than 10⁵, which leads to a potential average inter-marker distance of 30 kilobases. However, in reality the number of evenly distributed RFLPs which occur at a sufficient frequency in the population to make them useful for tracking of genetic polymorphisms is very limited.

The second generation of genetic markers were VNTRs, which can be categorized as either minisatellites or microsatellites. Minisatellites are tandemly repeated DNA sequences present in units of 5-50 repeats which are distributed along regions of the human chromosomes ranging from 0.1 to 20 kilobases in length. Since they present many possible alleles, their informative content is very high. Minisatellites are scored by performing Southern blots to identify the number of tandem repeats present in a nucleic acid sample from the individual being tested. However, there are only 10⁴ potential VNTRs that can be typed by Southern blotting. Thus, the number of easily typed informative markers in these maps is far too small for the average distance between informative markers to fulfill the requirements for a useful genetic map. Moreover, both RFLP and VNTR markers are costly and time-consuming to develop and assay in large numbers.

Initial attempts to construct genetic maps based on non-RFLP biallelic markers have focused on identifying biallelic markers lying within sequence tagged sites (STS), pieces of genomic DNA having a known sequence and averaging about 250 bases in length. More than 30,000 STSs have been identified and ordered along the genome (Hudson et al., Science 270:1945-1954 (1995); Schuler et al., Science 274:540-546 (1996), the disclosures of which are incorporated herein by reference in their entireties). For example, the Whitehead Institute and Genethon's integrated map contains 15,086 STSs.

These sequence tagged sites can be screened to identify polymorphisms, preferably Single Nucleotide Polymorphisms (SNPs), more preferably non RFLP biallelic markers therein. Generally polymorphisms are identified by determining the sequence of the STSs in 5 to 10 individuals.

Wang et al. (Cold Spring harbor laboratory: Abstracts of papers presented on genome Mapping and sequencing p. 17 (May 14-18, 1997), the disclosure of which is incorporated herein by reference in its entirety) recently announced the identification and mapping of 750 Single Nucleotide Polymorphisms issued from the sequencing of 12,000 STSs from the Whitehead/MIT map, in eight unrelated individuals. The map was assembled using a high throughput system based on the utilization of DNA chip technology available from Affymetrix (Chee et al., Science 274:610-614 (1996), the disclosure of which is incorporated herein by reference in its entirety).

However, according to experimental data and statistical calculations, less than one out of 10 of all STSs mapped today will contain an informative Single Nucleotide Polymorphism. This is primarily due to the short length of existing STSs (usually less than 250 bp). If one assumes 10⁶ informative SNPs spread along the human genome, there would on average be one marker of interest every 3×10⁹/10⁶, i.e. every 3,000 bp. The probability that one such marker is present on a 250 bp stretch is thus less than 1/10.

While it could produce a high density map, the STS approach based on currently existing markers does not put any systematic effort into making sure that the markers obtained are optimally distributed throughout the entire genome. Instead, polymorphisms are limited to those locations for which STSs are available.

The even distribution of markers along the chromosomes or particular chromosomal region of interest is critical to the future success of genetic analyses. In particular, a high density map having appropriately spaced markers is essential for conducting association studies on sporadic cases, aiming at identifying genes responsible for detectable traits such as those which are described below.

As will be further explained below, genetic studies have mostly relied in the past on a statistical approach called linkage analysis, which took advantage of microsatellite markers to study their inheritance pattern within families from which a sufficient number of individuals presented the studied trait. Because of intrinsic limitations of linkage analysis, which will be further detailed below, and because these studies necessitate the recruitment of adequate family pedigrees, they are not well suited to the genetic analysis of all traits, particularly those for which only sporadic cases are available (e.g. drug response traits), or those which have a low penetrance within the studied population.

Association studies enabled by the biallelic markers of the present invention offer an alternative to linkage analysis. Combined with the use of a high density map of appropriately spaced, sufficiently informative markers, association studies, including linkage disequilibrium-based genome wide association studies, will enable the identification of most genes involved in complex traits.

Single nucleotide polymorphism or biallelic markers can be used in the same manner as RFLPs and VNTRs but offer several advantages. Single nucleotide polymorphisms are densely spaced in the human genome and represent the most frequent type of variation. An estimated number of more than 10⁷ sites are scattered along the 3×10⁹ base pairs of the human genome. Therefore, single nucleotide polymorphisms occur at a greater frequency and with greater uniformity than RFLP or VNTR markers which means that there is a greater probability that such a marker will be found in close proximity to a genetic locus of interest. Single nucleotide polymorphisms are less variable than VNTR markers but are mutationally more stable.

Also, the different forms of a characterized single nucleotide polymorphism, such as the biallelic markers of the present invention, are often easier to distinguish and can therefore be typed easily on a routine basis. Biallelic markers have single nucleotide based alleles and they have only two common alleles, which allows highly parallel detection and automated scoring. The biallelic markers of the present invention offer the possibility of rapid, high-throughput genotyping of a large number of individuals.

Biallelic markers are densely spaced in the genome, sufficiently informative and can be assayed in large numbers. The combined effects of these advantages make biallelic markers extremely valuable in genetic studies. Biallelic markers can be used in linkage studies in families, in allele sharing methods, in linkage disequilibrium studies in populations, in association studies of case-control populations. An important aspect of the present invention is that biallelic markers allow association studies to be performed to identify genes involved in complex traits. Association studies examine the frequency of marker alleles in unrelated case- and control-populations and are generally employed in the detection of polygenic or sporadic traits. Association studies may be conducted within the general population and are not limited to studies performed on related individuals in affected families (linkage studies). Biallelic markers in different genes can be screened in parallel for direct association with disease or response to a treatment. This multiple gene approach is a powerful tool for a variety of human genetic studies as it provides the necessary statistical power to examine the synergistic effect of multiple genetic factors on a particular phenotype, drug response, sporadic trait, or disease state with a complex genetic etiology.

Obesity Disorder Associated Regions

Obesity is a public health problem that is both serious and widespread. One-third of the population in industrialized countries has an excess weight of at least 20% relative to the ideal weight. The phenomenon continues to worsen, particularly in regions of the globe where economies are modemizing. In the United States, the number of obese people has escalated from 25% at the end of the 70s to 33% at the beginning of the 90s.

Obesity considerably increases the risk of developing cardiovascular or metabolic diseases. It is estimated that if the entire population had an ideal weight, the risk of coronary insufficiency would decrease by 25% and that of cardiac insufficiency and of cerebral vascular accidents by 35%. Coronary insufficiency, atheromatous disease and cardiac insufficiency are at the forefront of the cardiovascular complications induced by obesity. For an excess weight greater than 30%, the incidence of coronary diseases is doubled in subjects under 50 years. Studies carried out for other diseases are equally eloquent. For an excess weight of 20%, the risk of high blood pressure is doubled. For an excess weight of 30%, the risk of developing a non-insulin-dependent diabetes is tripled, and that of hyperlipidemias is multiplied six-fold.

The list of diseases having onsets promoted by obesity includes: hyperuricemia (11.4% in obese subjects, against 3.4% in the general population), digestive pathologies, abnormalities in hepatic functions, and even certain cancers.

Whether the physiological changes in obesity are characterized by an increase in the number of adipose cells, or by an increase in the quantity of triglycerides stored in each adipose cell, or by both, this excess weight results mainly from an imbalance between the quantities of calories consumed and the quantity of calories used by the body. Studies on the causes of this imbalance have been in several directions. Some have focused on studying the mechanism of absorption of foods, and therefore the molecules that control food intake and the feeling of satiety. Other studies have characterized the pathways through which the body uses its calories.

The proposed treatments for obesity are of five types. (1) Food restriction is the most frequently used. The obese individuals are advised to change their dietary habits so as to consume fewer calories. Although this type of treatment is effective in the short-term, the recidivation rate is very high. (2) Increased calorie use through physical exercise is also proposed. This treatment is ineffective when applied alone, but it improves weight-loss in subjects on a low-calorie diet. (3) Gastrointestinal surgery, which reduces the absorption of the calories ingested, is effective, but has been virtually abandoned because of the side effects it causes. (4) The medicinal approach uses either the anorexigenic action of molecules involved at the level of the central nervous system, or the effect of molecules that increase energy use by increasing the production of heat. The prototypes of this type of molecule are the thyroid hormones that uncouple oxidative phosphorylations of the mitochondrial respiratory chain. The side effects and the toxicity of this type of treatment make their use dangerous. (5) An approach that aims to reduce the absorption of dietary lipids by sequestering them in the lumen of the digestive tube is also in place. However, it induces physiological imbalances which are difficult to tolerate: deficiency in the absorption of fat-soluble vitamins, flatulence and steatorrhoea. Whatever the envisaged therapeutic approach, the treatments of obesity are all characterized by an extremely high recidivation rate.

The molecular mechanisms responsible for obesity in man are complex and involve genetic and environmental factors. Because of the low efficiency of the current treatments, it is urgent to define the genetic mechanisms which determine obesity, so as to be able to develop better targeted medicaments.

More than 20 genes have been studied as possible candidates, either because they have been implicated in diseases of which obesity is one of the clinical manifestations, or because they are homologues of genes involved in obesity in animal models. Situated in the 3q27 chromosomal region, the human adipocyte-specific APM1 gene encodes a secretory protein of the adipose tissue and is likely to play a role in the pathogenesis of obesity. Knowledge of the APM1 genomic sequence, and particularly of both promoter and splice junction sequences, allows the design of novel diagnostics and therapeutic tools that act on lipid metabolism, and are useful for diagnosing and treating obesity disorders.

Hager, J. et al., Nature Genetics (1998) November;20:304-308 conducted a genome wide scan in affected sibpairs to identify chromosomal regions linked to obesity in a collection of French families. Model-free multipoint linkage analysis revealed evidence for linkage to a region on chromosome 10p (MLS=4.85). This MLS value for the region exceeds the suggested reference threshold given for linkage (Lander, E. et al. Nature Genet. 11, 241-247 (1995)).

LSR is a multimeric receptor encoded by the products of a single gene located on chromosome 19q13.1 and fully described in Yen, F. et al. J. Biol. Chem. “Molecular Cloning of a Lipolysis Stimulated Remnant Receptor Expressed in the Liver,” in press, PCT Patent No: WO IB98/01256 and PCT Patent No: WO IB98/01257. On the basis of data obtained using cell biology, animal physiology, molecular biology and classical biochemistry techniques, applicants have demonstrated that LSR serves two primary functions: the cellular uptake of triglyceride rich lipoproteins and the binding of leptin.

LSR, as a multimer constituted of α and β subunits organized with a stoichiometry that ranges between α1/β2 and α1/β5 with an average of α1/β3, serves for the cellular binding, uptake and degradation of triglyceride-rich lipoproteins. Because LSR is primarily expressed in the liver and appears rate limiting for the clearance of dietary TG, this pathway is instrumental in the partitioning of dietary lipids between the liver and peripheral tissue. It appears that a genetic defect in LSR leads to excess delivery of dietary lipids to the adipose tissue. An effect in the hepatic clearance of dietary TG may lead to several disorder relating to metabolism, transport, and storage such as diabetes, hypertension and atherosclerosis. If the amount delivered to these storage sites exceed their FFA (free fatty acid) releasing capacity; their size will increase, causing obesity and eventually a series of metabolic complications.

Clearly there remains a need for novel medicaments that are useful for reducing body weight in humans. Such pharmaceutical compositions advantageously would help to control obesity and thereby alleviate many of the cardiovascular consequences associated with this condition.

The discovery of new genes which are associated to obesity would also allow the design of novel diagnostic and therapeutic tools acting on the lipid metabolism, useful for diagnosing and treating obesity disorders.

The present invention relates to a high density linkage disequilibrium-based genetic map of the human genome which comprise the map-related biallelic markers of the invention and will allow the identification of genes responsible for detectable traits using genome-wide association studies and linkage disequilibrium mapping.

### SUMMARY OF THE INVETION

The present invention is based on the discovery of a set of novel map-related biallelic markers. See Table 1a. The position of these markers and knowledge of the surrounding sequence has been used to design polynucleotide compositions which are useful in high density mapping of the human genome as well as in determining the identity of nucleotides at the marker position, and more complex association and baplotyping studies which are useful in determining the genetic basis for disease states. In addition, the compositions and methods of the invention find use in the identification of the targets for the development of pharmaceutical agents and diagnostic methods, as well as the characterization of the differential efficacious responses to and side effects from pharmaceutical agents acting on a disease as well as other treatments.

A first embodiment of the present invention is a map of the human genome, or a region of the human genome, comprising an ordered array of biallelic markers, wherein at least 1, 2, 3, 4, 5, 10, 15, 20, 25, 30, 40, 50, 70, 85, 100 or all of said biallelic markers are map-related biallelic markers. In addition, the maps of the present invention encompass maps with any further limitation described in this disclosure, or those following, specified alone or in any combination: optionally, said map-related biallelic marker may be selected individually or in any combination from the group consisting of the biallelic markers of SEQ ID Nos. 1 to 171, 1 to 100, 101 to 162, 163 to 171 and the complements thereof; optionally said ordered array comprises at least 20,000, 40,000, 60,000, 80,000, 100,000, or 120,000 biallelic markers; optionally, wherein said biallelic markers are separated from one another by an average distance of 10 kb-200 kb, 15 kb-150 kb, 20 kb-100 kb, 100 kb-150 kb, 50-100 kb, or 25 kb-50 kb in the human genome; optionally, said biallelic markers are distributed at an average density of at least one biallelic marker every 150 kb, 50 kb, or 30 kb in the human genome; or optionally, wherein, all of said biallelic markers are selected to have a heterozygosity rates of at least about 0.18, 0.32, or 0.42. The present invention also relates to a map of one or more regions of the human genome, preferably a high density map of one or more regions of the human genome, comprising an ordered array of biallelic markers, wherein at least 1, 2, 3, 4, 5, 10, 15, 20, 25, 30, 40, 50, 70, 85, 100 or all of said biallelic markers are map-related biallelic markers. Said map-related biallelic markers may comprise any number or combination of map-related biallelic markers localized on obesity disorder-associated chromosomal regions on chromosomes 3, 10, 19, and are further described herein. Optionally, a biallelic marker map comprises at least 1, 2, 3, 4, 5, 10, 15, 20, 25, 30, 40, 50, 70, 85, 100 biallelic markers, wherein at least 1, 2, 3, 4, 5, 10, 15, 20, 25, 30, 40 or 50 of said biallelic markers are selected from the group of biallelic markers consisting of:

chromosome 3 biallelic markers: (a) SEQ ID Nos. 8, 10, 12, 13, 14, 15, 16, 17, 18, 19, 20, 23, 24, 25, 26, 27, 70, 72, 73, 74, 75, 76, 77; and (b) SEQ ID Nos. 102, 105, 106, 107, 110, 111, 117, 118, 119, 120, 121, 122, 123, 124, 125, 126, 127, 159, 160, 161; and (c) 163, 166, 167;

chromosome 10 biallelic markers: (a) SEQ ID Nos. 1, 2, 3, 4, 5, 6, 7, 9, 11, 21, 22, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 71, 78, 79, 80, 81, 82, 83, 84, 85, 86, 87, 88, 89, 90, 91, 92, 93, 94, 95, 96, 97, 98, 99, 100; (b) SEQ ID Nos. 101, 103, 104, 108, 109, 112, 113, 114, 115, 116, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158; and (c) SEQ ID Nos. 164, 165, 168, 169, 170, 171; and

chromosome 19 biallelic marker: (a) SEQ ID No. 162.

A second embodiment of the invention encompasses isolated, purified or recombinant polynucleotides consisting of, consisting essentially of, or comprising a contiguous span of nucleotides of a sequence selected as an individual or in any combination from the group consisting of SEQ ID No. 1 to 171, 1 to 100, 101 to 162, 163 to 171, 172 to 513, 172 to 271, 272 to 333, 334 to 342, 343 to 442, 443 to 504 and 505 to 513, or the complements thereof, wherein said contiguous span is at least 8, 10, 12, 15, 18, 19, 20, 21, 22, 23, 24, 25, 30, 35, 43, 44, 45, 46 or 47 nucleotides in length, to the extent that a contiguous span of these lengths is consistent with the lengths of the particular Sequence ID. The present invention also relates to polynucleotides hybridizing under stringent or intermediate conditions to a sequence selected from the group consisting of SEQ ID No. 1 to 171, 1 to 100, 101 to 162, 163 to 171, 172 to 513, 172 to 271, 272 to 333, 334 to 342, 343 to 442, 443 to 504 and 505 to 513 and the complements thereof. In addition, the polynucleotides of the invention encompass polynucleotides with any further limitation described in this disclosure, or those following, specified alone or in any combination: said contiguous span may optionally comprise a map-related biallelic marker; optionally either the 1^{st} or the 2^{nd} allele of the respective SEQ ID No., as indicated in Table 1a, may be specified as being present at said map-related biallelic marker; optionally, said biallelic marker may be within 6, 5, 4, 3, 2, or 1 nucleotides of the center of said polynucleotide or at the center of said polynucleotide; optionally, said polynucleotide may comprise, consist of, or consist essentially of a contiguous span which ranges in length from 8, 10, 12, 15, 18 or 20 to 21, 25, 35, 40, 43, or 47 nucleotides; optionally, said polynucleotide may comprise, consist of, or consist essentially of a contiguous span which ranges in length from 8, 10, 12, 15, 18 or 20 to 21, 25, 35, 40, 43, or 47 nucleotides, or be specified as being 12, 15, 18, 20, 25, 35, 40, 43, or 47 nucleotides in length and including an map-related biallelic marker of said sequence, and optionally the 1^{st} allele of Table 1a is present at said biallelic marker; optionally, the 3′ end of said contiguous span may be present at the 3′ end of said polynucleotide; optionally, biallelic marker may be present at the 3′ end of said polynucleotide; optionally, the 3′ end of said polynucleotide may be located within or at least 2, 4, 6, 8, or 10 nucleotides upstream of a map-related biallelic marker in said sequence, to the extent that such a distance is consistent with the lengths of the particular Sequence ID; optionally, the 3′ end of said polynucleotide may be located 1 nucleotide upstream of a map-related biallelic marker in said sequence; and optionally, said polynucleotide may further comprise a label.

Further embodiments of the invention include isolated nucleic acid molecules that comprise, or alternatively consist of, a polynucleotide having a nucleotide sequence at least 90% identical, and more preferably at least 95%, 96%, 97%, 98% or 99% identical, to any of the nucleotide sequences of the invention, or a polynucleotide which hybridizes under stringent hybridization conditions to a polynucleotide above.

A third embodiment of the invention encompasses any polynucleotide of the invention attached to a solid support. In addition, the polynucleotides of the invention which are attached to a solid support encompass polynucleotides with any further limitation described in this disclosure, or those following, specified alone or in any combination: optionally, said polynucleotides may be specified as attached individually or in groups of at least 2, 5, 8, 10, 12, 15, 20, 25, 50, 100, 200, or 500 distinct polynucleotides of the inventions to a single solid support; optionally, polynucleotides other than those of the invention may attached to the same solid support as polynucleotides of the invention; optionally, when multiple polynucleotides are attached to a solid support they may be attached at random locations, or in an ordered array; optionally, said ordered array may be addressable.

A fourth embodiment of the invention encompasses the use of any polynucleotide for, or any polynucleotide for use in, determining the identity of nucleotides at a map-related biallelic marker. In addition, the polynucleotides of the invention for use in determining the identity of nucleotides at a map-related biallelic marker encompass polynucleotides with any further limitation described in this disclosure, or those following, specified alone or in any combination: optionally, said map-related biallelic marker may be selected individually or in any combination from the group consisting of the biallelic markers of SEQ ID No. 1 to 171, 1 to 100, 101 to 162, 163 to 171 and the complements thereof; optionally, said polynucleotide may comprise a sequence disclosed in the present specification; optionally, said polynucleotide may comprise, consist of, or consist essentially of any polynucleotide described in the present specification; optionally, said determining may be performed in a hybridization assay, sequencing assay, microsequencing assay, or an enzyme-based mismatch detection assay; optionally, said polynucleotide may be attached to a solid support, array, or addressable array; optionally, said polynucleotide may be labeled.

A fifth embodiment of the invention encompasses the use of any polynucleotide for, or any polynucleotide for use in, amplifying a segment of nucleotides comprising a map-related biallelic marker. In addition, the polynucleotides of the invention for use in amplifying a segment of nucleotides comprising a map-related biallelic marker encompass polynucleotides with any further limitation described in this disclosure, or those following, specified alone or in any combination: optionally, said map-related biallelic marker may be selected individually or in any combination from the group consisting of the biallelic markers of SEQ ID Nos. 1 to 171, 1 to 100, 101 to 162, 163 to 171 and the complements thereof; optionally, said polynucleotide may comprise, consist of, consist essentially of, or comprise a sequence selected individually or in any combination from the group consisting of SEQ ID No. 172 to 513, 172 to 271, 272 to 333, 334 to 342, 343 to 442, 443 to 504 and 505 to 513; optionally, said polynucleotide may comprise, consist of, or consist essentially of any polynucleotide described in the present specification; optionally, said amplifying may be performed by a PCR or LCR. Optionally, said polynucleotide may be attached to a solid support, array, or addressable array. Optionally, said polynucleotide may be labeled.

A sixth embodiment of the invention encompasses methods of genotyping a biological sample comprising determining the identity of a nucleotide at a map-related biallelic marker. In addition, the genotyping methods of the invention encompass methods with any further limitation described in this disclosure, or those following, specified alone or in any combination: optionally, said map-related biallelic marker may be selected individually or in any combination from the group consisting of the biallelic markers of SEQ ID No. 1 to 171, 1 to 100, 101 to 162, 163 to 171 and the complements thereof; optionally, said method further comprises determining the identity of a second nucleotide at said biallelic marker, wherein said first nucleotide and second nucleotide are not base paired (by Watson & Crick base pairing) to one another; optionally, said biological sample is derived from a single individual or subject; optionally, said method is performed in vitro; optionally, said biallelic marker is determined for both copies of said biallelic marker present in said individual's genome; optionally, said biological sample is derived from multiple subjects or individuals; optionally, said method further comprises amplifying a portion of said sequence comprising the biallelic marker prior to said determining step; optionally, wherein said amplifying is performed by PCR, LCR, or replication of a recombinant vector comprising an origin of replication and said portion in a host cell; optionally, wherein said determining is performed by a hybridization assay, sequencing assay, microsequencing assay, or an enzyme-based mismatch detection assay.

A seventh embodiment of the invention comprises methods of estimating the frequency of an allele in a population comprising genotyping individuals from said population for a map-related biallelic marker and determining the proportional representation of said biallelic marker in said population. In addition, the methods of estimating the frequency of an allele in a population of the invention encompass methods with any further limitation described in this disclosure, or those following, specified alone or in any combination: optionally, said map-related biallelic marker may be selected individually or in any combination from the group consisting of the biallelic markers of SEQ ID Nos. 1 to 171, 1 to 100, 101 to 162, 163 to 171 and the complements thereof; optionally, determining the frequency of a biallelic marker allele in a population may be accomplished by determining the identity of the nucleotides for both copies of said biallelic marker present in the genome of each individual in said population and calculating the proportional representation of said nucleotide at said map-related biallelic marker for the population; optionally, determining the frequency of a biallelic marker allele in a population may be accomplished by performing a genotyping method on a pooled biological sample derived from a representative number of individuals, or each individual, in said population, and calculating the proportional amount of said nucleotide compared with the total.

An eighth embodiment of the invention comprises methods of detecting an association between an allele and a phenotype, comprising the steps of a) determining the frequency of at least one map-related biallelic marker allele in a trait positive population, b) determining the frequency of said map-related biallelic marker allele in a control population and; c) determining whether a statistically significant association exists between said genotype and said phenotype. In addition, the methods of detecting an association between an allele and a phenotype of the invention encompass methods with any further limitation described in this disclosure, or those following, specified alone or in any combination: optionally, said map-related biallelic marker may be selected individually or in any combination from the group consisting of the biallelic markers of SEQ ID Nos. 1 to 171, 1 to 100, 101 to 162, 163 to 171 and the complements thereof; optionally, said control population may be a trait-negative population, or a random population; optionally, wherein said phenotype is selected from the group consisting of disease, treatment response, treatment efficacy, drug response, drug efficacy, and drug toxicity; optionally, the determining steps a) and b) are performed on all of the biallelic markers of SEQ ID Nos. 1 to 171.

An ninth embodiment of the present invention encompasses methods of estimating the frequency of a haplotype for a set of biallelic markers in a population, comprising the steps of: a) genotyping each individual in said population for at least one map-related biallelic marker, b) genotyping each individual in said population for a second biallelic marker by determining the identity of the nucleotides at said second biallelic marker for both copies of said second biallelic marker present in the genome; and c) applying a haplotype determination method to the identities of the nucleotides determined in steps a) and b) to obtain an estimate of said frequency. In addition, the methods of estimating the frequency of a haplotype of the invention encompass methods with any further limitation described in this disclosure, or those following, specified alone or in any combination: optionally said haplotype determination method is selected from the group consisting of asymmetric PCR amplification, double PCR amplification of specific alleles, the Clark method, or an expectation maximization algorithm; optionally, said map-related biallelic marker may be selected individually or in any combination from the group consisting of the biallelic markers of SEQ ID Nos. 1 to 171, 1 to 100, 101 to 162, 163 to 171 and the complements thereof; optionally, said second biallelic marker is a map-related biallelic marker; optionally, the identity of the nucleotides at the biallelic markers in every one of the sequences of SEQ ID Nos. 1 to 171 is determined in steps a) and b).

A tenth embodiment of the present invention encompasses methods of detecting an association between a haplotype and a phenotype, comprising the steps of: a) estimating the frequency of at least one haplotype in a trait positive population according to a method of estimating the frequency of a haplotype of the invention; b) estimating the frequency of said haplotype in a control population according to the method of estimating the frequency of a haplotype of the invention; and c) determining whether a statistically significant association exists between said haplotype and said phenotype. In addition, the methods of detecting an association between a haplotype and a phenotype of the invention encompass methods with any further limitation described in this disclosure, or those following, specified alone or in any combination: optionally, said map-related biallelic marker may be in a sequence selected individually or in any combination from the group consisting of SEQ ID No. 1 to 171, 1 to 100, 101 to 162, 163 to 171 and the complements thereof; optionally, said control population may be a trait-negative population, or a random population; optionally, wherein said phenotype is selected from the group consisting of disease, treatment response, treatment efficacy, drug response, drug efficacy, and drug toxicity; optionally, the identity of the nucleotides at the biallelic markers in every one of the following sequences: SEQ ID Nos. 1 to 171 is included in the estimating steps a) and b).

An eleventh embodiment of the present invention is a method of identifying a gene associated with a detectable trait comprising the steps of: a) determining the frequency of each allele of at least one map-related biallelic marker in individuals having the detectable trait and individuals lacking the detectable trait; b) identifying at least one allele of one or more biallelic markers having a statistically significant association with the detectable trait; and c) identifying a gene in linkage disequilibrium with said allele. In addition, the methods of the present invention for identifying a gene associated with a detectable trait encompass methods with any further limitation described in this disclosure, or those following, specified alone or in any combination: optionally, wherein the method further comprises d) identifying a mutation in the gene identified in step c) which is associated with the detectable trait; optionally, wherein the individuals having the detectable trait and the individuals lacking the detectable trait are readily distinguishable from one another; optionally, wherein the individuals having the detectable trait and the individuals lacking the detectable trait are selected from a bimodal population; optionally, wherein the individuals having the detectable trait are at one extreme of the population and the individuals lacking the detectable trait are at the other extreme of the population; optionally, said map-related biallelic marker may be in a sequence selected individually or in any combination from the group consisting of SEQ ID No. 1 to 171, 1 to 100, 101 to 162, 163 to 171 and the complements thereof; optionally, wherein said detectable trait is selected from the group consisting of disease, treatment response, treatment efficacy, drug response, drug efficacy, and drug toxicity.

A twelfth embodiment of the present invention is a method of identifying biallelic markers associated with a detectable trait comprising the steps of: a) determining the frequencies of a set of biallelic markers comprising at least one map-related biallelic marker in individuals who express said detectable trait and individuals who do not express said detectable trait; and b) identifying one or more biallelic markers in said set which are statistically associated with the expression of said detectable trait. In addition, the methods of the present invention for identifying biallelic markers associated with a detectable trait encompass methods with any further limitation described in this disclosure, or those following, specified alone or in any combination: optionally, said map-related biallelic marker may be in a sequence selected individually or in any combination from the group consisting of SEQ ID No. 1 to 171, 1 to 100, 101 to 162, 163 to 171 and the complements thereof; optionally, wherein said detectable trait is selected from the group consisting of disease, treatment response, treatment efficacy, drug response, drug efficacy, and drug toxicity.

A thirteenth embodiment of the present invention is a method of identifying biallelic marker(s) in linkage disequilibrium with a trait causing allele or in linkage disequilibrium with a trait-associated biallelic marker comprising the steps of: a) selecting at least one map-related biallelic marker which is in the genomic region suspected of containing the trait-causing allele or the trait-associated biallelic marker; and b) determining which of the map-related biallelic markers are associated with the trait-causing allele or in linkage disequilibrium with the trait-associated biallelic marker. In addition, the methods of the present invention for identifying biallelic marker(s) in linkage disequilibrium with a trait causing allele or in linkage disequilibrium with a trait-associated biallelic marker encompass methods with any further limitation described in this disclosure, or those following, specified alone or in any combination: optionally, said map-related biallelic marker may be in a sequence selected individually or in any combination from the group consisting of SEQ ID No. 1 to 171, 1 to 100, 101 to 162, 163 to 171 and the complements thereof; optionally, wherein said detectable trait is selected from the group consisting of disease, treatment response, treatment efficacy, drug response, drug efficacy, and drug toxicity.

A fourteenth embodiment of the present invention is a method for determining whether an individual is at risk of developing a detectable trait or suffers from a detectable trait comprising the steps of: a) obtaining a nucleic acid sample from the individual; b) screening the nucleic acid sample with at least one map-related biallelic marker; and c) determining whether the nucleic acid sample contains at least one allele of said map-related biallelic marker statistically associated with the detectable trait. In addition, the methods of the present invention for determining whether an individual is at risk of developing a detectable trait or suffers from a detectable trait encompass methods with any further limitation described in this disclosure, or those following, specified alone or in any combination: optionally, said map-related biallelic marker may be in a sequence selected individually or in any combination from the group consisting of SEQ ID No. 1 to 171, 1 to 100, 101 to 162, 163 to 171 and the complements thereof; optionally, wherein said detectable trait is selected from the group consisting of disease, treatment response, treatment efficacy, drug response, drug efficacy, and drug toxicity.

A fifteenth embodiment of the present invention is a method of administering a drug or a treatment comprising the steps of: a) obtaining a nucleic acid sample from an individual; b) determining the identity of the polymorphic base of at least one map-related biallelic marker which is associated with a positive response to the treatment or the drug; or at least one biallelic map-related marker which is associated with a negative response to the treatment or the drug; and c) administering the treatment or the drug to the individual if the nucleic acid sample contains said biallelic marker associated with a positive response to the treatment or the drug or if the nucleic acid sample lacks said biallelic marker associated with a negative response to the treatment or the drug. In addition, the methods of the present invention for administering a drug or a treatment encompass methods with any further limitation described in this disclosure, or those following, specified alone or in any combination: optionally, said map-related biallelic marker may be in a sequence selected individually or in any combination from the group consisting of SEQ ID No. 1 to 171, 1 to 100, 101 to 162, 163 to 171 and the complements thereof; or optionally, the administering step comprises administering the drug or the treatment to the individual if the nucleic acid sample contains said biallelic marker associated with a positive response to the treatment or the drug and the nucleic acid sample lacks said biallelic marker associated with a negative response to the treatment or the drug.

A sixteenth embodiment of the present invention is a method of selecting an individual for inclusion in a clinical trial of a treatment or drug comprising the steps of: a) obtaining a nucleic acid sample from an individual; b) determining the identity of the polymorphic base of at least one map-related biallelic marker which is associated with a positive response to the treatment or the drug, or at least one map-related biallelic marker which is associated with a negative response to the treatment or the drug in the nucleic acid sample, and c) including the individual in the clinical trial if the nucleic acid sample contains said map-related biallelic marker associated with a positive response to the treatment or the drug or if the nucleic acid sample lacks said biallelic marker associated with a negative response to the treatment or the drug. In addition, the methods of the present invention for selecting an individual for inclusion in a clinical trial of a treatment or drug encompass methods with any further limitation described in this disclosure, or those following, specified alone or in any combination; optionally, said map-related biallelic marker may be in a sequence selected individually or in any combination from the group consisting of SEQ ID No. 1 to 171, 1 to 100, 101 to 162, 163 to 171 and the complements thereof; optionally, the including step comprises administering the drug or the treatment to the individual if the nucleic acid sample contains said biallelic marker associated with a positive response to the treatment or the drug and the nucleic acid sample lacks said biallelic marker associated with a negative response to the treatment or the drug.

A seventeenth embodiment of the present invention is a method of identifying a gene associated with a detectable trait comprising the steps of: a) selecting a gene suspected of being associated with a detectable trait; and b) identifying at least one map-related biallelic marker within said gene which is associated with said detectable trait. In addition, the methods of the present invention for identifying a gene associated with a detectable trait encompass methods with any further limitation described in this disclosure, or those following, specified alone or in any combination: optionally, said map-related biallelic marker may be in a sequence selected individually or in any combination from the group consisting of SEQ ID No. 1 to 171, 1 to 100, 101 to 162, 163 to 171 and the complements thereof; optionally, the identifying step comprises determining the frequencies of the map-related biallelic marker(s) in individuals who express said detectable trait and individuals who do not express said detectable trait and identifying one or more biallelic markers which are statistically associated with the expression of the detectable trait.

Additional embodiments are set forth in the Detailed Description of the Invention and in the Examples.

### BRIEF DESCRIPTION OF THE DRAWINGS

FIG. 1 is a cytogenetic map of chromosome 21.

FIG. 2A shows the results of a computer simulation of the distribution of inter-marker spacing on a randomly distributed set of biallelic markers indicating the percentage of biallelic markers which will be spaced a given distance apart for 1, 2, or 3 markers/13AC in a genomic map (assuming a set of 20,000 minimally overlapping BACs covering the genome are evaluated).

FIG. 2B shows the results of a computer simulation of the distribution of inter-marker spacing on a randomly distributed set of biallelic markers indicating the percentage of biallelic markers which will be spaced a given distance apart for 1, 3, or 6 markers/BAC in a genomic map (assuming a set of 20,000 minimally overlapping BACs covering the genome are evaluated).

FIG. 3 shows, for a series of hypothetical sample sizes, the p-value significance obtained in association studies performed using individual markers from the high-density biallelic map, according to various hypotheses regarding the difference of allelic frequencies between the trait-positive and trait-negative samples.

FIG. 4 is a hypothetical association analysis conducted with a map comprising about 3,000 biallelic markers.

FIG. 5 is a hypothetical association analysis conducted with a map-comprising about 20,000 biallelic markers.

FIG. 6 is a hypothetical association analysis conducted with a map comprising about 60,000 biallelic markers.

FIG. 7 is a haplotype analysis using biallelic markers in the Apo E region.

FIG. 8 is a simulated haplotype analysis using the biallelic markers in the Apo E region included in the haplotype analysis of FIG. 7.

FIG. 9 shows a minimal array of overlapping clones which was chosen for further studies of biallelic markers associated with prostate cancer, the positions of STS markers known to map in the candidate genomic region along the contig, and the locations of biallelic markers along the BAC contig harboring a genomic region harboring a candidate gene associated with prostate cancer which were identified using the methods of the present invention.

FIG. 10 is a rough localization of a candidate gene for prostate cancer which was obtained by determining the frequencies of the biallelic markers of FIG. 9 in affected and unaffected populations.

FIG. 11 is a further refinement of the localization of the candidate gene for prostate cancer using additional biallelic markers which were not included in the rough localization illustrated in FIG. 10.

FIG. 12 is a haplotype analysis using the biallelic markers in the genomic region of the gene associated with prostate cancer.

FIG. 13 is a simulated haplotype using the six markers included in haplotype 5 of FIG. 12.

FIGS. 14A and 14B show the chromosomal localization and genomic organization of the LSR gene. FIG. 14A is a schematic diagram of chromosome 19 and of the genomic organization of LSR. The exon and intron lengths in bp are indicated as normal and italicized numbers, respectively. The location of USF2 further downstream is also shown. FIG. 14B shows SNPs on 19q13.1 and identifies those used for the association studies (highlighted in boxes).

FIGS. 15A, 15B, and 15C are graphical representations of an association study of plasma lipid values with LSR SNPs. Differences in genotype frequency in two groups of adolescent girls that were separated according to their plasma TG (FIG. 15A), total cholesterol (FIG. 15B) and free fatty acid (FIG. 15C) values being greater or lower than the mean of the entire population (Table 6) were analyzed by 3×2 χ² (chi square) analysis. χ² values for each test marker are represented as bars. The mean χ² value obtained with the 18 random markers is shown as a solid line; the calculated 99.99% confidence interval of this mean is shown as a dotted line for each parameter.

FIGS. 16A, 16B, 16C, and 16D show a graphical representation of the effect of the LSR exon 6 coding mutation on postprandial lipemia in obese adolescent girls. Thirty-four overnight-fasted obese adolescent girls consumed a high-fat test meal. Plasma TG were determined before, 2, and 4 hr after this meal. The genotypes of LSR markers #1, 2, and 3 were determined as described herein. The postprandial response (mean±SEM) as a function of genotype difference at each polymorphic site is shown in FIGS. 16A, 16B, and 16C. FIG. 16D is a plot of postprandial lipemic response taking into account the genotype of both LSR SNPs #1 and #3. Statistical comparison of the differences between means was first performed by analysis of variance. Significant results were then tested by unpaired t-test. The significance of the t-test is indicated on the graph. The data are presented using the pooled samples of hetero- and homozygous subjects in order to obtain a sufficient number of subjects in each group.

FIGS. 17A and 17B show the effect of LSR polymorphisms on the insulin to BMI relationship in obese adolescent girls. Fasting plasma insulin levels were determined in a population of obese adolescent girls and were plotted against their BMI and a regression line was generated (FIG. 17A). Genotype frequencies of the 5 LSR markers were compared based on whether individuals were above or below the regression line and presented as a χ² analysis (FIG. 17B). The results show that LSR marker 2 significantly influences the relationship between insulin and BMI in obese adolescent girls. The mean and 99.99% confidence interval of random markers are shown as solid and dotted lines, respectively.

FIGS. 18A, 18B, 18C, and 18D show the effect of the LSR polymorphism on glucose tolerance in obese adolescent girls. Glucose and insulin concentrations were determined on plasma samples taken before (to) and 2 h after (t120) a glucose tolerance test and the relative increase of plasma glucose compared with the increase in plasma insulin was calculated and plotted as a function of SNP genotype. SNP #1 is shown in FIG. 18A, SNP #2 in FIG. 18B, SNP #3 in FIG. 18C, and SNP #4 in FIG. 18D. The data show that only the polymorphism at LSR marker 2 significantly influences the ratio of the relative increase of plasma glucose to that of the relative increase in plasma insulin

FIG. 19 is a block diagram of an exemplary computer system.

FIG. 20 is a flow diagram illustrating one embodiment of a process 200 for comparing a new nucleotide or protein sequence with a database of sequences in order to determine the homology levels between the new sequence and the sequences in the database.

FIG. 21 is a flow diagram illustrating one embodiment of a process 250 in a computer for determining whether two sequences are homologous.

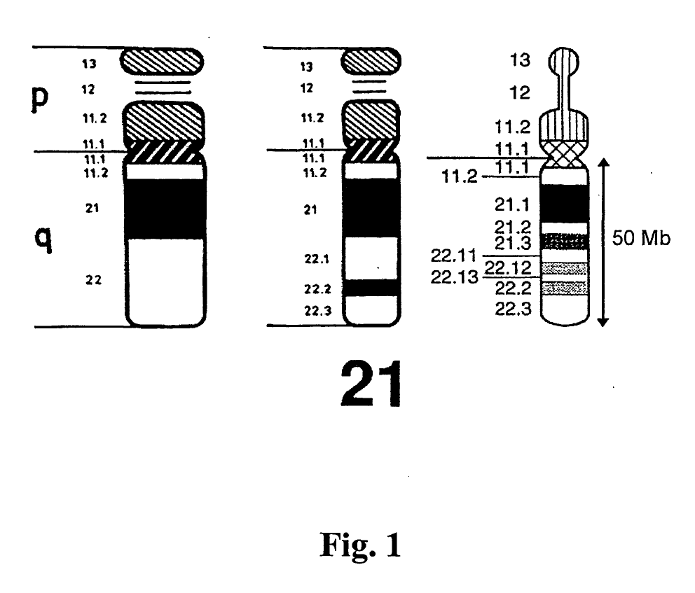

### BRIEF DESCRIPTION OF THE SEQUENCE LISTING

SEQ ID Nos. 1 to 171 contain nucleotide sequences comprising map-related biallelic markers.

SEQ ID Nos. 172 to 342 contain nucleotide sequences of upstream amplification primers (PU) designed to amplify sequences containing the biallelic markers of SEQ ID Nos. 1 to 171.

SEQ ID Nos. 343 to 513 contain nucleotide sequences of downstream amplification primers (RP) designed to amplify sequences containing the biallelic markers of SEQ ID Nos. 1 to 171.

SEQ ID Nos. 514 to 519 contain nucleotide sequences comprising a portion of the map-related biallelic markers which are shown to be associated with Alzheimer's Disease as described in Example 7.

SEQ ID Nos. 520 to 531 contain nucleotide sequences comprising a portion of the map-related biallelic markers which are shown to be associated with prostate cancer as described in Examples 10-22.

SEQ ID Nos. 532 to 535 contain nucleotide sequences comprising a portion of the map-related biallelic markers which are shown to be associated with elevated plasma TG in obese adolescents in Examples 23-26.

SEQ ID Nos. 536 to 557 contain nucleotide sequences of upstream amplification primers (PU) designed to amplify sequences containing the biallelic markers of SEQ ID Nos. 514 to 535.

SEQ ID Nos. 558 to 579 contain nucleotide sequences of downstream amplification primers (RP) designed to amplify sequences containing the biallelic markers of SEQ ID Nos. 514 to 535.

In accordance with the regulations relating to Sequence Listings, the following codes have been used in the Sequence Listing to indicate the locations of biallelic markers within the sequences and to identify each of the alleles present at the polymorphic base. The code “r” in the sequences indicates that one allele of the polymorphic base is a guanine, while the other allele is an adenine. The code “y” in the sequences indicates that one allele of the polymorphic base is a thymine, while the other allele is a cytosine. The code “m” in the sequences indicates that one allele of the polymorphic base is an adenine, while the other allele is an cytosine. The code “k” in the sequences indicates that one allele of the polymorphic base is a guanine, while the other allele is a thymine. The code “s” in the sequences indicates that one allele of the polymorphic base is a guanine, while the other allele is a cytosine. The code “w” in the sequences indicates that one allele of the polymorphic base is an adenine, while the other allele is an thymine.

### DETAILED DESCRIPTION OF THE EMBODIMENTS

Before describing the invention in greater detail, the following definitions are set forth to illustrate and define the meaning and scope of the terms used to describe the invention herein.

Definitions

As used interchangeably herein, the terms “nucleic acids” “oligonucleotides,” and “polynucleotides” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modifications (a) an alternative linking group, (b) an analogous form of purine, (c) an analogous form of pyrimidine, or (d) an analogous sugar, for examples of analogous linking groups, purine, pyrimidines, and sugars see for example PCT publication No. WO 95/04064. However, the polynucleotides of the invention are preferably comprised of greater than 50% conventional deoxyribose nucleotides, and most preferably greater than 90% conventional deoxyribose nucleotides. The polynucleotide sequences of the invention may be prepared by any known method, including synthetic, recombinant, ex vivo generation, or a combination thereof, as well as utilizing any purification methods known in the art.

As used herein, the term “purified” does not require absolute purity; rather, it is intended as a relative definition. Individual 5′ polynucleotide clones isolated from a cDNA library have been conventionally purified to electrophoretic homogeneity. The sequences obtained from these clones could not be obtained directly either from the library or from total human DNA. The cDNA clones are not naturally occurring as such, but rather are obtained via manipulation of a partially purified naturally occurring substance (messenger RNA). The conversion of mRNA into a cDNA library involves the creation of a synthetic substance (cDNA) and pure individual cDNA clones can be isolated from the synthetic library by clonal selection. Thus, creating a cDNA library from messenger RNA and subsequently isolating individual clones from that library results in an approximately 10⁴-10⁶ fold purification of the native message. Purification of starting material or natural material to at least one order of magnitude, preferably two or three orders, and more preferably four or five orders of magnitude is expressly contemplated. Alternatively, purification may be expressed as “at least” a percent purity relative to heterologous polynucleotides (DNA, RNA or both). As a preferred embodiment, the polynucleotides of the present invention are at least; 10%, 20%, 30%, 40%, 50%, 60%, 70%, 80%, 90%, 95%, 96%, 96%, 98%, 99%, or 100% pure relative to heterologous polynucleotides. As a further preferred embodiment the polynucleotides have an “at least” purity ranging from any number, to the thousandth position, between 90% and 100% (e.g., 5′ POLYNUCLEOTIDE at least 99.995% pure) relative to heterologous polynucleotides. Additionally, purity of the polynucleotides may be expressed as a percentage (as described above) relative to all materials and compounds other than the carrier solution. Each number, to the thousandth position, may be claimed as individual species of purity.

As used herein, the term “isolated” requires that the material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated. Specifically excluded from the definition of “isolated” are: naturally occurring chromosomes (e.g., chromosome spreads) artificial chromosome libraries, genomic libraries, and cDNA libraries that exist either as an in vitro nucleic acid preparation or as a transfected/transformed host cell preparation, wherein the host cells are either an in vitro heterogeneous preparation or plated as a heterogeneous population of single colonies. Also specifically excluded are the above libraries wherein the 5′ POLYNUCLEOTIDE makes up less than 5% of the number of nucleic acid inserts in the vector molecules. Further specifically excluded are whole cell genomic DNA or whole cell RNA preparations (including said whole cell preparations which are mechanically sheared or enzymaticly digested). Further specifically excluded are the above whole cell preparations as either an in vitro preparation or as a heterogeneous mixture separated by electrophoresis (including blot transfers of the same) wherein the polynucleotide of the invention have not been further separated from the heterologous polynucleotides in the electrophoresis medium (e.g., further separating by excising a single band from a heterogeneous band population in an agarose gel or nylon blot).

“Stringent,” “moderate,” and “low” hybridization conditions are as defined below.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified.

The terms “detectable trait” “trait” and “phenotype” are used interchangeably herein and refer to any visible, detectable or otherwise measurable property of an organism such as symptoms of, or susceptibility to a disease for example. Typically the terms “detectable trait” “trait” or “phenotype” are used herein to refer to symptoms of, or susceptibility to a disease; or to refer to an individual's response to an agent, drug, or treatment acting on a disease; or to refer to symptoms of, or susceptibility to side effects to an agent acting on a disease.

The term “treatment” is used herein to encompass any medical intervention known in the art including, for example, the administration of pharmaceutical agents, medically prescribed changes in diet, or habits such as a reduction in smoking or drinking, surgery, the application of medical devices, and the application or reduction of certain physical conditions, for example, light or radiation.

The term “allele” is used herein to refer to variants of a nucleotide sequence. A biallelic polymorphism has two forms; designated herein as the 1^{ST} allele and the 2^{ND} allele. Diploid organisms may be homozygous or heterozygous for an allelic form.

The term “heterozygosity rate” is used herein to refer to the incidence of individuals in a population, which are heterozygous at a particular allele. In a biallelic system the heterozygosity rate is on average equal to 2Pₐ(1-Pₐ), where Pₐ is the frequency of the least common allele. In order to be useful in genetic studies a genetic marker should have an adequate level of heterozygosity to allow a reasonable probability that a randomly selected person will be heterozygous.

The term “genotype” as used herein refers the identity of the alleles present in an individual or a sample. In the context of the present invention a genotype preferably refers to the description of the biallelic marker alleles present in an individual or a sample. The term “genotyping” a sample or an individual for a biallelic marker consists of determining the specific allele or the specific nucleotide carried by an individual at a biallelic marker.

The term “mutation” as used herein refers to a difference in DNA sequence between or among different genomes or individuals which has a frequency below 1%.

The term “haplotype” refers to a combination of alleles present in an individual or a sample. In the context of the present invention a haplotype preferably refers to a combination of biallelic marker alleles found in a given individual and which may be associated with a phenotype.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A single nucleotide polymorphism is a single base pair change. Typically a single nucleotide polymorphism is the replacement of one nucleotide by another nucleotide at the polymorphic site. Deletion of a single nucleotide or insertion of a single nucleotide, also give rise to single nucleotide polymorphisms. In the context of the present invention “single nucleotide polymorphism” preferably refers to a single nucleotide substitution. Typically, between different genomes or between different individuals, the polymorphic site may be occupied by two different nucleotides.

The terms “biallelic polymorphism” and “biallelic marker” are used interchangeably herein to refer to a polymorphism having two alleles at a fairly high frequency in the population, preferably a single nucleotide polymorphism. A “biallelic marker allele” refers to the nucleotide variants present at a biallelic marker site. Typically the frequency of the less common allele of the biallelic markers of the present invention has been validated to be greater than 1%, preferably the frequency is greater than 10%, more preferably the frequency is at least 20% (i.e. heterozygosity rate of at least 0.32), even more preferably the frequency is at least 30% (i.e. heterozygosity rate of at least 0.42). A biallelic marker wherein the frequency of the less common allele is 30% or more is termed a “high quality biallelic marker.”

The location of nucleotides in a polynucleotide with respect to the center of the polynucleotide are described herein in the following manner. When a polynucleotide has an odd number of nucleotides, the nucleotide at an equal distance from the 3′ and 5′ ends of the polynucleotide is considered to be “at the center” of the polynucleotide, and any nucleotide immediately adjacent to the nucleotide at the center, or the nucleotide at the center itself is considered to be “within 1 nucleotide of the center.” With an odd number of nucleotides in a polynucleotide any of the five nucleotides positions in the middle of the polynucleotide would be considered to be within 2 nucleotides of the center, and so on. When a polynucleotide has an even number of nucleotides, there would be a bond and not a nucleotide at the center of the polynucleotide. Thus, either of the two central nucleotides would be considered to be “within 1 nucleotide of the center” and any of the four nucleotides in the middle of the polynucleotide would be considered to be “within 2 nucleotides of the center”, and so on. For polymorphisms which involve the substitution, insertion or deletion of 1 or more nucleotides, the polymorphism, allele or biallelic marker is “at the center” of a polynucleotide if the difference between the distance from the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within 1 nucleotide of the center.” If the difference is 0 to 5, the polymorphism is considered to be “within 2 nucleotides of the center.” If the difference is 0 to 7, the polymorphism is considered to be “within 3 nucleotides of the center,” and so on. For polymorphisms which involve the substitution, insertion or deletion of 1 or more nucleotides, the polymorphism, allele or biallelic marker is “at the center” of a polynucleotide if the difference between the distance from the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within 1 nucleotide of the center.” If the difference is 0 to 5, the polymorphism is considered to be “within 2 nucleotides of the center.” If the difference is 0 to 7, the polymorphism is considered to be “within 3 nucleotides of the center,” and so on.

The term “upstream” is used herein to refer to a location which, is toward the 5′ end of the polynucleotide from a specific reference point.

The terms “base paired” and “Watson & Crick base paired” are used interchangeably herein to refer to nucleotides which can be hydrogen bonded to one another be virtue of their sequence identities in a manner like that found in double-helical DNA with thymine or uracil residues linked to adenine residues by two hydrogen bonds and cytosine and guanine residues linked by three hydrogen bonds (See Stryer, L., Biochemistry, 4th edition, 1995).

The terms “complementary” or “complement thereof” are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

As used herein the term “map-related biallelic marker” relates to a biallelic marker in linkage disequilibrium with any of the sequences disclosed in SEQ ID Nos. 1 to 171 which contain a biallelic marker of the map. The term map-related biallelic marker encompasses all of the biallelic markers disclosed in SEQ ID Nos. 1 to 171. The preferred map-related biallelic marker alleles of the present invention include each one of the alleles selected individually or in any combination from the biallelic markers of SEQ ID Nos. 1 to 171, as identified in field <223> of the allele feature in the appended Sequence Listing, individually or in groups consisting of all the possible combinations of the alleles.

The terms “1^{ST} allele” and “2^{ND} allele” refer to the nucleotide located at the polymorphic base of a polynucleotide sequence containing a biallelic marker, as identified in field <222> of the allele feature in the appended Sequence Listing for each Sequence ID number. As used herein, the polymorphic base is generally located at nucleotide position 23 for each of SEQ ID Nos. 1 to 171, as described in Table 1a.

I. Biallelic Markers And Polynucleotides Comprising Biallelic Markers

Polynucleotides of the Present Invention

The present invention encompasses polynucleotides for use as primers and probes in the methods of the invention. All of the polynucleotides of the invention may be specified as being isolated, purified or recombinant. These polynucleotides may consist of, consist essentially of, or comprise a contiguous span of nucleotides of a sequence from any sequence in the Sequence Listing as well as sequences which are complementary thereto (“complements thereof”). The “contiguous span” maybe at least 8, 10, 12, 15, 18, 19, 20, 22, 23, 24, 25, 30, 35, 43, 44, 45, 46 or 47 nucleotides in length, to the extent that a contiguous span of these lengths is consistent with the lengths of the particular Sequence ID. It should be noted that the polynucleotides of the present invention are not limited to having the exact flanking sequences surrounding the polymorphic bases which are enumerated in the Sequence Listing. Rather, it will be appreciated that the flanking sequences surrounding the biallelic markers, or any of the primers of probes of the invention which, are more distant from the markers, may be lengthened or shortened to any extent compatible with their intended use and the present invention specifically contemplates such sequences. It will be appreciated that the polynucleotides referred to in the Sequence Listing may be of any length compatible with their intended use. Also the flanking regions outside of the contiguous span need not be homologous to native flanking sequences which actually occur in human subjects. The addition of any nucleotide sequence, which is compatible with the nucleotides intended use is specifically contemplated. The contiguous span may optionally include the map-related biallelic marker in said sequence. Biallelic markers generally consist of a polymorphism at one single base position. Each biallelic marker therefore corresponds to two forms of a polynucleotide sequence which, when compared with one another, present a nucleotide modification at one position. Usually, the nucleotide modification involves the substitution of one nucleotide for another. Optionally either the 1^{ST} allele or the 2^{ND} allele of the biallelic markers of SEQ ID Nos. 1 to 171, 1 to 100, 101 to 162, 163 to 171 may be specified as being present at the map-related biallelic marker.

Preferred polynucleotides may consist of, consist essentially of, or comprise a contiguous span of nucleotides of a sequence from SEQ ID Nos. 1 to 100 as well as sequences which are complementary thereto. The “contiguous span” may be at least 8, 10, 12, 15, 18, 19, 20, 22, 23, 24, 25, 30, 35, 43, 44, 45, 46 or 47 nucleotides in length, to the extent that a contiguous span of these lengths is consistent with the lengths of the particular Sequence ID. Particularly preferred are polynucleotides which consist of, consist essentially of, or comprise a contiguous span of nucleotides of a sequence of any of SEQ ID Nos. 1 to 100, or the complements thereof, wherein the 1^{ST} allele of the biallelic marker of the SEQ ID No. is present at the map-related biallelic marker. Other preferred polynucleotides consist of, consist essentially of, or comprise a contiguous span of nucleotides of any of SEQ ID Nos. 1 to 100, or the complements thereof, wherein the 2^{ND} allele of the biallelic marker of the SEQ ID No. is present at the map-related biallelic marker. Preferred polynucleotides may consist of, consist essentially of, or comprise a contiguous span of at least 8; 10, 12, 15, 18, 19, 20, 22, 23, 24, 25, 30, 35, 43, 44, 45, 46 or 47 nucleotides in length, to the extent that a contiguous span of these lengths is consistent with the lengths of the particular Sequence ID No., of a sequence from SEQ ID Nos. 101 to 162 as well as sequences which are complementary thereto. Particularly preferred are polynucleotides which consist of, consist essentially of, or comprise a contiguous span of nucleotides of a sequence of any of SEQ ID Nos. 101 to 162, or the complements thereof, wherein the 1^{ST} allele of the biallelic marker of the SEQ ID No. is present at the map-related biallelic marker. Other preferred polynucleotides consist of, consist essentially of, or comprise a contiguous span of nucleotides of any of SEQ ID Nos. 101 to 162, or the complements thereof, wherein the 2ND allele of the biallelic marker of the SEQ ID No. is present at the map-related biallelic marker. Preferred polynucleotides may consist of, consist essentially of, or comprise a contiguous span of at least 8, 10, 12, 15, 18, 19, 20, 22, 23, 24, 25, 30, 35, 43, 44, 45, 46 or 47 nucleotides in length, to the extent that a contiguous span of these lengths is consistent with the lengths of the particular Sequence ID No., of a sequence from SEQ ID Nos. 163 to 171 as well as sequences which are complementary thereto. Particularly preferred are polynucleotides which consist of, consist essentially of, or comprise a contiguous span of nucleotides of a sequence of any of SEQ ID Nos. 163 to 171, or the complements thereof, wherein the 1^{ST} allele of the biallelic marker of the SEQ ID No. is present at the map-related biallelic marker. Other preferred polynucleotides consist of, consist essentially of, or comprise a contiguous span of nucleotides of any of SEQ ID Nos. 163 to 171, or the complements thereof, wherein the 2ND allele of the biallelic marker of the SEQ ID No. is present at the map-related biallelic marker.

The present invention also relates to biallelic markers or sets of biallelic markers located in chromosomal regions and subregions associated with obesity disorders. The invention therefore encompasses polynucleotides comprising the polymorphic base at a chromosome 3 map-related biallelic marker; a chromosome 10 map-related biallelic marker; and a chromosome 19 map-related biallelic marker. It will be appreciated that the invention also encompasses methods of genotyping and polynucleotides for use in amplification and genotyping at the map-related biallelic markers described herein, optionally with any further limitation described in this disclosure.

In further embodiments, a biallelic marker map comprises one or more, or all, of said map-related markers which are localized on chromosome 3, 10 or 19. Particularly preferred map-related biallelic markers are listed as follows, and polynucleotides of the invention may thus consist of, consist essentially of, or comprise a contiguous span of nucleotides of a sequence, or the sequences complementary thereto, from a SEQ ID selected from the group consisting of:

chromosome 3 biallelic markers: (a) SEQ ID Nos. 8, 10, 12, 13, 14, 15, 16, 17, 18, 19, 20, 23, 24, 25, 26, 27, 70, 72, 73, 74, 75, 76, 77; and (b) SEQ ID Nos. 102, 105, 106, 107, 110, 111, 117, 118, 119, 120, 121, 122, 123, 124, 125, 126, 127, 159, 160, 161; and (c) 163, 166, 167;

chromosome 10 biallelic markers: (a) SEQ ID Nos. 1, 2, 3, 4, 5, 6, 7, 9, 11, 21, 22, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 71, 78, 79, 80, 81, 82, 83, 84, 85, 86, 87, 88, 89, 90, 91, 92, 93, 94, 95, 96, 97, 98, 99, 100; (b) SEQ ID Nos. 101, 103, 104, 108, 109, 112, 113, 114, 115, 116, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158; and (c) SEQ ID Nos. 164, 165, 168, 169, 170, 171; and

chromosome 19 biallelic marker: (a) SEQ ID No. 162.

The “contiguous span” may be at least 8, 10, 12, 15, 18, 19, 20, 22, 23, 24, 25, 30, 35, 43, 44, 45, 46 or 47 nucleotides in length, to the extent that a contiguous span of these lengths is consistent with the lengths of the particular Sequence ID.

Optionally, any biallelic markers, sets of biallelic markers, polynucleotides or nucleic acid codes described throughout the present specification maybe selected from a group specifically excluding one or more of said chromosome 3, 10 and 19 map-related biallelic markers of SEQ ID numbers listed above, individually or in any combination.

The invention also relates to polynucleotides that hybridize, under conditions of high or intermediate stringency, to a polynucleotide of a sequence from any of SEQ ID Nos. 1 to 171, 1 to 100, 101 to 162, 163 to 171 as well as sequences which are complementary thereto. Preferably such polynucleotides are at least 8, 10, 12, 15, 18, 19, 20, 22, 23, 24, 25, 30, 35, 43, 44, 45, 46 or 47 nucleotides in length, to the extent that a polynucleotide of these lengths is consistent with the lengths of the particular Sequence ID. Preferred polynucleotides comprise a map-related biallelic marker. Optionally either the 1^{ST} or the 2^{ND} allele of the biallelic markers disclosed in the SEQ ID No. may be specified as being present at the map-related biallelic marker. Conditions of high and intermediate stringency are further described herein.

The primers of the present invention may be designed from the disclosed sequences using any method known in the art. A preferred set of primers is fashioned such that the 3′ end of the contiguous span of identity with the sequences of the Sequence Listing is present at the 3′ end of the primer. Such a configuration allows the 3′ end of the primer to hybridize to a selected nucleic acid sequence and dramatically increases the efficiency of the primer for amplification or sequencing reactions.

In a preferred set of primers the contiguous span is found in one of the sequences described in SEQ ID Nos. 1 to 171, 1 to 100, 101 to 162, 163 to 171, 172 to 513, 172 to 271, 272 to 333, 334 to 342, 343 to 442, 443 to 504 and 505 to 513 or the complements thereof. The invention also relates to polynucleotides consisting of, consisting essentially of, or comprising a contiguous span of nucleotides of a sequence from SEQ ID No. 172 to 513, 172 to 271, 272 to 333, 334 to 342, 343 to 442, 443 to 504 and 505 to 513, as well as sequences which are complementary thereto, wherein the “contiguous span” may be at least 8, 10, 12, 15, 18, 19, 20, or 21 nucleotides in length, to the extent that a contiguous span of these lengths is consistent with the lengths of the particular Sequence ID No.

Allele specific primers may be designed such that a biallelic marker is at the 3′ end of the contiguous span and the contiguous span is present at the 3′ end of the primer. Such allele specific primers tend to selectively prime an amplification or sequencing reaction so long as they are used with a nucleic acid sample that contains one of the two alleles present at a biallelic marker. The 3′ end of primer of the invention may be located within or at least 2, 4, 6, 8, 10, to the extent that this distance is consistent with the particular Sequence ID, nucleotides upstream of a map-related biallelic marker in said sequence or at any other location which is appropriate for their intended use in sequencing, amplification or the location of novel sequences or markers. Primers with their 3′ ends located 1 nucleotide upstream of a map-related biallelic marker have a special utility as microsequencing assays. Preferred microsequencing primers are described in SEQ ID Nos. 1 to 171, 1 to 100, 101 to 162, 163 to 171, where for each of SEQ ID Nos. 1 to 171, 1 to 100, 101 to 162, 163 to 171, the sense microsequencing primer contains the complement of the 19 nucleotides having their 3′ ends located 1 nucleotide upstream of the polymorphic base of the respective SEQ ID No, and where the antisense microsequencing primer contains the complement of the 19 nucleotides of the complementary strand, nucleotides of the primer having their 3′ end located 1 nucleotide upstream of the polymorphic base on the complementary strand to the respective SEQ ID No. The most preferred of said microsequencing primers for each of SEQ ID Nos. 1 to 171, 1 to 100, 101 to 162, 163 to 171 are microsequencing primers indicated as “A” or “S” in Table 1a, which have been validated in microsequencing experiments.

The probes of the present invention may be designed from the disclosed sequences for any method known in the art, particularly methods which allow for testing if a particular sequence or marker disclosed herein is present. A preferred set of probes may be designed for use in the hybridization assays of the invention in any manner known in the art such that they selectively bind to one allele of a biallelic marker, but not the other under any particular set of assay conditions. Preferred hybridization probes may consist of, consist essentially of, or comprise a contiguous span of SEQ ID Nos. 1 to 171, 1 to 100, 101 to 162, 163 to 171, or the complement thereof, which ranges in length from least 8, 10, 12, 15, 18, 19, 20, 22, 23, 24, 25, 30, 35, 43, 44, 45, 46 or 47 nucleotides, to the extent that a contiguous span of these lengths is consistent with the lengths of the particular Sequence ID No., or be specified as being 12, 15, 18, 19, 20, 25, 35, 40, 43, 44, 45, 46 or 47 nucleotides in length and including the map-related biallelic marker of said sequence. Optionally the 1^{st} allele or 2^{nd} allele of SEQ ID Nos. 1 to 171, 1 to 100, 101 to 162, 163 to 171 may be specified as being present at the biallelic marker site. Optionally, said biallelic marker may be within 6, 5, 4, 3, 2, or 1 nucleotides of the center of the hybridization probe or at the center of said probe.

Any of the polynucleotides of the present invention can be labeled, if desired, by incorporating a label detectable by spectroscopic, photochemical, biochemical, immunochemical, or chemical means. For example, useful labels include radioactive substances, fluorescent dyes or biotin. Preferably, polynucleotides are labeled at their 3′ and 5′ ends. A label can also be used to capture the primer, so as to facilitate the immobilization of either the primer or a primer extension product, such as amplified DNA, on a solid support. A capture label is attached to the primers or probes and can be a specific binding member which forms a binding pair with the solid's phase reagent's specific binding member (e.g. biotin and streptavidin). Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleic acid sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleic acid on a solid phase. DNA Labeling techniques are well known to the skilled technician.

Any of the polynucleotides, primers and probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes® and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes® and other configurations known to those of ordinary skill in the art. The polynucleotides of the invention can be attached to or immobilized on a solid support individually or in groups of at least 2, 5, 8, 10, 12, 15, 20, or 25 distinct polynucleotides of the inventions to a single solid support. In addition, polynucleotides other than those of the invention may attached to the same solid support as one or more polynucleotides of the invention.

Any polynucleotide provided herein may be attached in overlapping areas or at random locations on the solid support. Alternatively the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array technology known in the art can be employed with the polynucleotides of the invention. One particular embodiment of these polynucleotide arrays is known as the Genechips™, and has been generally described in U.S. Pat. No. 5,143,854; PCT publications WO 90/15070 and 92/10092. These arrays may generally be produced using mechanical synthesis methods or light directed synthesis methods, which incorporate a combination of photolithographic methods and solid phase oligonucleotide synthesis (Fodor et al., Science, 251:767-777, 1991, the disclosure of which is incorporated herein by reference in its entirety). The immobilization of arrays of oligonucleotides on solid supports has been rendered possible by the development of a technology generally identified as “Very Large Scale Immobilized Polymer Synthesis” (VLSIPS™) in which, typically, probes are immobilized in a high density array on a solid surface of a chip. Examples of VLSIPS™ technologies are provided in U.S. Pat. Nos. 5,143,854 and 5,412,087 and in PCT Publications WO 90/15070, WO 92/10092 and WO 95/11995, the disclosures of which are incorporated herein by reference in their entirety, which describe methods for forming oligonucleotide arrays through techniques such as light-directed synthesis techniques. In designing strategies aimed at providing arrays of nucleotides immobilized on solid supports, further presentation strategies were developed to order and display the oligonucleotide arrays on the chips in an attempt to maximize hybridization patterns and sequence information. Examples of such presentation strategies are disclosed in PCT Publications WO 94/12305, WO 94/11530, WO 97/29212 and WO 97/31256, the disclosures of which are incorporated herein by reference in their entireties.

Oligonucleotide arrays may comprise at least one of the sequences selected from the group consisting of SEQ ID Nos. 1 to 171, 1 to 100, 101 to 162, 163 to 171 and the sequences complementary thereto, or a fragment thereof of at least 8, 10, 12, 15, 18, 19, 20, 22, 23, 24, 25, 30, 35, 43, 44, 45, 46 or 47 consecutive nucleotides, to the extent that fragments of these lengths is consistent with the lengths of the particular Sequence ID, for determining whether a sample contains one or more alleles of the biallelic markers of the present invention. Oligonucleotide arrays may also comprise at least one of the sequences selected from the group consisting of SEQ ID Nos. 1 to 171, 1 to 100, 101 to 162, 163 to 171, and the sequences complementary thereto, or a fragment thereof of at least 8, 10, 12, 15, 18, 19, 20, 22, 23, 24, 25, 30, 35, 43, 44, 45, 46 or 47 consecutive nucleotides, to the extent that fragments of these lengths is consistent with the lengths of the particular Sequence ID, for amplifying one or. more alleles of the biallelic markers of SEQ ID Nos. 1 to 171, 1 to 100, 101 to 162, 163 to 171. In other embodiments, arrays may also comprise at least one of the sequences selected from the group consisting of SEQ ID Nos. 1 to 171, 1 to 100, 101 to 162, 163 to 171 and the sequences complementary thereto, or a fragment thereof of at 8, 10, 12; 15, 18, 19, 20, 22, 23, 24, 25, 30, 35, 43, 44, 45, 46 or 47 consecutive nucleotides, to the extent that fragments of these lengths is consistent with the lengths of the particular Sequence ID, for conducting microsequencing analyses to determine whether a sample contains one or more alleles of the biallelic markers of the invention. In still further embodiments, the oligonucleotide array may comprise at least one of the sequences selected from the group consisting of SEQ ID Nos. 1 to 171, 1 to 100, 101 to 162, 163 to 171 and the sequences complementary thereto, or a fragment thereof of at least 8, 10, 12, 15, 18, 19, 20, 22, 23, 24, 25, 30, 35, 43, 44, 45, 46 or 47 nucleotides in length, to the extent that fragments of these lengths is consistent with the lengths of the particular Sequence ID, for determining whether a sample contains one or more alleles of the biallelic markers of the present invention.

In designing strategies aimed at providing arrays of nucleotides immobilized on solid supports, further presentation strategies were developed to order and display the probe arrays on the chips in an attempt to maximize hybridization patterns and sequence information. Examples of such presentation strategies are disclosed in PCT Publications WO 94/12305, WO 94/11530, WO 97/29212 and WO 97/31256, the disclosures of which are incorporated herein by reference in their entireties.

Each DNA chip can contain thousands to millions of individual synthetic DNA probes arranged in a grid-like pattern and miniaturized to the size of a dime. In some embodiments, the efficiency of hybridization of nucleic acids in the sample with the probes attached to the chip may be improved by using polyacrylamide gel pads isolated from one another by hydrophobic regions in which the DNA probes are covalently linked to an acrylamide matrix.

The polymorphic bases present in the biallelic marker or markers of the sample nucleic acids are determined as follows. Probes which contain at least a portion of one or more of the biallelic markers of the present invention are synthesized either in situ or by conventional synthesis and immobilized on an appropriate chip using methods known to the skilled technician.

Any one or more alleles of the biallelic markers described herein (SEQ ID Nos. 1 to 171, 1 to 100, 101 to 162, 163 to 171 or the sequences complementary thereto) or fragments thereof containing the polymorphic bases, may be fixed to a solid support, such as a microchip or other immobilizing surface. The fragments of these nucleic acids may comprise at least 10, at least 15, at least 20, at least 25, or more than 25 consecutive nucleotides of the biallelic markers described herein. Preferably, the fragments include the polymorphic bases of the biallelic markers.

A nucleic acid sample is applied to the immobilizing surface and analyzed to determine the identities of the polymorphic bases of one or more of the biallelic markers. In some embodiments, the solid support may also include one or more of the amplification primers described herein, or fragments comprising at least 10, at least 15, or at least 20 consecutive nucleotides thereof, for generating an amplification product containing the polymorphic bases of the biallelic markers to be analyzed in the sample.

Another embodiment of the present invention is a solid support which includes one or more of the microsequencing primers of the invention, or fragments comprising at least 10, at least 15, or at least 20 consecutive nucleotides thereof and having a 3′ terminus immediately upstream of the polymorphic base of the corresponding biallelic marker, for determining the identity of the polymorphic base of the one or more biallelic markers fixed to the solid support.

For example, one embodiment of the present invention is an array of nucleic acids fixed to a solid support, such as a microchip, bead, or other immobilizing surface, comprising one or more of the biallelic markers in the maps of the present invention or a fragment comprising at least 10, at least 15, at least 20, at least 25, or more than 25 consecutive nucleotides thereof including the polymorphic base. For example, the array may comprise 1, 2, 3, 4, 5, 10, 15, 20, 25, 30, 40, 50, 70, 85, 100 of the biallelic markers selected from the group consisting of SEQ ID Nos.: 1 to 171, 1 to 100, 101 to 162, 163 to 171 or the sequences complementary thereto, or a fragment comprising at least 10, at least 15, at least 20, at least 25, or more than 25 consecutive nucleotides thereof including the polymorphic base.

Another embodiment of the present invention is an array comprising amplification primers for generating amplification products containing the polymorphic bases of one or more, at least five, at least 10, at least 20, at least 100, at least 200, at least 300, at least 400, or more than 400 of the biallelic markers in the maps of the present invention. For example, the array may comprise amplification primers for generating amplification products containing the polymorphic bases of at least 1, 2, 3, 4, 5, 10, 15, 20, 25, 30, 40, 50, 70, 85, 100 or all of the biallelic markers selected from the group consisting of SEQ ID Nos.: 1 to 171, 1 to 100, 101 to 162, 163 to 171 or the sequences complementary thereto. In such arrays, the amplification primers included in the array are capable of amplifying the biallelic marker sequences to be detected in the nucleic acid sample applied to the array (i.e. the amplification primers correspond to the biallelic markers affixed to the array—see Table 1a). Thus, the arrays may include one or more of the amplification primers of SEQ ID Nos.: 172 to 513, 172 to 271, 272 to 333, 334 to 342, 343 to 442, 443 to 504 and 505 to 513 corresponding to the one or more biallelic markers of SEQ ID Nos. 1 to 171, 1 to 100, 101 to 162, 163 to 171 which are included in the array.

Another embodiment of the present invention is an array which includes microsequencing primers capable of determining the identity of the polymorphic bases of at least 1, 2, 3, 4, 5, 10, 15, 20, 25, 30, 40, 50, 70, 85, 100 or all of the biallelic markers selected from the group consisting of SEQ ID Nos.: 1 to 171, 1 to 100, 101 to 162, 163 to 171 or the sequences complementary thereto. For example, the array may comprise microsequencing primers capable of determining the identity of the polymorphic bases of one or more, at least five, at least 10, at least 20, at least 100, at least 200, at least 300, at least 400, or more than 400 of the biallelic markers of SEQ ID Nos. 1 to 171, 1 to 100, 101 to 162, 163 to 171 or the sequences complementary thereto.

Arrays containing any combination of the above nucleic acids which permits the specific detection or identification of the polymorphic bases of the biallelic markers in the maps of the present invention, including any combination of biallelic markers of SEQ ID Nos. 1 to 171, 1 to 100, 101 to 162, 163 to 171 or the sequences complementary thereto are also within the scope of the present invention. For example, the array may comprise both the biallelic markers and amplification primers capable of generating amplification products containing the polymorphic bases of the biallelic markers. Alternatively, the array may comprise both amplification primers capable of generating amplification products containing the polymorphic bases of the biallelic markers and microsequencing primers capable of determining the identities of the polymorphic bases of these markers.

Although the above examples describe arrays comprising specific groups of biallelic markers and, in some embodiments, specific amplification primers and microsequencing primers, it will be appreciated that the present invention encompasses arrays including any biallelic marker, group of biallelic markers, amplification primer, group of amplification primers, microsequencing primer, or group of amplification primers described herein, as well as any combination of the preceding nucleic acids.

The present invention also encompasses diagnostic kits comprising one or more polynucleotides of the invention, optionally with a portion or all of the necessary reagents and instructions for genotyping a test subject by determining the identity of a nucleotide at a map-related biallelic marker. The polynucleotides of a kit may optionally be attached to a solid support, or be part of an array or addressable array of polynucleotides. The kit may provide for the determination of the identity of the nucleotide at a marker position by any method known in the art including, but not limited to, a sequencing assay method, a microsequencing assay method, a hybridization assay method, or an allele specific amplification method. Optionally such a kit may include instructions for scoring the results of the determination with respect to the test subjects' risk of contracting a diseases involving a disease, likely response to an agent acting on a disease, or chances of suffering from side effects to an agent acting on a disease.

II. Methods For De Novo Identification Of Biallelic Markers

Any of a variety of methods can be used to screen a genomic fragment for single nucleotide polymorphisms such as differential hybridization with oligonucleotide probes, detection of changes in the mobility measured by gel electrophoresis or direct sequencing of the amplified nucleic acid. A preferred method for identifying biallelic markers involves comparative sequencing of genomic DNA fragments from an appropriate number of unrelated individuals.

In a first embodiment, DNA samples from unrelated individuals are pooled together, following which the genomic DNA of interest is amplified and sequenced. The nucleotide sequences thus obtained are then analyzed to identify significant polymorphisms. One of the major advantages of this method resides in the fact that the pooling of the DNA samples substantially reduces the number of DNA amplification reactions and sequencing reactions, which must be carried out. Moreover, this method is sufficiently sensitive so that a biallelic marker obtained thereby usually demonstrates a sufficient frequency of its less common allele to be useful in conducting association studies. Usually, the frequency of the least common allele of a biallelic marker identified by this method is at least 10%.

In a second embodiment, the DNA samples are not pooled and are therefore amplified and sequenced individually. This method is usually preferred when biallelic markers need to be identified in order to perform association studies within candidate genes. Preferably, highly relevant gene regions such as promoter regions or exon regions may be screened for biallelic markers. A biallelic marker obtained using this method may show a lower degree of informativeness for conducting association studies, e.g. if the frequency of its less frequent allele may be less than about 10%. Such a biallelic marker will however be sufficiently informative to conduct association studies and it will further be appreciated that including less informative biallelic markers in the genetic analysis studies of the present invention, may allow in some cases the direct identification of causal mutations, which may, depending on their penetrance, be rare mutations.

The following is a description of the various parameters of a preferred method used by the inventors for the identification of the biallelic markers of the present invention.

II.A. Genomic DNA Samples

The genomic DNA samples from which the biallelic markers of the present invention are generated are preferably obtained from unrelated individuals corresponding to a heterogeneous population of known ethnic background. The number of individuals from whom DNA samples are obtained can vary substantially, preferably from about 10 to about 1000, more preferably from about 50 to about 200 individuals. Usually, DNA samples are collected from at least about 100 individuals in order to have sufficient polymorphic diversity in a given population to identify as many markers as possible and to generate statistically significant results.

As for the source of the genomic DNA to be subjected to analysis, any test sample can be foreseen without any particular limitation. These test samples include biological samples, which can be tested by the methods of the present invention described herein, and include human and animal body fluids such as whole blood, serum, plasma, cerebrospinal fluid, urine, lymph fluids, and various external secretions of the respiratory, intestinal and genitourinary tracts, tears, saliva, milk, white blood cells, myelomas and the like; biological fluids such as cell culture supernatants; fixed tissue specimens including tumor and non-tumor tissue and lymph node tissues; bone marrow aspirates and fixed cell specimens. The preferred source of genomic DNA used in the present invention is from peripheral venous blood of each donor. Techniques to prepare genomic DNA from biological samples are well known to the skilled technician. Details of a preferred embodiment are provided in Example 12. The person skilled in the art can choose to amplify pooled or unpooled DNA samples.

II.B. DNA Amplification

The identification of biallelic markers in a sample of genomic DNA may be facilitated through the use of DNA amplification methods. DNA samples can be pooled or unpooled for the amplification step. DNA amplification techniques are well known to those skilled in the art. Various methods to amplify DNA fragments carrying biallelic markers are further described hereinafter in III.B. The PCR technology is the preferred amplification technique used to identify new biallelic markers.

In a first embodiment, biallelic markers are identified using genomic sequence information generated by the inventors. Genomic DNA fragments, such as the inserts of the BAC clones described above, are sequenced and used to design primers for the amplification of 500 bp fragments. These 500 bp fragments are amplified from genomic DNA and are scanned for biallelic markers. Primers may be designed using the OSP software (Hillier L. and Green P., 1991). All primers may contain, upstream of the specific target bases, a common oligonucleotide tail that serves as a sequencing primer. Those skilled in the art are familiar with primer extensions, which can be used for these purposes.

In another embodiment of the invention, genomic sequences of candidate genes are available in public databases allowing direct screening for biallelic markers. Preferred primers, useful for the amplification of genomic sequences encoding the candidate genes, focus on promoters, exons and splice sites of the genes. A biallelic marker present in these functional regions of the gene have a higher probability to be a causal mutation.

Preferred primers include those disclosed in SEQ ID No. 172 to 513, 172 to 271, 272 to 333, 334 to 342, 343 to 442, 443 to 504 and 505 to 513.

II.C. Sequencing of Amplified Genomic DNA and Identification of Single Nucleotide Polymorphisms

The amplification products generated as described above, are then sequenced using any method known and available to the skilled technician. Methods for sequencing DNA using either the dideoxy-mediated method (Sanger method) or the Maxam-Gilbert method are widely known to those of ordinary skill in the art. Such methods are for example disclosed in Maniatis et al. (Molecular Cloning, A Laboratory Manual, Cold Spring Harbor Press, Second Edition, 1989 the disclosure of which is incorporated herein by reference in its entirety). Alternative approaches include hybridization to high-density DNA probe arrays as described in Chee et al. (Science 274, 610, 1996, the disclosure of which is incorporated herein by reference in its entirety).

Preferably, the amplified DNA is subjected to automated dideoxy terminator sequencing reactions using a dye-primer cycle sequencing protocol. The products of the sequencing reactions are run on sequencing gels and the sequences are determined using gel image analysis. The polymorphism search is based on the presence of superimposed peaks in the electrophoresis pattern resulting from different bases occurring at the same position. Because each dideoxy terminator is labeled with a different fluorescent molecule, the two peaks corresponding to a biallelic site present distinct colors corresponding to two different nucleotides at the same position on the sequence. However, the presence of two peaks can be an artifact due to background noise. To exclude such an artifact, the two DNA strands are sequenced and a comparison between the peaks is carried out. In order to be registered as a polymorphic sequence, the polymorphism has to be detected on both strands.

The above procedure permits those amplification products, which contain biallelic markers to be identified. The detection limit for the frequency of biallelic polymorphisms detected by sequencing pools of 100 individuals is approximately 0.1 for the minor allele, as verified by sequencing pools of known allelic frequencies. However, more than 90% of the biallelic polymorphisms detected by the pooling method have a frequency for the minor allele higher than 0.25. Therefore, the biallelic markers selected by this method have a frequency of at least 0.1 for the minor allele and less than 0.9 for the major allele. Preferably at least 0.2 for the minor allele and less than 0.8 for the major allele, more preferably at least 0.3 for the minor allele and less than 0.7 for the major allele, thus a heterozygosity rate higher than 0.18, preferably higher than 0.32, more preferably higher than 0.42.

In another embodiment, biallelic markers are detected by sequencing individual DNA samples, the frequency of the minor allele of such a biallelic marker may be less than 0.1.

The markers carried by the same fragment of genomic DNA, such as the insert in a BAC clone, need not necessarily be ordered with respect to one another within the genomic fragment to conduct association studies. However, in some embodiments of the present invention, the order of biallelic markers carried by the same fragment of genomic DNA are determined.

II.D. Validation of the Biallelic Markers of the Present Invention

The polymorphisms are evaluated for their usefulness as genetic markers by validating that both alleles are present in a population. Validation of the biallelic markers is accomplished by genotyping a group of individuals by a method of the invention and demonstrating that both alleles are present. Microsequencing is a preferred method of genotyping alleles. The validation by genotyping step may be performed on individual samples derived from each individual in the group or by genotyping a pooled sample derived from more than one individual. The group can be as small as one individual if that individual is heterozygous for the allele in question. Preferably the group contains at least three individuals, more preferably the group contains five or six individuals, so that a single validation test will be more likely to result in the validation of more of the biallelic markers that are being tested. It should be noted, however, that when the validation test is performed on a small group it may result in a false negative result if as a result of sampling error none of the individuals tested carries one of the two alleles. Thus, the validation process is less useful in demonstrating that a particular initial result is an artifact, than it is at demonstrating that there is a bona fide biallelic marker at a particular position in a sequence. All of the genotyping, haplotyping, association, and interaction study methods of the invention may optionally be performed solely with validated biallelic markers.

II.E. Evaluation of the Frequency of the Biallelic Markers of the Present Invention

The validated biallelic markers are further evaluated for their usefulness as genetic markers by determining the frequency of the least common allele at the biallelic marker site. The determination of the least common allele is accomplished by genotyping a group of individuals by a method of the invention and demonstrating that both alleles are present. This determination of frequency by genotyping step may be performed on individual samples derived from each individual in the group or by genotyping a pooled sample derived from more than one individual. The group must be large enough to be representative of the population as a whole. Preferably the group contains at least 20 individuals, more preferably the group contains at least 50 individuals, most preferably the group contains at least 100 individuals. Of course the larger the group the greater the accuracy of the frequency determination because of reduced sampling error. A biallelic marker wherein the frequency of the less common allele is 30% or more is termed a “high quality biallelic marker.” All of the genotyping, haplotyping, association, and interaction study methods of the invention may optionally be performed solely with high quality biallelic markers.

III. Methods Of Genotyping an Individual for Biallelic Markers

Methods are provided to genotype a biological sample for one or more biallelic markers of the present invention, all of which may be performed in vitro. Such methods of genotyping comprise determining the identity of a nucleotide at a map-related biallelic marker by any method known in the art. These methods find use in genotyping case-control populations in association studies as well as individuals in the context of detection of alleles of biallelic markers which, are known to be associated with a given trait, in which case both copies of the biallelic marker present in individual's genome are determined so that an individual may be classified as homozygous or heterozygous for a particular allele.

These genotyping methods can be performed nucleic acid samples derived from a single individual or pooled DNA samples.

Genotyping can be performed using similar methods as those described above for the identification of the biallelic markers, or using other genotyping methods such as those further described below. In preferred embodiments, the comparison of sequences of amplified genomic fragments from different individuals is used to identify new biallelic markers whereas microsequencing is used for genotyping known biallelic markers in diagnostic and association study applications.

III.A. Source of DNA for Genotyping

Any source of nucleic acids, in purified or non-purified form, can be utilized as the starting nucleic acid, provided it contains or is suspected of containing the specific nucleic acid sequence desired. DNA or RNA may be extracted from cells, tissues, body fluids and the like as described above in II.A. While nucleic acids for use in the genotyping methods of the invention can be derived from any mammalian source, the test subjects and individuals from which nucleic acid samples are taken are generally understood to be human.

III.B. Amplification of DNA Fragments Comprising Biallelic Markers

Methods and polynucleotides are provided to amplify a segment of nucleotides comprising one or more biallelic marker of the present invention. It will be appreciated that amplification of DNA fragments comprising biallelic markers may be used in various methods and for various purposes and is not restricted to genotyping. Nevertheless, many genotyping methods, although not all, require the previous amplification of the DNA region carrying the biallelic marker of interest. Such methods specifically increase the concentration or total number of sequences that span the biallelic marker or include that site and sequences located either distal or proximal to it. Diagnostic assays may also rely on amplification of DNA segments carrying a biallelic marker of the present invention.

Amplification of DNA may be achieved by any method known in the art. The established PCR (polymerase chain reaction) method or by developments thereof or alternatives. Amplification methods which can be utilized herein include but are not limited to Ligase Chain Reaction (LCR) as described in EP A 320 308 and EP A 439 182, Gap LCR (Wolcott, M. J., Clin. Microbiol. Rev. 5:370-386), the so-called “NASBA” or “3SR” technique described in Guatelli J. C. et al. (Proc. Natl. Acad. Sci. USA 87:1874-1878, 1990) and in Compton J. (Nature 350:91-92, 1991), Q-beta amplification as described in European Patent Application no 4544610, strand displacement amplification as described in Walker et al. (Clin. Chem. 42:9-13, 1996) and EP A 684 315 and, target mediated amplification as described in PCT Publication WO 9322461, the disclosures of which are incorporated herein by reference in their entireties.

LCR and Gap LCR are exponential amplification techniques, both depend on DNA ligase to join adjacent primers annealed to a DNA molecule. In Ligase Chain Reaction (LCR), probe pairs are used which include two primary (first and second) and two secondary (third and fourth) probes, all of which are employed in molar excess to target. The first probe hybridizes to a first segment of the target strand and the second probe hybridizes to a second segment of the target strand, the first and second segments being contiguous so that the primary probes abut one another in 5′ phosphate-3′ hydroxyl relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. Of course, if the target is initially double stranded, the secondary probes also will hybridize to the target complement in the first instance. Once the ligated strand of primary probes is separated from the target strand, it will hybridize with the third and fourth probes which can be ligated to form a complementary, secondary ligated product. It is important to realize that the ligated products are functionally equivalent to either the target or its complement. By repeated cycles of hybridization and ligation, amplification of the target sequence is achieved. A method for multiplex LCR has also been described (WO 9320227, the disclosure of which is incorporated herein by reference in its entirety). Gap LCR (GLCR) is a version of LCR where the probes are not adjacent but are separated by 2 to 3 bases.

For amplification of mRNAs, it is within the scope of the present invention to reverse transcribe mRNA into cDNA followed by polymerase chain reaction (RT-PCR); or, to use a single enzyme for both steps as described in U.S. Pat. No. 5,322,770, the disclosure of which is incorporated herein by reference in its entirety, or, to use Asymmetric Gap LCR (RT-AGLCR) as described by Marshall R. L. et al. (PCR Methods and Applications 4:80-84, 1994, the disclosure of which is incorporated herein by reference in its entirety). AGLCR is a modification of GLCR that allows the amplification of RNA.

Some of these amplification methods are particularly suited for the detection of single nucleotide polymorphisms and allow the simultaneous amplification of a target sequence and the identification of the polymorphic nucleotide as it is further described in III.C.

The PCR technology is the preferred amplification technique used in the present invention. A variety of PCR techniques are familiar to those skilled in the art. For a review of PCR technology, see Molecular Cloning to Genetic Engineering White, B. A. Ed. in Methods in Molecular Biology 67: Humana Press, Totowa (1997) and the publication entitled “PCR Methods and Applications” (1991, Cold Spring Harbor Laboratory Press, the disclosure of which is incorporated herein by reference in its entirety). In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188, the disclosure of which is incorporated herein by reference in its entirety.

The identification of biallelic markers as described above allows the design of appropriate oligonucleotides, which can be used as primers to amplify DNA fragments comprising the biallelic markers of the present invention. Amplification can be performed using the primers initially used to discover new biallelic markers which are described herein or any set of primers allowing the amplification of a DNA fragment comprising a biallelic marker of the present invention. Primers can be prepared by any suitable method. As for example, direct chemical synthesis by a method such as the phosphodiester method of Narang S. A. et al. (Methods Enzymol. 68:90-98, 1979), the phosphodiester method of Brown E. L. et al. (Methods Enzymol. 68:109-151, 1979), the diethylphosphorarnidite method of Beaucage et al. (Tetrahedron Lett. 22:1859-1862, 1981) and the solid support method described in EP 0 707 592, the disclosures of which are incorporated herein by reference in their entireties.

In some embodiments the present invention provides primers for amplifying a DNA fragment containing one or more biallelic markers of the present invention. Preferred amplification primers are listed in SEQ ID No. 172 to 513, 172 to 271, 272 to 333, 334 to 342, 343 to 442, 443 to 504 and 505 to 513. It will be appreciated that the primers listed are merely exemplary and that any other set of primers which produce amplification products containing one or more biallelic markers of the present invention.

The primers are selected to be substantially complementary to the different strands of each specific sequence to be amplified. The length of the primers of the present invention can range from 8 to 100 nucleotides, preferably from 8 to 50, 8 to 30 or more preferably 8 to 25 nucleotides. Shorter primers tend to lack specificity for a target nucleic acid sequence and generally require cooler temperatures to form sufficiently stable hybrid complexes with the template. Longer primers are expensive to produce and can sometimes self-hybridize to form hairpin structures. The formation of stable hybrids depends on the melting temperature (Tm) of the DNA. The Tm depends on the length of the primer, the ionic strength of the solution and the G+C content. The higher the G+C content of the primer, the higher is the melting temperature because G:C pairs are held by three H bonds whereas A:T pairs have only two. The G+C content of the amplification primers of the present invention preferably ranges between 10 and 75%, more preferably between 35 and 60%, and most preferably between 40 and 55%. The appropriate length for primers under a particular set of assay conditions may be empirically determined by one of skill in the art.

The spacing of the primers determines the length of the segment to be amplified. In the context of the present invention amplified segments carrying biallelic markers can range in size from at least about 25 bp to 35 kbp. Amplification fragments from 25-3000 bp are typical, fragments from 50-1000 bp are preferred and fragments from 100-600 bp are highly preferred. It will be appreciated that amplification primers for the biallelic markers may be any sequence which allow the specific amplification of any DNA fragment carrying the markers. Amplification primers may be labeled or immobilized on a solid support as described in I.

III.C. Methods of Genotyping DNA Samples for Biallelic Markers

Any method known in the art can be used to identify the nucleotide present at a biallelic marker site. Since the biallelic marker allele to be detected has been identified and specified in the present invention, detection will prove simple for one of ordinary skill in the art by employing any of a number of techniques. Many genotyping methods require the previous amplification of the DNA region carrying the biallelic marker of interest. While the amplification of target or signal is often preferred at present, ultrasensitive detection methods which do not require amplification are also encompassed by the present genotyping methods. Methods well-known to those skilled in the art that can be used to detect biallelic polymorphisms include methods such as, conventional dot blot analyzes, single strand conformational polymorphism analysis (SSCP) described by Orita et al. (Proc. Natl. Acad. Sci. U.S.A. 86:27776-2770, 1989, the disclosure of which is incorporated herein by reference in its entirety), denaturing gradient gel electrophoresis (DGGE), heteroduplex analysis, mismatch cleavage detection, and other conventional techniques as described in Sheffield, V. C. et al. (Proc. Natl. Acad. Sci. USA 49:699-706, 1991), White et al. (Genomics 12:301-306, 1992), Grompe, M. et al. (Proc. Natl. Acad. Sci USA 86:5855-5892, 1989) and Grompe, M. (Nature Genetics 5:111-117, 1993, the disclosures of which are incorporated herein by reference in their entireties). Another method for determining the identity of the nucleotide present at a particular polymorphic site employs a specialized exonuclease-resistant nucleotide derivative as described in U.S. Pat. No. 4,656,127, the disclosure of which is incorporated herein by reference in its entirety.

Preferred methods involve directly determining the identity of the nucleotide present at a biallelic marker site by sequencing assay, enzyme-based mismatch detection assay, or hybridization assay. The following is a description of some preferred methods. A highly preferred method is the microsequencing technique. The term “sequencing assay” is used herein to refer to polymerase extension of duplex primer/template complexes and includes both traditional sequencing and microsequencing.

1) Sequencing Assays

The nucleotide present at a polymorphic site can be determined by sequencing methods. In a preferred embodiment, DNA samples are subjected to PCR amplification before sequencing as described above. DNA sequencing methods are described in IIC.

Preferably, the amplified DNA is subjected to automated dideoxy terminator sequencing reactions using a dye-primer cycle sequencing protocol. Sequence analysis allows the identification of the base present at the biallelic marker site.

2) Microsequencing Assays

In microsequencing methods, a nucleotide at the polymorphic site that is unique to one of the alleles in a target DNA is detected by a single nucleotide primer extension reaction. This method involves appropriate microsequencing primers which, hybridize just upstream of a polymorphic base of interest in the target nucleic acid. A polymerase is used to specifically extend the 3′ end of the primer with one single ddNTP (chain terminator) complementary to the selected nucleotide at the polymorphic site. Next the identity of the incorporated nucleotide is determined in any suitable way.

Typically, microsequencing reactions are carried out using fluorescent ddNTPs and the extended microsequencing primers are analyzed by electrophoresis on ABI 377 sequencing machines to determine the identity of the incorporated nucleotide as described in EP 412 883, the disclosure of which is incorporated herein by reference in its entirety. Alternatively capillary electrophoresis can be used in order to process a higher number of assays simultaneously. An example of a typical microsequencing procedure that can be used in the context of the present invention is provided in Example 8.

Different approaches can be used to detect the nucleotide added to the microsequencing primer. A homogeneous phase detection method based on fluorescence resonance energy transfer has been described by Chen and Kwok (Nucleic Acids Research 25:347-353 1997) and Chen et al. (Proc. Natl. Acad. Sci. USA 94/20 10756-10761,1997, the disclosures of which are incorporated herein by reference in their entireties). In this method amplified genomic DNA fragments containing polymorphic sites are incubated with a 5′-fluorescein-labeled primer in the presence of allelic dye-labeled dideoxyribonucleoside triphosphates and a modified Taq polymerase. The dye-labeled primer is extended one base by the dye-terminator specific for the allele present on the template. At the end of the genotyping reaction, the fluorescence intensities of the two dyes in the reaction mixture are analyzed directly without separation or purification. All these steps can be performed in the same tube and the fluorescence changes can be monitored in real time. Alternatively, the extended primer may be analyzed by MALDI-TOF Mass Spectrometry. The base at the polymorphic site is identified by the mass added onto the microsequencing primer (see Haff L. A. and Smirnov I. P., Genome Research, 7:378-388, 1997, the disclosure of which is incorporated herein by reference in its entirety).

Microsequencing may be achieved by the established microsequencing method or by developments or derivatives thereof. Alternative methods include several solid-phase microsequencing techniques. The basic microsequencing protocol is the same as described previously, except that the method is conducted as a heterogeneous phase assay, in which the primer or the target molecule is immobilized or captured onto a solid support. To simplify the primer separation and the terminal nucleotide addition analysis, oligonucleotides are attached to solid supports or are modified in such ways that permit affinity separation as well as polymerase extension. The 5′ ends and internal nucleotides of synthetic oligonucleotides can be modified in a number of different ways to permit different affinity separation approaches, e.g., biotinylation. If a single affinity group is used on the oligonucleotides, the oligonucleotides can be separated from the incorporated terminator regent. This eliminates the need of physical or size separation. More than one oligonucleotide can be separated from the terminator reagent and analyzed simultaneously if more than one affinity group is used. This permits the analysis of several nucleic acid species or more nucleic acid sequence information per extension reaction. The affinity group need not be on the priming oligonucleotide but could alternatively be present on the template. For example, immobilization can be carried out via an interaction between biotinylated DNA and streptavidin-coated microtitration wells or avidin-coated polystyrene particles. In the same manner oligonucleotides or templates may be attached to a solid support in a high-density format. In such solid phase microsequencing reactions, incorporated ddNTPs can be radiolabeled (Syvänen, Clinica Climica Acta 226:225-236, 1994, the disclosure of which is incorporated herein by reference in its entirety), or linked to fluorescein (Livak and Hainer, Human Mutation 3:379-385,1994, the disclosure of which is incorporated herein by reference in its entirety). The detection of radiolabeled ddNTPs can be achieved through scintillation-based techniques. The detection of fluorescein-linked ddNTPs can be based on the binding of antifluorescein antibody conjugated with alkaline phosphatase, followed by incubation with a chromogenic substrate (such as p-nitrophenyl phosphate). Other possible reporter-detection pairs include: ddNTP linked to dinitrophenyl (DNP) and anti-DNP alkaline phosphatase conjugate (Haiju et al., Clin. Chem. 39/11 2282-2287, 1993, the disclosure of which is incorporated herein by reference in its entirety), or biotinylated ddNTP and horseradish peroxidase-conjugated streptavidin with o-phenylenediamine as a substrate (WO 92/15712, the disclosure of which is incorporated herein by reference in its entirety). As yet another alternative solid-phase microsequencing procedure, Nyren et al. (Analytical Biochemistry 208:171-175, 1993, the disclosure of which is incorporated herein by reference in its entirety), described a method relying on the detection of DNA polymerase activity by an enzymatic luminometric inorganic pyrophosphate detection assay (ELIDA).

Pastinen et al. (Genome research 7:606-614, 1997, the disclosure of which is incorporated herein by reference in its entirety), describe a method for multiplex detection of single nucleotide polymorphism in which the solid phase minisequencing principle is applied to an oligonucleotide array format. High-density arrays of DNA probes attached to a solid support (DNA chips) are further described in III.C.5.

In one aspect the present invention provides polynucleotides and methods to genotype one or more biallelic markers of the present invention by performing a microsequencing assay. In the preferred embodiment, it will be appreciated that any primer having a 3 ′ end immediately adjacent to a polymorphic nucleotide may be used as a microsequencing primer. Similarly, it will be appreciated that microsequencing analysis may be performed for any biallelic marker or any combination of biallelic markers of the present invention. One aspect of the present invention is a solid support which includes one or more microsequencing primers comprising nucleotides complementary to the nucleotide sequences of SEQ ID Nos. 1 to 171, 1 to 100, 101 to 162, 163 to 171 or the complements thereof, or fragments comprising at least 8, at least 12, at least 15, or at least 20 consecutive nucleotides thereof and having a 3′ terminus immediately upstream of the corresponding biallelic marker, for determining the identity of a nucleotide at bialielic marker site.

3) Mismatch Detection Assays Based on Polymerases and Ligases

In one aspect the present invention provides polynucleotides and methods to determine the allele of one or more biallelic markers of the present invention in a biological sample, by mismatch detection assays based on polymerases and/or ligases. These assays are based on the specificity of polymerases and ligases. Polymerization reactions places particularly stringent requirements on correct base pairing of the 3′ end of the amplification primer and the joining of two oligonucleotides hybridized to a target DNA sequence is quite sensitive to mismatches close to the ligation site, especially at the 3′ end. The terms “enzyme based mismatch detection assay” are used herein to refer to any method of determining the allele of a biallelic marker based on the specificity of ligases and polymerases. Preferred methods are described below. Methods, primers and various parameters to amplify DNA fragments comprising biallelic markers of the present invention are further described above in III.B.

Allele Specific Amplification

Discrimination between the two alleles of a biallelic marker can also be achieved by allele specific amplification, a selective strategy, whereby one of the alleles is amplified without amplification of the other allele. This is accomplished by placing a polymorphic base at the 3′ end of one of the amplification primers. Because the extension forms from the 3 ′ end of the primer, a mismatch at or near this position has an inhibitory effect on amplification. Therefore, under appropriate amplification conditions, these primers only direct amplification on their complementary allele. Designing the appropriate allele-specific primer and the corresponding assay conditions are well with the ordinary skill in the art.

Ligation/Amplification Based Methods

The “Oligonucleotide Ligation Assay” (OLA) uses two oligonucleotides which are designed to be capable of hybridizing to abutting sequences of a single strand of a target molecules. One of the oligonucleotides is biotinylated, and the other is detectably labeled. If the precise complementary sequence is found in a target molecule, the oligonucleotides will hybridize such that their termini abut, and create a ligation substrate that can be captured and detected. OLA is capable of detecting biallelic markers and may be advantageously combined with PCR as described by Nickerson D. A. et al. (Proc. Natl. Acad. Sci. U.S.A. 87:8923-8927, 1990, the disclosure of which is incorporated herein by reference in its entirety). In this method, PCR is used to achieve the exponential amplification of target DNA, which is then detected using OLA.

Other methods which are particularly suited for the detection of biallelic markers include LCR (ligase chain reaction), Gap LCR (GLCR) which are described above in III.B. As mentioned above LCR uses two pairs of probes to exponentially amplify a specific target. The sequences of each pair of oligonucleotides, is selected to permit the pair to hybridize to abutting sequences of the same strand of the target. Such hybridization forms a substrate for a template-dependant ligase. In accordance with the present invention, LCR can be performed with oligonucleotides having the proximal and distal sequences of the same strand of a biallelic marker site. In one embodiment, either oligonucleotide will be designed to include the biallelic marker site. In such an embodiment, the reaction conditions are selected such that the oligonucleotides can be ligated together only if the target molecule either contains or lacks the specific nucleotide(s) that is complementary to the biallelic marker on the oligonucleotide. In an alternative embodiment, the oligonucleotides will not include the biallelic marker, such that when they hybridize to the target molecule, a “gap” is created as described in WO 90/01069, the disclosure of which is incorporated herein by reference in its entirety. This gap is then “filled” with complementary dNTPs (as mediated by DNA polymerase), or by an additional pair of oligonucleotides. Thus at the end of each cycle, each single strand has a complement capable of serving as a target during the next cycle and exponential allele-specific amplification of the desired sequence is obtained.

Ligase/Polymerase-mediated Genetic Bit Analysis™ is another method for determining the identity of a nucleotide at a preselected site in a nucleic acid molecule (WO 95/21271, the disclosure of which is incorporated herein by reference in its entirety). This method involves the incorporation of a nucleoside triphosphate that is complementary to the nucleotide present at the preselected site onto the terminus of a primer molecule, and their subsequent ligation to a second oligonucleotide. The reaction is monitored by detecting a specific label attached to the reaction's solid phase or by detection in solution.

4) Hybridization Assay Methods

A preferred method of determining the identity of the nucleotide present at a biallelic marker site involves nucleic acid hybridization. The hybridization probes, which can be conveniently used in such reactions, preferably include the probes defined herein. Any hybridization assay may be used including Southern hybridization, Northern hybridization, dot blot hybridization and solid-phase hybridization (see Sambrook et al., Molecular Cloning—A Laboratory Manual, Second Edition, Cold Spring Harbor Press, N.Y., 1989, the disclosure of which is incorporated herein by reference in its entirety).

Hybridization refers to the formation of a duplex structure by two single stranded nucleic acids due to complementary base pairing. Hybridization can occur between exactly complementary nucleic acid strands or between nucleic acid strands that contain minor regions of mismatch. Specific probes can be designed that hybridize to one form of a biallelic marker and not to the other and therefore are able to discriminate between different allelic forms. Allele-specific probes are often used in pairs, one member of a pair showing perfect match to a target sequence containing the original allele and the other showing a perfect match to the target sequence containing the alternative allele. Hybridization conditions should be sufficiently stringent that there is a significant difference in hybridization intensity between alleles, and preferably an essentially binary response, whereby a probe hybridizes to only one of the alleles. Stringent, sequence specific hybridization conditions, under which a probe will hybridize only to the exactly complementary target sequence are well known in the art (Sambrook et al., Molecular Cloning—A Laboratory Manual, Second Edition, Cold Spring Harbor Press, N.Y., 1989, the disclosure of which is incorporated herein by reference in its entirety). Stringent conditions are sequence dependent and will be different in different circumstances. Generally, stringent conditions are selected to be about 5° C. lower than the thermal melting point (Tm) for the specific sequence at a defined ionic strength and pH. By way of example and not limitation, procedures using conditions of high stringency are as follows: Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C., the preferred hybridization temperature, in prehybridization mixture containing 100 μg/ml denatured salmon sperm DNA and 5-20×10⁶ cpm of ³²P-labeled probe. Alternatively, the hybridization step can be performed at 65° C. in the presence of SSC buffer, 1×SSC corresponding to 0.15M NaCl and 0.05 M Na citrate. Subsequently, filter washes can be done at 37° C. for 1 h in a solution containing 2×SSC, 0.01% PVP, 0.01% Ficoll, and 0.01% BSA, followed by a wash in 0.1×SSC at 50° C. for 45 min. Alternatively, filter washes can be performed in a solution containing 2×SSC and 0.1% SDS, or 0.5×SSC and 0.1% SDS, or 0.1×SSC and 0.1% SDS at 68° C. for 15 minute intervals. Following the wash steps, the hybridized probes are detectable by autoradiography. By way of example and not limitation, procedures using conditions of intermediate stringency are as follows: Filters containing DNA are prehybridized, and then hybridized at a temperature of 60° C. in the presence of a 5×SSC buffer and labeled probe. Subsequently, filters washes are performed in a solution containing 2×SSC at 50° C. and the hybridized probes are detectable by autoradiography. Other conditions of high and intermediate stringency which may be used are well known in the art and as cited in Sambrook et al. (Molecular Cloning—A Laboratory Manual, Second Edition, Cold Spring Harbor Press, N.Y., 1989) and Ausubel et al. (Current Protocols in Molecular Biology, Green Publishing Associates and Wiley Interscience, N.Y., 1989, the disclosure of which is incorporated herein by reference in its entirety).

Although such hybridizations can be performed in solution, it is preferred to employ a solid-phase hybridization assay. The target DNA comprising a biallelic marker of the present invention may be amplified prior to the hybridization reaction. The presence of a specific allele in the sample is determined by detecting the presence or the absence of stable hybrid duplexes formed between the probe and the target DNA. The detection of hybrid duplexes can be carried out by a number of methods. Various detection assay formats are well known which utilize detectable labels bound to either the target or the probe to enable detection of the hybrid duplexes. Typically, hybridization duplexes are separated from unhybridized nucleic acids and the labels bound to the duplexes are then detected. Those skilled in the art will recognize that wash steps may be employed to wash away excess target DNA or probe. Standard heterogeneous assay formats are suitable for detecting the hybrids using the labels present on the primers and probes.

Two recently developed assays allow hybridization-based allele discrimination with no need for separations or washes (see Landegren U. et al., Genome Research, 8:769-776,1998, the disclosure of which is incorporated herein by reference in its entirety). The TaqMan assay takes advantage of the 5′ nuclease activity of Taq DNA polymerase to digest a DNA probe annealed specifically to the accumulating amplification product. TaqMan probes are labeled with a donor-acceptor dye pair that interacts via fluorescence energy transfer. Cleavage of the TaqMan probe by the advancing polymerase during amplification dissociates the donor dye from the quenching acceptor dye, greatly increasing the donor fluorescence. All reagents necessary to detect two allelic variants can be assembled at the beginning of the reaction and the results are monitored in real time (see Livak et al., Nature Genetics, 9:341-342, 1995, the disclosure of which is incorporated herein by reference in its entirety). In an alternative homogeneous hybridization-based procedure, molecular beacons are used for allele discriminations. Molecular beacons are hairpin-shaped oligonucleotide probes that report the presence of specific nucleic acids in homogeneous solutions. When they bind to their targets they undergo a conformational reorganization that restores the fluorescence of an internally quenched fluorophore (Tyagi et al., Nature Biotechnology, 16:49-53, 1998, the disclosure of which is incorporated herein by reference in its entirety).

The polynucleotides provided herein can be used in hybridization assays for the detection of biallelic marker alleles in biological samples. These probes are characterized in that they preferably comprise between 8 and 50 nucleotides, and in that they are sufficiently complementary to a sequence comprising a biallelic marker of the present invention to hybridize thereto and preferably sufficiently specific to be able to discriminate the targeted sequence for only one nucleotide variation. The GC content in the probes of the invention usually ranges between 10 and 75%, preferably between 35 and 60%, and more preferably between 40 and 55%. The length of these probes can range from 10, 15, 20, or 30 to at least 100 nucleotides, preferably from 10 to 50, more preferably from 18 to 35 nucleotides. A particularly preferred probe is 25 nucleotides in length. Preferably the biallelic marker is within 4 nucleotides of the center of the polynucleotide probe. In particularly preferred probes the biallelic marker is at the center of said polynucleotide. Shorter probes may lack specificity for a target nucleic acid sequence and generally require cooler temperatures to form sufficiently stable hybrid complexes with the template. Longer probes are expensive to produce and can sometimes self-hybridize to form hairpin structures. Methods for the synthesis of oligonucleotide probes have been described above and can be applied to the probes of the present invention.

Preferably the probes of the present invention are labeled or immobilized on a solid support. Labels and solid supports are further described in I. Detection probes are generally nucleic acid sequences or uncharged nucleic acid analogs such as, for example peptide nucleic acids which are disclosed in International Patent Application WO 92/20702, morpholino analogs which are described in U.S. Pat. Nos. 5,185,444; 5,034,506 and 5,142,047. The probe may have to be rendered “non-extendable” in that additional dNTPs cannot be added to the probe. In and of themselves analogs usually are non-extendable and nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group simply can be cleaved, replaced or modified, U.S. patent application Ser. No. 07/049,061 filed Apr. 19, 1993 describes modifications, which can be used to render a probe non-extendable.

The probes of the present invention are useful for a number of purposes. They can be used in Southern hybridization to genomic DNA or Northern hybridization to mRNA. The probes can also be used to detect PCR amplification products. By assaying the hybridization to an allele specific probe, one can detect the presence or absence of a biallelic marker allele in a given sample.

High-Throughput parallel hybridizations in array format are specifically encompassed within “hybridization assays” and are described below.

Hybridization to Addressable Arrays of Oligonucleotides

Hybridization assays based on oligonucleotide arrays rely on the differences in hybridization stability of short oligonucleotides to perfectly matched and mismatched target sequence variants. Efficient access to polymorphism information is obtained through a basic structure comprising high-density arrays of oligonucleotide probes attached to a solid support (the chip) at selected positions. Each DNA chip can contain thousands to millions of individual synthetic DNA probes arranged in a grid-like pattern and miniaturized to the size of a dime.

The chip technology has already been applied with success in numerous cases. For example, the screening of mutations has been undertaken in the BRCA1 gene, in S. cerevisiae mutant strains, and in the protease gene of HIV-1 virus (Hacia et al., Nature Genetics, 14(4):441-447, 1996; Shoemaker et al., Nature Genetics, 14(4):450456, 1996; Kozal et al., Nature Medicine, 2:753-759, 1996, the disclosures of which are incorporated herein by reference in their entireties). Chips of various formats for use in detecting biallelic polymorphisms can be produced on a customized basis by Affymetrix (GeneChip™), Hyseq (HyChip and HyGnostics), and Protogene Laboratories.

In general, these methods employ arrays of oligonucleotide probes that are complementary to target nucleic acid sequence segments from an individual which, target sequences include a polymorphic marker. EP785280, the disclosure of which is incorporated herein by reference in its entirety, describes a tiling strategy for the detection of single nucleotide polymorphisms. Briefly, arrays may generally be “tiled” for a large number of specific polymorphisms. By “tiling” is generally meant the synthesis of a defined set of oligonucleotide probes which is made up of a sequence complementary to the target sequence of interest, as well as preselected variations of that sequence, e.g., substitution of one or more given positions with one or more members of the basis set of monomers, i.e. nucleotides. Tiling strategies are further described in PCT application No. WO 95/11995, the disclosure of which is incorporated herein by reference in its entirety. In a particular aspect, arrays are tiled for a number of specific, identified biallelic marker sequences. In particular the array is tiled to include a number of detection blocks, each detection block being specific for a specific biallelic marker or a set of biallelic markers. For example, a detection block may be tiled to include a number of probes, which span the sequence segment that includes a specific polymorphism. To ensure probes that are complementary to each allele, the probes are synthesized in pairs differing at the biallelic marker. In addition to the probes differing at the polymorphic base, monosubstituted probes are also generally tiled within the detection block. These monosubstituted probes have bases at and up to a certain number of bases in either direction from the polymorphism, substituted with the remaining nucleotides (selected from A, T, G, C and U). Typically the probes in a tiled detection block will include substitutions of the sequence positions up to and including those that are 5 bases away from the biallelic marker. The monosubstituted probes provide internal controls for the tiled array, to distinguish actual hybridization from artefactual cross-hybridization. Upon completion of hybridization with the target sequence and washing of the array, the array is scanned to determine the position on the array to which the target sequence hybridizes. The hybridization data from the scanned array is then analyzed to identify which allele or alleles of the biallelic marker are present in the sample. Hybridization and scanning may be carried out as described in PCT application No. WO 92/10092 and WO 95/11995 and U.S. Pat. No. 5,424,186, the disclosures of which are incorporated herein by reference in their entireties.

Thus, in some embodiments, the chips may comprise an array of nucleic acid sequences of fragments of about 15 nucleotides in length. In further embodiments, the chip may comprise an array including at least one of the sequences selected from the group consisting of SEQ ID No. 1 to 171, 1 to 100, 101 to 162, 163 to 171 and the sequences complementary thereto, or a fragment thereof at least about 8 consecutive nucleotides, preferably 10, 15, 20, more preferably least 30, 35, 43, 44, 45, 46 or 47 consecutive nucleotides, to the extent that a contiguous span of these lengths is consistent with the lengths of the particular Sequence ID. In some embodiments, the chip may comprise an array of at least 2, 3, 4, 5, 6, 7, 8 or more of these polynucleotides of the invention. Solid supports and polynucleotides of the present invention attached to solid supports are further described in I.

5) Integrated Systems

Another technique, which may be used to analyze polymorphisms, includes multicomponent integrated systems, which miniaturize and compartmentalize processes such as PCR and capillary electrophoresis reactions in a single functional device. An example of such technique is disclosed in U.S. Pat. No. 5,589,136, which describes the integration of PCR amplification and capillary electrophoresis in chips.

Integrated systems can be envisaged mainly when microfluidic systems are used. These systems comprise a pattern of microchannels designed onto a glass, silicon, quartz, or plastic wafer included on a microchip. The movements of the samples are controlled by electric, electroosmotic or hydrostatic forces applied across different areas of the microchip. For genotyping biallelic markers, the microfluidic system may integrate nucleic acid amplification, microsequencing, capillary electrophoresis and a detection method such as laser-induced fluorescence detection.

IV. Methods of Genetic Analysis Using the Biallelic Markers of the Present Invention

Different methods are available for the genetic analysis of complex traits (see Lander and Schork, Science, 265, 2037-2048, 1994). The search for disease-susceptibility genes is conducted using two main methods: the linkage approach in which evidence is sought for cosegregation between a locus and a putative trait locus using family studies, and the association approach in which evidence is sought for a statistically significant association between an allele and a trait or a trait causing allele (Khoury J. et al., Fundamentals of Genetic Epidemiology, Oxford University Press, NY, 1993, the disclosure of which is incorporated herein by reference in its entirety). In general, the biallelic markers of the present invention find use in any method known in the art to demonstrate a statistically significant correlation between a genotype and a phenotype. The biallelic markers may be used in parametric and non-parametric linkage analysis methods. Preferably, the biallelic markers of the present invention are used to identify genes associated with detectable traits using association studies, an approach which does not require the use of affected families and which permits the identification of genes associated with complex and sporadic traits.

The genetic analysis using the biallelic markers of the present invention may be conducted on any scale. The whole set of biallelic markers of the present invention or any subset of biallelic markers of the present invention may be used. In some embodiments a subset of biallelic markers corresponding to one or several candidate genes may be used. In other embodiments a subset of biallelic markers corresponding to candidate genes from a particular disease pathway may be used. Alternatively, a subset of biallelic markers of the present invention localised on a specific chromosome segment may be used. Further, any set of genetic markers including a biallelic marker of the present invention may be used. A set of biallelic polymorphisms that, could be used as genetic markers in combination with the biallelic markers of the present invention, has been described in WO 98/20165, the disclosure of which is incorporated herein by reference in its entirety. As mentioned above, it should be noted that the biallelic markers of the present invention may be included in any complete or partial genetic map of the human genome. These different uses are specifically contemplated in the present invention and claims.

IV.A. Linkage Analysis

Linkage analysis is based upon establishing a correlation between the transmission of genetic markers and that of a specific trait throughout generations within a family. Thus, the aim of linkage analysis is to detect marker loci that show cosegregation with a trait of interest in pedigrees.

Parametric Methods

When data are available from successive generations there is the opportunity to study the degree of linkage between pairs of loci. Estimates of the recombination fraction enable loci to be ordered and placed onto a genetic map. With loci that are genetic markers, a genetic map can be established, and then the strength of linkage between markers and traits can be calculated and used to indicate the relative positions of markers and genes affecting those traits (Weir, B.S., Genetic data Analysis II: Methods for Discrete population genetic Data, Sinauer Assoc., Inc., Sunderland, Mass., USA, 1996, the disclosure of which is incorporated herein by reference in its entirety). The classical method for linkage analysis is the logarithm of odds (lod) score method (see Morton N. E., Am. J. Hum. Genet., 7:277-318, 1955; Ott J., Analysis of Human Genetic Linkage, John Hopkins University Press, Baltimore, 1991, the disclosures of which are incorporated herein by reference in their entireties). Calculation of lod scores requires specification of the mode of inheritance for the disease (parametric method). Generally, the length of the candidate region identified using linkage analysis is between 2 and 20 Mb. Once a candidate region is identified as described above, analysis of recombinant individuals using additional markers allows further delineation of the candidate region. Linkage analysis studies have generally relied on the use of a maximum of 5,000 microsatellite markers, thus limiting the maximum theoretical attainable resolution of linkage analysis to about 600 kb on average.

Linkage analysis has been successfully applied to map simple genetic traits that show clear Mendelian inheritance patterns and which have a high penetrance (i.e., the ratio between the number of trait positive carriers of allele a and the total number of a carriers in the population). However, parametric linkage analysis suffers from a variety of drawbacks. First, it is limited by its reliance on the choice of a genetic model suitable for each studied trait. Furthermore, as already mentioned, the resolution attainable using linkage analysis is limited, and complementary studies are required to refine the analysis of the typical 2 Mb to 20 Mb regions initially identified through linkage analysis. In addition, parametric linkage analysis approaches have proven difficult when applied to complex genetic traits, such as those due to the combined action of multiple genes and/or environmental factors. It is very difficult to model these factors adequately in a lod score analysis. In such cases, too large an effort and cost are needed to recruit the adequate number of affected families required for applying linkage analysis to these situations, as recently discussed by Risch, N. and Merikangas, K. (Sciece, 273:1516-1517, 1996, the disclosure of which is incorporated herein by reference in its entirety).

Non-Parametric Methods

The advantage of the so-called non-parametric methods for linkage analysis is that they do not require specification of the mode of inheritance for the disease, they tend to be more useful for the analysis of complex traits. In non-parametric methods, one tries to prove that the inheritance pattern of a chromosomal region is not consistent with random Mendelian segregation by showing that affected relatives inherit identical copies of the region more often than expected by chance. Affected relatives should show excess “allele sharing” even in the presence of incomplete penetrance and polygenic inheritance. In non-parametric linkage analysis the degree of agreement at a marker locus in two individuals can be measured either by the number of alleles identical by state (IBS) or by the number of alleles identical by descent (IBD). Affected sib pair analysis is a well-known special case and is the simplest form of these methods.

The biallelic markers of the present invention may be used in both parametric and non-parametric linkage analysis. Preferably biallelic markers may be used in non-parametric methods which allow the mapping of genes involved in complex traits. The biallelic markers of the present invention may be used in both IBD- and IBS-methods to map genes affecting a complex trait. In such studies, taking advantage of the high density of biallelic markers, several adjacent biallelic marker loci may be pooled to achieve the efficiency attained by multi-allelic markers (Zhao et al., Am. J. Hum. Genet., 63:225-240, 1998, the disclosure of which is incorporated herein by reference in its entirety).

However, both parametric and non-parametric linkage analysis methods analyse affected relatives, they tend to be of limited value in the genetic analysis of drug responses or in the analysis of side effects to treatments. This type of analysis is impractical in such cases due to the lack of availability of familial cases. In fact, the likelihood of having more than one individual in a family being exposed to the same drug at the same time is extremely low.

IV.B. Population Association Studies

The present invention comprises methods for identifying one or several genes among a set of candidate genes that are associated with a detectable trait using the biallelic markers of the present invention. In one embodiment the present invention comprises methods to detect an association between a biallelic marker allele or a biallelic marker haplotype and a trait. Further, the invention comprises methods to identify a trait causing allele in linkage disequilibrium with any biallelic marker allele of the present invention.

As described above, alternative approaches can be employed to perform association studies: genome-wide association studies, candidate region association studies and candidate gene association studies. In a preferred embodiment, the biallelic markers of the present invention are used to perform candidate gene association studies. Further, the biallelic markers of the present invention may be incorporated in any map of genetic markers of the human genome in order to perform genome-wide association studies. Methods to generate a high-density map of biallelic markers has been described in U.S. patent application Ser. No. 09/8422,978. The biallelic markers of the present invention may further be incorporated in any map of a specific candidate region of the genome (a specific chromosome or a specific chromosomal segment for example).

As mentioned above, association studies may be conducted within the general population and are not limited to studies performed on related individuals in affected families. Association studies are extremely valuable as they permit the analysis of sporadic or multifactor traits. Moreover, association studies represent a powerful method for fine-scale mapping enabling much finer mapping of trait causing alleles than linkage studies. Studies based on pedigrees often only narrow the location of the trait causing allele. Association studies using the biallelic markers of the present invention can therefore be used to refine the location of a trait causing allele in a candidate region identified by Linkage Analysis methods. Moreover, once a chromosome segment of interest has been identified, the presence of a candidate gene such as a candidate gene of the present invention, in the region of interest can provide a shortcut to the identification of the trait causing allele. Biallelic markers of the present invention can be used to demonstrate that a candidate gene is associated with a trait. Such uses are specifically contemplated in the present invention and claims.

1) Determining the Frequency of a Biallelic Marker Allele or of a Biallelic Marker Haplotype in a Population

Association studies explore the relationships among frequencies for sets of alleles between loci.

Determining the Frequency of an Allele in a Population

Allelic frequencies of the biallelic markers in a population can be determined using one of the methods described above under the heading “Methods for genotyping an individual for biallelic markers”, or any genotyping procedure suitable for this intended purpose. Genotyping pooled samples or individual samples can determine the frequency of a biallelic marker allele in a population. One way to reduce the number of genotypings required is to use pooled samples. A major obstacle in using pooled samples is in terms of accuracy and reproducibility for determining accurate DNA concentrations in setting up the pools. Genotyping individual samples provides higher sensitivity, reproducibility and accuracy and; is the preferred method used in the present invention. Preferably, each individual is genotyped separately and simple gene counting is applied to determine the frequency of an allele of a biallelic marker or of a genotype in a given population.

Determining the Frequency of a Haplotype in a Population

The gametic phase of haplotypes is unknown when diploid individuals are heterozygous at more than one locus. Using genealogical information in families gametic phase can sometimes be inferred (Perlin et al., Am. J. Hum. Genet., 55:777-787, 1994, the disclosure of which is incorporated herein by reference in its entirety). When no genealogical information is available different strategies may be used. One possibility is that the multiple-site heterozygous diploids can be eliminated from the analysis, keeping only the homozygotes and the single-site heterozygote individuals, but this approach might lead to a possible bias in the sample composition and the underestimation of low-frequency haplotypes. Another possibility is that single chromosomes can be studied independently, for example, by asymmetric PCR amplification (see Newton et al., Nucleic Acids Res., 17:2503-2516, 1989; Wu et al., Proc. Natl. Acad. Sci. USA, 86:2757, 1989, the disclosures of which are incorporated herein by reference in their entireties) or by isolation of single chromosome by limit dilution followed by PCR amplification (see Ruano et al., Proc. Natl. Acad. Sci. USA, 87:6296-6300, 1990, the disclosure of which is incorporated herein by reference in its entirety). Further, a sample may be haplotyped for sufficiently close biallelic markers by double PCR amplification of specific alleles (Sarkar, G. and Sommer S. S., Biotechniques, 1991, the disclosure of which is incorporated herein by reference in its entirety). These approaches are not entirely satisfying either because of their technical complexity, the additional cost they entail, their lack of generalisation at a large scale, or the possible biases they introduce. To overcome these difficulties, an algorithm to infer the phase of PCR-amplified DNA genotypes introduced by Clark A. G. (Mol. Biol. Evol., 7:111-122, 1990, the disclosure of which is incorporated herein by reference in its entirety) may be used. Briefly, the principle is to start filling a preliminary list of haplotypes present in the sample by examining unambiguous individuals, that is, the complete homozygotes and the single-site heterozygotes. Then other individuals in the same sample are screened for the possible occurrence of previously recognised haplotypes. For each positive identification, the complementary baplotype is added to the list of recognised haplotypes, until the phase information for all individuals is either resolved or identified as unresolved. This method assigns a single haplotype to each multiheterozygous individual, whereas several haplotypes are possible when there are more than one heterozygous site. Alternatively, one can use methods estimating haplotype frequencies in a population without assigning haplotypes to each individual. Preferably, a method based on an expectation-maximization (EM) algorithm (Dempster et al., J. R. Stat. Soc., 39B: 1-38, 1977, the disclosure of which is incorporated herein by reference in its entirety) leading to maximum-likelihood estimates of haplotype frequencies under the assumption of Hardy-Weinberg proportions (random mating) is used (see Excoffier L. and Slatkin M., Mol. Biol. Evol., 12(5): 921-927, 1995, the disclosure of which is incorporated herein by reference in its entirety). The EM algorithm is a generalised iterative maximum-likelihood approach to estimation that is useful when data are ambiguous and/or incomplete. The EM algorithm is used to resolve heterozygotes into haplotypes. Haplotype estimations are further described below under the heading “Statistical methods”. Any other method known in the art to determine or to estimate the frequency of a haplotype in a population may also be used.

2) Linkage Disequilibrium Analysis

Linkage disequilibrium is the non-random association of alleles at two or more loci and represents a powerful tool for mapping genes involved in disease traits (see Ajioka R. S. et al., Am. J. Hum. Genet., 60:1439-1447, 1997, the disclosure of which is incorporated herein by reference in its entirety). Biallelic markers, because they are densely spaced in the human genome and can be genotyped in more numerous numbers than other types of genetic markers (such as RFLP or VNTR markers), are particularly useful in genetic analysis based on linkage disequilibrium. The biallelic markers of the present invention may be used in any linkage disequilibrium analysis method known in the art.

Briefly, when a disease mutation is first introduced into a population (by a new mutation or the immigration of a mutation carrier), it necessarily resides on a single chromosome and thus on a single “background” or “ancestral” haplotype of linked markers. Consequently, there is complete disequilibrium between these markers and the disease mutation: one finds the disease mutation only in the presence of a specific set of marker alleles. Through subsequent generations recombinations occur between the disease mutation and these marker polymorphisms, and the disequilibrium gradually dissipates. The pace of this dissipation is a function of the recombination frequency, so the markers closest to the disease gene will manifest higher levels of disequilibrium than those that are further away. When not broken up by recombination, “ancestral” haplotypes and linkage disequilibrium between marker alleles at different loci can be tracked not only through pedigrees but also through populations. Linkage disequilibrium is usually seen as an association between one specific allele at one locus and another specific allele at a second locus.

The pattern or curve of disequilibrium between disease and marker loci is expected to exhibit a maximum that occurs at the disease locus. Consequently, the amount of linkage disequilibrium between a disease allele and closely linked genetic markers may yield valuable information regarding the location of the disease gene. For fine-scale mapping of a disease locus, it is useful to have some knowledge of the patterns of linkage disequilibrium that exist between markers in the studied region. As mentioned above the mapping resolution achieved through the analysis of linkage disequilibrium is much higher than that of linkage studies. The high density of biallelic markers combined with linkage disequilibrium analysis provides powerful tools for fine-scale mapping. Different methods to calculate linkage disequilibrium are described below under the heading “Statistical Methods”.

3) Population-Based Case-Control Studies of Trait-Marker Associations

As mentioned above, the occurrence of pairs of specific alleles at different loci on the same chromosome is not random and the deviation from random is called linkage disequilibrium. Association studies focus on population frequencies and rely on the phenomenon of linkage disequilibrium. If a specific allele in a given gene is directly involved in causing a particular trait, its frequency will be statistically increased in an affected (trait positive) population, when compared to the frequency in a trait negative population or in a random control population. As a consequence of the existence of linkage disequilibrium, the frequency of all other alleles present in the haplotype carrying the trait-causing allele will also be increased in trait positive individuals compared to trait negative individuals or random controls. Therefore, association between the trait and any allele (specifically a biallelic marker allele) in linkage disequilibrium with the trait-causing allele will suffice to suggest the presence of a trait-related gene in that particular region. Case-control populations can be genotyped for biallelic markers to identify associations that narrowly locate a trait causing allele. As any marker in linkage disequilibrium with one given marker associated with a trait will be associated with the trait. Linkage disequilibrium allows the relative frequencies in case-control populations of a limited number of genetic polymorphisms (specifically biallelic markers) to be analysed as an alternative to screening all possible functional polymorphisms in order to find trait-causing alleles. Association studies compare the frequency of marker alleles in unrelated casecontrol populations, and represent powerful tools for the dissection of complex traits.

Case-Control Populations (Inclusion Criteria)

Population-based association studies do not concern familial inheritance but compare the prevalence of a particular genetic marker, or a set of markers, in case-control populations. They are case-control studies based on comparison of unrelated case (affected or trait positive) individuals and unrelated control (unaffected or trait negative or random) individuals. Preferably the control group is composed of unaffected or trait negative individuals. Further, the control group is ethnically matched to the case population. Moreover, the control group is preferably matched to the case-population for the main known confusion factor for the trait under study (for example age-matched for an age-dependent trait). Ideally, individuals in the two samples are paired in such a way that they are expected to differ only in their disease status. In the following “trait positive population”, “case population” and “affected population” are used interchangeably.

An important step in the dissection of complex traits using association studies is the choice of case-control populations (see Lander and Schork, Science, 265, 2037-2048, 1994, the disclosure of which is incorporated herein by reference in its entirety). A major step in the choice of case-control populations is the clinical definition of a given trait or phenotype. Any genetic trait may be analysed by the association method proposed here by carefully selecting the individuals to be included in the trait positive and trait negative phenotypic groups. Four criteria are often useful: clinical phenotype, age at onset, family history and severity. The selection procedure for continuous or quantitative traits (such as blood pressure for example) involves selecting individuals at opposite ends of the phenotype distribution of the trait under study, so as to include in these trait positive and trait negative populations individuals with non-overlapping phenotypes. Preferably, case-control populations consist of phenotypically homogeneous populations. Trait positive and trait negative populations consist of phenotypically uniform populations of individuals representing each between 1 and 98%, preferably between 1 and 80%, more preferably between 1 and 50%, and more preferably between 1 and 30%, most preferably between 1 and 20% of the total population under study, and selected among individuals exhibiting non-overlapping phenotypes. The clearer the difference between the two trait phenotypes, the greater the probability of detecting an association with biallelic markers. The selection of those drastically different but relatively uniform phenotypes enables efficient comparisons in association studies and the possible detection of marked differences at the genetic level, provided that the sample sizes of the populations under study are significant enough.

In preferred embodiments, a first group of between 50 and 300 trait positive individuals, preferably about 100 individuals, are recruited according to their phenotypes. A similar number of trait negative individuals are included in such studies.

Association Analysis

The general strategy to perform association studies using biallelic markers derived from a region carrying a candidate gene is to scan two groups of individuals (case-control populations) in order to measure and statistically compare the allele frequencies of the biallelic markers of the present invention in both groups.

If a statistically significant association with a trait is identified for at least one or more of the analysed biallelic markers, one can assume that: either the associated allele is directly responsible for causing the trait (the associated allele is the trait causing allele), or more likely the associated allele is in linkage disequilibrium with the trait causing allele. The specific characteristics of the associated allele with respect to the candidate gene function usually gives further insight into the relationship between the associated allele and the trait (causal or in linkage disequilibrium). If the evidence indicates that the associated allele within the candidate gene is most probably not the trait causing allele but is in linkage disequilibrium with the real trait causing allele, then the trait causing allele can be found by sequencing the vicinity of the associated marker.

Association studies are usually run in two successive steps. In a first phase, the frequencies of a reduced number of biallelic markers from one or several candidate genes are determined in the trait positive and trait negative populations. In a second phase of the analysis, the identity of the candidate gene and the position of the genetic loci responsible for the given trait is further refined using a higher density of markers from the relevant region. However, if the candidate gene under study is relatively small in length, as it is the case for many of the candidate genes analysed included in the present invention, a single phase may be sufficient to establish significant associations.

Haplotype Analysis

As described above, when a chromosome carrying a disease allele first appears in a population as a result of either mutation or migration, the mutant allele necessarily resides on a chromosome having a set of linked markers: the ancestral haplotype. This haplotype can be tracked through populations and its statistical association with a given trait can be analysed. Complementing single point (allelic) association studies with multi-point association studies also called haplotype studies increases the statistical power of association studies. Thus, a haplotype association study allows one to define the frequency and the type of the ancestral carrier haplotype. A haplotype analysis is important in that it increases the statistical power of an analysis involving individual markers.

In a first stage of a haplotype frequency analysis, the frequency of the possible haplotypes based on various combinations of the identified biallelic markers of the invention is determined. The haplotype frequency is then compared for distinct populations of trait positive and control individuals. The number of trait positive individuals, which should be, subjected to this analysis to obtain statistically significant results usually ranges between 30 and 300, with a preferred number of individuals ranging between 50 and 150. The same considerations apply to the number of unaffected individuals (or random control) used in the study. The results of this first analysis provide haplotype frequencies in case-control populations, for each evaluated haplotype frequency a p-value and an odd ratio are calculated. If a statistically significant association is found the relative risk for an individual carrying the given haplotype of being affected with the trait under study can be approximated.

Interaction Analysis

The biallelic markers of the present invention may also be used to identify patterns of biallelic markers associated with detectable traits resulting from polygenic interactions. The analysis of genetic interaction between alleles at unlinked loci requires individual genotyping using the techniques described herein. The analysis of allelic interaction among a selected set of biallelic markers with appropriate level of statistical significance can be considered as a haplotype analysis. Interaction analysis consists in stratifying the case-control populations with respect to a given haplotype for the first loci and performing a haplotype analysis with the second loci with each subpopulation.

Statistical methods used in association studies are further described below in IV.C.

4) Testing for Linkage in the Presence of Association

The biallelic markers of the present invention may further be used in TDT (transmission/disequilibrium test). TDT tests for both linkage and association and is not affected by population stratification. TDT requires data for affected individuals and their parents or data from unaffected sibs instead of from parents (see Spielmann S. et al., Am. J. Hum. Genet., 52:506-516, 1993; Schaid D. J. et al., Genet. Epideiniol., 13:423-450, 1996, Spielmann S. and Ewens W. J., Am. J. Hum. Genet., 62:450-458, 1998, the disclosures of which are incorporated herein by reference in their entireties). Such combined tests generally reduce the false—positive errors produced by separate analyses.

IV.C. Statistical Methods

In general, any method known in the art to test whether a trait and a genotype show a statistically significant correlation may be used.

1) Methods in Linkage Analysis

Statistical methods and computer programs useful for linkage analysis are well-known to those skilled in the art (see Terwilliger J. D. and Ott J., Handbook of Human Genetic Linkage, John Hopkins University Press, London, 1994; Ott J., Analysis of Human Genetic Linkage, John Hopkins University Press, Baltimore, 1991, the disclosures of which are incorporated herein by reference in their entireties).

2) Methods to Estimate Haplotype Frequencies in a Population

As described above, when genotypes are scored, it is often not possible to distinguish heterozygotes so that haplotype frequencies cannot be easily inferred. When the gametic phase is not known, haplotype frequencies can be estimated from the multilocus genotypic data. Any method known to person skilled in the art can be used to estimate haplotype frequencies (see Lange K., Mathematical and Statistical Methods for Genetic Analysis, Springer, N.Y., 1997; Weir, B. S., Genetic data Analysis II: Methods for Discrete population genetic Data, Sinauer Assoc., Inc., Sunderland, Mass., USA, 1996, the disclosures of which are incorporated herein by reference in their entireties) Preferably, maximum-likelihood haplotype frequencies are computed using an Expectation-Maximization (EM) algorithm (see Dempster et al., J. R. Stat. Soc., 39B:1-38, 1977; Excoffier L. and Slatkin M., Mol. Biol. Evol., 12(5): 921-927, 1995, the disclosures of which are incorporated herein by reference in their entireties). This procedure is an iterative process aiming at obtaining maximum-likelihood estimates of haplotype frequencies from multi-locus genotype data when the gametic phase is unknown. Haplotype estimations are usually performed by applying the EM algorithm using for example the EM-HAPLO program (Hawley M. E. et al., Am. J. Phys. Anthropol., 18:104, 1994, the disclosure of which is incorporated herein by reference in its entirety) or the Arlequin program (Schneider et al., Arlequin: a software for population genetics data analysis, University of Geneva, 1997, the disclosure of which is incorporated herein by reference in its entirety). The EM algorithm is a generalised iterative maximum likelihood approach to estimation and is briefly described below.

In what follows, phenotypes will refer to multi-locus genotypes with unknown haplotypic phase. Genotypes will refer to mutli-locus genotypes with known haplotypic phase.

Suppose one has a sample of Nunrelated individuals typed for K markers. The data observed are the unknown-phase K-locus phenotypes that can be categorized with F different phenotypes. Further, suppose that we have H possible haplotypes (in the case of K biallelic markers, we have for the maximum number of possible haplotypes H=2^{K}).

For phenotype j with cⱼ possible genotypes, we have:

$${P}_{j} = {\sum }_{i = 1}^{{c}_{j}} P ( genotype ( i ) ) = {\sum }_{i = 1}^{{c}_{j}} P ( {h}_{k} , {h}_{l} ) . \qquad Equation 1$$

Here, Pⱼ is the probability of the j^{th} phenotype, and P(hₖhₗ) is the probability of the i^{th} genotype composed of haplotypes hₖ and hₗ. Under random mating (i.e. Hardy-Weinberg Equilibrium), P(hₖhₗ) is expressed as:

P(hₖ, hₗ)=P(hₖ)² for hₖ=hₗ, and

P(hₖ, hₗ)=2P(hₖ)P(hₗ) for hₖ≠hₗ. Equation 2

The E-M algorithm is composed of the following steps: First, the genotype frequencies are estimated from a set of initial values of haplotype frequencies. These haplotype frequencies are denoted P₁⁽⁰⁾, P₂⁽⁰⁾, P₃⁽⁰⁾, . . . , P_{H}⁽⁰⁾. The initial values for the haplotype frequencies may be obtained from a random number generator or in some other way well known in the art. This step is referred to the Expectation step. The next step in the method, called the Maximization step, consists of using the estimates for the genotype frequencies to re-calculate the haplotype frequencies. The first iteration haplotype frequency estimates are denoted by P₁⁽¹⁾, P₂⁽¹⁾, P₃⁽¹⁾, . . . , P_{H}⁽¹⁾. In general, the Expectation step at the s^{th} iteration consists of calculating the probability of placing each phenotype into the different possible genotypes based on the haplotype frequencies of the previous iteration:

$${P ( {h}_{k} , {h}_{l} )}^{( s )} = \frac{{n}_{j}}{N} [ \frac{{{P}_{j} ( {h}_{k} , {h}_{l} )}^{( s )}}{{P}_{j}} ] , \qquad Equation 3$$

where nⱼ is the number of individuals with the j^{th} phenotype and Pⱼ (hₖ, hₗ)^{(s)} is the probability of genotype hₖ,hₗ in phenotype j. In the Maximization step, which is equivalent to the gene-counting method (Smith, Ann. Hum. Genet., 21:254-276, 1957), the haplotype frequencies are re-estimated based on the genotype estimates:

$${P}_{t}^{( s + 1 )} = \frac{1}{2} {\sum }_{j = 1}^{F} {\sum }_{i = 1}^{{c}_{j}} {δ}_{it} {{P}_{j} ( {h}_{k} , {h}_{l} )}^{( s )} . \qquad Equation 4$$

Here, δᵢₜ is an indicator variable which counts the number of occurrences that haplotype t is present in i^{th} genotype; it takes on values 0, 1, and 2.

The E-M iterations cease when the following criterion has been reached. Using Maximum Likelihood Estimation (MLE) theory, one assumes that the phenotypes j are distributed multinomially. At each iteration s, one can compute the likelihood function L. Convergence is achieved when the difference of the log-likehood between two consecutive iterations is less than some small number, preferably 10^{-7.}

3) Methods to Calculate Linkage Disequilibrium Between Markers

A number of methods can be used to calculate linkage disequilibrium between any two genetic positions, in practice linkage disequilibrium is measured by applying a statistical association test to haplotype data taken from a population.

Linkage disequilibrium between any pair of biallelic markers comprising at least one of the biallelic markers of the present invention (Mᵢ, Mⱼ) having alleles (aᵢ/bᵢ) at marker Mᵢ and alleles (aⱼ/bⱼ) at marker Mⱼ can be calculated for every allele combination (aᵢ,aⱼ; aᵢ,bⱼ; bᵢ,aⱼ and bᵢ,bⱼ), according to the Piazza formula: Δₐᵢₐⱼ=√θ4−√(θ4+θ3)(θ4+θ2), where:

θ4=−−=frequency of genotypes not having allele aᵢ at Mᵢ and not having allele aⱼ at Mⱼ

θ3=−+=frequency of genotypes not having allele aᵢ at Mᵢ and having allele aⱼ at Mⱼ

θ2=+−=frequency of genotypes having allele aᵢ at Mᵢ and not having allele aⱼ at Mⱼ

Linkage disequilibrium (LD) between pairs of biallelic markers (Mᵢ, Mⱼ) can also be calculated for every allele combination (ai,aj; ai,bj; bᵢ,aⱼ and bᵢ,bⱼ), according to the maximum-likelihood estimate (MLE) for delta (the composite genotypic disequilibrium coefficient), as described by Weir (Weir B. S., Genetic Data Analysis, Sinauer Ass. Eds, 1996, the disclosure of which is incorporated herein by reference in its entirety). The MLE for the composite linkage disequilibrium is:

Dₐᵢₐⱼ=(2n₁+n₂+n₃+n₄/2)/N−2(pr(aᵢ).pr(aⱼ))

Where n₁=Σ phenotype (aᵢ/aᵢ, aⱼ/aⱼ), n₂=Σ phenotype (aᵢ/aᵢ, aⱼ/bⱼ), n₃=Σ phenotype (aᵢ/bᵢ, aⱼ/aⱼ), n4=Σ phenotype (aᵢ/bᵢ, aⱼ/bⱼ) and N is the number of individuals in the sample.

This formula allows linkage disequilibrium between alleles to be estimated when only genotype, and not haplotype, data are available.

Another means of calculating the linkage disequilibrium between markers is as follows. For a couple of biallelic markers, Mᵢ (aᵢ/bᵢ) and Mⱼ (aⱼ/bⱼ), fitting the Hardy-Weinberg equilibrium, one can estimate the four possible haplotype frequencies in a given population according to the approach described above. The estimation of gametic disequilibrium between ai and aj is simply:

Dₐⱼₐⱼ=pr(haplotype(aᵢ,aⱼ))−pr(aᵢ).pr(aⱼ).

Where pr(aᵢ) is the probability of allele aᵢ and pr(aⱼ) is the probability of allele aⱼ and where pr(haplotype (aᵢ, aⱼ)) is estimated as in Equation 3 above.

For a couple of biallelic marker only one measure of disequilibrium is necessary to describe the association between Mᵢ and Mⱼ.

Then a normalised value of the above is calculated as follows:

D′ₐᵢₐⱼ=Dₐᵢₐⱼ/max (−pr(aᵢ).pr(aⱼ), −pr(bᵢ).pr(bⱼ)) with Dₐᵢₐⱼ<0

D′ₐᵢₐⱼ=Dₐᵢₐⱼ/max (pr(bᵢ).pr(aⱼ), pr(aᵢ).pr(bⱼ)) with Dₐᵢₐⱼ>0

The skilled person will readily appreciate that other LD calculation methods can be used without undue experimentation.

Linkage disequilibrium among a set of biallelic markers having an adequate heterozygosity rate can be determined by genotyping between 50 and 1000 unrelated individuals, preferably between 75 and 200, more preferably around 100.

4) Testing for Association

Methods for determining the statistical significance of a correlation between a phenotype and a genotype, in this case an allele at a biallelic marker or a haplotype made up of such alleles, may be determined by any statistical test known in the art and with any accepted threshold of statistical significance being required. The application of particular methods and thresholds of significance are well with in the skill of the ordinary practitioner of the art.

Testing for association is performed by determining the frequency of a biallelic marker allele in case and control populations and comparing these frequencies with a statistical test to determine if their is a statistically significant difference in frequency which would indicate a correlation between the trait and the biallelic marker allele under study. Similarly, a haplotype analysis is performed by estimating the frequencies of all possible haplotypes for a given set of biallelic markers in case and control populations, and comparing these frequencies with a statistical test to determine if their is a statistically significant correlation between the haplotype and the phenotype (trait) under study. Any statistical tool useful to test for a statistically significant association between a genotype and a phenotype may be used. Preferably the statistical test employed is a chi-square test with one degree of freedom. A p-value is calculated (the p-value is the probability that a statistic as large or larger than the observed one would occur by chance).

Statistical Significance

In preferred embodiments, significance for diagnosis purposes, either as a positive basis for further diagnostic tests or as a preliminary starting point for early preventive therapy, the p value related to a biallelic marker association is preferably about 1×10⁻² or less, more preferably about 1×10⁻⁴ or less, for a single biallelic marker analysis and about 1×10⁻³ or less, still more preferably 1×10⁻⁶ or less and most preferably of about 1×10⁻⁸ or less, for a haplotype analysis involving several markers. These values are believed to be applicable to any association studies involving single or multiple marker combinations.

The skilled person can use the range of values set forth above as a starting point in order to carry out association studies with biallelic markers of the present invention. In doing so, significant associations between the biallelic markers of the present invention and diseases can be revealed.

Phenotypic Permutation

In order to confirm the statistical significance of the first stage haplotype analysis described above, it might be suitable to perform further analyses in which genotyping data from case-control individuals are pooled and randomised with respect to the trait phenotype. Each individual genotyping data is randomly allocated to two groups, which contain the same number of individuals as the case-control populations used to compile the data obtained in the first stage. A second stage haplotype analysis is preferably run on these artificial groups, preferably for the markers included in the haplotype of the first stage analysis showing the highest relative risk coefficient. This experiment is reiterated preferably at least between 100 and 10000 times. The repeated iterations allow the determination of the percentage of obtained haplotypes with a significant p-value level.

Assessment of Statistical Association

To address the problem of false positives similar analysis may be performed with the same case-control populations in random genomic regions. Results in random regions and the candidate region are compared as described in US Provisional Patent Application entitled “Methods, software and apparati for identifying genomic regions harbouring a gene associated with a detectable trait”.

5) Evaluation of Risk Factors

The association between a risk factor (in genetic epidemiology the risk factor is the presence or the absence of a certain allele or haplotype at marker loci) and a disease is measured by the odds ratio (OR) and by the relative risk (RR). If P(R⁺) is the probability of developing the disease for individuals with R and P(R⁻) is the probability for individuals without the risk factor, then the relative risk is simply the ratio of the two probabilities, that is: RR=P(R⁺)P(R⁻)

In case-control studies, direct measures of the relative risk cannot be obtained because of the sampling design. However, the odds ratio allows a good approximation of the relative risk for low-incidence diseases and can be calculated:

$$OR = [ \frac{{F}^{+}}{1 - {F}^{+}} ] / [ \frac{{F}^{-}}{( 1 - {F}^{-} )} ]$$

F⁺ is the frequency of the exposure to the risk factor in cases and F is the frequency of the exposure to the risk factor in controls. F⁺ and F⁻ are calculated using the allelic or haplotype frequencies of the study and further depend on the underlying genetic model (dominant, recessive, additive . . . ).

One can further estimate the attributable risk (AR) which describes the proportion of individuals in a population exhibiting a trait due to a given risk factor. This measure is important in quantitating the role of a specific factor in disease etiology and in terms of the public health impact of a risk factor. The public health relevance of this measure lies in estimating the proportion of cases of disease in the population that could be prevented if the exposure of interest were absent. AR is determined as follows:

AR=P_{E}(RR−1)/(PE(RR−1)+1)

AR is the risk attributable to a biallelic marker allele or a biallelic marker haplotype. P_{E} is the frequency of exposure to an allele or a haplotype within the population at large; and RR is the relative risk which, is approximated with the odds ratio when the trait under study has a relatively low incidence in the general population.

IV.F. Identification Of Biallelic Markers in Linkage Disequilibrium with the Biallelic Markers of the Invention

Once a first biallelic marker has been identified in a genomic region of interest, the practitioner of ordinary skill in the art, using the teachings of the present invention, can easily identify additional biallelic markers in linkage disequilibrium with this first marker. As mentioned before any marker in linkage disequilibrium with a first marker associated with a trait will be associated with the trait. Therefore, once an association has been demonstrated between a given biallelic marker and a trait, the discovery of additional biallelic markers associated with this trait is of great interest in order to increase the density of biallelic markers in this particular region. The causal gene or mutation will be found in the vicinity of the marker or set of markers showing the highest correlation with the trait.

Identification of additional markers in linkage disequilibrium with a given marker involves: (a) amplifying a genomic fragment comprising a first biallelic marker from a plurality of individuals; (b) identifying of second biallelic markers in the genomic region harboring said first biallelic marker; (c) conducting a linkage disequilibrium analysis between said first biallelic marker and second biallelic markers; and (d) selecting said second biallelic markers as being in linkage disequilibrium with said first marker. Subcombinations comprising steps (b) and (c) are also contemplated.

Methods to identify biallelic markers and to conduct linkage disequilibrium analysis are described herein and can be carried out by the skilled person without undue experimentation. The present invention then also concerns biallelic markers which are in linkage disequilibrium with any of the specific biallelic markers of SEQ ID Nos. 1 to 171, 1 to 100, 101 to 162, 163 to 171 and which are expected to present similar characteristics in terms of their respective association with a given trait.

Example 5 illustrates the measurement of linkage disequilibrium between a publicly known biallelic marker, the “ApoE Site A”, located within the Alzheimer's related ApoE gene, and other biallelic markers randomly derived from the genomic region containing the ApoE gene.

IV.G. Identification of Functional Mutations

Once a positive association is confirmed with a biallelic marker of the present invention, the associated candidate gene can be scanned for mutations by comparing the sequences of a selected number of trait positive and trait negative individuals. In a preferred embodiment, functional regions such as exons and splice sites, promoters and other regulatory regions of the candidate gene are scanned for mutations. Preferably, trait positive individuals carry the haplotype shown to be associated with the trait and trait negative individuals do not carry the haplotype or allele associated with the trait. The mutation detection procedure is essentially similar to that used for biallelic site identification.

The method used to detect such mutations generally comprises the following steps: (a) amplification of a region of the candidate gene comprising a biallelic marker or a group of biallelic markers associated with the trait from DNA samples of trait positive patients and trait negative controls; (b) sequencing of the amplified region; (c) comparison of DNA sequences from trait-positive patients and trait-negative controls; and (d) determination of mutations specific to trait-positive patients. Subcombinations which comprise steps (b) and (c) are specifically contemplated.

It is preferred that candidate polymorphisms be then verified by screening a larger population of cases and controls by means of any genotyping procedure such as those described herein, preferably using a microsequencing technique in an individual test format. Polymorphisms are considered as candidate mutations when present in cases and controls at frequencies compatible with the expected association results.

V. Biallelic Markers of the Invention in Diagnostics, Prevention and Treatment of Disease

Biallelic Markers of the Invention in Methods of Genetic Diagnostics

The biallelic markers of the present invention can also be used to develop diagnostics tests capable of identifying individuals who express a detectable trait as the result of a specific genotype or individuals whose genotype places them at risk of developing a detectable trait at a subsequent time. The trait analyzed using the present diagnostics may be any detectable trait, including a disease, a response to an agent acting on a disease, or side effects to an agent acting on a disease.

The diagnostic techniques of the present invention may employ a variety of methodologies to determine whether a test subject has a biallelic marker pattern associated with an increased risk of developing a detectable trait or whether the individual suffers from a detectable trait as a result of a particular mutation, including methods which enable the analysis of individual chromosomes for haplotyping, such as family studies, single sperm DNA analysis or somatic hybrids.

The present invention provides diagnostic methods to determine whether an individual is at risk of developing a disease or suffers from a disease resulting from a mutation or a polymorphism in a candidate gene of the present invention. The present invention also provides methods to determine whether an individual is likely to respond positively to an agent acting on a disease or whether an individual is at risk of developing an adverse side effect to an agent acting on a disease.

These methods involve obtaining a nucleic acid sample from the individual and, determining, whether the nucleic acid sample contains at least one allele or at least one biallelic marker haplotype, indicative of a risk of developing the trait or indicative that the individual expresses the trait as a result of possessing a particular candidate gene polymorphism or mutation (trait-causing allele).

Preferably, in such diagnostic methods, a nucleic acid sample is obtained from the individual and this sample is genotyped using methods described above in III. The diagnostics may be based on a single biallelic marker or a on group of biallelic markers.

In each of these methods, a nucleic acid sample is obtained from the test subject and the biallelic marker pattern of one or more of the biallelic markers of SEQ ID Nos. 1 to 171, 1 to 100, 101 to 162, 163 to 171 is determined.

In one embodiment, a PCR amplification is conducted on the nucleic acid sample to amplify regions in which polymorphisms associated with a detectable phenotype have been identified. The amplification products are sequenced to determine whether the individual possesses one or more polymorphisms associated with a detectable phenotype. The primers used to generate amplification products may comprise the primers of SEQ ID No. 172 to 513, 172 to 271, 272 to 333, 334 to 342, 343 to 442, 443 to 504 and 505 to 513. Alternatively, the nucleic acid sample is subjected to microsequencing reactions as described above to determine whether the individual possesses one or more polymorphisms associated with a detectable phenotype resulting from a mutation or a polymorphism in a candidate gene. In another embodiment, the nucleic acid sample is contacted with one or more allele specific oligonucleotide probes which, specifically hybridize to one or more candidate gene alleles associated with a detectable phenotype.

These diagnostic methods are extremely valuable as they can, in certain circumstances, be used to initiate preventive treatments or to allow an individual carrying a significant haplotype to foresee warning signs such as minor symptoms. In diseases in which attacks may be extremely violent and sometimes fatal if not treated on time, such as disease, the knowledge of a potential predisposition, even if this predisposition is not absolute, might contribute in a very significant manner to treatment efficacy. Similarly, a diagnosed predisposition to a potential side effect could immediately direct the physician toward a treatment for which such side effects have not been observed during clinical trials.

Diagnostics, which analyze and predict response to a drug or side effects to a drug, may be used to determine whether an individual should be treated with a particular drug. For example, if the diagnostic indicates a likelihood that an individual will respond positively to treatment with a particular drug, the drug may be administered to the individual. Conversely, if the diagnostic indicates that an individual is likely to respond negatively to treatment with a particular drug, an alternative course of treatment may be prescribed. A negative response may be defined as either the absence of an efficacious response or the presence of toxic side effects.

Clinical drug trials represent another application for the markers of the present invention. One or more markers indicative of response to an agent acting on a disease or to side effects to an agent acting on a disease may be identified using the methods described above. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems.

Prevention and Treatment of Disease Using Biallelic Markers

The detection of susceptibility to disease in individuals is very important. For example, in some obesity disorders, treatments may be available to prevent or at least slow disease progression and obesity-related disorders such as diabetes and heart disease.

Consequently, the invention concerns a method for the treatment of a disease, wherein disease is understood to comprise any disorder, comprising the following steps:

selecting an individual whose DNA comprises alleles of a map-related biallelic marker or of a group of map-related biallelic markers associated with disease;

following up said individual for the appearance (and optionally the development) of the symptoms related to a disease; and

administering a treatment acting against said disease or against symptoms thereof to said individual at an appropriate stage of the disease.

Another embodiment of the present invention comprises a method for the treatment of a disease comprising the following steps:

selecting an individual whose DNA comprises alleles of a map-related biallelic marker or of a group of map-related biallelic markers associated with a disease;

administering a preventive treatment for said disease to said individual.

In a further embodiment, the present invention concerns a method for the treatment of a disease comprising the following steps:

selecting an individual whose DNA comprises alleles of a map-related biallelic marker or of a group of map-related biallelic markers associated with a disease;

administering a preventive treatment of said disease to said individual;

following up said individual for the appearance and the development of disease symptoms; and optionally

administering a treatment acting against said disease or against symptoms thereof to said individual at the appropriate stage of the disease.

For use in the determination of the course of treatment of an individual suffering from disease, the present invention also concerns a method for the treatment of a disease comprising the following steps:

selecting an individual suffering from a disease whose DNA comprises alleles of a map-related biallelic marker or of a group of map-related biallelic markers associated with an obesity disorder or of the symptoms thereof; and

administering a treatment acting against said obesity disorder or symptoms thereof to said individual.

The invention also concerns a method for the treatment of a disease in a selected population of individuals. The method comprises:

selecting an individual suffering from an obesity disorder and whose DNA comprises alleles of a map-related biallelic marker or of a group of map-related biallelic markers associated with a positive response to treatment with an effective amount of a medicament acting against said disease or symptoms thereof,

and/or whose DNA does not comprise alleles of a map-related biallelic marker or of a group of map-related biallelic markers associated with a negative response to treatment with said medicament; and

administering at suitable intervals an effective amount of said medicament to said selected individual.

In the context of the present invention, a “positive response” to a medicament can be defined as comprising a reduction of the symptoms related to the disease. In the context of the present invention, a “negative response” to a medicament can be defined as comprising either a lack of positive response to the medicament which does not lead to a symptom reduction or which leads to a side-effect observed following administration of the medicament.

The invention also relates to a method of determining whether a subject is likely to respond positively to treatment with a medicament. The method comprises identifying a first population of individuals who respond positively to said medicament and a second population of individuals who respond negatively to said medicament. One or more biallelic markers is identified in the first population which is associated with a positive response to said medicament or one or more biallelic markers is identified in the second population which is associated with a negative response to said medicament. The biallelic markers may be identified using the techniques described herein.

A DNA sample is then obtained from the subject to be tested. The DNA sample is analyzed to determine whether it comprises alleles of one or more biallelic markers associated with a positive response to treatment with the medicament and/or alleles of one or more biallelic markers associated with a negative response to treatment with the medicament.

In some embodiments, the medicament may be administered to the subject in a clinical trial if the DNA sample contains alleles of one or more map-related biallelic markers associated with a positive response to treatment with the medicament and/or if the DNA sample lacks alleles of one or more map-related biallelic markers associated with a negative response to treatment with the medicament. In preferred embodiments, the medicament is a drug acting against an obesity disorder.

Using the method of the present invention, the evaluation of drug efficacy may be conducted in a population of individuals likely to respond favorably to the medicament.

Another aspect of the invention is a method of using a medicament comprising obtaining a DNA sample from a subject, determining whether the DNA sample contains alleles of one or more map-related biallelic markers associated with a positive response to the medicament and/or whether the DNA sample contains alleles of one or more map-related biallelic markers associated with a negative response to the medicament, and administering the medicament to the subject if the DNA sample contains alleles of one or more map-related biallelic markers associated with a positive response to the medicament and/or if the DNA sample lacks alleles of one or more map-related biallelic markers associated with a negative response to the medicament.

The invention also concerns a method for the clinical testing of a medicament, preferably a medicament acting against a disease or symptoms thereof, more preferably an obesity disorder. The method comprises the following steps:

administering a medicament, preferably a medicament susceptible of acting against a disease or symptoms thereof to a heterogeneous population of individuals,

identifying a first population of individuals who respond positively to said medicament and a second population of individuals who respond negatively to said medicament,

identifying map-related biallelic markers in said first population which are associated with a positive response to said medicament,

selecting individuals whose DNA comprises map-related biallelic markers associated with a positive response to said medicament, and

administering said medicament to said individuals.

In any of the methods for the prevention, diagnosis and treatment of disease, including methods of using a medicament, clinical testing of a medicament, determining whether a subject is likely to respond positively to treatment with a medicament, said map-related biallelic marker or set of map-related biallelic markers may encompass biallelic markers and sets or maps of biallelic markers with any further limitation described in this disclosure. As described herein, preferably said map-related biallelic marker comprises a biallelic marker of SEQ ID Nos. 1 to 171, 1 to 100, 101 to 162, 163 to 171. Optionally, said map-related biallelic marker or set of map-related biallelic markers comprises at least one biallelic marker selected from the group consisting of a chromosome 3 map-related biallelic marker; a chromosome 10 map-related biallelic marker; and a chromosome 19 map-related biallelic marker.

Preferably, said chromosome 3, 10 and 19 map-related biallelic markers are selected from the group consisting of:

chromosome 3 biallelic markers: (a) SEQ ID Nos. 8, 10, 12, 13, 14, 15, 16, 17, 18, 19, 20, 23, 24, 25, 26, 27, 70, 72, 73, 74, 75, 76, 77; and (b) SEQ ID Nos. 102, 105, 106, 107, 110, 111, 117, 118, 119, 120, 121, 122, 123, 124, 125, 126, 127, 159, 160, 161; and (c) 163, 166, 167;

chromosome 10 biallelic markers: (a) SEQ ID Nos. 1, 2, 3, 4, 5, 6, 7, 9, 11, 21, 22, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 71, 78, 79, 80, 81, 82, 83, 84, 85, 86, 87, 88, 89, 90, 91, 92, 93, 94, 95, 96, 97, 98, 99, 100; (b) SEQ ID Nos. 101, 103, 104, 108, 109, 112, 113, 114, 115, 116, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158; and (c) SEQ ID Nos. 164, 165, 168, 169, 170, 171; and

chromosome 19 biallelic marker: (a) SEQ ID No. 162.

Such methods are deemed to be extremely useful to increase the benefit/risk ratio resulting from the administration of medicaments which may cause undesirable side effects and/or be inefficacious to a portion of the patient population to which it is normally administered.

Once an individual has been diagnosed as suffering from a disease, selection tests are carried out to determine whether the DNA of this individual comprises alleles of a biallelic marker or of a group of biallelic markers associated with a positive response to treatment or with a negative response to treatment which may include either side effects or unresponsiveness.

The selection of the patient to be treated using the method of the present invention can be carried out through the detection methods described above. The individuals which are to be selected are preferably those whose DNA does not comprise alleles of a biallelic marker or of a group of biallelic markers associated with a negative response to treatment. The knowledge of an individual's genetic predisposition to unresponsiveness or side effects to particular medicaments allows the clinician to direct treatment toward appropriate drugs against a disease or symptoms thereof.

Once the patient's genetic predispositions have been determined, the clinician can select appropriate treatment for which negative response, particularly side effects, has not been reported or has been reported only marginally for the patient.

In preferred embodiments of this section titled “Prevention and treatment of disease using biallelic markers,” a disease comprises an obesity disorder. The biallelic markers of the invention are located in genomic regions suspected to contain a genetic determinant of an obesity disorder. It will be appreciated that the prevention, diagnostic, prognosis and treatment methods described above may be used in the context of a wide variety of obesity disorders. For example, biallelic markers located in particular genomic regions may be used in the context of an obesity disorder as described in a reference, providing evidence for a disease locus, some of which are cited above. By way of example and not limitation, examples of obesity disorders may comprise obesity-related atherosclerosis, obesity-related insulin resistance, obesity-related hypertension, microangiopathic lesions resulting from obesity-related Type II diabetes, ocular lesions caused by microangiopathy in. obese individuals with Type II diabetes, and renal lesions caused by microangiopathy in obese individuals with Type II diabetes. Obesity-related disorders may also include hyperinsulinemia and hyperglycemia.

Said genomic regions may, however, also contain genetic determinants for non-obesity disorders. The present invention thus comprises any of the prevention, diagnostic, prognosis and treatment methods described herein using the map-related biallelic markers of the invention in methods of preventing, diagnosing, managing and treating any disorder.

VI. Computer-Related Embodiments

In some embodiments of the present invention a computer to based system may support the on-line coordination between the identification of biallelic markers and the corresponding analysis of their frequency in the different groups.

As used herein the term “nucleic acid codes of SEQ ID Nos. 1 to 171, 1 to 100, 101 to 162, 163 to 171, 172 to 513, 172 to 271, 272 to 333, 334 to 342, 343 to 442, 443 to 504 and 505 to 513” encompasses the nucleotide sequences of SEQ ID Nos. 1 to 171, 1 to 100, 101 to 162, 163 to 171, 172 to 513, 172 to 271, 272 to 333, 334 to 342, 343 to 442, 443 to 504 and 505 to 513, fragments of SEQ ID Nos. 1 to 171, 1 to 100, 101 to 162, 163 to 171, 172 to 513, 172 to 271, 272 to 333, 334 to 342, 343 to 442, 443 to 504 and 505 to 513, nucleotide sequences homologous to SEQ ID Nos. 1 to 171, 1 to 100, 101 to 162, 163 to 171, 172 to 513, 172 to 271, 272 to 333, 334 to 342, 343 to 442, 443 to 504 and 505 to 513 orhomologous to fragments of SEQ ID Nos. 1 to 171, 1 to 100, 101 to 162, 163 to 171, 172 to 513, 172 to 271, 272 to 333, 334 to 342, 343 to 442, 443 to 504 and 505 to 513, and sequences complementary to all of the preceding sequences. As used herein the term “nucleic acid codes of SEQ ID Nos. 1 to 171, 1 to 100, 101 to 162, 163 to 171, 172 to 513, 172 to 271, 272 to 333, 334 to 342, 343 to 442, 443 to 504 and 505 to 513” further encompasses the nucleotide sequences comprising, consisting essentially of, or consisting of any one of the following:

a) a contiguous span of at least 8, 10, 12, 15, 18, 19, 20, 22, 23, 24, 25, 30, 35, 43, 44, 45, 46 or 47 nucleotides, to the extent that a contiguous span of these lengths is consistent with the lengths of the particular Sequence ID, of any of SEQ ID Nos. 1 to 100, 101 to 162 and 163 to 171, or the complements thereof;

b) a contiguous span of at least 8, 10, 12, 15, 18, 19, 20, 22, 23, 24, 25, 30, 35, 43, 44, 45, 46 or 47 nucleotides, to the extent that a contiguous span of these lengths is consistent with the lengths of the particular Sequence ID, of any of SEQ ID Nos. 1 to 100, 101 to 162 and 163 to 171 or the complements thereof, further comprising the 1^{ST} allele of the polymorphic base of the respective SEQ ID number;

c) a contiguous span of at least 8, 10, 12, 15, 18, 19, 20, 22, 23, 24, 25, 30, 35, 43, 44, 45, 46 or 47 nucleotides, to the extent that a contiguous span of these lengths is consistent with the lengths of the particular Sequence ID, of any of SEQ ID Nos. 1 to 100, 101 to 162 and 163 to 171 or the complements thereof, further comprising the 2^{ND} allele of the polymorphic base of the respective SEQ ID number;

d) a contiguous span of at least 8, 10, 12, 15, 18, 19, 20, or 21 nucleotides, to the extent that a contiguous span of these lengths is consistent with the lengths of the particular Sequence ID, of any of SEQ ID No. 172 to 513, 172 to 271, 272 to 333, 334 to 342, 343 to 442, 443 to 504 and 505 to 513 or the complements thereof.

The “nucleic acid codes of SEQ ID Nos. 1 to 171, 1 to 100, 101 to 162, 163 to 171, 172 to 513, 172 to 271, 272 to 333, 334 to 342, 343 to 442, 443 to 504 and 505 to 513” further encompass nucleotide sequences homologous to:

a) a contiguous span of at least 8, 10, 12, 15, 18, 19, 20, 22, 23, 24, 25, 30, 35, 43, 44, 45, 46 or 47 nucleotides, to the extent that a contiguous span of these lengths is consistent with the lengths of the particular Sequence ID, of any of SEQ ID Nos. 1 to 100, 101 to 162 and 163 to 171 or the complements thereof;

b) a contiguous span of at least 8, 10, 12, 15, 18, 19, 20, 22, 23, 24, 25, 30, 35, 43, 44, 45, 46 or 47 nucleotides, to the extent that a contiguous span of these lengths is consistent with the lengths of the particular Sequence ID, of any of SEQ ID Nos. 1 to 100, 101 to 162 and 163 to 171 or the complements thereof, further comprising the 1^{ST} allele of the polymorphic base of the respective SEQ ID number;

c) a contiguous span of at least 8, 10, 12, 15, 18, 19, 20, 22, 23, 24, 25, 30, 35, 43, 44, 45, 46 or 47 nucleotides, to the extent that a contiguous span of these lengths is consistent with the lengths of the particular Sequence ID, of any of SEQ ID Nos. 1 to 100, 101 to 162 and 163 to 171 or the complements thereof, further comprising the 2^{ND} allele of the polymorphic base of the respective SEQ ID number;

d) a contiguous span of at least 8, 10, 12, 15, 18, 19, 20, or 21 nucleotides, to the extent that a contiguous span of these lengths is consistent with the lengths of the particular Sequence ID, of any of SEQ ID No. 172 to 513, 172 to 271, 272 to 333, 334 to 342, 343 to 442, 443 to 504 and 505 to 513 or the complements thereof.

Homologous sequences refer to a sequence having at least 99%, 98%, 97%, 96%, 95%, 90%, 85%, 80%, or 75% homology to these contiguous spans. Homology may be determined using any method described herein, including BLAST2N with the default parameters or with any modified parameters. Homologous sequences also may include RNA sequences in which uridines replace the thymines in the nucleic acid codes of the invention. It will be appreciated that the nucleic acid codes of the invention can be represented in the traditional single character format (See the inside back cover of Stryer, Lubert. Biochemistry, 3^{rd} edition. W. H Freeman & Co., New York.) or in any other format or code which records the identity of the nucleotides in a sequence.

It should be noted that the nucleic acid codes of the invention further encompass all of the polynucleotides disclosed, described or claimed in the present application. Moreover, the present invention specifically contemplates computer readable media and computer systems wherein such codes are stored individually or in any combination.

It should also be noted that any of the computer embodiments may comprise sets or maps of the nucleic acid codes described above. In particular, any of the embodiments may comprise a set of at least 1, 2, 3, 4, 5, 10, 15, 20, 25, 30, 40, 50, 70, 85, 100 or 1132 nucleic acid codes selected from the group of consisting of SEQ ID Nos. 1 to 100, 101 to 162 and 163 to 171. Optionally, said nucleic acid codes are selected from the group of consisting of:

chromosome 3 biallelic markers: (a) SEQ ID Nos. 8, 10, 12, 13, 14, 15, 16, 17, 18, 19, 20, 23, 24, 25, 26, 27, 70, 72, 73, 74, 75, 76, 77; and (b) SEQ ID Nos. 102, 105, 106, 107, 110, 111, 117, 118, 119, 120, 121, 122, 123, 124, 125, 126, 127, 159, 160, 161; and (c) 163, 166, 167;

chromosome 10 biallelic markers: (a) SEQ ID Nos. 1, 2, 3, 4, 5, 6, 7, 9, 11, 21, 22, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 71, 78, 79, 80, 81, 82, 83, 84, 85, 86, 87, 88, 89, 90, 91, 92, 93, 94, 95, 96, 97, 98, 99, 100; (b) SEQ ID Nos. 101, 103, 104, 108, 109, 112, 113, 114, 115, 116, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158; and (c) SEQ ID Nos. 164, 165, 168, 169, 170, 171; and

chromosome 19 biallelic marker: (a) SEQ ID No. 162.

It will be appreciated by those skilled in the art that the nucleic acid codes of SEQ ID Nos. 1 to 171, 1 to 100, 101 to 162, 163 to 171, 172 to 513, 172 to 271, 272 to 333, 334 to 342, 343 to 442, 443 to 504 and 505 to 513 can be stored, recorded, and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate manufactures comprising one or more of the nucleic acid codes of the invention. Another aspect of the present invention is a computer readable medium having recorded thereon at least 2, 5, 10, 15, 20, 25, 30, 50, 100, 200, 500, 1000 or all of nucleic acid codes of SEQ ID Nos. 1 to 171, 1 to 100, 101 to 162, 163 to 171, 172 to 513, 172 to 271, 272 to 333, 334 to 342, 343 to 442, 443 to 504 and 505 to 513.

In other embodiments, one or more features of a biallelic marker of the invention can be stored, recorded and manipulated on any medium which can be read and accessed by a computer. Examples of features which may be stored, recorded and manipulated on a medium include but are not limited to references designating a biallelic marker of the invention, allelic frequency of a biallelic marker allele of the invention in a population, the type (such as deletion, single nucleotide polymorphism) of a biallelic marker of the invention, chromosomal localization in the human genome of a biallelic marker of the invention, localization in a contig, localization in a gene, association with a trait or linkage disequilibrium with a genetic element. Preferably, a nucleic acid code of the invention corresponding to a biallelic marker and a feature corresponding to said biallelic marker are stored on said medium.

In further embodiments, results of genotyping assays using the biallelic markers of the invention are stored on any medium which can be read and accessed by a computer. In particular, a genotype of a biallelic marker of the invention at least one individual displaying or affected by a trait or one control individual can be stored, recorded and manipulated on any medium which can be read and accessed by a computer. Genotypes of at least 1, 2, 5, 10, 50, 100, 200, 300, 500, 1000, 2000 or 5000 individuals displaying or affected by a trait or control individuals at 2, 5, 10, 15, 20, 25, 30, 50, 100, 200, 500, 1000 or all of the map-related biallelic markers of SEQ ID Nos. 1 to 100, 101 to 162, 163 to 171 can be stored, recorded and manipulated on any medium which can be read and accessed by a computer. Preferably, a nucleic acid code of the invention corresponding to a genotype at a map-related biallelic of an individual is stored on said medium; optionally, any reference, designation or nucleic acid code corresponding to a map-related marker and the identity of the allele or indication of the genotype of an individual at the biallelic marker are stored on said medium

Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art.

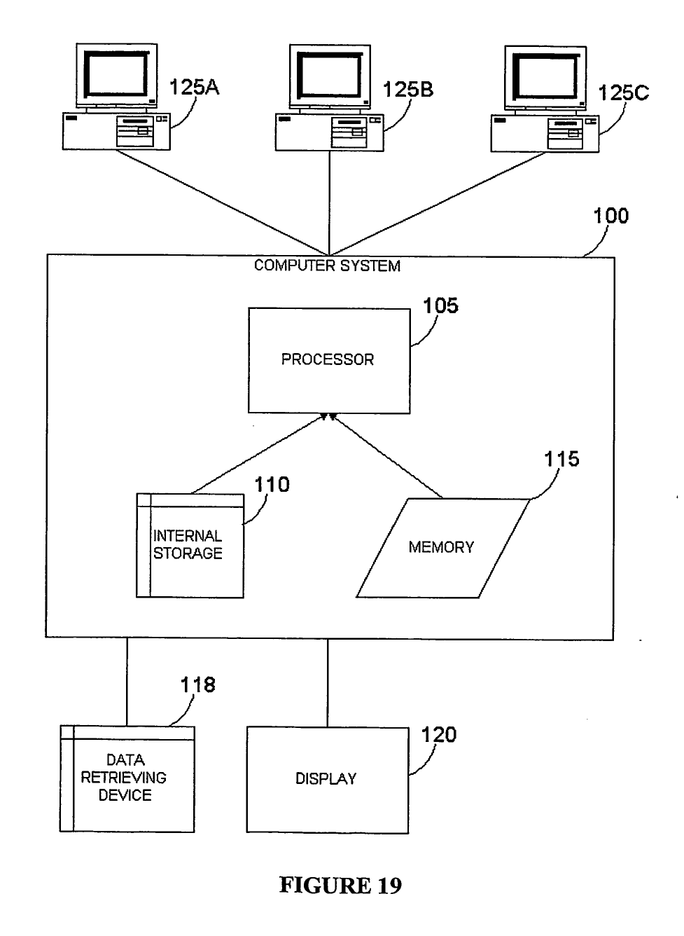

Embodiments of the present invention include systems, particularly computer systems which store and manipulate the sequence, feature and genotyping information of the biallelic markers of the invention described herein. One example of a computer system 100 is illustrated in block diagram form in FIG. 19. As used herein, “a computer system” refers to the hardware components, software components, and data storage components used to analyze the nucleotide sequences of the nucleic acid codes of the invention or the amino acid sequences of the polypeptide codes of the invention. In one embodiment, the computer system 100 is a Sun Enterprise 1000 server (Sun Microsystems, Palo Alto, Calif.). The computer system 100 preferably includes a processor for processing, accessing and manipulating the sequence, feature and genotyping data. The processor 105 can be any well-known type of central processing unit, such as the Pentium III from Intel Corporation, or similar processor from Sun, Motorola, Compaq or International Business Machines.

Preferably, the computer system 100 is a general purpose system that comprises the processor 105 and one or more internal data storage components 110 for storing data, and one or more data retrieving devices for retrieving the data stored on the data storage components. A skilled artisan can readily appreciate that any one of the currently available computer systems are suitable.

In one particular embodiment, the computer system 100 includes a processor 105 connected to a bus which is connected to a main memory 115 (preferably implemented as RAM) and one or more internal data storage devices 110, such as a hard drive and/or other computer readable media having data recorded thereon. In some embodiments, the computer system 100 further includes one or more data retrieving device 118 for reading the data stored on the internal data storage devices 110.

The data retrieving device 118 may represent, for example, a floppy disk drive, a compact disk drive, a magnetic tape drive, etc. In some embodiments, the internal data storage device 110 is a removable computer readable medium such as a floppy disk, a compact disk, a magnetic tape, etc. containing control logic and/or data recorded thereon. The computer system 100 may advantageously include or be programmed by appropriate software for reading the control logic and/or the data from the data storage component once inserted in the data retrieving device.

The computer system 100 includes a display 120 which is used to display output to a computer user. It should also be noted that the computer system 100 can be linked to other computer systems 125a-c in a network or wide area network to provide centralized access to the computer system 100.

Software such as search tools, compare tools, genome mapping and diagramming tools, and modeling tools etc., for accessing and processing the nucleotide sequences of the nucleic acid codes of the invention or feature and genotyping information may reside in main memory 115 during execution.

The present invention also encompasses the use of said computer readable media and computer systems according to methods described below, and/or with any further limitation described in this specification.

Accordingly, the present invention concerns methods for accessing, processing and selecting map-related biallelic markers with the use of a computer program. In one aspect, the invention comprises accessing a nucleic acid code, feature and/or genotyping information corresponding to a map-related biallelic marker of SEQ ID Nos. 1 to 171, 1 to 100, 101 to 162 and 163 to 171 through the use of a computer program.

In another aspect, the invention involves reading a nucleic acid code, feature and/or genotyping information corresponding to a map-related biallelic marker through the use of a computer program; and identifying or selecting a biallelic marker located in a specified chromosomal region, a specified contig or a specified gene; wherein said map-related biallelic marker is selected from the biallelic markers of SEQ ID Nos. 1 to 171, 1 to 100, 101 to 162 and 163 to 171.

In another aspect, the invention involves reading a nucleic acid code, feature and/or genotyping information corresponding to a biallelic marker through the use of a computer program; and identifying or selecting a biallelic marker located in a specified chromosomal region, a specified contig or a specified gene at a specified distance from a map-related biallelic marker; wherein said map-related biallelic marker is selected from the biallelic markers of SEQ ID Nos. 1 to 171, 1 to 100, 101 to 162 and 163 to 171.

In another aspect, the invention involves reading a nucleic acid code, feature and/or genotyping information corresponding to a map-related biallelic marker through the use of a computer program; and identifying or selecting a biallelic marker having a specified allelic frequency, preferably minimum or maximum allelic frequency, for an allele thereof; wherein said map-related biallelic marker is selected from the biallelic markers of SEQ ID Nos. 1 to 171, 1 to 100, 101 to 162 and 163 to 171.

The present invention also concerns methods for constructing a map or set of biallelic markers, such as for use in conducting genetic analyses. Said maps of biallelic markers can then be used for example in forensic applications, or in disease association studies, as described further herein. In one aspect, a set of biallelic markers is selected from biallelic markers stored on a computer readable medium. Biallelic markers may be selected according to a desired criteria, as described above, such as their localization in desired regions of the genome. Markers can also selected such that they are separated by a specified average distance in the genome, or in a selected genomic region, contig, or gene. In another example, biallelic markers can be selected such that they have a specified heterozygosity rate.

Thus, any of the embodiments listed above may apply to the construction of biallelic marker maps, wherein the methods for accessing, processing and selecting map-related biallelic markers comprises selecting or identifying at least 2, 5, 10, 15, 20, 25, 30, 50, 100, 200, 500, 1000 or 10000 biallelic markers. In particular, the invention encompasses a method of constructing a biallelic marker map comprising reading a nucleic acid code, feature and/or genotyping information corresponding to a map-related biallelic marker through the use of a computer program; and identifying or selecting at least 2, 5, 10, 15, 20, 25, 30, 50, 100, 200, 500, 1000 biallelic markers located in a specified chromosomal region, a specified contig or a specified gene; wherein said map-related biallelic marker is selected from the biallelic markers of SEQ ID Nos. 1 to 171, 1 to 100, 101 to 162-and 163 to 171.

It will be appreciated that biallelic marker maps and the methods of constructing them may comprise any further limitations to biallelic markers and maps described herein. The maps and methods of constructing maps may also further comprise methods of genotyping and/or any methods of using biallelic markers maps. It will also be appreciated that any suitable designation or reference sequence may be used to specify a chromosomal region.

In another aspect, the invention encompasses methods of genetic analysis using the biallelic markers of the invention. Genotyping information of any number of individuals at a map-related biallelic marker may be stored on a computer readable medium. Genotyping information may be stored as the genotype for an individual or as a frequency in a population, for example. In one aspect, one or more biallelic markers and any individuals which have been genotyped for said biallelic marker can be specified, such that genotyping results from the one or more individuals at one or more of said biallelic markers are provided, and can then be further analyzed in a genetic analysis method such as those described herein.

Thus, the invention encompasses a method for providing genotyping information for use in genetic analysis comprising specifying a map-related biallelic marker; specifying an individual; and providing the genotype of said individual through the use of a computer program which accesses a computer readable medium comprising genotyping information for said individual. Preferably said map-related biallelic marker comprises at least 1, 2, 5, 10, 15, 20, 25, 30, 50, 100, 200, 500, 1000 biallelic markers selected from the group consisting of SEQ ID Nos. 1 to 171, 1 to 100, 101 to 162 and 163 to 171. Preferably at least 1, 2, 5, 10, 50, 100, 200, 300, 500, 1000, 2000 or 5000 individuals are specified.

The genotyping information at one or more map-related biallelic markers of the invention is then useful in genetic analysis methods as further described herein, such as in association studies. In a first example, the genetic variation in a candidate gene between an affected group of individuals displaying a detectable trait and an unaffected control group can be compared, in order to implicate or absolve the candidate gene as a factor in the trait. A map of several biallelic markers, preferably providing the order and relative location for the markers could serve to compare genetic variation. Said map allows the construction of haplotypes using the natural order of markers given on the map, and these haplotypes, comprising a portrait of the genetic variation on each of the two chromosome carried by the individual, can be compared between those affected and controls for evidence of association. By providing genotyping information at selected biallelic markers of a biallelic marker map on a computer readable medium, haplotypes can be compared through the use of a computer program. In addition, linkage disequilibria (LD) may be calculated for pairs of adjacent markers. The LD value would permit one to predict whether a genetic variant near the pair of biallelic markers, but not mapped itself, might be detectable in association studies of the markers.

In another example, instead of selecting a candidate gene or genomic region, a biallelic marker map of the genome could allow one to map the approximate location of a gene influencing a disease or trait through association studies. Positive association results with certain biallelic markers would indicate a potential disease gene variant in the general location of the biallelic markers and thus would serve to focus further research on this specific area of the genome or biallelic marker map.

Thus, the present invention encompasses a method of estimating the frequency of an allele in a population comprising: a) reading the genotypes of individuals from said population for a biallelic marker; and b) determining the proportional representation of said biallelic marker in said population.

In another aspect, the invention involves a method of detecting an association between a genotype and a phenotype, comprising the steps of: a) reading the genotype of at least one individual at one or more map-related biallelic marker in a trait positive population; b) reading the genotype of said map-related biallelic marker in a control population; and c) determining whether a statistically significant association exists between said genotype and said phenotype.

The invention also involves a method of estimating the frequency of a haplotype for a set of biallelic markers in a population, comprising: a) reading the genotype of at least one individual at one or more least one map-related biallelic marker in a trait positive population; b) reading the identity of the nucleotides at a second biallelic marker for both copies of said second biallelic marker present in the genome of each individual in said population; and c) applying a haplotype determination method to the identities of the nucleotides determined in steps a) and b) to obtain an estimate of said frequency. Preferably, said haplotype determination method is selected from the group consisting of asymmetric PCR amplification, double PCR amplification of specific alleles, the Clark algorithm, or an expectation-maximization algorithm. Preferably, a map-related biallelic marker is selected from the group of consisting of the biallelic markers of SEQ ID Nos. 1 to 171, 1 to 100, 101 to 162 and 163 to 171.

The invention further encompasses a method of detecting an association between a haplotype and a phenotype, comprising the steps of: a) estimating the frequency of at least one haplotype in a trait positive population according to the method described above; b) estimating the frequency of said haplotype in a control population according to the method described above; and c) determining whether a statistically significant association exists between said haplotype and said phenotype.

In some embodiments, the computer system described herein 100 may further comprise a sequence comparer for comparing the above-described nucleic acid codes of the invention stored on a computer readable medium to reference nucleotide sequences stored on a computer readable medium. A “sequence comparer” refers to one or more programs which are implemented on the computer system 100 to compare a nucleotide sequence with other nucleotide sequences stored within the data storage means. For example, the sequence comparer may compare the nucleotide sequences of nucleic acid codes of the invention stored on a computer readable medium to reference sequences stored on a computer readable medium to identify homologies. The various sequence comparer programs identified elsewhere in this patent specification are particularly contemplated for use in this aspect of the invention.

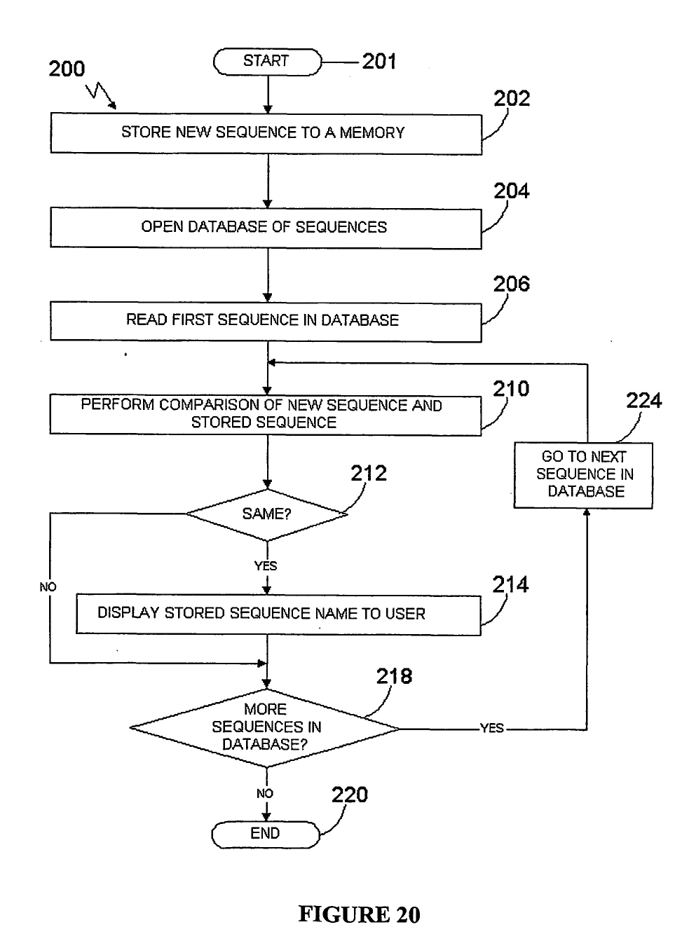

FIG. 20 is a flow diagram illustrating one embodiment of a process 200 for comparing a new nucleotide sequence with a database of sequences in order to determine the homology levels between the new sequence and the sequences in the database. The database of sequences can be a private database stored within the computer system 100, or a public database such as those available through the Internet.

The process 200 begins at a start state 201 and then moves to a state 202 wherein the new sequence to be compared is stored to a memory in a computer system 100. As discussed above, the memory could be any type of memory, including RAM or an internal storage device.

The process 200 then moves to a state 204 wherein a database of sequences is opened for analysis and comparison. The process 200 then moves to a state 206 wherein the first sequence stored in the database is read into a memory on the computer. A comparison is then performed at a state 210 to determine if the first sequence is the same as the second sequence. It is important to note that this step is not limited to performing an exact comparison between the new sequence and the first sequence in the database. Well-known methods are known to those of skill in the art for comparing two nucleotide sequences, even if they are not identical. For example, gaps can be introduced into one sequence in order to raise the homology level between the two tested sequences. The parameters that control whether gaps or other features are introduced into a sequence during comparison are normally entered by the user of the computer system.

Once a comparison of the two sequences has been performed at the state 210, a determination is made at a decision state 210 whether the two sequences are the same. Of course, the term “same” is not limited to sequences that are absolutely identical. Sequences that are within the homology parameters entered by the user will be marked as “same” in the process 200.

If a determination is made that the two sequences are the same, the process 200 moves to a state 214 wherein the name of the sequence from the database is displayed to the user. This state notifies the user that the sequence with the displayed name fulfills the homology constraints that were entered. Once the name of the stored sequence is displayed to the user, the process 200 moves to a decision state 218 wherein a determination is made whether more sequences exist in the database. If no more sequences exist in the database, then the process 200 terminates at an end state 220. However, if more sequences do exist in the database, then the process 200 moves to a state 224 wherein a pointer is moved to the next sequence in the database so that it can be compared to the new sequence. In this manner, the new sequence is aligned and compared with every sequence in the database.

It should be noted that if a determination had been made at the decision state 212 that the sequences were not homologous, then the process 200 would move immediately to the decision state 218 in order to determine if any other sequences were available in the database for comparison.

Accordingly, one aspect of the present invention is a computer system comprising a processor, a data storage device having stored thereon a nucleic acid code of the invention, a data storage device having retrievably stored thereon reference nucleotide sequences to be compared to the nucleic acid code of the invention and a sequence comparer for conducting the comparison. The sequence comparer may indicate a homology level between the sequences compared or identify structural motifs in the nucleic acid code of the invention or it may identify structural motifs in sequences which are compared to these nucleic acid codes. In some embodiments, the data storage device may have stored thereon the sequences of at least 2, 5, 10, 15, 20, 25, 30, 50, 100, 200, 500, 1000 or all of the nucleic acid codes of the invention.

In one aspect, the methods and systems allow the identification of nucleotide sequences, including nucleotide sequences comprised in specific genes and/or nucleotide sequence contigs which contain sequence homologous to a nucleic acid code of the invention. The methods and systems may be used for example to position a biallelic marker of the invention in the human genome, on a contig or within a gene. The methods may also be used in identifying biallelic markers of the invention that are located on a particular sequence, as well as to identify further genetic markers, including further biallelic markers located on said contig or gene sequence which contains a nucleic acid code of the invention.

The invention thus encompasses a method for determining the position of a map-related biallelic marker on a nucleotide sequence comprising the steps of a) reading a first sequence and a second sequence comprising a map-related biallelic marker of the invention through the use of a computer program which compares sequences; b) determining if said biallelic marker is localized on said first sequence. Optionally, the method comprises determining the position of the polymorphic base of said first sequence. Step b) preferably comprises determining differences between said first and second sequence with said computer program. The method may further comprise determining the position of the second sequence within the first sequence. Preferably, said second sequence comprises at least 8, 10, 12, 15, 18, 20, 25, 30, 47 nucleotides of a map-related biallelic marker selected from the group of consisting of the biallelic markers of SEQ ID Nos. 1 to 171, 1 to 100, 101 to 162 and 163 to 171.

Another aspect of the present invention is a method for determining the level of homology between a nucleic acid code of the invention and a reference nucleotide sequence, comprising the steps of reading the nucleic acid code and the reference nucleotide sequence through the use of a computer program which determines homology levels and determining homology between the nucleic acid code and the reference nucleotide sequence with the computer program. The computer program may be any of a number of computer programs for determining homology levels, including those specifically enumerated herein, including BLAST2N with the default parameters or with any modified parameters. The method may be implemented using the computer systems described above. The method may also be performed by reading 2, 5, 10, 15, 20, 25, 30, 50, 100, 200, 500, 1000 or all of the above described nucleic acid codes of the invention through the use of the computer program and determining homology between the nucleic acid codes and reference nucleotide sequences.

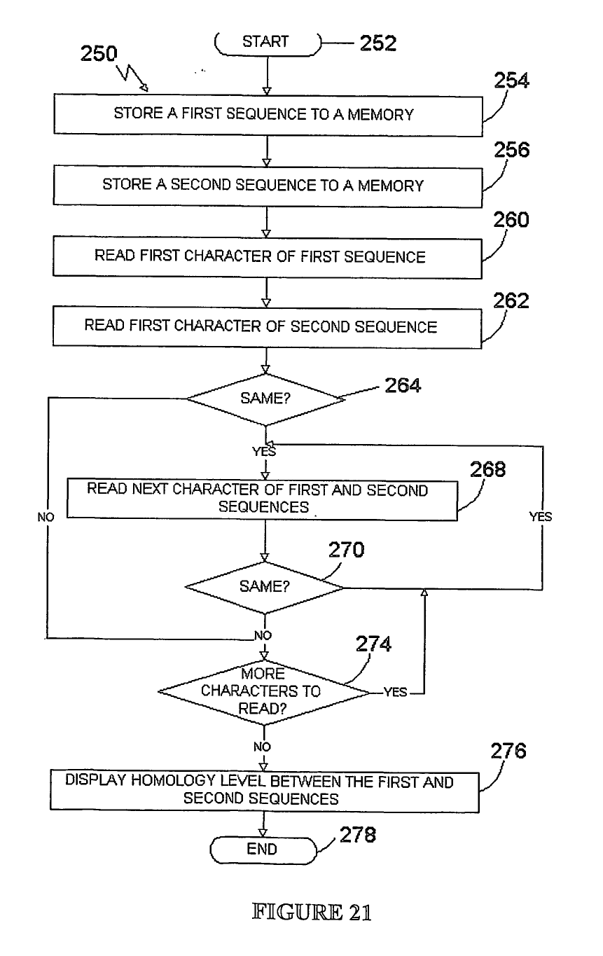

FIG. 21 is a flow diagram illustrating one embodiment of a process 250 in a computer for determining whether two sequences are homologous. The process 250 begins at a start state 252 and then moves to a state 254 wherein a first sequence to be compared is stored to a memory. The second sequence to be compared is then stored to a memory at a state 256. The process 250 then moves to a state 260 wherein the first character in the first sequence is read and then to a state 262 wherein the first character of the second sequence is read. It should be understood that if the sequence is a nucleotide sequence, then the character would normally be either A, T, C, G or U.

A determination is then made at a decision state 264 whether the two characters are the same. If they are the same, then the process 250 moves to a state 268 wherein the next characters in the first and second sequences are read. A determination is then made whether the next characters are the same. If they are, then the process 250 continues this loop until two characters are not the same. If a determination is made that the next two characters are not the same, the process 250 moves to a decision state 274 to determine whether there are any more characters either sequence to read.

If there aren't any more characters to read, then the process 250 moves to a state 276 wherein the level of homology between the first and second sequences is displayed to the user. The level of homology is determined by calculating the proportion of characters between the sequences that were the same out of the total number of sequences in the first sequence. Thus, if every character in a first 100 nucleotide sequence aligned with a every character in a second sequence, the homology level would be 100%.

Alternatively, the computer program may be a computer program which compares the nucleotide sequences of the nucleic acid codes of the present invention, to reference nucleotide sequences in order to determine whether the nucleic acid code of the invention differs from a reference nucleic acid sequence at one or more positions. Optionally such a program records the length and identity of inserted, deleted or substituted nucleotides with respect to the sequence of either the reference polynucleotide or the nucleic acid code of the invention. In one embodiment, the computer program may be a program which determines whether a reference nucleotide sequence contains one or more single nucleotide polymorphisms (SNP) with respect to the nucleotide sequences of the nucleic acid codes of the invention. These single nucleotide polymorphisms may each comprise a single base substitution, insertion, or deletion.

Accordingly, another aspect of the present invention is a method for determining whether a nucleic acid code of the invention differs at one or more nucleotides from a reference nucleotide sequence comprising the steps of reading the nucleic acid code and the reference nucleotide sequence through use of a computer program which identifies differences between nucleic acid sequences and identifying differences between the nucleic acid code and the reference nucleotide sequence with the computer program. In some embodiments, the computer program is a program which identifies single nucleotide polymorphisms in a reference nucleotide sequence. The method may be implemented by the computer systems described above and the method illustrated in FIG. 21. The method may also be performed by reading at least 2, 5, 10, 15, 20, 25, 30, 50, 100, 200, 500, 1000 or all of the nucleic acid codes of the invention and the reference nucleotide sequences through the use of the computer program and identifying differences between the nucleic acid codes and the reference nucleotide sequences with the computer program.

In other embodiments the computer based systems described above may further comprise a primer or probe generator for identifying a nucleotide sequence which may serve as primer or probe for use in assays for genotyping a biallelic marker of the invention. Methods thus include reading the polynucleotide code of the invention through use of a computer program which identifies primer or probe sequences, and identifying a primer or probe using the computer program.

The nucleic acid codes of the invention or the polypeptide codes of the invention may be stored and manipulated in a variety of data processor programs in a variety of formats. For example, they may be stored as text in a word processing file, such as MicrosoftWORD or WORDPERFECT or as an ASCII file in a variety of database programs familiar to those of skill in the art, such as DB2, SYBASE, or ORACLE. In addition, many computer programs and databases may be used as sequence comparers, identifiers, or sources of reference nucleotide or polypeptide sequences to be compared to the nucleic acid codes of the invention or the polypeptide codes of the invention. The following list is intended not to limit the invention but to provide guidance to programs and databases which are useful with the nucleic acid codes of the invention or the polypeptide codes of the invention. The programs and databases which may be used include, but are not limited to: MacPattern (EMBL), DiscoveryBase (Molecular Applications Group), GeneMine Molecular Applications Group), Look (Molecular Applications Group), MacLook (Molecular Applications Group), BLAST and BLAST2 (NCB1), BLASTN and BLASTX (Altschul et al, 1990), FASTA (Pearson and Lipman, 1988), FASTDB (Brutlag et al., 1990), Profile hidden Markov models such as HMMER (HMMs: R. Durbin, S. Eddy, A. Krogh, and G. Mitchison, Biological sequence analysis: probabilistic models of proteins and nucleic acids, Cambridge University Press, 1998), Catalyst (Molecular Simulations Inc.), Catalyst/SHAPE (Molecular Simulations Inc.), Cerius².DBAccess (Molecular Simulations Inc.), HypoGen (Molecular Simulations Inc.), Insight II, (Molecular Simulations Inc.), Discover (Molecular Simulations Inc.), CHARMM (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), ISIS (Molecular Simulations Inc.), WebLab (Molecular Simulations Inc.), WebLab Diversity Explorer (Molecular Simulations Inc.), Gene Explorer (Molecular Simulations Inc.), the MDL Available Chemicals Directory database, the MDL Drug Data Report data base, the Comprehensive Medicinal Chemistry database, Derwents's World Drug Index database, the BioByteMasterFile database, the Genbank database, and the Genseqn database. Many other programs and databases would be apparent to one of skill in the art given the present disclosure.

It should be noted that the nucleic acid codes of the invention further encompass all of the polynucleotides disclosed, described or claimed in the present application. Moreover, the present invention specifically contemplates the storage of such codes on computer readable media and computer systems individually or in any combination, as well as the use of such codes and combinations in the methods of VI.

VII. Mapping and Maps Comprising the Biallelic Markers of the Invention

The human haploid genome contains an estimated 80,000 to 100,000 or more genes scattered on a 3×10⁹ base-long double stranded DNA shared among the 24 chromosomes. Each human being is diploid, i.e. possesses two haploid genomes, one from paternal origin, the other from maternal origin. The sequence of the human genome varies among individuals in a population About 10⁷ sites scattered along the 3×10⁹ base pairs of DNA are polymorphic, existing in at least two variant forms called alleles. Most of these polymorphic sites are generated by single base substitution mutations and are biallelic. Less than 10⁵ polymorphic sites are due to more complex changes and are very often multi-allelic, i.e. exist in more than two allelic forms. At a given polymorphic site, any individual (diploid), can be either homozygous (twice the same allele) or heterozygous (two different alleles). A given polymorphism or rare mutation can be either neutral (no effect on trait), or functional, i.e. responsible for a particular genetic trait.

Genetic Maps

The first step towards the identification of genes associated with a detectable trait, such as a disease or any other detectable trait, consists in the localization of genomic regions containing trait-causing genes using genetic mapping methods. The preferred traits contemplated within the present invention relate to fields of therapeutic interest; in particular embodiments, they will be disease traits and/or drug response traits, reflecting drug efficacy or toxicity. Traits can either be “binary”, e.g. diabetic vs. non diabetic, or “quantitative”, e.g. elevated blood pressure. Individuals affected by a quantitative trait can be classified according to an appropriate scale of trait values, e.g. blood pressure ranges. Each trait value range can then be analyzed as a binary trait. Patients showing a trait value within one such range will be studied in comparison with patients showing a trait value outside of this range. In such a case, genetic analysis methods will be applied to subpopulations of individuals showing trait values within defined ranges.

Genetic mapping involves the analysis of the segregation of polymorphic loci in trait positive and trait-negative populations. Polymorphic loci constitute a small fraction of the human genome (less than 1%), compared to the vast majority of human genomic DNA which is identical in sequence among the chromosomes of different individuals. Among all existing human polymorphic loci, genetic markers can be defined as genome-derived polynucleotides which are sufficiently polymorphic to allow a reasonable probability that a randomly selected person will be heterozygous, and thus informative for genetic analysis by methods such as linkage analysis or association studies.

A genetic map consists of a collection of polymorphic markers which have been positioned on the human chromosomes. Genetic maps may be combined with physical maps, collections of ordered overlapping fragments of genomic DNA whose arrangement along the human chromosomes is known. The optimal genetic map should possess the following characteristics:

the density of the genetic markers scattered along the genome should be sufficient to allow the identification and localization of any trait-related polymorphism,

each marker should have an adequate level of heterozygosity, so as to be informative in a large percentage of different meioses,

all markers should be easily typed on a routine basis, at a reasonable expense, and in a reasonable amount of time,

the entire set of markers per chromosome should be ordered in a highly reliable fashion.

However, while the above maps are optimal, it will be appreciated that the maps of the present invention may be used in the individual marker and haplotype association analyses described below without the necessity of determining the order of biallelic markers derived from a single BAC with respect to one another.

Construction of a Physical Map

The first step in constructing a high density genetic map of biallelic markers is the construction of a physical map. Physical maps consist of ordered, overlapping cloned fragments of genomic DNA covering a portion of the genome, preferably covering one or all chromosomes. Obtaining a physical map of the genome entails constructing and ordering a genomic DNA library. For an example of a complete explanation of the construction of a physical map from a BAC library see related PCT Application No. PCT/IB98/00 193 filed Jul. 17, 1998, the disclosure of which is incorporated herein by reference in its entirety. The methods disclosed therein can be used to generate larger more complete sets of markers and entire maps of the human genome comprising the map-relate biallelic markers of the invention.

Biallelic Markers

It will be appreciated that the ordered DNA fragments containing these groups of biallelic markers need not completely cover the genomic regions of these lengths but may instead be incomplete contigs having one or more gaps therein. As discussed in further detail below, biallelic markers may be used in single marker and haplotype association analyses regardless of the completeness of the corresponding physical contig harboring them.

Using the procedures above, 171 biallelic markers, each having two alleles, were identified using sequences obtained from BACs which had been localized on the genome. In some cases, markers were identified using pooled BACs and thereafter reassigned to individual BACs using STS screening procedures such as those described in Examples 1 and 2. The sequences of these biallelic markers are provided in the accompanying Sequence Listing as SEQ ID Nos. 1 to 171. Although the sequences of SEQ ID Nos. 1 to 171 will be used as exemplary markers throughout the present application, these markers are not limited to markers having the exact flanking sequences surrounding the polymorphic bases which are enumerated in SEQ ID Nos. 1 to 171. Rather, it will be appreciated that the flanking sequences surrounding the polymorphic bases of SEQ ID Nos. 1 to 171 may be lengthened or shortened to any extent compatible with their intended use and the present invention specifically contemplates such sequences. The sequences of these biallelic markers may be used to construct genomic maps as well as in the gene identification and diagnostic techniques described herein. It will be appreciated that the biallelic markers referred to herein may be of any length compatible with their intended use provided that the markers include the polymorphic base, and the present invention specifically contemplates such sequences.

In preferred embodiments, a biallelic marker map comprises at least 1, 2, 3, 4, 5, 10, 15, 20, 25, 30, 40, 50, 70, 85, 100 or all of the biallelic markers of SEQ ID Nos.: 1 to 171 or the sequences complementary thereto. In another embodiment, a biallelic marker map comprises at least 1, 2, 3, 4, 5, 10, 15, 20, 25, 30, 40, 50, 70, 85, 100, 200, 300, 500, 700, or 1000 biallelic markers selected from the group consisting of biallelic markers which are in linkage disequilibrium with the biallelic markers of SEQ ID Nos.: 1 to 171 or the sequences complementary thereto. In some embodiments a biallelic marker map comprises 1, 2, 3, 4, 5, 10, 15, 20, 25, 30, 40, 50, 70, 85, 100 or all of the biallelic markers of SEQ ID Nos.: 1 to 100 or the sequences complementary thereto. In another embodiment, a biallelic marker map comprises at least 1, 2, 3, 4, 5, 10, 15, 20, 25, 30, 40, 50, 70, 85, 100 biallelic markers selected from the group consisting of biallelic markers which are in linkage disequilibrium with the biallelic markers of SEQ ID Nos.: 1 to 100 or the sequences complementary thereto. In some embodiments a biallelic marker map comprises at least 1, 2, 3, 4, 5, 10, 15, 20, 25, 30, 40, 50 or all of the biallelic markers of SEQ ID Nos.: 101 to 162 or the sequences complementary thereto. In another embodiment, a biallelic marker map comprises at least 1, 2, 3, 4, 5, 10, 15, 20, 25, 30, 40, or 50 biallelic markers selected from the group consisting of biallelic markers which are in linkage disequilibrium with the biallelic markers of SEQ ID Nos.: 101 to 162 or the sequences complementary thereto. In some embodiments a biallelic marker map comprises at least 1, 5, 8, or all of the biallelic markers of SEQ ID Nos.: 163 to 171 or the sequences complementary thereto. In another embodiment, a biallelic marker map comprises at least 1, 2, 3, 4, 5, 10, 15, 20, 25, 30, 40, 50, 70, 85, 100, 200, 300, 500, 700 or 1000 biallelic markers selected from the group consisting of biallelic markers which are in linkage disequilibrium with the biallelic markers of SEQ ID Nos.: 163 to 171 or the sequences complementary thereto. In yet another embodiment, a biallelic marker map further comprises at least 1, 2, 3, 4, 5, 10, 15, 20, 25, 30, 40, 50, 70, 85, 100, 200, 300, 500, 700, or 1000 biallelic markers selected from the group consisting of the biallelic markers of SEQ ID Nos. 1 to 3908 of copending U.S. patent application Ser. No. 09/422,978, titled “Biallelic markers for use in constructing a high density disequilibrium map of the human genome”.

In further embodiments, a biallelic marker map comprises one or more, or all, of said map-related markers which are localized on chromosome 3, 10 or 19. In particular, a biallelic marker map comprises at least 1, 2, 3, 4, 5, 10, 15, 20, 25, 30, 40, 50, 70, 85, 100 biallelic markers, wherein at least 1, 2, 3, 4, 5, 10, 15, 20, 25, 30, 40, 50, 100, 150 of said biallelic markers are selected from the group of biallelic markers consisting of:

chromosome 3 biallelic markers: (a) SEQ ID Nos. 8, 10, 12, 13, 14, 15, 16, 17, 18, 19, 20, 23, 24, 25, 26, 27, 70, 72, 73, 74, 75, 76, 77; and (b) SEQ ID Nos. 102, 105, 106, 107, 110, 111, 117, 118, 119, 120, 121, 122, 123, 124, 125, 126, 127, 159, 160, 161; and (c) 163, 166, 167;

chromosome 10 biallelic markers: (a) SEQ ID Nos. 1, 2, 3, 4, 5, 6, 7, 9, 11, 21, 22, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 71, 78, 79, 80, 81, 82, 83, 84, 85, 86, 87, 88, 89, 90, 91, 92, 93, 94, 95, 96, 97, 98, 99, 100; (b) SEQ ID Nos. 101, 103, 104, 108, 109, 112, 113, 114, 115, 116, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158; and (c) SEQ ID Nos. 164, 165, 168, 169, 170, 171; and

chromosome 19 biallelic marker: (a) SEQ ID No. 162.

Ordering of Biallelic Markers

Biallelic markers can be ordered to determine their positions along chromosomes, preferably subchromosomal regions, by methods known in the art as well as those disclosed in PCT Application No. PCT/IB98/00193 filed Jul. 17, 1998, and U.S. patent application Ser. No. 09/8422,978, the disclosures of which are incorporated herein in their entireties.

The positions of the biallelic markers along chromosomes may be determined using a variety of methodologies. In one approach, radiation hybrid mapping is used. Radiation hybrid (RH) mapping is a somatic cell genetic approach that can be used for high resolution mapping of the human genome. In this approach, cell lines containing one or more human chromosomes are lethally irradiated, breaking each chromosome into fragments whose size depends on the radiation dose. These fragments are rescued by fusion with cultured rodent cells, yielding subclones containing different portions of the human genome. This technique is described by Benham et al. (Genomics 4:509-517, 1989) and Cox et al., (Science 250:245-250, 1990), the entire contents of which are hereby incorporated by reference. The random and independent nature of the subclones permits efficient mapping of any human genome marker. Human DNA isolated from a panel of 80-100 cell lines provides a mapping reagent for ordering biallelic markers. In this approach, the frequency of breakage between markers is used to measure distance, allowing construction of fine resolution maps as has been done for ESTs (Schuler et al., Science 274:540-546, 1996, hereby incorporated herein by reference in its entirety).

RH mapping has been used to generate a high-resolution whole genome radiation hybrid map of human chromosome 17q22-q25.3 across the genes for growth hormone (GH) and thymidine kinase (TK) (Foster et al., Genomics 33:185-192, 1996), the region surrounding the Gorlin syndrome gene (Obermayr et al., Eur. J. Hum. Genet. 4:242-245, 1996), 60 loci covering the entire short arm of chromosome 12 (Raeymaekers et al., Genomics 29:170-178, 1995), the region of human chromosome 22 containing the neurofibromatosis type 2 locus (Frazer et al., Genomics 14:574-584, 1992) and 13 loci on the long arm of chromosome 5 (Warrington et al., Genomics 11:701-708, 1991).

Alternatively, PCR based techniques and human-rodent somatic cell hybrids may be used to determine the positions of the biallelic markers on the chromosomes. In such approaches, oligonucleotide primer pairs which are capable of generating amplification products containing the polymorphic bases of the biallelic markers are designed. Preferably, the oligonucleotide primers are 18-23 bp in length and are designed for PCR amplification. The creation of PCR primers from known sequences is well known to those with skill in the art. For a review of PCR technology see Erlich, H.A., PCR Technology: Principles and Applications for DNA Amplification. 1992. W. H. Freeman and Co., New York.

The primers are used in polymerase chain reactions (PCR) to amplify templates from total human genomic DNA. PCR conditions are as follows: 60 ng of genomic DNA is used as a template for PCR with 80 ng of each oligonucleotide primer, 0.6 unit of Taq polymerase, and 1 mCu of a ³²P-labeled deoxycytidine triphosphate. The PCR is performed in a microplate thermocycler (Techne) under the following conditions: 30 cycles of 94° C., 1.4 min; 55° C., 2 min; and 72° C., 2 min; with a final extension at 72° C. for 10 min. The amplified products are analyzed on a 6% polyacrylamide sequencing gel and visualized by autoradiography. If the length of the resulting PCR product is identical to the length expected for an amplification product containing the polymorphic base of the biallelic marker, then the PCR reaction is repeated with DNA templates from two panels of human-rodent somatic cell hybrids, BIOS PCRable DNA (BIOS Corporation) and NIGMS Human-Rodent Somatic Cell Hybrid Mapping Panel Number 1 (NIGMS, Camden, N.J.).

PCR is used to screen a series of somatic cell hybrid cell lines containing defined sets of human chromosomes for the presence of a given biallelic marker. DNA is isolated from the somatic hybrids and used as starting templates for PCR reactions using the primer pairs from the biallelic marker. Only those somatic cell hybrids with chromosomes containing the human sequence corresponding to the biallelic marker will yield an amplified fragment. The biallelic markers are assigned to a chromosome by analysis of the segregation pattern of PCR products from the somatic hybrid DNA templates. The single human chromosome present in all cell hybrids that give rise to an amplified fragment is the chromosome containing that biallelic marker. For a review of techniques and analysis of results from somatic cell gene mapping experiments. (See Ledbetter et al., Genomics 6:475481 (1990 the disclosure of which is incorporated herein by reference in its entirety).)

Example 2 describes a preferred method for positioning of biallelic markers on clones, such as BAC clones, obtained from genomic DNA libraries. Using such procedures, a number of BAC clones carrying selected biallelic markers can be isolated. The position of these BAC clones on the human genome can be defined by performing STS screening as described in Example 1. Preferably, to decrease the number of STSs to be tested, each BAC can be localized on chromosomal or subchromosomal regions by procedures such as those described in Examples 3 and 4. This localization will allow the selection of a subset of STSs corresponding to the identified chromosomal or subchromosomal region. Testing each BAC with such a subset of STSs and taking account of the position and order of the STSs along the genome will allow a refined positioning of the corresponding biallelic marker along the genome.

In other embodiments, if the DNA library used to isolate BAC inserts or any type of genomic DNA fragments harboring the selected biallelic markers already constitute a physical map of the genome or any portion thereof, using the known order of the DNA fragments will allow the order of the biallelic markers to be established.

As discussed above, it will be appreciated that markers carried by the same fragment of genomic DNA, such as the insert in a BAC clone, need not necessarily be ordered with respect to one another within the genomic fragment to conduct single point or haplotype association analyses. However, in other embodiments of the present maps, the order of biallelic markers carried by the same fragment of genomic DNA may be determined.

While the subchromosomal locations of the map-related biallelic markers of the invention for which are provided herein, the positions of further biallelic markers used to construct the maps of the present invention may be assigned to using Fluorescence In Situ Hybridization (FISH) (Cherif et al., Proc. Natl. Acad. Sci U.S.A., 87:6639-6643 (1990), the disclosure of which is incorporated herein by reference in its entirety). FISH analysis is described in Example 3.

The ordering analyses may be conducted to generate an integrated genome wide genetic map comprising about 20,000, 40,000, 60,000, 80,000, 100,000, 120,000 biallelic markers with a roughly consistent number of biallelic marker per BAC. In some embodiments, the map includes one or more markers selected from the group consisting of the sequences of SEQ ID Nos. 1 to 171, 1 to 100, 101 to 162, 163 to 171 or the sequences complementary thereto.

Alternatively, maps having the above-specified average numbers of biallelic markers per BAC which comprise smaller portions of the genome, such as a set of chromosomes, a single chromosome, a particular subchromosomal region, or any other desired portion of the genome, may also be constructed using the procedures provided herein.

In some embodiments, the biallelic markers in the map are separated from one another by an average distance of 10-200 kb, 15-150 kb, 20-100 kb, 100-150 kb, 50-100 kb, or 25-50 kb. Maps having the above-specified intermarker distances which comprise smaller portions of the genome, such as a set of chromosomes, a single chromosome, a particular subchromosomal region, or any other desired portion of the genome, may also be constructed using the procedures provided herein.

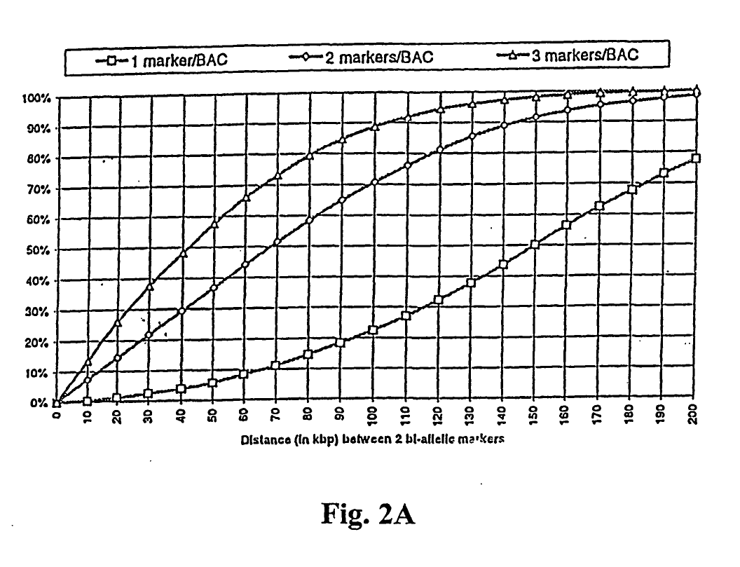

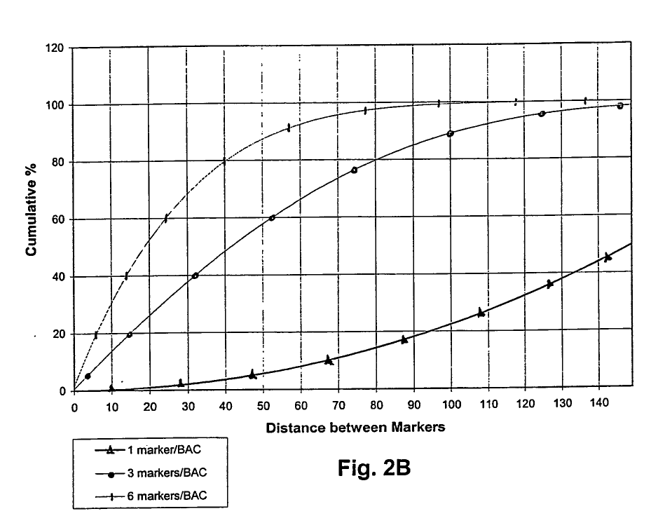

FIG. 2, showing the results of computer simulations of the distribution of inter-marker spacing on a randomly distributed set of biallelic markers, indicates the percentage of biallelic markers which will be spaced a given distance apart for a given number of markers/BAC in the genomic map (assuming 20,000 BACs constituting a minimally overlapping array covering the entire genome are evaluated). One hundred iterations were performed for each simulation (20,000 marker map, 40,000 marker map, 60,000 marker map, 120,000 marker map).

As illustrated in FIG. 2A, 98% of inter-marker distances will be lower than 150 kb provided 60,000 evenly distributed markers are generated (3 per BAC); 90% of inter-marker distances will be lower than 150 kb provided 40,000 evenly distributed markers are generated (2 per BAC); and 50% of inter-marker distances will be lower than 150 kb provided 20,000 evenly distributed markers are generated (1 per BAC).

As illustrated in FIG. 2B, 98% of inter-marker distances will be lower than 80 kb provided 120,000 evenly distributed markers are generated (6 per BAC); 80% of inter-marker distances will be lower than 80 kb provided 60,000 evenly distributed markers are generated (3 per BAC); and 15% of inter-marker distances will be lower than 80 kb provided 20,000 evenly distributed markers are generated (1 per BAC).

As already mentioned, high density biallelic marker maps allow association studies to be performed to identify genes involved in complex traits.

Table 7 provides the genomic location of biallelic markers described herein. Listed are chromosomal regions and subregions to which biallelic markers were assigned using the methods of Example 3 and by screening BAC sequences against published and unpublished STSs. The locations of markers listed in Table 7 are locations for which adjacent STSs are publicly available. The column “adjacent STS” provides the public accession numbers of STSs localized on the same BAC with the subject biallelic marker as well as aliases for said STSs. As noted above, all of the marker localizations provided in Table 7 are confirmed by fluorescence in situ hybridization methods and public STS screening.

Linkage Disequilibrium

The present invention then also concerns biallelic markers in linkage disequilibrium with the specific biallelic markers described above and which are expected to present similar characteristics in terms of their respective association with a given trait. In a preferred embodiment, the present invention concerns the biallelic markers that are in linkage disequilibrium with the biallelic markers of SEQ ID Nos. 1 to 171, 1 to 100, 101 to 162, 163 to 171 or the sequences complementary thereto.

LD among a set of biallelic markers having an adequate heterozygosity rate can be determined by genotyping between 50 and 1000 unrelated individuals, preferably between 75 and 200, more preferably around 100. Genotyping a biallelic marker consists of determining the specific allele carried by an individual at the given polymorphic base of the biallelic marker. Genotyping can be performed using similar methods as those described above for the generation of the biallelic markers, or using other genotyping methods such as those further described below.

Genome-wide linkage disequilibrium mapping aims at identifying, for any trait-causing allele being searched, at least one biallelic marker in linkage disequilibrium with said trait-causing allele. Preferably, in order to enhance the power of linkage disequilibrium maps, in some embodiments, the biallelic markers therein have average inter-marker distances of 150 kb or less, 75 kb or less, or 50 kb or less, 30 kb or less, or 25 kb or less to accommodate the fact that, in some regions of the genome, the detection of linkage disequilibrium requires lower inter-marker distances.

The present invention provides methods to generate biallelic marker maps with average inter-marker distances of 150 kb or less. In some embodiments, the mean distance between biallelic markers constituting the high density map will be less than 75 kb, preferably less than 50 kb. Further preferred maps according to the present invention contain markers that are less than 37.5 kb apart. In highly preferred embodiments, the average inter-marker spacing for the biallelic markers constituting very high density maps is less than 30 kb, most preferably less than 25 kb.

Genetic maps containing biallelic markers (including the biallelic markers of SEQ ID Nos. 1 to 171, 1 to 100, 101 to 162, 163 to 171 or the sequences complementary thereto) may be used to identify and isolate genes associated with detectable traits. The use of the genetic maps of the present invention is described in more detail below.

VIII. Use of High Density Biallelic Marker Maps to Identify Genes Associated with Detectable Traits

One embodiment of the present invention comprises methods for identifying and isolating genes associated with a detectable trait using the biallelic marker maps of the present invention.

In the past, the identification of genes linked with detectable traits has relied on a statistical approach called linkage analysis. Linkage analysis is based upon establishing a correlation between the transmission of genetic markers and that of a specific trait throughout generations within a family. In this approach, all members of a series of affected families are genotyped with a few hundred markers, typically microsatellite markers, which are distributed at an average density of one every 10 Mb. By comparing genotypes in all family members, one can attribute sets of alleles to parental haploid genomes (haplotyping or phase determination). The origin of recombined fragments is then determined in the offspring of all families. Those that co-segregate with the trait are tracked. After pooling data from all families, statistical methods are used to determine the likelihood that the marker and the trait are segregating independently in all families. As a result of the statistical analysis, one or several regions having a high probability of harboring a gene linked to the trait are selected as candidates for further analysis. The result of linkage analysis is considered as significant (i.e. there is a high probability that the region contains a gene involved in a detectable trait) when the chance of independent segregation of the marker and the trait is lower than 1 in 1000 (expressed as a LOD score >3). Generally, the length of the candidate region identified using linkage analysis is between 2 and 20 Mb.

Once a candidate region is identified as described above, analysis of recombinant individuals using additional markers allows further delineation of the candidate linked region.

Linkage analysis studies have generally relied on the use of a maximum of 5,000 microsatellite markers, thus limiting the maximum theoretical attainable resolution of linkage analysis to ca. 600 kb on average.

Linkage analysis has been successfully applied to map simple genetic traits that show clear Mendelian inheritance patterns and which have a high penetrance (penetrance is the ratio between the number of trait-positive carriers of allele a and the total number of a carriers in the population). About 100 pathological trait-causing genes were discovered using linkage analysis over the last 10 years. In most of these cases, the majority of affected individuals had affected relatives and the detectable trait was rare in the general population (frequencies less than 0.1%). In about 10 cases, such as Alzheimer's Disease, breast cancer, and Type II diabetes, the detectable trait was more common but the allele associated with the detectable trait was rare in the affected population. Thus, the alleles associated with these traits were not responsible for the trait in all sporadic cases.

Linkage analysis suffers from a variety of drawbacks. First, linkage analysis is limited by its reliance on the choice of a genetic model suitable for each studied trait. Furthermore, as already mentioned, the resolution attainable using linkage analysis is limited, and complementary studies are required to refine the analysis of the typical 2 Mb to 20 Mb regions initially identified through linkage analysis.

In addition, linkage analysis approaches have proven difficult when applied to complex genetic traits, such as those due to the combined action of multiple genes and/or environmental factors. In such cases, too large an effort and cost are needed to recruit the adequate number of affected families required for applying linkage analysis to these situations, as recently discussed by Risch, N. and Merikangas, K. (Science 273:1516-1517 (1996), the disclosure of which is incorporated herein by reference in its entirety).

Finally, linkage analysis cannot be applied to the study of traits for which no large informative families are available. Typically, this will be the case in any attempt to identify trait-causing alleles involved in sporadic cases, such as alleles associated with positive or negative responses to drug treatment.

The present genetic maps and biallelic markers (including the biallelic markers of SEQ ID Nos. 1 to 171, 1 to 100, 101 to 162, 163 to 171 or the sequences complementary thereto) may be used to identify and isolate genes associated with detectable traits using association studies, an approach which does not require the use of affected families and which permits the identification of genes associated with sporadic traits.

Association Studies

As already mentioned, any gene responsible or partly responsible for a given trait will be in linkage disequilibrium with some flanking markers. To map such a gene, specific alleles of these flanking markers which are associated with the gene or genes responsible for the trait are identified. Although the following discussion of techniques for finding the gene or genes associated with a particular trait using linkage disequilibrium mapping, refers to locating a single gene which is responsible for the trait, it will be appreciated that the same techniques may also be used to identify genes which are partially responsible for the trait.

As further described herein, it will be appreciated the present invention may be used to identify genes responsible for any given trait. However, as the biallelic markers of the invention are located in genomic regions suspected to contain a genetic determinant of obesity or a related disorder, the detectable trait may preferably be an obesity disorder. As described above, by way of example and not limitation, examples of obesity disorders may comprise obesity-related atherosclerosis, obesity-related insulin resistance, obesity-related hypertension, microangiopathic lesions resulting from obesity-related Type II diabetes, ocular lesions caused by microangiopathy in obese individuals with Type II diabetes, and renal lesions caused by microangiopathy in obese individuals with Type II diabetes. Obesity-related disorders may also include hyperinsulinemia and hyperglycemia.

Association studies may be conducted within the general population (as opposed to the linkage analysis techniques discussed above which are limited to studies performed on related individuals in one or several affected families).

Association between a biallelic marker A and a trait T may primarily occur as a result of three possible relationships between the biallelic marker and the trait.

First, allele a of biallelic marker A may be directly responsible for trait T (e.g., Apo Eε4 site A and Alzheimer's disease). However, since the majority of the biallelic markers used in genetic mapping studies are selected randomly, they mainly map outside of genes. Thus, the likelihood of allele a being a functional mutation directly related to trait T is very low.

Second, an association between a biallelic marker A and a trait T may also occur when the biallelic marker is very closely linked to the trait locus. In other words, an association occurs when allele a is in linkage disequilibrium with the trait-causing allele. When the biallelic marker is in close proximity to a gene responsible for the trait, more extensive genetic mapping will ultimately allow a gene to be discovered near the marker locus which carries mutations in people with trait T (i.e. the gene responsible for the trait or one of the genes responsible for the trait). As will be further exemplified below, using a group of biallelic markers which are in close proximity to the gene responsible for the trait the location of the causal gene can be deduced from the profile of the association curve between the biallelic markers and the trait. The causal gene will usually be found in the vicinity of the marker showing the highest association with the trait.

Finally, an association between a biallelic marker and a trait may occur when people with the trait and people without the trait correspond to genetically different subsets of the population who, coincidentally, also differ in the frequency of allele a (population stratification). This phenomenon may be avoided by using ethnically matched large heterogeneous samples.

Association studies are particularly suited to the efficient identification of genes that present common polymorphisms, and are involved in multifactorial traits whose frequency is relatively higher than that of diseases with monofactorial inheritance.

Association studies mainly consist of four steps: recruitment of trait-positive (T+) and control populations, preferably trait-negative (T−) populations with well-defined phenotypes, identification of a candidate region suspected of harboring a trait causing gene, identification of said gene among candidate genes in the region, and finally validation of mutation(s) responsible for the trait in said trait causing gene.

In a first step, the trait-positive should be well-defined, preferably the control phenotype is a well-defined trait-negative phenotype as well. In order to perform efficient and significant association studies such as those described herein, the trait under study should preferably follow a bimodal distribution in the population under study, presenting two clear non-overlapping phenotypes, trait-positive and trait-negative.

Nevertheless, in the absence of such a bimodal distribution (as may in fact be the case for complex genetic traits), any genetic trait may still be analyzed using the association method proposed herein by carefully selecting the individuals to be included in the trait-positive group and preferably the trait-negative phenotypic group as well. The selection procedure ideally involves selecting individuals at opposite ends of the non-bimodal phenotype spectrum of the trait under study, so as to include in these trait-positive and trait-negative populations individuals who clearly represent non-overlapping, preferably extreme phenotypes.

As discussed above, the definition of the inclusion criteria for the trait-positive and control populations is an important aspect of the present invention.

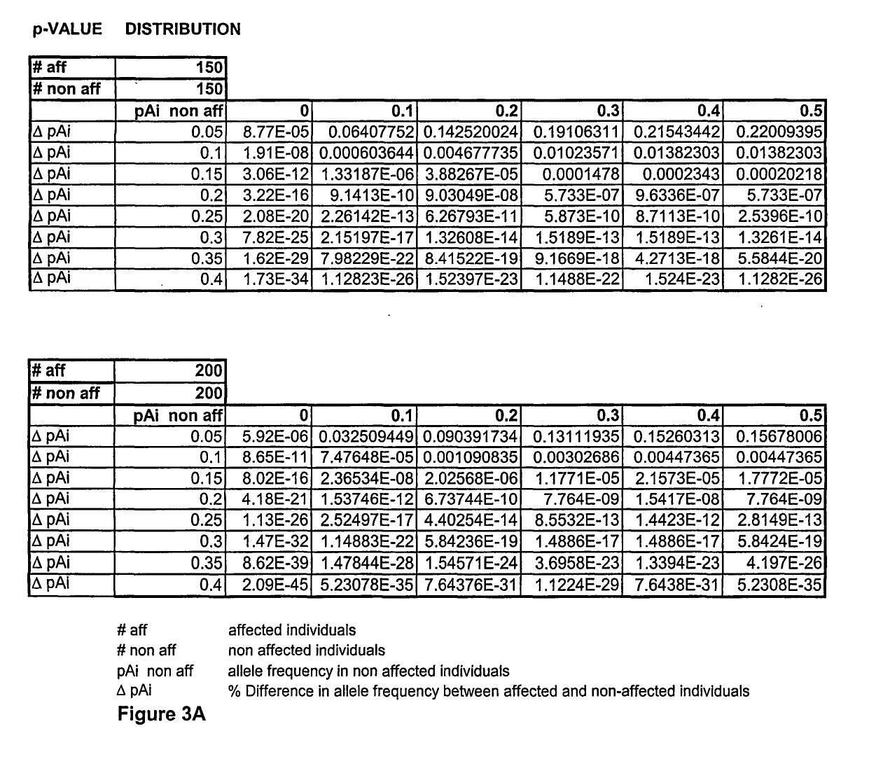

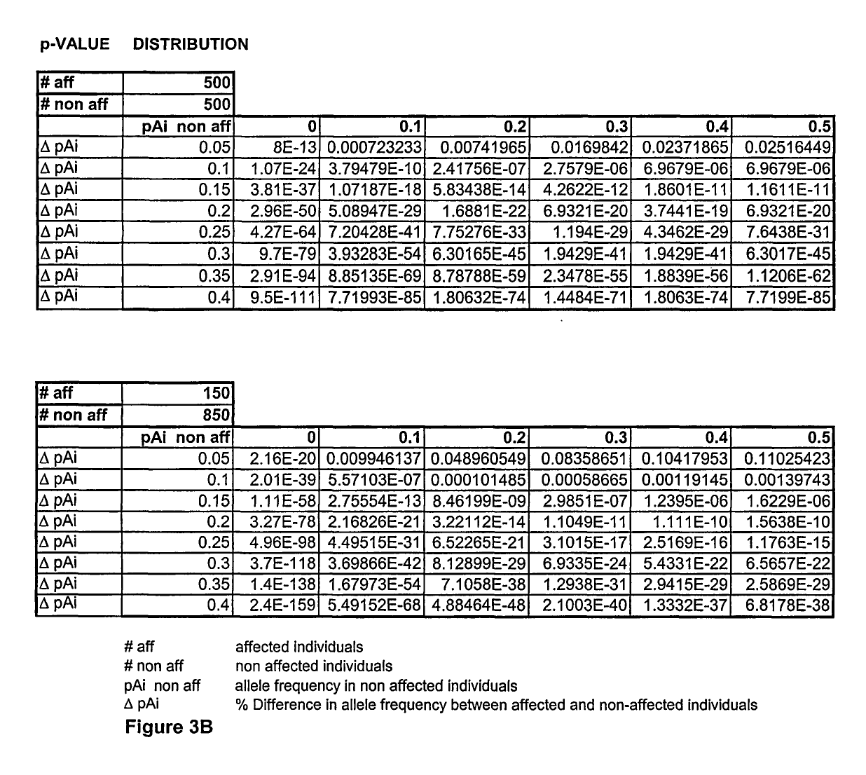

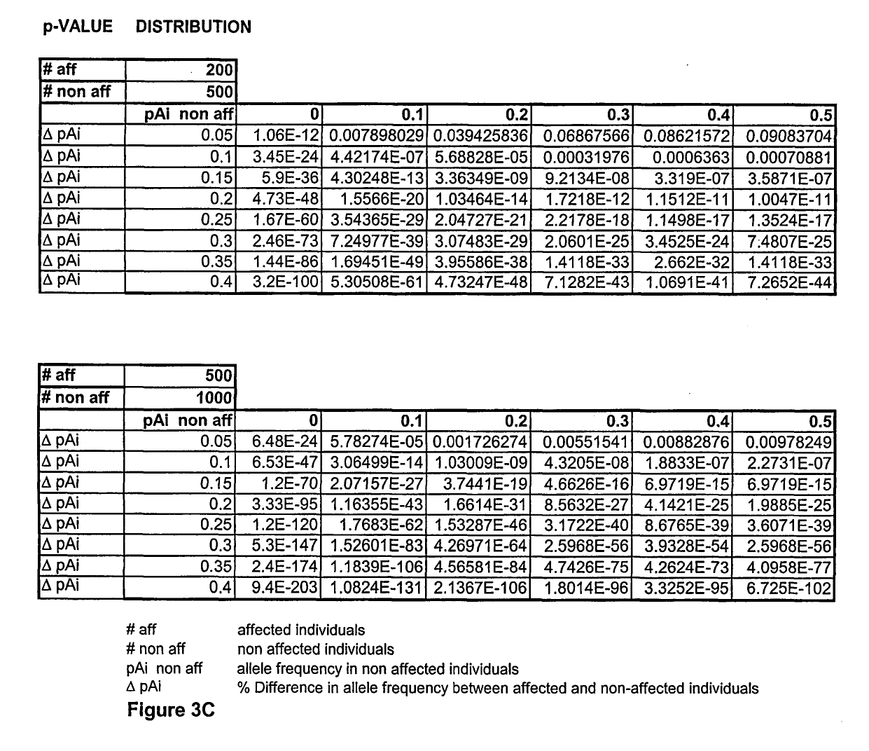

FIG. 3 shows, for a series of hypothetical sample sizes, the p-value significance obtained in association studies performed using individual markers from the high-density biallelic map, according to various hypotheses regarding the difference of allelic frequencies between the trait-positive and trait-negative samples. It indicates that, in all cases, samples ranging from 150 to 500 individuals are numerous enough to achieve statistical significance. It will be appreciated that bigger or smaller groups can be used to perform association studies according to the methods of the present invention.

In a second step, a marker/trait association study is performed that compares the genotype frequency of each biallelic marker in the above described trait-positive and trait-negative populations by means of a chi square statistical test (one degree of freedom). In addition to this single marker association analysis, a haplotype association analysis is performed to define the frequency and the type of the ancestral carrier haplotype. Haplotype analysis, by combining the informativeness of a set of biallelic markers increases the power of the association analysis, allowing false positive and/or negative data that may result from the single marker studies to be eliminated.

Genotyping can be performed using any method described in III, including the microsequencing procedure described in Example 8.

If a positive association with a trait is identified using an array of biallelic markers having a high enough density, the causal gene will be physically located in the vicinity of the associated markers, since the markers showing positive association with the trait are in linkage disequilibrium with the trait locus. Regions harboring a gene responsible for a particular trait which are identified through association studies using high density sets of biallelic markers will, on average, be 20-40 times shorter in length than those identified by linkage analysis.

Once a positive association is confirmed as described above, a third step consists of completely sequencing the BAC inserts harboring the markers identified in the association analyzes. These BACs are obtained through screening human genomic libraries with the markers probes and/or primers, as described above. Once a candidate region has been sequenced and analyzed, the functional sequences within the candidate region (e.g. exons, splice sites, promoters, and other potential regulatory regions) are scanned for mutations which are responsible for the trait by comparing the sequences of the functional regions in a selected number of trait-positive and trait-negative individuals using appropriate software. Tools for sequence analysis are further described in Example 9.

Finally, candidate mutations are then validated by screening a larger population of trait-positive and trait-negative individuals using genotyping techniques described below. Polymorphisms are confirmed as candidate mutations when the validation population shows association results compatible with those found between the mutation and the trait in the test population.

In practice, in order to define a region bearing a candidate gene, the trait-positive and trait-negative populations are genotyped using an appropriate number of biallelic markers. The markers may include one or more of the markers of SEQ ID Nos: 1 to 171, 1 to 100, 101 to 162, 163 to 171 or the sequences complementary thereto.

The markers used to define a region bearing a candidate gene may be distributed at an average density of 1 marker per 10-200 kb. Preferably, the markers used to define a region bearing a candidate gene are distributed at an average density of 1 marker every 15-150 kb. In further preferred embodiments, the markers used to define a region bearing a candidate gene are distributed at an average density of 1 marker every 20-100 kb. In yet another preferred embodiment, the markers used to define a region bearing a candidate gene are distributed at an average density of 1 marker every 100 to 150 kb. In a further highly preferred embodiment, the markers used to define a region bearing a candidate gene are distributed at an average density of 1 marker every 50 to 100 kb. In yet another embodiment, the biallelic markers used to define a region bearing a candidate gene are distributed at an average density of 1 marker every 25-50 kilobases. As mentioned above, in order to enhance the power of linkage disequilibrium based maps, in a preferred embodiment, the marker density of the map will be adapted to take the linkage disequilibrium distribution in the genomic region of interest into account.

In some embodiments, such as where performed on a genome-wide scale, the initial identification of a candidate genomic region harboring a gene associated with a detectable phenotype may be conducted using a preliminary map containing a few thousand biallelic markers. Thereafter, the genomic region harboring the gene responsible for the detectable trait may be better delineated using a map containing a larger number of biallelic markers. Furthermore, the genomic region harboring the gene responsible for the detectable trait may be further delineated using a high density map of biallelic markers. Finally, the gene associated with the detectable trait may be identified and isolated using a very high density biallelic marker map.

In other embodiments, a candidate genomic region suspected of harboring a gene associated with a detectable phenotype is delineated using a high density map or a large number of biallelic markers located in one or more genomic regions of interest. In particular, the genomic region selected may be a genomic region described above in the Background of the Invention section. Furthermore, the phenotype may be an obesity disorder.

Example 6 describes a procedure for identifying a candidate region harboring a gene associated with a detectable trait and provides simulated results for this procedure. It will be appreciated that although Example 6 compares the results of simulated analyzes using markers derived from maps having 3,000, 20,000, and 60,000 markers, the number of markers contained in the map is not restricted to these exemplary figures. Rather, Example 6 exemplifies the increasing refinement of the candidate region with increasing marker density. As increasing numbers of markers are used in the analysis, points in the association analysis become broad peaks. The gene associated with the detectable trait under investigation will lie within or near the region under the peak.

The statistical power of linkage disequilibrium mapping using a high density marker map is also reinforced by complementing the single point association analysis described above with a multi-marker association analysis of haplotype analysis described in IV. To improve the statistical power of the individual marker association analyses conducted as described above using maps of increasing marker densities, haplotype studies can be performed using groups of markers located in proximity to one another within regions of the genome. For example, using the methods described above in which the association of an individual marker with a detectable phenotype was analyzed using maps of 3,000 markers, 20,000 markers, and 60,000 markers, a series of haplotype studies can be performed using groups of contiguous markers from such maps or from maps having higher marker densities.

In a preferred embodiment, a series of successive haplotype studies including groups of markers spanning regions of more than 1 Mb may be performed. In some embodiments, the biallelic markers included in each of these groups may be located within a genomic region spanning less than 1 kb, from 1 to 5 kb, from 5 to 10 kb, from 10 to 25 kb, from 25 to 50 kb, from 50 to 150 kb, from 150 to 250 kb, from 250 to 500 kb, from 500 kb to 1 Mb, or more than 1 Mb. Preferably, the genomic regions containing the groups of biallelic markers used in the successive haplotype analyses are overlapping. It will be appreciated that the groups of biallelic markers need not completely cover the genomic regions of the above-specified lengths but may instead be obtained from incomplete contigs having one or more gaps therein. As discussed in further detail below, biallelic markers may be used in single point and haplotype association analyses regardless of the completeness of the corresponding physical contig harboring them.

Genome-wide mapping using association studies with dense enough arrays of markers permit a case-by-case best estimate of p-value significance thresholds. Given a test population comprising two ethnically matched trait-positive and control groups of about 50 to about 500 individuals or more, conducting the above described association studies will allow a p-value “cut-off” to be established by, for example, analyzing significant numbers of allele frequency differences or, in some cases where appropriate, running computer simulations or control studies as described in Examples 6, 15, and 26.

For a p-value above the threshold, a corresponding association between the trait and a studied marker will be deemed not significant, while for a p-value below such a threshold, said association will be deemed significant. If the p-value is significant, the genomic region around the marker will be further scrutinized for a trait-causing gene.

It is preferred that p-value significance thresholds be assessed for each case/control population comparison. Both the genetic distance between sampled population-“stratification”-and the dispersion due to random selection of samples may indeed influence the p-value significance thresholds.

It will be appreciated that the above approaches may be conducted on any scale (i.e. over the whole genome, a set of chromosomes, a single chromosome, a particular subchromosomal region, or any other desired portion of the genome). As mentioned above, once significance thresholds have been assessed, population sample sizes may be adapted as exemplified in FIG. 3.

Examples 7 and 15 below illustrates the increase in statistical power brought to an association study by a haplotype analysis.

The results described in Examples 5 and 7, generated from individual and haplotype studies using a biallelic marker set of an average density equal to ca. 40 kb in the region of an Alzheimer's disease trait causing gene, indicate that all biallelic markers of sufficient informative content located within a ca. 200 kb genomic region around a trait-causing allele can potentially be successfully used to localize a trait causing gene with the methods provided by the present invention. This conclusion is further supported by the results obtained through measuring the linkage disequilibrium between markers 99-365-344 or 99-359-308 and ApoE 4 Site A marker within Alzheimer's patients: as one could predict since linkage disequilibrium is the supporting basis for association studies, linkage disequilibrium between these pairs of markers was enhanced in the diseased population vs. the control population. In a similar way as the haplotype analysis enhanced the significance of the corresponding association studies.

Once a given polymorphic site has been found and characterized as a biallelic marker according to the methods of the present invention, several methods can be used in order to determine the specific allele carried by an individual at the given polymorphic base as described in III.

Location of a Gene Associated with Detectable Traits Once the candidate region has been delineated using the high density biallelic marker map, a sequence analysis process will allow the detection of all genes located within said region, together with a potential functional characterization of said genes. The identified functional features may allow preferred trait-causing candidates to be chosen from among the identified genes. More biallelic markers may then be generated within said candidate genes, and used to perform refined association studies that will support the identification of the trait causing gene. Sequence analysis processes are described in Example 9.

Examples 10-22 illustrate the application of the above methods using biallelic markers to identify a gene associated with a complex disease, prostate cancer, within a large candidate region. Additional details of the identification of the gene associated with prostate cancer are provided in the U.S. Patent Application titled “Biallelic markers for use in constructing a high density disequilibrium map of the human genome,” filed Oct. 20, 1999, the disclosure of which is incorporated herein by reference in its entirety.

The above methods were also used to identify biallelic markers in a gene which was an attractive candidate for a gene associated with obesity. Examples 23-26 show how the use of methods of the present invention allowed this gene to be identified as a gene responsible, at least partially, for obesity and obesity-related disorders in the studied populations. Additional details of the identification of the gene associated with obesity are provided in U.S. Patent Application entitled, “Polymorphic markers of the LSR gene,” filed Feb. 10, 2000, the disclosure of which are incorporated herein by reference in its entirety.

Alternatively, genes associated with detectable traits may be identified as follows. Candidate genomic regions suspected of harboring a gene associated with the trait may be identified using techniques such as those described herein. In such techniques, the allelic frequencies of biallelic markers are compared in nucleic acid samples derived from individuals expressing the detectable trait and individuals who do not express the detectable trait. In this manner, candidate genomic regions suspected of harboring a gene associated with the detectable trait under investigation are identified.

The existence of one or more genes associated with the detectable trait within the candidate region is confirmed by identifying more biallelic markers lying in the candidate region. A first haplotype analysis is performed for each possible combination of groups of biallelic markers within the genomic region suspected of harboring a trait-associated gene. For example, each group may comprise three biallelic markers. For each of the groups of markers, the frequency of each possible haplotype (for groups of three markers there are 8 possible haplotypes) in individuals expressing the trait and individuals who do not express the trait is estimated. For example, the a haplotype estimation method is applied as described in IV for example the haplotype frequencies may be estimated using the Expectation-Maximization method of Excoffier L and Slatkin M, Mol. Biol. Evol. 12:921-927 (1995), the disclosure of which is incorporated herein by reference in its entirety.

The frequencies of each of the possible haplotypes of the grouped markers (or each allele of individual markers) in individuals expressing the trait and individuals who do not express the trait are compared. For example, the frequencies may be compared by performing a chi-squared analysis. Within each group, the haplotype (or the allele of each individual marker) having the greatest association with the trait is selected. This process is repeated for each group of biallelic markers (or each allele of the individual markers) to generate a distribution of association values, which will be referred to herein as the “trait-associated” distribution.

A second haplotype analysis is performed for each possible combination of groups of biallelic markers within the genomic regions which are not suspected of harboring a trait-associated gene. For example, each group may comprise three biallelic markers. For each of the groups of markers, the frequency of each possible haplotype (for groups of three markers there are 8 possible haplotypes) in individuals expressing the trait and individuals who do not express the trait is estimated.

The frequencies of each of the possible haplotypes of the grouped markers (or each allele of individual markers) in individuals expressing the trait and individuals who do not express the trait are compared. For example, the frequencies may be compared by performing a chi-squared analysis. Within each group, the haplotype (or the allele of each individual marker) having the greatest association with the trait is selected. This process is repeated for each group of biallelic markers (or each allele of the individual markers) to generate a distribution of association values, which will be referred to herein as the “random” distribution.

The trait-associated distribution and the random distribution are then compared to one another to determine if there are significant differences between them. For example, the trait-associated distribution and the random distribution can be compared using either the Wilcoxon rank test (Noether, G. E. (1991) Introduction to statistics: “The nonparametric way”, Springer-Verlag, New York, Berlin, the disclosure of which is incorporated herein by reference in its entirety) or the Kolmogorov-Smirnov test (Saporta, G. (1990) “Probalites, analyse des donnees et statistiques” Technip editions, Paris, the disclosure of which is incorporated herein by reference in its entirety) or both the Wilcoxon rank test and the Kolmogorov-Smirnov test.

If the trait-associated distribution and the random distribution are found to be significantly different, the candidate genomic region is highly likely to contain a gene associated with the detectable trait. Accordingly, the candidate genomic region is evaluated more fully to isolate the trait-associated gene. Alternatively, if the trait-associated distribution and the random distribution are equal using the above analyses, the candidate genomic region is unlikely to contain a gene associated with the detectable trait. Accordingly, no further analysis of the candidate genomic region is performed.

While Examples 10 to 26 illustrate the use of the maps and markers of the present invention for identifying a new gene associated with a complex disease within a large genomic region for establishing that a candidate gene is, at least partially, responsible for a disease, the maps and markers of the present invention may also be used to identify one or more biallelic markers or one or more genes associated with other detectable phenotypes, including drug response, drug toxicity, or drug efficacy. The biallelic markers used in such drug response analyses or shown, using the methods of the present invention to be associated with such traits, may lie within or near genes responsible for or partly responsible for a particular disease, for example a disease against which the drug is meant to act, or may lie within genomic regions which are not responsible for or partly responsible for a disease. In the context of the present invention, a “positive response” to a medicament can be defined as comprising a reduction of the symptoms related to the disease or condition to be treated. In the context of the present invention, a “negative response” to a medicament can be defined as comprising either a lack of positive response to the medicament which does not lead to a symptom reduction or to a side-effect observed following administration of the medicament.

Drug efficacy, response and tolerance/toxicity can be considered as multifactorial traits involving a genetic component in the same way as complex diseases such as Alzheimer's disease, prostate cancer, hypertension or diabetes. As such, the identification of genes involved in drug efficacy and toxicity could be achieved following a positional cloning approach; e.g. performing linkage analysis within families in order to obtain the subchromosomal location of the gene(s). However, this type of analysis is actually impractical in the case of drug responsiveness, due to the lack of availability of familial cases. In fact, the likelihood of having more than one individual in a particular family being exposed to the same drug at the same time is very low. Therefore, drug efficacy and toxicity can only be analyzed as sporadic traits.

In order to conduct association studies to analyze the individual response to a given drug in groups of patients affected with a disease, up to four groups are screened to determine their patterns of biallelic markers using the techniques described above. The four groups are:

Non-diseased or random controls,

Diseased patients/drug responders,

Diseased patients/drug non-responders, and

Diseased patients/drug side effects.

In preferred embodiments, the above mentioned groups are recruited according to phenotyping criteria having the characteristics described above, so that the phenotypes defining the different groups are non-overlapping, preferably extreme phenotypes. In highly preferred embodiments, such phenotyping criteria have the bimodal distribution described above.

The final number and composition of the groups for each drug association study is adapted to the distribution of the above described phenotypes within the studied population.

After selecting a suitable population, association and haplotype analyses may be performed as described herein to identify one or more biallelic markers associated with drug response, preferably drug toxicity or drug efficacy. The identification of such one or more biallelic markers allows one to conduct diagnostic tests to determine whether the administration of a drug to an individual will result in drug response, preferably drug toxicity, or drug efficacy.

The methods described above for identifying a gene associated with prostate cancer and biallelic markers indicative of a risk of suffering from asthma may be utilized to identify genes associated with other detectable phenotypes. In particular, the above methods may be used with any marker or combination of markers included in the maps of the present invention, including the biallelic markers of SEQ ID Nos.: 1 to 171 or the sequences complementary thereto. As described above, the general strategy to perform the association studies using the maps and markers of the present invention is to scan two groups of individuals (trait-positive individuals and trait-negative controls) characterized by a well defined phenotype in order to measure the allele frequencies of the biallelic markers in each of these groups. Preferably, the frequencies of markers with inter-marker spacing of about 150 kb are determined in each group. More preferably, the frequencies of markers with inter-marker spacing of about 75 kb are determined in each group. Even more preferably, markers with inter-marker spacing of about 50 kb, about 37.5 kb, about 30 kb, or about 25 kb will be tested in each population.

In some embodiments the frequencies of 1, 2, 3, 4, 5, 10, 15, 20, 25, 30, 40, 50, 70, 85, 100 or all of the biallelic markers selected from the group consisting of SEQ ID Nos.: 1 to 171, 1 to 100, 101 to 162 and 163 to 171 or the sequences complementary thereto are measured in each population. In another embodiment, the frequencies of at least 1, 2, 3, 4, 5, 10, 15, 20, 25, 30, 40, 50, 70, 85 or 100 biallelic markers selected from the group consisting of biallelic markers which are in linkage disequilibrium with the biallelic markers of SEQ ID Nos.: 1 to 171, 1 to 100, 101 to 162 and 163 to 171 or the sequences complementary thereto are measured in each population.

In some embodiments, the frequencies of about 20,000, or about 40,000 biallelic markers are determined in each population. In a highly preferred embodiment, the frequencies of about 60,000, about 80,000, about 100,000, or about 120,000 biallelic markers are determined in each population. In some embodiments, haplotype analyses may be run using groups of markers located within regions spanning less than 1 kb, from 1 to 5 kb, from 5 to 10 kb, from 10 to 25 kb, from 25 to 50 kb, from 50 to 150 kb, from 150 to 250 kb, from 250 to 500 kb, from 500 kb to 1 Mb, or more than 1 Mb.

Allele frequency can be measured using any genotyping method described herein including microsequencing techniques; preferred high throughput microsequencing procedures are further exemplified in III; it will be further appreciated that any other large scale genotyping method suitable with the intended purpose contemplated herein may also be used.

It will be appreciated that it is not necessary to use a full high density biallelic marker map in order to start a genome-wide association study. Maps having higher densities of biallelic markers (two or more markers per BAC, average inter-marker spacing of about 75 kb or less) may then be generated by starting first on those BACs for which a candidate association has been established at the first step. Chromosomal regions for which candidate associations have been suggested or established, and for which biallelic markers have been generated are further described in the “Background of the Invention”.

In cases when one or more candidate regions have previously been delineated, such as cases where a particular gene or genomic region is suspected of being associated with a trait, local excerpts of biallelic marker maps having densities above one marker per 150 kb may be exploited using BACs harboring said genomic regions, or genes, or portions thereof. In these cases also, successive association studies may be performed using sets of biallelic markers showing increasing densities, preferably from about one every 150 kb to about one every 75 kb; more preferably, sets of markers with inter-marker spacing below about 50 kb, below about 37.5 kb, below about 30 kb, most preferably below about 25 kb, will be used.

Haplotype analyses may also be conducted using groups of biallelic markers within the candidate region. The biallelic markers included in each of these groups may be located within a genomic region spanning less than 1 kb, from 1 to 5 kb, from 5 to 10 kb, from 10 to 25 kb, from 25 to 50 kb, from 50 to 150 kb, from 150 to 250 kb, from 250 to 500 kb, from 500 kb to 1 Mb, or more than 1 Mb. It will be appreciated that the ordered DNA fragments containing these groups of biallelic markers need not completely cover the genomic regions of these lengths but may instead be incomplete contigs having one or more gaps therein. As discussed in further detail below, biallelic markers may be used in association studies and haplotype analyses regardless of the completeness of the corresponding physical contig harboring them, provided linkage disequilibrium between the markers can be assessed.

As described above, if a positive association with a trait, such as a disease, or a drug efficacy and/or toxicity, is identified using the biallelic markers and maps of the present invention, the maps will provide not only the confirmation of the association, but also a shortcut towards the identification of the gene involved in the trait under study. As described above, since the markers showing positive association to the trait are in linkage disequilibrium with the trait loci, the causal gene will be physically located in the vicinity of these markers. Regions identified through association studies using high density maps will on average have a 20-40 times shorter length than those identified by linkage analysis (2 to 20 Mb).

As described above, once a positive association is confirmed with the high density biallelic marker maps of the present invention, BACs from which the most highly associated markers were derived are completely sequenced and the mutations in the causal gene are searched by applying genomic analysis tools. As described above, once a region harboring a gene associated with a detectable trait has been sequenced and analyzed, the candidate functional regions (e.g. exons and splice sites, promoters and other regulatory regions) are scanned for mutations by comparing the sequences of a selected number of controls and cases, using adequate software.

In some embodiments, trait-positive samples being compared to identify causal mutations are selected among those carrying the ancestral haplotype; in these embodiments, control samples are chosen from individuals not carrying said ancestral haplotype.

In further embodiments, trait-positive samples being compared to identify causal mutations are selected among those showing haplotypes that are as close as possible to the ancestral haplotype; in these embodiments, control samples are chosen from individuals not carrying any of the haplotypes selected for the case population.

The maps and biallelic markers of the present invention may also be used to identify patterns of biallelic markers associated with detectable traits resulting from polygenic interactions. The analysis of genetic interaction between alleles at unlinked loci requires individual genotyping using the techniques described herein. The analysis of allelic interaction among a selected set of biallelic markers with appropriate p-values can be considered as a haplotype analysis, similar to those described in further details within the present invention.

IX. Use of Biallelic Markers to Identify Individuals Likely to Exhibit a Detectable Trait Associated with a Particular Allele of a Known Gene

In addition to their utility in searches for genes associated with detectable traits on a genome-wide, chromosome-wide, or subchromosomal level, the maps and biallelic markers of the present invention may be used in more targeted approaches for identifying individuals likely to exhibit a particular detectable trait or individuals who exhibit a particular detectable trait as a consequence of possessing a particular allele of a gene associated with the detectable trait. For example, the biallelic markers and maps of the present invention may be used to identify individuals who carry an allele of a known gene that is suspected of being associated with a particular detectable trait. In particular, the target genes may be genes having alleles which predispose an individual to suffer from a specific disease state. In other cases, the target genes may be genes having alleles that predispose an individual to exhibit a desired or undesired response to a drug or other pharmaceutical composition, a food, or any administered compound. The known gene may encode any of a variety of types of biomolecules. For example, the known genes targeted in such analyzes may be genes known to be involved in a particular step in a metabolic pathway in which disruptions may cause a detectable trait Alternatively, the target genes may be genes encoding receptors or ligands which bind to receptors in which disruptions may cause a detectable trait, genes encoding transporters, genes encoding proteins with signaling activities, genes encoding proteins involved in the immune response, genes encoding proteins involved in hematopoesis, or genes encoding proteins involved in wound healing. It will be appreciated that the target genes are not limited to those specifically enumerated above, but may be any gene known to be or suspected of being associated with a detectable trait.

As previously mentioned, the maps and markers of the present invention may be used to identify genes associated with drug response. The biallelic markers of the present invention may also be used to select individuals for inclusion in the clinical trials of a drug. In some embodiments, the markers of SEQ ID Nos.: 1 to 171, 1 to 100, 101 to 162, 163 to 171 or the sequences complementary thereto may be used in targeted approaches to identify individuals at risk of developing a detectable trait, for example a complex disease or desired/undesired drug response, or to identify individuals exhibiting said trait. The present invention provides methods to establish putative associations between any of the biallelic markers described herein and any detectable traits, including those specifically described herein.

To use the maps and markers of the present invention in further targeted approaches, biallelic markers which are in linkage disequilibrium with any of the above disclosed markers may be identified. In cases where one or more biallelic markers of the present invention have been shown to be associated with a detectable trait, more biallelic markers in linkage disequilibrium with said associated biallelic markers may be generated and used to perform targeted approaches aiming at identifying individuals exhibiting, or likely to exhibit, said detectable trait, according to the methods provided herein.

Furthermore, in cases where a candidate gene is suspected of being associated with a particular detectable trait or suspected of causing the detectable trait, biallelic markers in linkage disequilibrium with said candidate gene may be identified and used in targeted approaches, such as the approaches utilized above for the Apo E gene.

Biallelic markers that are in linkage disequilibrium with markers associated with a detectable trait, or with genes associated with a detectable trait, or suspected of being so, are identified by performing single marker analyzes, haplotype association analyzes, or linkage disequilibrium measurements on samples from trait-positive and trait-negative individuals as described above using biallelic markers lying in the vicinity of the target marker or gene. In this manner, a single biallelic marker or a group of biallelic markers may be identified which indicate that an individual is likely to possess the detectable trait or does possess the detectable trait as a consequence of a particular allele of the target marker or gene.

Nucleic acid samples from individuals to be tested for predisposition to a detectable trait or possession of a detectable trait as a consequence of a particular allele of the target gene may be examined using the diagnostic methods described above.

X. DNA Typing Methods and Systems

The present invention also encompasses a DNA typing system having a much higher discriminatory power than currently available typing systems. The systems and associated methods are particularly applicable in the identification of individuals for forensic science and paternity determinations. These applications have become increasingly important; in forensic science, for example, the identification of individuals by polymorphism analysis has become widely accepted by courts as evidence.

While forensic geneticists have developed many techniques to compare homologous segments of DNA to determine if the segments are identical or if they differ in one or more nucleotides, each technique still has certain disadvantages. In particular, the techniques vary widely in terms of expense of analysis, time required to carry out an analysis and statistical power.

RFLP Analysis Methods

The best known and most widespread method in forensic DNA typing is the restriction fragment length polymorphism (RFLP) analysis. In RFLP testing, a repetitive DNA sequence referred to as a variable number tandem repeat (VNTR) which varies between individuals is analyzed. The core repeat is typically a sequence of about 15 base pairs in length, and highly polymorphic VNTR loci can have an average of about 20 alleles. DNA restriction sites located on either site of the VNTR are exploited to create DNA fragments from about 0.5 Kb to less than 1 OKb which are then separated by electrophoresis, indicating the number of repeats found in the individual at the particular loci. RFLP methods generally consist of (1) extraction and isolation of DNA, (2) restriction endonuclease digestion; (3) separation of DNA fragments by electrophoresis; (4) capillary transfer; (5) hybridization with radiolabelled probes; (6) autoradiography; and (7) interpretation of results (Lee, H. C. et al., Am. J. Forensic. Med. Pathol. 15(4): 269-282 (1994)). RFLP methods generally combine analysis at about 5 loci and have much higher discriminate potential than other available test due the highly polymorphic nature of the VNTRs. However, autoradiography is costly and time consuming and an analysis generally takes weeks or months for turnaround. Additionally, a large amount of sample DNA is required, which is often not available at a crime scene. Furthermore, the reliability of the system and its credibility as evidence is decreased because the analysis of tightly spaced bands on electrophoresis results in a high rate of error.

PCR Methods

PCR based methods offer an alternative to RFLP methods. In a first method called AmpFLP, DNA fragments containing VNTRs are amplified and then separated electrophoretically, without the restriction step of RFLP method. While this method allows small quantities of sample DNA to be used, decreases analysis time by avoiding autoradiography, and retains high discriminatory potential, it nevertheless requires electrophoretic separation which takes substantial time and introduces an significant error rate. In another AmpFLP method, short tandem repeats (STRs) of 2 to 8 base pairs are analyzed. STRs are more suitable to analysis of degraded DNA samples since they require smaller amplified fragments but have the disadvantage of requiring separation of the amplified fragments. While STRs are far less informative than longer repeats, similar discriminatory potential can be achieved if enough STRs are used in a single analysis.

Other methods include sequencing of mitochondrial DNA, which is especially suitable for situations where sample DNA is very degraded or in small quantities. However, only a small region of 1 Kb of the mitochondrial DNA referred to as the D-Loop locus has been found useful for typing because of its polymorphic nature, resulting in lower discriminatory potential than with RFLP or AmpFLP methods. Furthermore, DNA sequencing is expensive to carry out on a large number of samples.

Further available methods include dot-blot methods, which involve using allele specific oligonucleotide probes which hybridize sequence specifically to one allele of a polymorphic site. Systems include the HLA DQ-alpha kit developed by Cetus Corp. which has a discriminatory value of about 1 in 20, and a dot-blot strip referred to as the Polymarker strip combining five genetic loci for a discriminatory value of about one in a few thousand. (Weedn, V., Clinics in Lab. Med. 16(1): 187-196 (1996)).

In addition to difficulties in analysis and time consuming laboratory procedures, it remains desirable for all DNA typing systems to have a higher discriminatory power. Several applications exist in which even the most discriminating tests need improvement in order to remove the considerable remaining doubt resulting from such analyses. Table 1b below lists characteristics of currently available forensic testing systems (Weedn, (1996)) and compares them with the method of the invention.

TABLE 1b

| | | | | Sensitivity | |
|---|---|---|---|---|---|
| | | Turnaround | Discriminatory | (amount | |
| Test type | Technology | time | potential | DNA) | Sample |
| RFLP | VNTR | Weeks or | 10⁶ to 10⁹ | 10 ng | Highly intact |
| | (autoradiography) | months | | | DNA |
| AmpFLP | VNTR | Days | 10³ to 10⁶ | 100 pg | Moderate |
| | (PCR based) | | | | degradation |
| Dot blot (ex. | Sequence specific | Days | 10¹ to 10³ | 1 ng | Moderate |
| HLADQA1) | oligonucleotide | | | | degradation |
| | probes | | | | |
| Mitochondrial | D-loop sequence | Days | 10² | 1 pg | Severe |
| DNA | (PCR based) | | | | degradation |
| Present | Biallelic Markers | Hours to | 10⁶, 10⁴⁷, 10²³⁸ | 100 pg | Moderate |
| biallelic | (set of 13, set of | Days | | | degradation |
| marker map | 100, set of 500) | (throughput | | | |
| | | dependent) | | | |

Applications

As described above, an important application of DNA typing tests is to determine whether a DNA sample (e.g. from a crime scene) originated from an individual suspected of leaving said DNA sample.

There are several applications for DNA typing which require a particularly powerful genotyping system. In a first application, a high powered typing system is advantageous when for example a suspect is identified by searching a DNA profile database such as that maintained by the U.S. Federal Bureau of Investigation. Since databases may contain large numbers of data entries that are expected to increase consistently, currently used forensic systems can be expected to identify several matching DNA profiles due to their relative lack of power. While database searches generally reinforce the evidence by excluding other possible suspects, low powered typing systems resulting in the identification of several individuals may often tend to diminish the overall case against a defendant.

In another application, a target population is systematically tested to identify an individual having the same DNA profile as that of a DNA sample. In such a situation, a defendant is chosen at random based on DNA profile from a large population of innocent individuals. Since the population tested can often be large enough that at least one positive match is identified, and it is usually not possible to exhaustively test a population, the usefulness of the evidence will depend on the level of significance of the forensic test. In order to render such an application useful as a sole or primary source of evidence, DNA typing systems of extremely high discriminatory potential are required.

In yet another application, it is desirable to be able to discriminate between related individuals. Because related individuals will be expected to share a large portion of alleles at polymorphic sites, a very high powered DNA typing assay would be required to discriminate between them. This can have important effects if a sample is found to match the defendant's DNA profile and no evidence that the perpetrator is a relative can be found.

Accordingly, there a need in this art for a rapid, simple, inexpensive and accurate technique having a very high resolution value to determine relationships between individuals and differences in degree of relationships. Also, there is a need in the art for a very accurate genetic relationship test procedure which uses very small amounts of an original DNA sample, yet produces very accurate results.

The present invention thus involves methods for the identification of individuals comprising determining the identity of the nucleotides at set of genetic markers in a biological sample, wherein said set of genetic markers comprises at least one map-related biallelic marker. The present invention provides an extensive set of biallelic markers allowing a higher discriminatory potential than the genetic markers used in current forensic typing systems. Also, biallelic markers can be genotyped in individuals with much higher efficiency and accuracy than the genetic markers used in current forensic typing systems. In preferred embodiments, the invention comprises determining the identity of a nucleotide at a map-related biallelic marker by single nucleotide primer extension, which does not require electrophoresis as in techniques described above and results in lower rate of experimental error. As shown in Table 1b, above, in comparison with PCR based VNTR based methods which allow discriminatory potential of thousands to millions, and RFLP based methods which allow discriminatory potential of merely millions to billions under optimal assumptions, the biallelic marker based method of the present invention provides a radical increase in discriminatory potential.

Any suitable set of genetic markers and biallelic markers of the invention may be used, and may be selected according to the discriminatory power desired. Biallelic markers, sets of biallelic markers, probes, primers, and methods for determining the identity of said biallelic markers are further described herein.

Discriminatory Potential of Biallelic Marker Typing

Calculating Discriminatory Potential

The discriminatory potential of the forensic test can be determined in terms of the profile frequency, also referred to as the random match probability, by applying the product rule. The product rule involves multiplying the allelic frequencies of all the individual alleles tested, and multiplying by an additional factor of 2 for each heterozygous locus.

In one example discussed below, the discriminatory potential of biallelic marker typing can be considered in the context of forensic science. In order to determine the discriminatory potential with respect to the numbers of biallelic markers to be used in a genetic typing system, the formulas and calculations below assume that (1) the population under study is sufficiently large (so that we can assume no consanguinity); (2) all markers chosen are not correlated, so that the product rule (Lander and Budlowle (1992)) can be applied; and (3) the ceiling rule can be applied or that the allelic frequencies of markers in the population under study are known with sufficient accuracy.

As noted in Weir, B. S., Genetic data Analysis II: Methods for Discrete population genetic Data, Sinauer Assoc., Inc., Sunderland, Mass., USA, 1996, the example assumes a crime has been committed and a sample of DNA from the perpetrator (P) is available for analysis. The genotype of this DNA sample can be determined for several genetic markers, and the profile A of the perpetrator can thereby be determined.

In this example, one suspect (S) is available for typing. The same set of genetic markers, such as the biallelic markers of the invention, are typed and the same profile A is obtained for (S) and (P). Two hypotheses are thus presented as follows:

(1) either S is P (event C)

(2) either S is not P (event C).

The ratio L of both probabilities can then be calculated using the following equation:

$$L = \frac{pr ( S = A , P = A / C )}{pr ( S = A , P = A / \overline{C} )}$$

L can then further be calculated by the following equation:

$$L = \frac{1}{pr ( P = A / S = A , \overline{C} )} \qquad ( 1 ) Equation 1$$

These probabilities as well as L can be calculated in several settings, notably for different kinship coefficients between P and S for a genetic marker (see Weir, (1996)).

Assuming that all genetic markers chosen are independent of each other, the global ratio L for a set of genetic markers will be the product over each genetic marker of all L.

It is further possible to estimate the mean number of biallelic markers or VNTRs required to have a ratio L equal to 10⁸ or 10⁶ by calculating the expectancy of the random variable L using the following equation:

$$E ( L ) = {\prod }_{i = 1}^{N} E ( {L}_{i} )$$

where N is the number of loci

$$E ( {L}_{i} ) = {\sum }_{j = 1}^{{G}_{i}} pr ( P = {A}_{ij} / S = {A}_{ij} , \overline{C} ) \cdot {L}_{ij} ,$$

where Aᵢⱼ is the genotype j at the ith marker,

Lᵢⱼ the ratio associated with such genotype, Gᵢ being the number of genotypes at locus i. From equation 1, it can easily be derived that the expectancy of Lᵢ is Gᵢ, the number of possible genotypes of this marker.

The general expectancy for a set of genetic markers can then be expressed by the following equation:

$$E ( L ) = {\prod }_{i = 1}^{N} {G}_{i} \qquad ( 2 ) Equation 2$$

Biallelic Marker-Based DNA Typing Systems

Using the equations described above, it is possible to select biallelic marker-based DNA typing systems having a desired discriminatory potential.

Using biallelic markers, E(L) can thus be expressed as 3^{N}. When using VNTR-based DNA typing systems, assuming the VNTRs have 10 alleles, E(L) can be expressed as 55^{N}. Based on these results, the number of biallelic markers or VNTRs needed to obtain, in mean, a ratio of at least 10⁶ or 10⁸ can calculated, and are set forth below in Table 1c.

TABLE 1c

| Marker sets | L = 10⁶ | L = 10⁸ |
|---|---|---|
| Biallelic | 13 | 17 |
| 5-allele markers (e.g. VNTR) | 5 | 7 |
| 10-allele markers (e.g. VNTR) | 4 | 5 |

Thus, in a first embodiment, DNA typing systems and methods of the invention may comprise genotyping a set of at least 13 or at least 17 biallelic markers to obtain a ratio of at least 10⁶ or 10⁸, assuming a flat distribution of L across the biallelic markers. In preferred embodiments, a greater number of biallelic markers is genotyped to obtain a higher L value. Preferably at least 1, 2, 3, 4, 5, 10, 13, 15, 17, 20, 25, 30, 40, 50, 70, 85, 100, 150, or all of the map-related biallelic markers are genotyped. Said DNA typing systems of the invention would result in L values as listed in Table 1d below as an indication of the discriminate potential of the systems of the invention.

TABLE 1d

| Number of biallelic | |
|---|---|
| markers | L |
| 50 | 7.2 * 10²³ |
| 100 | 5 * 10⁴⁷ |
| 171 | 3.9 * 10⁸¹ |

In situations where the distribution of L is not flat, such as in the worst case when the perpetrator is homozygous for the major allele at each genetic locus and L thus takes the lowest value, a larger number of biallelic markers is required for the same discriminatory potential. Therefore, in preferred embodiments, DNA typing systems and methods of the invention using a larger number of biallelic markers allow for uneven distributions of L across the biallelic markers. For example, assuming unrelated individuals, a set of independent markers having an allelic frequency of 0.1/0.9, and the genetic profile of a homozygote at each genetic loci for the major allele, 66 biallelic markers are required to obtain a ratio of 10⁶, and 88 biallelic markers are required to obtain a ratio of 10⁸. Thus, in preferred embodiments based on the use of markers having a major allele of sufficiently high frequency, this is a first estimation of the upper bound of markers required in a DNA typing system.

In further embodiments, it is also desirable to have the ability to discriminate between relatives. Although unrelated individuals have a low probability of sharing genetic profiles, the probability is greatly increased for relatives. For example, the DNA profile of a suspect matches the DNA profile of a sample at a crime scene, and the probability of obtaining the same DNA profile if left by an untyped relative is required. Table 1e below (Weir (1996)) lists probabilities for several different types of relationships, assuming alleles Aᵢ and Aⱼ, and population frequencies pᵢ and pⱼ, and lists likelihood ratios assuming genetic loci having allele frequencies of 0.1.

TABLE 1e

| Genotype | Relationship | Pr(p = A\|S = A) | L |
|---|---|---|---|
| Aᵢ Aⱼ | Full brothers | (1 + pᵢ + pⱼ + 2pᵢ pⱼ)/4 | 3.3 |
| | Father and son | (pᵢ + pⱼ)/2 | 10.0 |
| | Half brothers | (pᵢ + pⱼ + 4pᵢ pⱼ)/4 | 16.7 |
| | Uncle and nephew | (1 + pᵢ + pⱼ + 2pᵢ pⱼ)/4 | 16.7 |
| | First cousins | (1 + pᵢ + pⱼ + 12pᵢ pⱼ)/8 | 25.0 |
| | Unrelated | 2pᵢ pⱼ | 50.0 |
| Aⱼ Aⱼ | Full brothers | (1 + pᵢ)2/4 | 3.3 |
| | Father and son | pᵢ | 10.0 |
| | Half brothers | pᵢ (1 + pᵢ)/2 | 18.2 |
| | Uncle and nephew | pᵢ (1 + pᵢ)/2 | 18.2 |
| | First cousins | pᵢ (1 + 3pᵢ)/4 | 30.8 |
| | Unrelated | pᵢ² | 100.0 |

In one example, where the suspect is the full brother of the perpetrator, the number of required biallelic markers will be 187 assuming the profile is that of a homozygote for the major allele at each biallelic marker.

In yet further embodiments, the DNA typing systems and methods of the present invention may further take into account effects of subpopulations on the discriminatory potential. In embodiments described above for example, DNA typing systems consider close familial relationships, but do not take into account membership in the same population. While population membership is expected to have little effect, the invention may further comprise genotyping a larger set of biallelic markers to achieve higher discriminatory potential. Alternatively, a larger set of biallelic markers may be optimized for typing selected populations; alternatively, the ceiling principle may be used to study allele frequencies from individuals in various populations of interest, taking for any particular genotype the maximum allele frequency found among the populations.

The invention thus encompasses methods for genotyping comprising determining the identity of a nucleotide at least 13, 15, 17, 20, 25, 30, 40, 50, 66, 70, 85, 88, 100, 187, 200, 300, 500, 700, 1000 or 2000 biallelic markers in a biological sample, wherein at least 1, 2, 3, 4, 5, 10, 13, 17, 20, 25, 30, 40, 50, 70, 85, 100, 150 or all of said biallelic markers are map-related biallelic markers selected from the group consisting of SEQ ID No. 1 to 171, 1 to 100, 101 to 162, 163 to 171.

Any markers known in the art may be used with the map-related biallelic markers of the present invention in the DNA typing methods and systems described herein, for example in anyone of the following web sites offering collections of SNPs and information about those SNPs:

The Genetic Annotation Initiative (http://cgap.nci.nih.gov/GAI/). An NIH run site which contains information on candidate SNPs thought to be related to cancer and tumorigenesis generally.

dbSNP Polymorphism Repository (http://www.ncbi.nlm nih.gov/SNP/). A more comprehensive NIH-run database containing information on SNPs with broad applicability in biomedical research.

HUGO Mutation Database Initiative (http://ariel.ucs.unimelb.edu.au:80/-cotton/mdi.htm). A database meant to provide systematic access to information about human mutations including SNPs. This site is maintained by the Human Genome Organisation (HUGO).

Human SNP Database (http://www-genome.wi.mit.edu/SNP/human/index.html). Managed by the Whitehead Institute for Biomedical Research Genome Institute, this site contains information about SNPs resulting from the many Whitehead research projects on mapping and sequencing.

SNPs in the Human-Genome SNP database (http://www.ibc.wustl.edu/SNP). This website provides access to SNPs that have been organized by chromosomes and cytogenetic location. The site is run by Washington University.

HGBase (http://hgbase.cgr.ki.se/). HGBASE is an attempt to summarize all known sequence variations in the human genome, to facilitate research into how genotypes affect common diseases, drug responses, and other complex phenotypes, and is run by the Karolinska Institute of Sweden.

The SNP Consortium Database (http://snp.cshl.org./db/snp/map). A collection of SNPs and related information resulting from the collaborative effort of a number of large pharmaceutical and information processing companies.

GeneSNPs (http://www.genome.utah.edu/genesnps/). Run by the University of Utah, this site contains information about SNPs resulting from the U.S. National Institute of Environmental Health's initiative to understand the relationship between genetic variation and response to environmental stimuli and xenobiotics.

In addition, biallelic markers provided in the following patents and patent applications may also be used with the map-realted biallelic markers of the invention in the DNA typing methods and systems described above: U.S. Serial No. 60/206,615, filed Mar. 24, 2000; U.S. Serial No. 60/216,745, filed Jun. 30, 2000; WIPO Serial No. PCT/IB00/00184, filed Feb. 11, 2000; WIPO Serial No. PCT/IB98/01193, filed Jul. 17, 1998; PCT Publication No. WO 99/54500, filed Apr. 21, 1999; and WIPO Serial No. PCT/IBOO/00403, filed Mar. 24, 2000.

Biallelic markers, sets of biallelic markers, probes, primers, and methods for determining the identity of a nucleotide at said biallelic markers are also encompassed and are further described herein, and may encompass any further limitation described in this disclosure, alone or in any combination.

Forensic matching by microsequencing is further described in Example 27 below.

Throughout this application, various publications, patents, and published patent applications are cited. The disclosures of the publications, patents, and published patent specifications referenced in this application are hereby incorporated by reference into the present disclosure in their entireties to more fully describe the state of the art to which this invention pertains.

### EXAMPLES

Several of the methods of the present invention are described in the following examples, which are offered by way of illustration and not by way of limitation. Many other modifications and variations of the invention as herein set forth can be made without departing from the spirit and scope thereof and therefore only such limitations should be imposed as are indicated by the appended claims.

### Example 1

### Ordering of a BAC Library: Screening Clones with STSs

The BAC library is screened with a set of PCR-typeable STSs to identify clones containing the STSs. To facilitate PCR screening of several thousand clones, for example 200,000 clones, pools of clones are prepared.

Three-dimensional pools of the BAC libraries are prepared as described in Chumakov et al. and are screened for the ability to generate an amplification fragment in amplification reactions conducted using primers derived from the ordered STSs. (Chumakov et al. (1995), supra). A BAC library typically contains 200,000 BAC clones. Since the average size of each insert is 100-300 kb, the overall size of such a library is equivalent to the size of at least about 7 human genomes. This library is stored as an array of individual clones in 518 384-well plates. It can be divided into 74 primary pools (7 plates each). Each primary pool can then be divided into 48 subpools prepared by using a three-dimensional pooling system based on the plate, row and column address of each clone (more particularly, 7 subpools consisting of all clones residing in a given microtiter plate; 16 subpools consisting of all clones in a given row; 24 subpools consisting of all clones in a given column).

Amplification reactions are conducted on the pooled BAC clones using primers specific for the STSs. For example, the three dimensional pools may be screened with 45,000 STSs whose positions relative to one another and locations along the genome are known. Preferably, the three dimensional pools are screened with about 30,000 STSs whose positions relative to one another and locations along the genome are known. In a highly preferred embodiment, the three dimensional pools are screened with about 20,000 STSs whose positions relative to one another and locations along the genome are known.

Amplification products resulting from the amplification reactions are detected by conventional agarose gel electrophoresis combined with automatic image capturing and processing. PCR screening for a STS involves three steps: (1) identifying the positive primary pools; (2) for each positive primary pool, identifying the positive plate, row and column ‘subpools’ to obtain the address of the positive clone; (3) directly confirming the PCR assay on the identified clone. PCR assays are performed with primers specifically defining the STS.

Screening is conducted as follows. First BAC DNA containing the genomic inserts is prepared as follows. Bacteria containing the BACs are grown overnight at 37° C. in 120 μl of LB containing chloramphenicol (12 μg/ml). DNA is extracted by the following protocol:

Centrifuge 10 min at 4° C. and 2000 rpm

Eliminate supernatant and resuspend pellet in 120 μl TE 10-2 (Tris HCl 10 mM, EDTA 2 mM)

Centrifuge 10 min at 4° C. and 2000 rpm

Eliminate supernatant and incubate pellet with 20 μl lyzozyme 1 mg/ml during 15 min at room temperature

Add 20 μl proteinase K 100 μg/ml and incubate 15 min at 60° C.

Add 8 μl DNAse 2U/μl and incubate 1 hr at room temperature

Add 100 μl TE 10-2 and keep at −80° C.

PCR assays are performed using the following protocol:

| | | |
|---|---|---|
| Final volume | 15 | μl |
| BAC DNA | 1.7 | ng/μl |
| MgCl₂ | 2 | mM |
| dNTP (each) | 200 | μM |
| primer (each) | 2.9 | ng/μl |
| Ampli Taq Gold DNA polymerase | 0.05 | unit/μl |
| PCR buffer (10× = 0.1 M TrisHCl pH 8.3 0.5 M KCl | 1× | |

The amplification is performed on a Genius II thermocycler. After heating at 95° C. for 10 min, 40 cycles are performed. Each cycle comprises: 30 sec at 95° C., 54° C. for 1 min, and 30 sec at 72° C. For final elongation, 10 min at 72° C. end the amplification. PCR products are analyzed on 1% agarose gel with 0.1 mg/ml ethidium bromide.

Alternatively, a YAC (Yeast Artificial Chromosome) library can be used. The very large insert size, of the order of 1 megabase, is the main advantage of the YAC libraries. The library can typically include about 33,000 YAC clones as described in Chumakov et al. (1995, supra). The YAC screening protocol may be the same as the one used for BAC screening.

The known order of the STSs is then used to align the BAC inserts in an ordered array (contig) spanning the whole human genome. If necessary new STSs to be tested can be generated by sequencing the ends of selected BAC inserts. Subchromosomal localization of the BACs can be established and/or verified by fluorescence in situ hybridization (FISH), performed on metaphasic chromosomes as described by Cherif et al. 1990 and in Example 3 below. BAC insert size may be determined by Pulsed Field Gel Electrophoresis after digestion with the restriction enzyme NotI.

Finally, a minimally overlapping set of BAC clones, with known insert size and subchromosomal location, covering the entire genome, a set of chromosomes, a single chromosome, a particular subchromosomal region, or any other desired portion of the genome is selected from the DNA library. For example, the BAC clones may cover at least 100 kb of contiguous genomic DNA, at least 250 kb of contiguous genomic DNA, at least 500 kb of contiguous genomic DNA, at least 2 Mb of contiguous genomic DNA, at least 5 Mb of contiguous genomic DNA, at least 10 Mb of contiguous genomic DNA, or at least 20 Mb of contiguous genomic DNA.

### Example 2

### Screening BAC Libraries with Biallelic Markers

Amplification primers enabling the specific amplification of DNA fragments carrying the biallelic markers, including the map-related biallelic markers of the invention, may be used to screen clones in any genomic DNA library, preferably the BAC libraries described above for the presence of the biallelic markers.

Pairs of primers of SEQ ID Nos: 172 to 513, 172 to 271, 272 to 333, 334 to 342, 343 to 442, 443 to 504 and 505 to 513 were designed which allow the amplification of fragments carrying the biallelic markers of SEQ ID Nos: 1 to 171, 1 to 100, 101 to 162, 163 to 171 or the sequences complementary thereto. The amplification primers of SEQ ID Nos: 172 to 513, 172 to 271, 272 to 333, 334 to 342, 343 to 442, 443 to 504 and 505 to 513 may be used to screen clones in a genomic DNA library for the presence of the biallelic markers of SEQ ID Nos: 1 to 171, 1 to 100, 101 to 162, 163 to 171 or the sequences complementary thereto.

It will be appreciated that amplification primers for the biallelic markers of SEQ ID Nos: 1 to 171, 1 to 100, 101 to 162, 163 to 171 need not be identical to the primers of SEQ ID Nos: 172 to 513, 172 to 271, 272 to 333, 334 to 342, 343 to 442, 443 to 504 and 505 to 513. Rather, they can be any other primers allowing the specific amplification of any DNA fragment carying the markers and may be designed using techniques familiar to those skilled in the art. The amplification primers may be oligonucleotides of 8, 10, 15, 20 or more bases in length which enable the amplification of any fragment carrying the polymorphic site in the markers. The polymorphic base may be in the center of the amplification product or, alternatively, it may be located off-center. For example, in some embodiments, the amplification product produced using these primers may be at least 100 bases in length (i.e. 50 nucleotides on each side of the polymorphic base in amplification products in which the polymorphic base is centrally located). In other embodiments, the amplification product produced using these primers may be at least 500 bases in length (i.e. 250 nucleotides on each side of the polymorphic base in amplification products in which the polymorphic base is centrally located). In still further embodiments, the amplification product produced using these primers may be at least 1000 bases in length (i.e. 500 nucleotides on each side of the polymorphic base in amplification products in which the polymorphic base is centrally located). Amplification primers such as those described above are included within the scope of the present invention.

The localization of biallelic markers on BAC clones is performed essentially as described in Example 1.

The BAC clones to be screened are distributed in three dimensional pools as described in Example 1.

Amplification reactions are conducted on the pooled BAC clones using primers specific for the biallelic markers to identify BAC clones which contain the biallelic markers, using procedures essentially similar to those described in Example 1.

Amplification products resulting from the amplification reactions are detected by conventional agarose gel electrophoresis combined with automatic image capturing and processing. PCR screening for a biallelic marker involves three steps: (1) identifying the positive primary pools; (2) for each positive primary pools, identifying the positive plate, row and column ‘subpools’ to obtain the address of the positive clone; (3) directly confirming the PCR assay on the identified clone. PCR assays are performed with primers defining the biallelic marker.

Screening is conducted as follows. First BAC DNA is isolated as follows. Bacteria containing the genomic inserts are grown overnight at 37° C. in 120 μl of LB containing chloramphenicol (12 μg/ml). DNA is extracted by the following protocol:

Centrifuge 10 min at 4° C. and 2000 rpm

Eliminate supernatant and resuspend pellet in 120 μl TE 10-2 (Tris HCl 10 mM, EDTA 2 mM)

Centrifuge 10 min at 4° C. and 2000 rpm

Eliminate supernatant and incubate pellet with 20 μl lyzozyme 1 mg/ml during 15 min at room temperature

Add 20 μl proteinase K 100 μg/ml and incubate 15 min at 60° C.

Add 8 μl DNAse 2U/μl and incubate 1 hr at room temperature

Add 100 μl TE 10-2 and keep at-80° C.

PCR assays are performed using the following protocol:

| | | |
|---|---|---|
| Final volume | 15 | μl |
| BAC DNA | 1.7 | ng/μl |
| MgCl₂ | 2 | mM |
| dNTP (each) | 200 | μM |
| primer (each) | 2.9 | ng/μl |
| Ampli Taq Gold DNA polymerase | 0.05 | unit/μl |
| PCR buffer (10× = 0.1 M TrisHCl pH 8.3 0.5 M KCl | 1× | |

The amplification is performed on a Genius II thermocycler. After heating at 95° C. for 10 min, 40 cycles are performed. Each cycle comprises: 30 sec at 95° C., 54° C. for 1 min, and 30 sec at 72° C. For final elongation, 10 min at 72° C. end the amplification. PCR products are analyzed on 1% agarose gel with 0.1 mg/ml ethidium bromide.

### Example 3

### Assignment of Biallelic Markers to Subchromosomal Regions

Metaphase chromosomes are prepared from phytohemagglutinin (PHA)-stimulated blood cell donors. PHA-stimulated lymphocytes from healthy males are cultured for 72 h in RPMI-1640 medium. For synchronization, methotrexate (10 mM) is added for 17 h, followed by addition of 5-bromodeoxyuridine (5-BudR, 0.1 mM) for 6 h. Colcemid (1 mg/ml) is added for the last 15 min before harvesting the cells. Cells are collected, washed in RPMI, incubated with a hypotonic solution of KCl (75 mM) at 37° C. for 15 min and fixed in three changes of methanol:acetic acid (3:1). The cell suspension is dropped onto a glass slide and air-dried.

BAC clones carrying the biallelic markers used to construct the maps of the present invention (including the biallelic markers of SEQ ID Nos: 1 to 171, 1 to 100, 101 to 162 and 163 to 171 or the sequences complementary thereto) can be isolated as described above. These BACs or portions thereof, including fragments carrying said biallelic markers, obtained for example from amplification reactions using pairs of primers of SEQ ID Nos: 172 to 513, 172 to 271, 272 to 333, 334 to 342, 343 to 442, 443 to 504 and 505 to 513, can be used as probes to be hybridized with metaphasic chromosomes. It will be appreciated that the hybridization probes to be used in the contemplated method may be generated using alternative methods well known to those skilled in the art. Hybridization probes may have any length suitable for this intended purpose.

Probes are then labeled with biotin-16 dUTP by nick translation according to the manufacturer's instructions (Bethesda Research Laboratories, Bethesda, Md.), purified using a Sephadex G-50 column (Pharmacia, Upssala, Sweden) and precipitated. Just prior to hybridization, the DNA pellet is dissolved in hybridization buffer (50% formamide, 2×SSC, 10% dextran sulfate, 1 mg/ml sonicated salmon sperm DNA, pH 7) and the probe is denatured at 70° C. for 5-10 min.

Slides kept at −20° C. are treated for 1 h at 37° C. with RNase A (100 mg/ml), rinsed three times in 2×SSC and dehydrated in an ethanol series. Chromosome preparations are denatured in 70% formamide, 2×SSC for 2 min at 70° C., then dehydrated at 4° C. The slides are treated with proteinase K (10 mg/100 ml in 20 mM Tris-HCl, 2 mM CaCl₂) at 37° C. for 8 min and dehydrated. The hybridization mixture containing the probe is placed on the slide, covered with a coverslip, sealed with rubber cement and incubated overnight in a humid chamber at 37° C. After hybridization and post-hybridization washes, the biotinylated probe is detected by avidin-FTTC and amplified with additional layers of biotinylated goat anti-avidin and avidin-FITC. For chromosomal localization, fluorescent R-bands are obtained as previously described (Cherif et al.,(1990) supra.). The slides are observed under a LEICA fluorescence microscope (DMRXA). Chromosomes are counterstained with propidium iodide and the fluorescent signal of the probe appears as two symmetrical yellow-green spots on both chromatids of the fluorescent R-band chromosome (red). Thus, a particular biallelic marker may be localized to a particular cytogenetic R-band on a given chromosome.

The rate at which biallelic markers may be assigned to subchromosomal regions may be enhanced through automation. For example, probe preparation may be performed in a microtiter plate format, using adequate robots. The rate at which biallelic markers may be assigned to subchromosomal regions may be enhanced using techniques which permit the in situ hybridization of multiple probes on a single microscope slide, such as those disclosed in Larin et al., Nucleic Acids Research 22: 3689-3692 (1994), the disclosure of which is incorporated herein by reference in its entirety. In the largest test format described, different probes were hybridized simultaneously by applying them directly from a 96-well microtiter dish which was inverted on a glass plate. Software for image data acquisition and analysis that is adapted to each optical system, test format, and fluorescent probe used, can be derived from the system described in Lichter et al. Science 247: 64-69 (1990), the disclosure of which is incorporated herein by reference in its entirety. Such software measures the relative distance between the center of the fluorescent spot corresponding to the hybridized probe and the telomeric end of the short arm of the corresponding chromosome, as compared to the total length of the chromosome. The rate at which biallelic markers are assigned to subchromosomal locations may be further enhanced by simultaneously applying probes labeled with different flouorescent tags to each well of the 96 well dish. A further benefit of conducting the analysis on one slide is that it facilitates automation, since a microscope having a moving stage and the capability of detecting fluorescent signals in different metaphase chromosomes could provide the coordinates of each probe on the metaphase chromosomes distributed on the 96 well dish.

Example 4 below describes an alternative method to position biallelic markers which allows their assignment to human chromosomes.

### Example 4

### Assignment of Biallelic Markers to Human Chromosomes

The biallelic markers used to construct the maps of the present invention, including the biallelic markers of SEQ D Nos. 1 to 171, 1 to 100, 101 to 162, 163 to 171 or the sequences complementary thereto, may be assigned to a human chromosome using monosomal analysis as described below.

The chromosomal localization of a biallelic marker can be performed through the use of somatic cell hybrid panels. For example 24 panels, each panel containing a different human chromosome, may be used (Russell et al., Somat Cell Mol. Genet 22:425-431(1996); Drwinga et al., Genomics 16:311-314 (1993), the disclosures of which are incorporated herein by reference in their entireties).

The biallelic markers are localized as follows. The DNA of each somatic cell hybrid is extracted and purified. Genomic DNA samples from a somatic cell hybrid panel are prepared as follows. Cells are lysed overnight at 42° C. with 3.7 ml of lysis solution composed of:

3 ml TE 10-2 (Tris HCl 101 mM, EDTA 2 m/NaCl 0.4 M

200 μl SDS 10%

500 μl K-proteinase (2 mg K-proteinase in TE 10-2/NaCl 0.4 M)

For the extraction of proteins, 1 ml saturated NaCl (6M) (1/3.5 v/v) is added. After vigorous agitation, the solution is centrifuged for 20 min at 10,000 rpm. For the precipitation of DNA, 2 to 3 volumes of 100% ethanol are added to the previous supematant, and the solution is centrifuged for 30 min at 2,000 rpm. The DNA solution is rinsed three times with 0.70% ethanol to eliminate salts, and centrifuged for 20 min at 2,000 rpm. The pellet is dried at 37° C., and resuspended in 1 ml TE 10-1 or 1 ml water. The DNA concentration is evaluated by measuring the OD at 260 nm (1 unit OD=50 μg/ml DNA). To determine the presence of proteins in the DNA solution, the OD₂₆₀/OD₂₈₀ ratio is determined. Only DNA preparations having a OD₂₆₀/OD₂₈₀ ratio between 1.8 and 2 are used in the PCR assay.

Then, a PCR assay is performed on genomic DNA with primers defining the biallelic marker. The PCR assay is performed as described above for BAC screening. The PCR products are analyzed on a 1% agarose gel containing 0.2 mg/ml ethidium bromide.

### Example 5

### Measurement of Linkage Disequilibrium

As originally reported by Strittmatter et al. and by Saunders et al. in 1993, the Apo E e4 allele is strongly associated with both late-onset familial and sporadic Alzheimer's disease. (Saunders, A. M. Lancet 342: 710-711 (1993) and Strittmater, W. J. et al., Proc. Natl. Acad. Sci. U.S.A. 90: 1977-1981 (1993), the disclosures of which are incorporated herein by reference in their entireties). The 3 major isoforms of human Apolipoprotein E (apoE2, -E3, and -E4), as identified by isoelectric focusing, are coded for by 3 alleles (e 2, 3, and 4). The e 2, e 3, and e 4 isoforms differ in amino acid sequence at 2 sites, residue 112 (called site A) and residue 158 (called site B). The ancestral isoform of the protein is Apo E3, which at sites A/B contains cysteine/arginine, while ApoE2 and -E4 contain cysteine/cysteine and arginine/arginine, respectively (Weisgraber, K. H. et al., J. Biol. Chem. 256: 9077-9083 (1981); Rall, S. C. et al., Proc. Natl. Acad. Sci. U.S.A. 79: 4696-4700 (1982), the disclosures of which are incorporated herein by reference in their entireties).

Apo E e 4 is currently considered as a major susceptibility risk factor for Alzheimer's disease development in individuals of different ethnic groups (specially in Caucasians and Japanese compared to Hispanics or African Americans), across all ages between 40 and 90 years, and in both men and women, as reported recently in a study performed on 5930 Alzheimer's disease patients and 8607 controls (Farrer et al., JAMA 278:1349-1356 (1997), the disclosure of which is incorporated herein by reference in its entirety). More specifically, the frequency of a C base coding for arginine 112 at site A is significantly increased in Alzheimer's disease patients.

Although the mechanistic link between Apo E e 4 and neuronal degeneration characteristic of Alzheimer's disease remains to be established, current hypotheses suggest that the Apo E genotype may influence neuronal vulnerability by increasing the deposition and/or aggregation of the amyloid beta peptide in the brain or by indirectly reducing energy availability to neurons by promoting atherosclerosis.

Using the methods of the present invention, biallelic markers that are in the vicinity of the Apo E site A were generated and the association of one of their alleles with Alzheimer's disease was analyzed. An Apo E public marker (stSG94) was used to screen a human genome BAC library as previously described. A BAC, which gave a unique FISH hybridization signal on chromosomal region 19q13.2.3, the chromosomal region harboring the Apo E gene, was selected for finding biallelic markers in linkage disequilibrium with the Apo E gene as follows.

This BAC contained an insert of 205 kb that was subcloned as previously described. Fifty BAC subclones were randomly selected and sequenced. Twenty five subclone sequences were selected and used to design twenty five pairs of PCR primers allowing 500 bp-amplicons to be generated. These PCR primers were then used to amplify the corresponding genomic sequences in a pool of DNA from 100 unrelated individuals (blood donors of French origin) as already described.

Amplification products from pooled DNA were sequenced and analyzed for the presence of biallelic polymorphisms, as already described. Five amplicons were shown to contain a polymorphic base in the pool of 100 unrelated individuals, and therefore these polymorphisms were selected as random biallelic markers in the vicinity of the Apo E gene. The sequences of both alleles of these biallelic markers (99-344-439; 99-366-274, 99-359-308; 99-355-219; 99-365-344;) correspond to SEQ ID Nos: 514 to 518. Corresponding pairs of amplification primers for generating amplicons containing these biallelic markers can be chosen from those listed as SEQ ID Nos: 536 to 540 and 558 to 562.

An additional pair of primers (SEQ ID Nos: 541 and 563) was designed that allows amplification of the genomic fragment carrying the biallelic polymorphism corresponding to the ApoE marker (99-2452-54; C/T; designated SEQ ID NO: 519 in the accompanying Sequence Listing; publicly known as Apo E site A (Weisgraber et al. (1981), supra; Rall et al. (1982), supra) to be amplified.

The five random biallelic markers plus the Apo E site A marker were physically ordered by PCR screening of the corresponding amplicons using all available BACs originally selected from the genomic DNA libraries, as previously described, using the public Apo E marker stSG94. The amplicon's order derived from this BAC screening is as follows: (99-344439/99-366-274)-(99-365-344/99-2452-54) -99-359-308-99-355-219, where parentheses indicate that the exact order of the respective amplicons couldn't be established.

Linkage disequilibrium among the six biallelic markers (five random markers plus the Apo E site A) was determined by genotyping the same 100 unrelated individuals from whom the random biallelic markers were identified.

DNA samples and amplification products from genomic PCR were obtained in similar conditions as those described above for the generation of biallelic markers, and subjected to automated microsequencing reactions using fluorescent ddNTPs (specific fluorescence for each ddNTP) and the appropriate microsequencing primers having a 3′ end immediately upstream of the polymorphic base in the biallelic markers. Once specifically extended at the 3′ end by a DNA polymerase using the complementary fluorescent dideoxynucleotide analog (thermal cycling), the microsequencing primer was precipitated to remove the unincorporated fluorescent ddNTPs. The reaction products were analyzed by electrophoresis on ABI 377 sequencing machines. Results were automatically analyzed by an appropriate software further described in Example 8.

Linkage disequilibrium (LD) between all pairs of biallelic markers (Mi, Mj) was calculated for every allele combination (Mi1,Mj1; Mi1,Mj2; Mi2,Mj1; Mi2,Mj2) according to the maximum likelihood estimate (MLE) for delta (the composite linkage disequilibrium coefficient). The results of the linkage disequilibrium analysis between the Apo E Site A marker and the five new biallelic markers (99-344439; 99-355-219; 99-359-308; 99-365-344; 99-366-274) are summarized in Table 2 below:

TABLE 2

| | d × 100 | | SEQ ID Nos of the |
|---|---|---|---|
| | APOE Site A | SEQ ID Nos of the | amplification |
| Markers | 99-2452-54 | biallelic Markers | Primers |
| ApoE Site A | | | |
| 99-2452-54 | | 519 | 541; 563 |
| 99-344-439 | 1 | 514 | 536; 558 |
| 99-366-274 | 1 | 515 | 537; 559 |
| 99-365-344 | 8 | 516 | 538; 560 |
| 99-359-308 | 2 | 517 | 539; 561 |
| 99-355-219 | 1 | 518 | 540; 562 |

The above linkage disequilibrium results indicate that among the five biallelic markers randomly selected in a region of about 200 kb containing the Apo E gene, marker 99-365-344T is in relatively strong linkage disequilibrium with the Apo E site A allele (99-2452-54C).

Therefore, since the Apo E site A allele is associated with Alzheimer's disease, one can predict that the T allele of marker 99-365-344 will probably be found associated with Alzheimer's disease. In order to test this hypothesis, the biallelic markers of SEQ ID Nos: 514 to 518 were used in association studies as described below.

225 Alzheimer's disease patients were recruited according to clinical inclusion criteria based on the MMSE test. The 248 control cases included in this study were both ethnically- and age-matched to the affected cases. Both affected and control individuals corresponded to unrelated cases. The identities of the polymorphic bases of each of the biallelic markers was determined in each of these idividuals using the methods described above. Techniques for conducting association studies are further described below.

The results of this study are summarized in Table 3 below:

TABLE 3

| | ASSOCIATION DATA | |
|---|---|---|
| | Difference in allele frequency | |
| | between individuals with Alzheimer's | Corresponding |
| MARKER | and control individuals | p-value |
| 99-344-439 | 3.3% | 9.54E−02 |
| 99-366-274 | 1.6% | 2.09E−01 |
| 99-365-344 | 17.7% | 6.9E−10 |
| 99-2452-54 | 23.8% | 3.95E−21 |
| (ApoE Site A) | | |
| 99-359-308 | 0.4% | 9.2E−01 |
| 99-355-219 | 2.5% | 2.54E−01 |

The frequency of the Apo E site A allele in both Alzheimer's disease cases and controls was found in agreement with that previously reported (ca. 10% in controls and ca. 34% in Alzheimer's disease cases, leading to a 24% difference in allele frequency), thus validating the Apo E e4 association in the populations used for this study.

Moreover, as predicted from the linkage disequilibrium analysis (Table 3), a significant association of the T allele of marker 99-365/344 with Alzheimer's disease cases (18% increase in the T allele frequency in Alzheimer's disease cases compared to controls, p value for this difference=6.9 E-10) was observed.

The above results indicate that any marker in linkage disequilibrium with one given marker associated with a trait will be associated with the trait. It will be appreciated that, though in this case the ApoE Site A marker is the trait-causing allele (TCA) itself, the same conclusion could be drawn with any other non trait-causing allele marker associated with the studied trait.

These results further indicate that conducting association studies with a set of biallelic markers randomly generated within a candidate region at a sufficient density (here about one biallelic marker every 40 kb on average), allows the identification of at least one marker associated with the trait.

In addition, these results correlate with the physical order of the six biallelic markers contemplated within the present example (see above): marker 99-365/344, which had been found to be the closest in terms of physical distance to the ApoE Site A marker, also shows the strongest linkage disequilibrium with the Apo E site A marker.

In order to further refine the relationship between physical distance and linkage disequilibrium between biallelic markers, a ca. 450 kb fragment from a genomic region on chromosome 8 was fully sequenced.

LD within ca. 230 pairs of biallelic markers derived therefrom was measured in a random French population and analyzed as a function of the known physical inter-marker spacing. This analysis confirmed that, on average, linkage disequilibrium between 2 biallelic markers correlates with the physical distance that separates them. It further indicated that linkage disequilibrium between 2 biallelic markers tends to decrease when their spacing increases. More particularly, linkage disequilibrium between 2 biallelic markers tends to decrease when their inter-marker distance is greater than 50 kb, and is further decreased when the inter-marker distance is greater than 75 kb. It was further observed that when 2 biallelic markers were further than 150 kb apart, most often no significant linkage disequilibrium between them could be evidenced. It will be appreciated that the size and history of the sample population used to measure linkage disequilibrium between markers may influence the distance beyond which linkage disequilibrium tends not to be detectable. Assuming that linkage disequilibrium can be measured between markers spanning regions up to an average of 150 kb long, biallelic marker maps will allow genome-wide linkage disequilibrium mapping, provided they have an average inter-marker distance lower than 150 kb.

### Example 6

### Identification of a Candidate Region Harboring a Gene Associated with a Detectable Trait

The initial identification of a candidate genomic region harboring a gene associated with a detectable trait may be conducted using a genome-wide map comprising about 20,000 biallelic markers. The candidate genomic region may be further defined using a map having a higher marker density, such as a map comprising about 40,000 markers, about 60,000 markers, about 80,000 markers, about 100,000 markers, or about 120,000 markers.

The use of high density maps such as those described above allows the identification of genes which are truly associated with detectable traits, since the coincidental associations will be randomly distributed along the genome while the true associations will map within one or more discrete genomic regions. Accordingly, biallelic markers located in the vicinity of a gene associated with a detectable trait will give rise to broad peaks in graphs plotting the frequencies of the biallelic markers in trait-positive individuals versus control individuals. In contrast, biallelic markers which are not in the vicinity of the gene associated with the detectable trait will produce unique points in such a plot. By determining the association of several markers within the region containing the gene associated with the detectable trait, the gene associated with the detectable trait can be identified using an association curve which reflects the difference between the allele frequencies within the trait-positive and control populations for each studied marker. The gene associated with the detectable trait will be found in the vicinity of the marker showing the highest association with the trait.

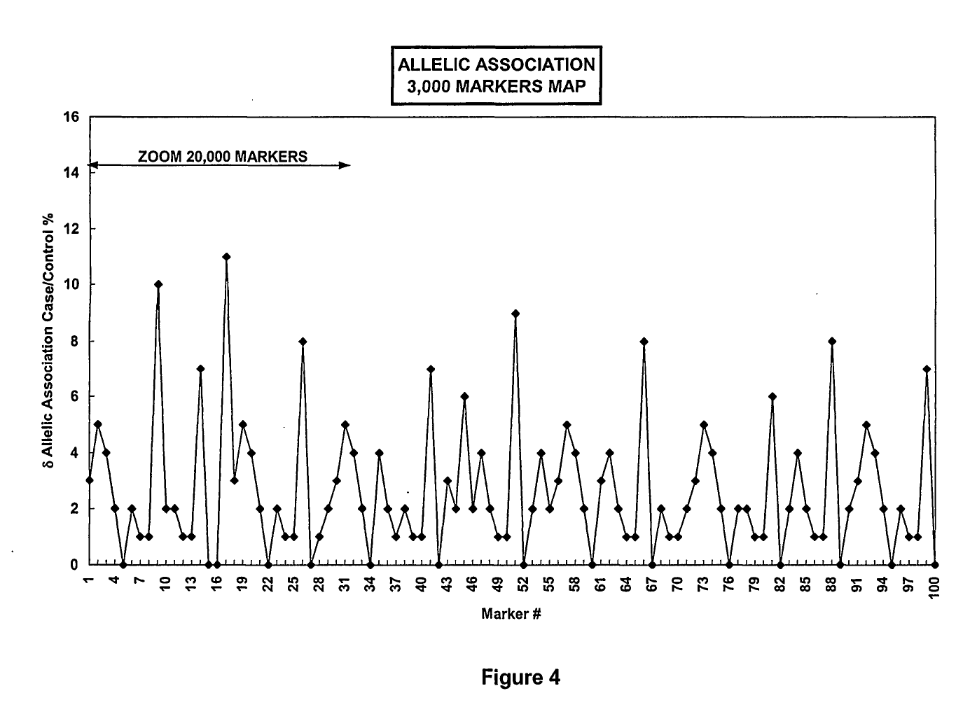

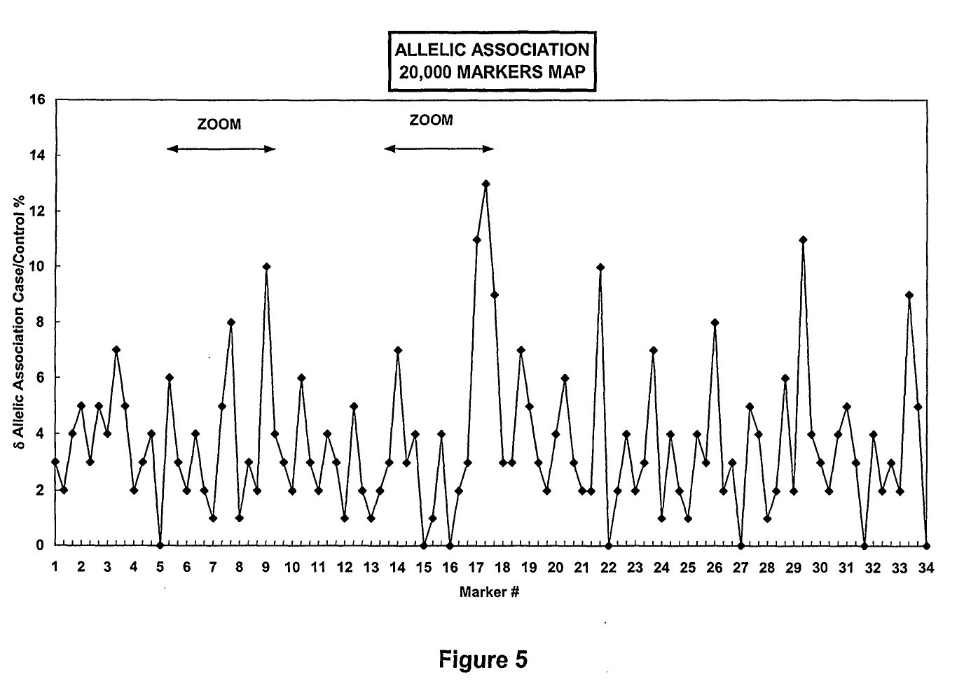

FIGS. 4, 5, and 6 provide a simulated illustration of the above principles. As illustrated in FIG. 4, an association analysis conducted with a map comprising about 3,000 biallelic markers yields a group of points. However, when an association analysis is performed using a denser map which includes additional biallelic markers, the points become broad peaks indicative of the location of a gene associated with a detectable trait. For example, the biallelic markers used in the initial association analysis may be obtained from a map comprising about 20,000 biallelic markers, as illustrated by the simulation results shown in FIG. 5. In some embodiments, one or more of the biallelic markers of SEQ ID Nos. 1 to 171, 1 to 100, 101 to 162, 163 to 171 or the sequences complementary thereto are used in the association analysis.

In the simulated results of FIG. 4, the association analysis with 3,000 markers suggests peaks near markers 9 and 17.

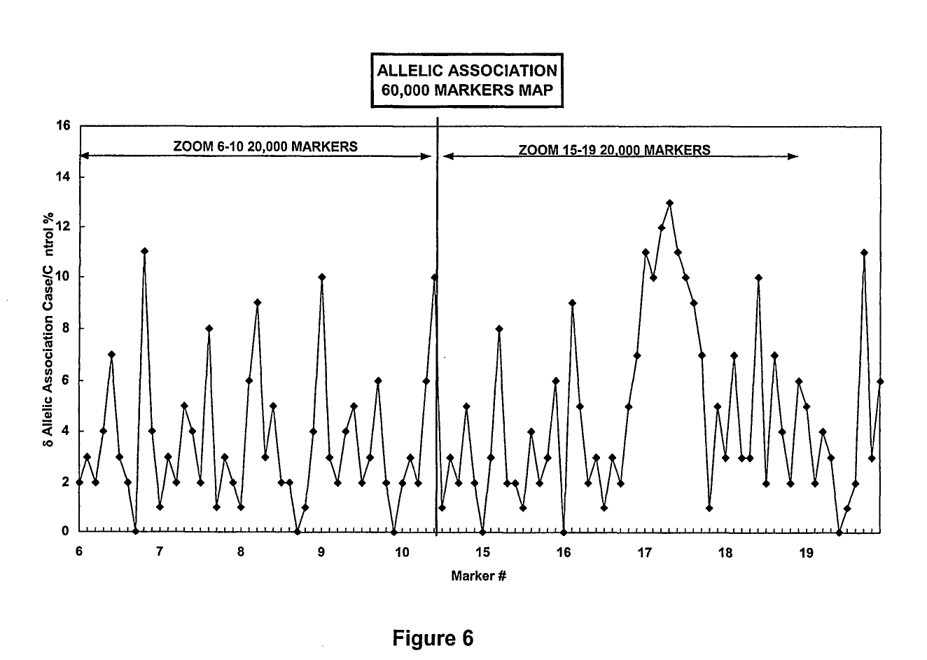

Next, a second analysis is performed using additional markers in the vicinity of markers 9 and 17, as illustrated in the simulated results of FIG. 5, using a map of about 20,000 markers. This step again indicates an association in the close vicinity of marker 17, since more markers in this region show an association with the trait. However, none of the additional markers around marker 9 shows a significant association with the trait, which makes marker 9 a potential false positive. In some embodiments, one or more of the biallelic markers selected from the group consisting of SEQ ID Nos. 1 to 171, 1 to 100, 101 to 162, 163 to 171 or the sequences complementary thereto are used in the second analysis. In order to further test the validity of these two suspected associations, a third analysis may be obtained with a map comprising about 60,000 biallelic markers. In some embodiments, one or more of the biallelic markers selected from the group consisting of SEQ ID Nos. 1 to 171, 1 to 100, 101 to 162, 163 to 171 or the sequences complementary thereto are used in the third association analysis. In the simulated results of FIG. 6, more markers lying around marker 17 exhibit a high degree of association with the detectable trait. Conversely, no association is confirmed in the vicinity of marker 9. The genomic region surrounding marker 17 can thus be considered a candidate region for the potential trait of this simulation.

### Example 7

### Haplotype Analysis: Identification of Biallelic Markers Delineating a Genomic Region Associated with Alzheimer's Disease (AD)

As shown in Table 3 within Example 5, at an average map density of one marker per 40 kb only one marker (99-365-344) out of five random biallelic markers from a ca. 200 kb genomic region around the Apo E gene showed a clear association to Alzheimer's disease (delta allelic frequency in cases and controls=18%; p value=6.9 E-10). The allelic frequencies of the other four random markers were not significantly different between Alzheimer's disease cases and controls (p-values≧E-01). However, since linkage disequilibrium can usually be detected between markers located further apart than an average 40 kb as previously discussed, one should expect that, performing an association study with a local excerpt of a biallelic marker map covering ca. 200 kb with an average inter-marker distance of ca. 40 kb should allow the identification of more than one biallelic marker associated with Alzheimer's disease.

A haplotype analysis was thus performed using the biallelic markers 99-344-439; 99-355-219; 99-359-308; 99-365-344; and 99-366-274 (of SEQ ID Nos: 514 to 518).

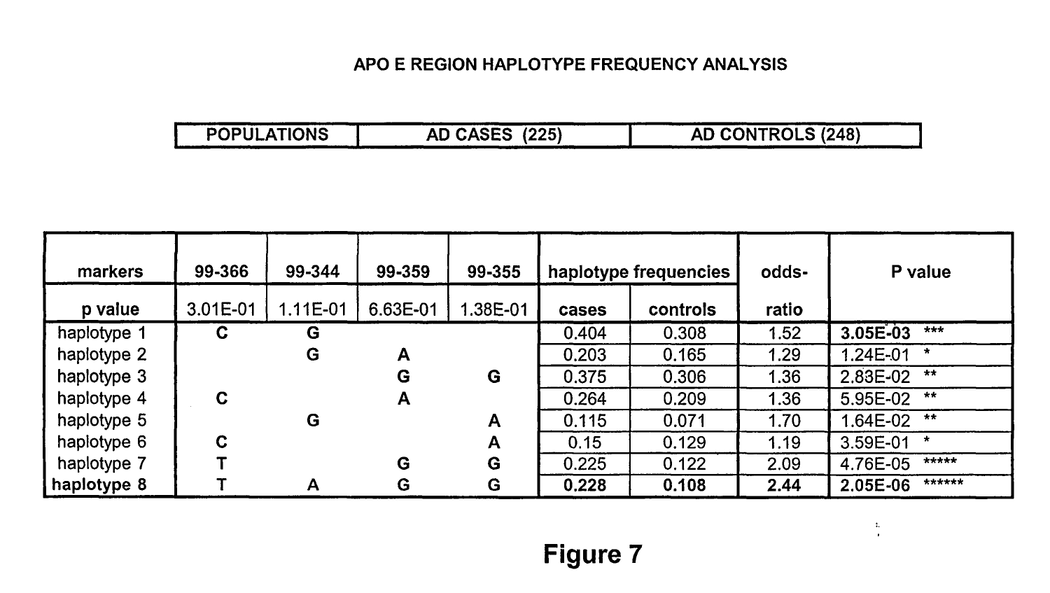

In a first step, marker 99-365-344 that was already found associated with Alzheimer's disease was not included in the haplotype study. Only biallelic markers 99-344439, 99-355-219, 99-359-308, and 99-366-274, which did not show any significant association with Alzheimer's disease when taken individually, were used. This first haplotype analysis measured frequencies of all possible two-, three-, or four-marker haplotypes in the Alzheimer's disease case and control populations. As shown in FIG. 7, there was one haplotype among all the potential different haplotypes based on the four individually non-significant markers (“haplotype 8”, TAGG comprising SEQ ID No.515 with the T allele of marker 99-366-274, SEQ D No. 514 with the A allele of marker 99-344439, SEQ ID No.516 with the G allele of marker 99-359-308 and SEQ ID No.517 which is the G allele of marker 99-355-219), that was present at statistically significant different frequencies in the Alzheimer's disease case and control populations (D=12%; p value=2.05 E-06). Moreover, a significant difference was already observed for a three-marker haplotype included in the above mentioned “haplotype 8” (“haplotype 7”, TGG, D=10%; p value=4.76 E-05). Haplotype 7 comprises SEQ ID No. 515 with the T allele of marker 99-366-274, SEQ ID No. 516 with the G allele of marker 99-359-308 and SEQ ID No. 517 with the G allele of marker 99-355-219). The haplotype association analysis thus clearly increased the statistical power of the individual marker association studies by more than four orders of magnitude when compared to single-marker analysis from p values≧E-01 for the individual markers to p value≦2 E-06 for the four-marker “haplotype 8”. See Table 3.

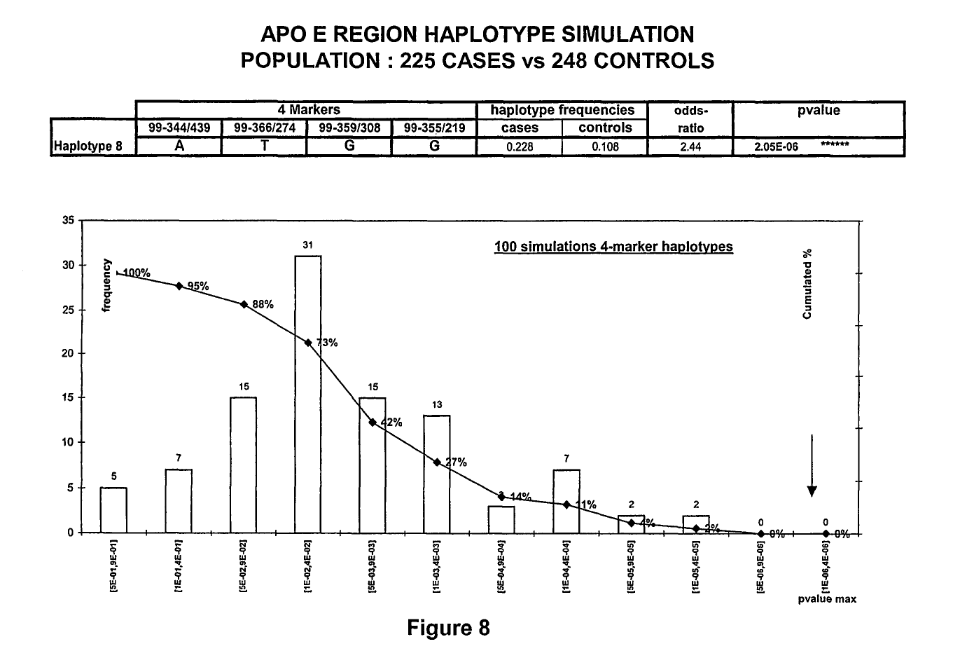

The significance of the values obtained for this haplotype association analysis was evaluated by the following computer simulation. The genotype data from the Alzheimer's disease cases and the unaffected controls were pooled and randomly allocated to two groups which contained the same number of individuals as the case/control groups used to produce the data summarized in FIG. 7. A four-marker haplotype analysis (99-344439; 99-355-219; 99-359-308; and 99-366-274) was run on these artificial groups. This experiment was reiterated 100 times and the results are shown in FIG. 8. No haplotype among those generated was found for which the p-value of the frequency difference between both populations was more significant than 1 E-05. In addition, only 4% of the generated haplotypes showed p-values lower than 1 E-04. Since both these p-value thresholds are less significant than the 2 E-06 p-value showed by “haplotype 8”, this haplotype can be considered significantly associated with Alzheimer's disease.

In a second step, marker 99-365-344 was included in the haplotype analyzes. The frequency differences between the affected and non affected populations was calculated for all two-, three-, four- or five-marker haplotypes involving markers: 99-344439; 99-355-219; 99-359-308; 99-366-274; and 99-365-344. The most significant p-values obtained in each category of haplotype (involving two, three, four or five markers) were examined depending on which markers were involved or not within the haplotype. This showed that all haplotypes which included marker 99-365-344 showed a significant association with Alzheimer's disease (p-values in the range of E-04 to E-11).

An additional way of evaluating the significance of the values obtained in the haplotype association analysis was to perform a similar Alzheimer's disease case-control study on biallelic markers generated from BACs containing inserts corresponding to genomic regions derived from chromosomes 13 or 21 and not known to be involved in Alzheimer's disease. Performing similar haplotype and individual association analyzes as those described above did not generate any significant association results (all p-values for haplotype analyzes were less significant than E-03; all p-values for single marker association studies were less significant than E-02).

### Example 8

### Genotyping of Biallelic Markers Using Microsequencing Procedures

Several microsequencing protocols conducted in liquid phase are well known to those skilled in the art. A first possible detection analysis allowing the allele characterization of the microsequencing reaction products relies on detecting fluorescent ddNTP-extended microsequencing primers after gel electrophoresis. A first alternative to this approach consists in performing a liquid phase microsequencing reaction, the analysis of which may be carried out in solid phase.

For example, the microsequencing reaction may be performed using 5′-biotinylated oligonucleotide primers and fluorescein-dideoxynucleotides. The biotinylated oligonucleotide is annealed to the target nucleic acid sequence immediately adjacent to the polymorphic nucleotide position of interest. It is then specifically extended at its 3′-end following a PCR cycle, wherein the labeled dideoxynucleotide analog complementary to the polymorphic base is incorporated. The biotinylated primer is then captured on a microtiter plate coated with streptavidin. The analysis is thus entirely carried out in a microtiter plate format. The incorporated ddNTP is detected by a fluorescein antibody—alkaline phosphatase conjugate.

In practice this microsequencing analysis is performed as follows. 20 μl of the microsequencing reaction is added to 80 μl of capture buffer (SSC 2×, 2.5% PEG 8000, 0.25 M Tris pH 7.5, 1.8% BSA, 0.05% Tween 20) and incubated for 20 minutes on a microtiter plate coated with streptavidin (Boehringer). The plate is rinsed once with washing buffer (0.1 M Tris pH 7.5, 0.1 M NaCl, 0.1% Tween 20). 100 μl of anti-fluorescein antibody conjugated with phosphatase alkaline, diluted 1/5000 in washing buffer containing 1.8% BSA is added to the microtiter plate. The antibody is incubated on the microliter plate for 20 minutes. After washing the microtiter plate four times, 100 μl of 4-methylumbelliferyl phosphate (Sigma) diluted to 0.4 mg/ml in 0.1 M diethanolamine pH 9.6, 10 mM MgCl₂ are added. The detection of the microsequencing reaction is carried out on a fluorimeter (Dynatech) after 20 minutes of incubation.

As another alternative, solid phase microsequencing reactions have been developed, for which either the oligonucleotide microsequencing primers or the PCR-amplified products derived from the DNA fragment of interest are immobilized. For example, immobilization can be carried out via an interaction between biotinylated DNA and streptavidincoated microtitration wells or avidin-coated polystyrene particles.

As a further alternative, the PCR reaction generating the amplicons to be genotyped can be performed directly in solid phase conditions, following procedures such as those described in WO 96/13609, the disclosure of which is incorporated herein by reference in its entirety.

In such solid phase microsequencing reactions, incorporated ddNTPs can either be radiolabeled (see Syvänen, Clin. Chim. Acta. 226:225-236 (1994), the disclosure of which is incorporated herein by reference in its entirety) or linked to fluorescein (see Livak and Hainer, Hum. Metat. 3:379-385 (1994), the disclosure of which is incorporated herein by reference in its entirety). The detection of radiolabeled ddNTPs can be achieved through scintillation-based techniques. The detection of fluorescein-linked ddNTPs can be based on the binding of antifluorescein antibody conjugated with alkaline phosphatase, followed by incubation with a chromogenic substrate (such as p-nitrophenyl phosphate).

Other possible reporter-detection couples for use in the above microsequencing procedures include:

ddNTP linked to dinitrophenyl (DNP) and anti-DNP alkaline phosphatase conjugate (see Harju et al., Clin Chem:39(11Pt 1):2282-2287 (1993), incorporated herein by reference in its entirety)

biotinylated ddNTP and horseradish peroxidase-conjugated streptavidin with o-phenylenediamine as a substrate (see WO 92/15712, incorporated herein by reference in its entirety).

A diagnosis kit based on fluorescein-linked ddNTP with antifluorescein antibody conjugated with alkaline phosphatase has been commercialized under the name PRONTO by GamidaGen Ltd.

As yet another alternative microsequencing procedure, Nyren et al. (Anal. Biochem. 208:171-175 (1993), the disclosure of which is incorporated herein by reference in its entirety) have described a solid-phase DNA sequencing procedure that relies on the detection of DNA polymerase activity by an enzymatic luminometric inorganic pyrophosphate detection assay (ELIDA). In this procedure, the PCR-amplified products are biotinylated and immobilized on beads. The microsequencing primer is annealed and four aliquots of this mixture are separately incubated with DNA polymerase and one of the four different ddNTPs. After the reaction, the resulting fragments are washed and used as substrates in a primer extension reaction with all four dNTPs present. The progress of the DNA-directed polymerization reactions is monitored with the ELIDA. Incorporation of a ddNTP in the first reaction prevents the formation of pyrophosphate during the subsequent dNTP reaction. In contrast, no ddNTP incorporation in the first reaction gives extensive pyrophosphate release during the dNTP reaction and this leads to generation of light throughout the ELIDA reactions. From the ELIDA results, the identity of the first base after the primer is easily deduced.

It will be appreciated that several parameters of the above-described microsequencing procedures may be successfully modified by those skilled in the art without undue experimentation. In particular, high throughput improvements to these procedures may be elaborated, following principles such as those described further below.

### EXAMPLE 9

### Sequence Analysis

DNA sequences, such as BAC inserts, containing the region carrying the candidate gene associated with the detectable trait are sequenced and their sequence is analyzed using automated software which eliminates repeat sequences while retaining potential gene sequences. The potential gene sequences are compared to numerous databases to identify potential exons using a set of scoring algorithms such as trained Hidden Markov Models, statistical analysis models (including promoter prediction tools) and the GRAIL neural network.

NRPU (Non-Redundant Protein-Unique) database: NRPU is a non-redundant merge of the publicly available NBRF/PIR, Genpept, and SwissProt databases. Homologies found with NRPU allow the identification of regions potentially coding for already known proteins or related to known proteins (translated exons).

NREST (Non-Redundant EST database): NREST is a merge of the EST subsection of the publicly available GenBank database. Homologies found with NREST allow the location of potentially transcribed regions (translated or non-translated exons).

NRN (Non-Redundant Nucleic acid database): NRN is a merge of GenBank, EMBL and their daily updates.

Any sequence giving a positive hit with NRPU, NREST or an “excellent” score using GRAIL or/and other scoring algorithms is considered a potential functional region, and is then considered a candidate for genomic analysis.

While this first screening allows the detection of the “strongest” exons, a semi-automatic scan is further applied to the remaining sequences in the context of the sequence assembly: That is, the sequences neighboring a 5′ site or an exon are submitted to another round of bioinformatics analysis with modified pararneters. In this way, new exon candidates are generated for genomic analysis.

Using the above procedures, genes associated with detectable traits may be identified.

### Example 10

### YAC Contig Construction in the Candidate Genomic Region

Substantial amounts of LOH data supported the hypothesis that genes associated with distinct cancer types are located within a particular region of the human genome. More specifically, this region was likely to harbor a gene associated with prostate cancer. Association studies were performed as described below in order to identify this prostate cancer gene. First, a YAC contig which contains the candidate genomic region was constructed as follows. The CEPH-Genethon YAC map for the entire human genome (Chumakov et al. (1995), supra) was used for detailed contig building in the genomic region containing genetic markers known to map in the candidate genomic region. Screening data available for several publicly available genetic markers were used to select a set of CEPH YACs localized within the candidate region. This set of YACs was tested by PCR with the above mentioned genetic markers as well as with other publicly available markers supposedly located within the candidate region. As a result of these studies, a YAC STS contig map was generated around genetic markers known to map in this genomic region. Two CEPH YACs were found to constitute a minimal tiling path in this region, with an estimated size of ca. 2 Megabases.

During this mapping effort, several publicly known STS markers were precisely located within the contig.

Example 11 below describes the identification of sets of biallelic markers within the candidate genomic region.

### Example 11

### BAC Contig Construction and Biallelic Markers Isolation within the Candidate Chromosomal Region

Next, a BAC contig covering the candidate genomic region was constructed as follows. BAC libraries were obtained as described in Woo et al., Nucleic Acids Res. 22:4922-4931 (1994), the disclosure of which is incorporated herein by reference in its entirety. Briefly, the two whole human genome BamHI and HindIII libraries already described in related WIPO application No. PCT/IB98/00193 were constructed using the pBeloBAC11 vector (Kim et al. (1996), supra).

The BAC libraries were then screened with all of the above mentioned STSs, following the procedure described in Example 1 above.

The ordered BACs selected by STS screening and verified by FISH, were assembled into contigs and new markers were generated by partial sequencing of insert ends from some of them. These markers were used to fill the gaps in the contig of BAC clones covering the candidate chromosomal region having an estimated size of 2 megabases.

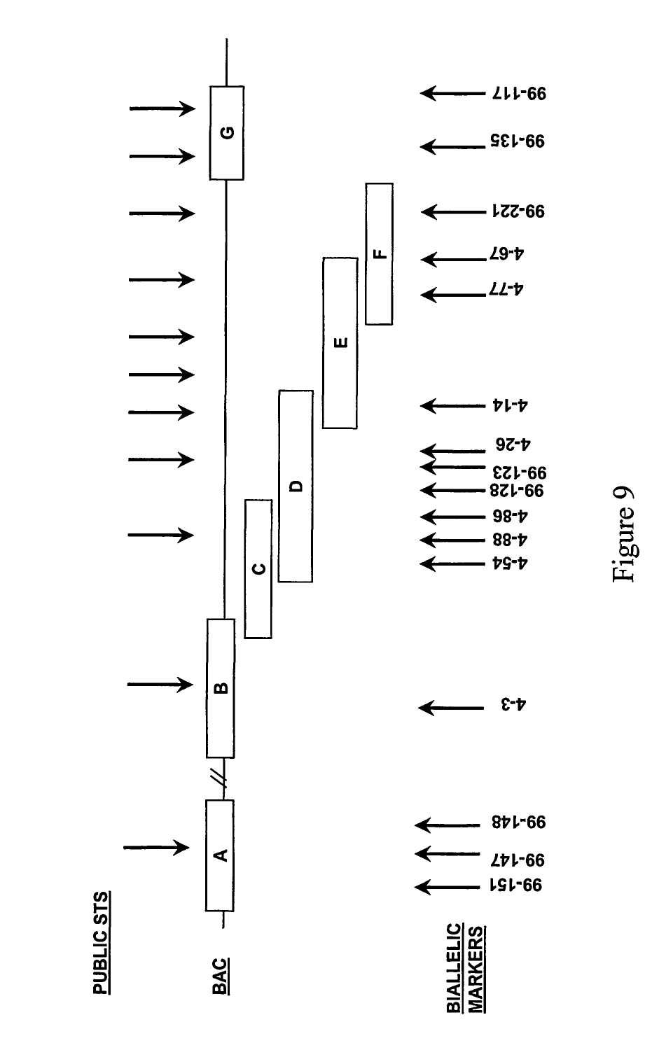

FIG. 9 illustrates a minimal array of overlapping clones which was chosen for further studies, and the positions of the publicly known STS markers along said contig.

Selected BAC clones from the contig were subcloned and sequenced, essentially following the procedures described in related WIPO application No. PCT/IB98/00193.

Biallelic markers lying along the contig were identified following the processes described in related WIPO application No. PCT/IB98/00193, the disclosure of which is incorporated herein by reference in its entirety.

FIG. 9 shows the locations of the biallelic markers along the BAC contig. This first set of markers corresponds to a medium density map of the candidate locus, with an inter-marker distance averaging 50 kb-150 kb.

A second set of biallelic markers was then generated as described above in order to provide a very high-density map of the region identified using the first set of markers which can be used to conduct association studies, as explained below. This very high density map has markers spaced on average every 2-50 kb.

The biallelic markers were then used in association studies. DNA samples were obtained from individuals suffering from prostate cancer and unaffected individuals as described in Example 12.

### Example 12

### Collection of DNA Samples from Affected and Non-Affected Individuals

Prostate cancer patients were recruited according to clinical inclusion criteria based on pathological or radical prostatectomy records. Control cases included in this study were both ethnically- and age-matched to the affected cases; they were checked for both the absence of all clinical and biological criteria defining the presence or the risk of prostate cancer; and for the absence of related familial prostate cancer cases. Both affected and control individuals were all unrelated.

The two following groups of independent individuals were used in the association studies. The first group, comprising individuals suffering from prostate cancer, contained 185 individuals. Of these 185 cases of prostate cancer, 47 cases were sporadic and 138 cases were familial. The control group contained 104 non-diseased individuals.

Haplotype analysis was conducted using additional diseased (total samples: 281) and control samples (total samples: 130), from individuals recruited according to similar criteria.

DNA was extracted from peripheral venous blood of all individuals as described in related WIPO application No. PCT/IB98/00193.

The frequencies of the biallelic markers in each population were determined as described in Example 13.

### Example 13

### Genotyping Affected and Control Individuals

Genotyping was performed using the following microsequencing procedure. Amplification was performed on each DNA sample using primers designed as previously explained. The pairs of primers of SEQ ID Nos.: 542 to 553 and 564 to 575 were used to generate amplicons harboring the biallelic markers of SEQ ID Nos: 520 to 531 or the sequences complementary thereto (markers 99-123-381, 4-26-29, 4-14-240, 4-77-151, 99-217-277, 4-67-40, 99-213-164, 99-221-377, 99-135-196, 99-1482-32, 4-73-134, and 4-65-324) using the protocols described in related WIPO application No. PCT/IB98/00193.

Microsequencing primers were designed for each of the biallelic markers, as previously described. After purification of the amplification products, the microsequencing reaction mixture was prepared by adding, in a 20 μl final volume: 10 μmol microsequencing oligonucleotide, 1 U Thermosequenase (Amersham E79000G), 1.25 μl Thermosequenase buffer (260 mM Tris HCl pH 9.5, 65 mM MgCl₂), and the two appropriate fluorescent ddNTPs (Perkm Elmer, Dye Terminator Set 401095) complementary to the nucleotides at the polymorphic site of each biallelic marker tested, following the manufacturer's recommendations. After 4 minutes at 94° C., 20 PCR cycles of 15 sec at 55° C., 5 sec at 72° C., and 10 sec at 94° C. were carried out in a Tetrad PTC-225 thermocycler (MJ Research). The unincorporated dye terminators were then removed by ethanol precipitation. Samples were finally resuspended in formamide-EDTA loading buffer and heated for 2 min at 95° C. before being loaded on a polyacrylamide sequencing gel. The data were collected by an ABI PRISM 377 DNA sequencer and processed using the GENESCAN software (Perkin Elmer).

Following gel analysis, data were automatically processed with software that allows the determination of the alleles of biallelic markers present in each amplified fragment.

The software evaluates such factors as whether the intensities of the signals resulting from the above microsequencing procedures are weak, normal, or saturated, or whether the signals are ambiguous. In addition, the software identifies significant peaks (according to shape and height criteria). Among the significant peaks, peaks corresponding to the targeted site are identified based on their position. When two significant peaks are detected for the same position, each sample is categorized as homozygous or heterozygous based on the height ratio.

Association analyzes were then performed using the biallelic markers as described below.

### Example 14

### Association Analysis

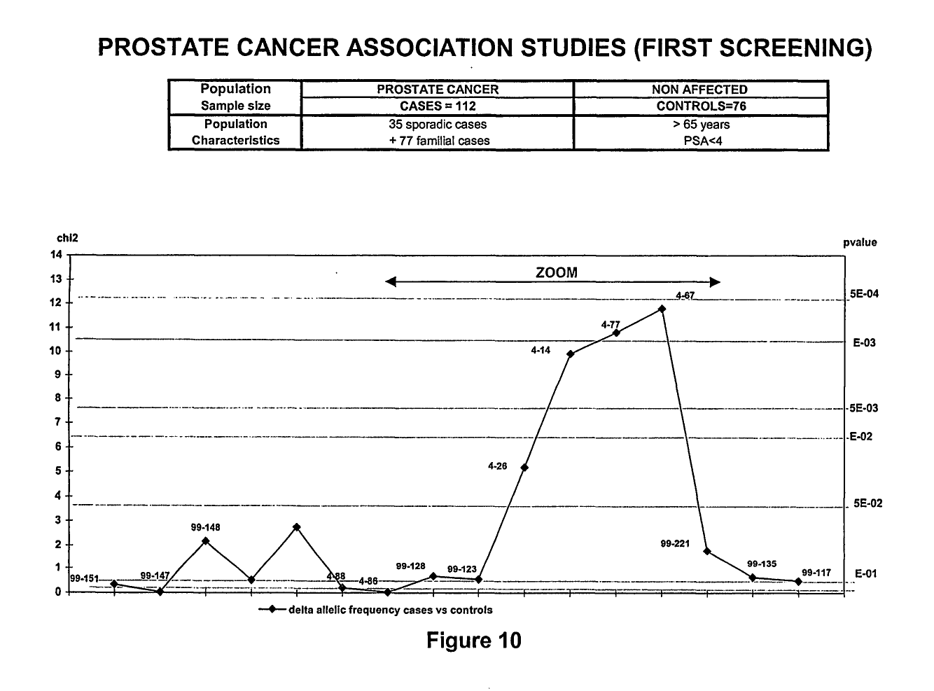

Association studies were run in two successive steps. In a first step, a rough localization of the candidate gene was achieved by determining the frequencies of the biallelic markers of FIG. 9 in the affected and unaffected populations. The results of this rough localization are shown in FIG. 10. This analysis indicated that a gene responsible for prostate cancer was located near the biallelic marker designated 4-67.

In a second phase of the analysis, the position of the gene responsible for prostate cancer was further refined using the very high density set of markers including the markers of SEQ ID Nos: 520 to 531 or the sequences complementary thereto (markers 99-123-381, 4-26-29, 4-14-240, 4-77-151, 99-217-277, 4-67-40, 99-213-164, 99-221-377, 99-135-196, 99-1482-32, 4-73-134, and 4-65-324).

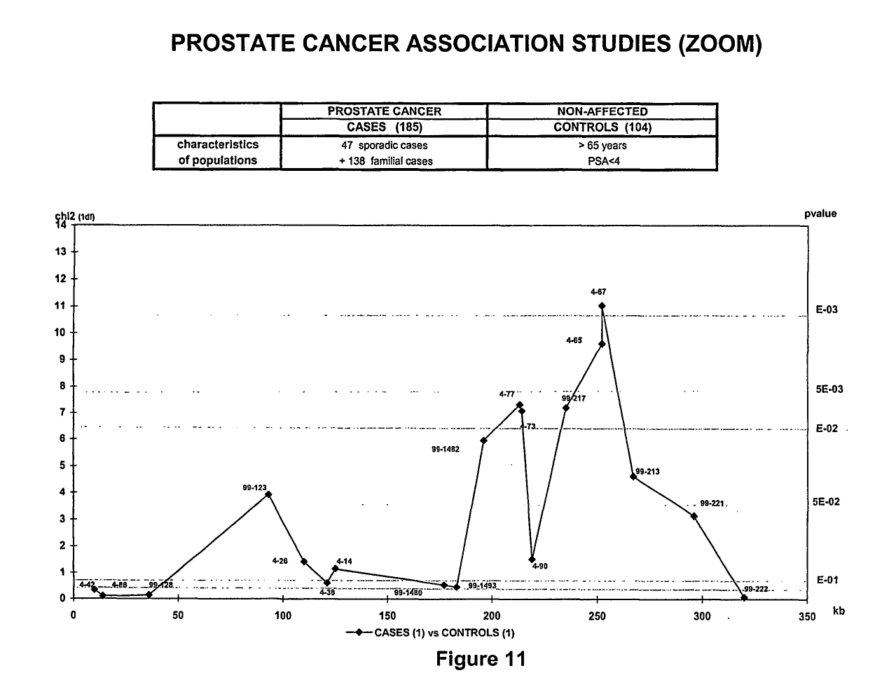

As shown in FIG. 11, the second phase of the analysis confirmed that the gene responsible for prostate cancer was near the biallelic marker designated 4-6740, most probably within a ca. 150 kb region comprising the marker.

A haplotype analysis was also performed as described in Example 15.

### Example 15

### Haplotype Analysis

The allelic frequencies of each of the alleles of biallelic rnarkers 99-123-381, 4-26-29, 4-14-240, 4-77-151, 99-217-277, 4-67-40, 99-213-164, 99-221-377, and 99-135-196 were determined in the affected and unaffected populations. Table 4 lists the internal identification numbers of the markers used in the haplotype analysis (SEQ ID Nos: 520-528), the alleles of each marker, the most frequent allele in both unaffected individuals and individuals suffering from prostate cancer, the least frequent allele in both unaffected individuals and individuals suffering from prostate cancer, and the frequencies of the least frequent alleles in each population.

TABLE 4

Frequency of least
frequent allele **

| Markers | Polymorphic base * | Cases | Controls |
|---|---|---|---|
| 99-123-381 | C/T | 0.35 | 0.3 |
| 4-26-29 | A/G | 0.39 | 0.45 |
| 4-14-240 | C/T | 0.35 | 0.41 |
| 4-77-151 | C/G | 0.33 | 0.24 |
| 99-217-277 | C/T | 0.31 | 0.23 |
| 4-67-40 | C/T | 0.26 | 0.16 |
| 99-213-164 | T/C | 0.45 | 0.38 |
| 99-221-377 | C/A | 0.43 | 0.43 |
| 99-135-196 | A/G | 0.25 | 0.3 |

* most frequent allele/least frequent allele
** standard deviations −0.023 to 0.031 for controls −0.018 to 0.021 for cases

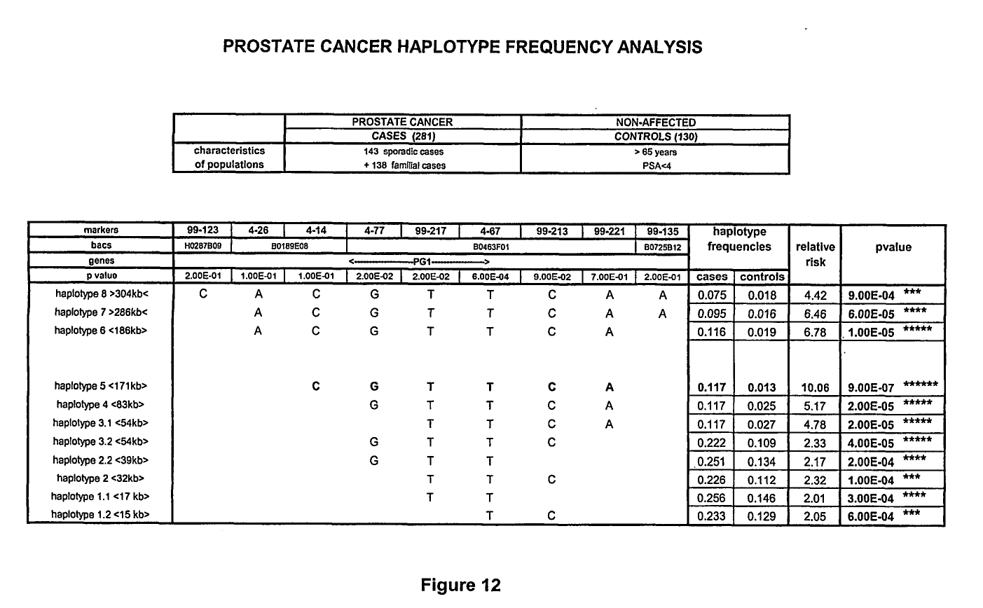

Among all the theoretical potential different haplotypes based on 2 to 9 markers, 11 haplotypes showing a strong association with prostate cancer were selected. The results of these haplotype analyzes are shown in FIG. 12.

FIGS. 11 and 12 aggregate association analysis results with sequencing results—generated following the procedures further described in Example 16, which permitted the physical order and the distance between markers to be estimated.

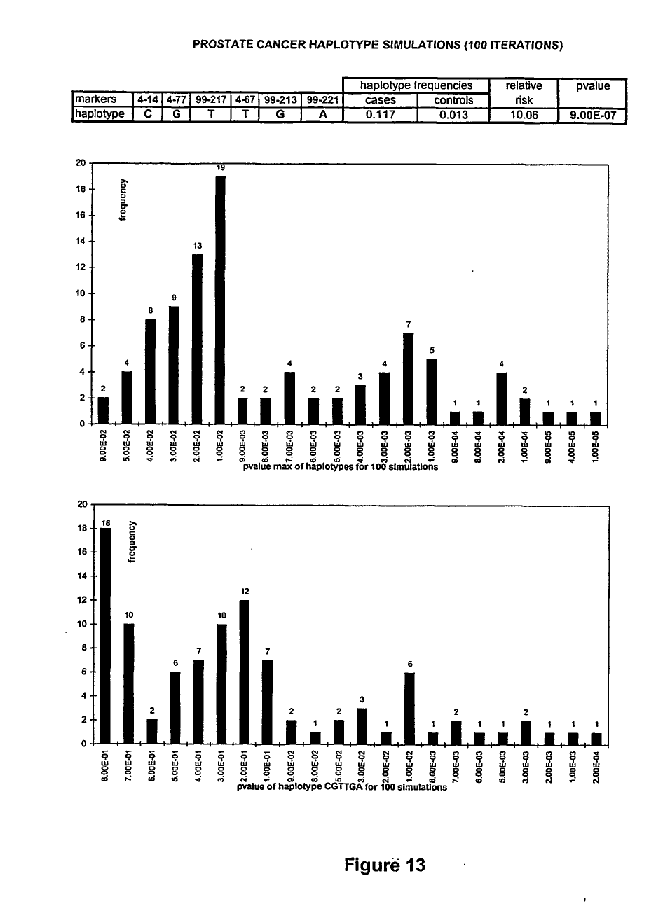

The significance of the values obtained in FIG. 12 are underscored by the following results of computer simulations. For the computer simulations, the data from the affected individuals and the unaffected controls were pooled and randomly allocated to two groups which contained the same number of individuals as the affected and unaffected groups used to compile the data summarized in FIG. 12. A haplotype analysis was run on these artificial groups for the six markers included in haplotype 5 of FIG. 12. This experiment was reiterated 100 times and the results are shown in FIG. 13. Among 100 iterations, only 5% of the obtained haplotypes are present with a p-value less significant than E-04 as compared to the p-value of 9E-07 for haplotype 5 of FIG. 12. Furthermore, for haplotype 5 of FIG. 12, only 6% of the obtained haplotypes have a significance level below 5^{E}-03, while none of them show a significance level below 5E-03.

Thus, using the data of FIG. 13 and evaluating the associations for single marker alleles or for haplotypes will permit estimation of the risk a corresponding carrier has to develop prostate cancer. It will be appreciated that significance thresholds of relative risks will be more finely assessed according to the population tested.

Diagnostic techniques for determining an individual's risk of developing prostate cancer may be implemented as described below for the markers in the maps of the present invention, including the markers of SEQ ID Nos: 520 to 528 (markers 99-123-381, 426-29, 4-14-240, 4-77-151, 99-217-277,4-6740, 99-213-164, 99-221-377, and 99-135-196).

The above haplotype analysis indicated that 171 kb of genomic DNA between biallelic markers 4-14-240 and 99-221-377 totally or partially contains a gene responsible for prostate cancer. Therefore, the protein coding sequences lying within this region were characterized to locate the gene associated with prostate cancer. This analysis, described in further detail below, revealed a single protein coding sequence in the 171 kb genomic region, which was designated as the PG1 gene.

### Example 16

### Identification of the Genomic Sequence in the Candidate Region

Template DNA for sequencing the PG1 gene was obtained as follows. BACs E and F from FIG. 9 were subcloned as previously described. Plasmid inserts were first amplified by PCR on PE 9600 thermocyclers (Perkin-Elmer), using appropriate primers, AmpliTaqGold (Perkin-Elmer), dNTPs (Boehringer), buffer and cycling conditions as recommended by the Perkin-Elmer Corporation.

PCR products were then sequenced using automatic ABI Prism 377 sequencers (Perkin Elmer, Applied Biosystems Division, Foster City, Calif.). Sequencing reactions were performed using PE 9600 thermocyclers (Perldn Elmer) with standard dye-primer chemistry and ThermoSequenase (Amersham Life Science). The primers were labeled with the JOE, FAM, ROX and TAMRA dyes. The dNTPs and ddNTPs used in the sequencing reactions were purchased from Boehringer. Sequencing buffer, reagent concentrations and cycling conditions were as recommended by Amersham.

Following the sequencing reaction, the samples were precipitated with EtOH, resuspended in formamide loading buffer, and loaded on a standard 4% acrylamide gel. Electrophoresis was performed for 2.5 hours at 3000V on an ABI 377 sequencer, and the sequence data were collected and analyzed using the ABI Prism DNA Sequencing Analysis Software, version 2.1.2.

The sequence data obtained as described above were transferred to a proprietary database, where quality control and validation steps were performed. A proprietary base-caller flagged suspect peaks, taking into account the shape of the peaks, the inter-peak resolution, and the noise level. The proprietary base-caller also performed an automatic trimming. Any stretch of 25 or fewer bases having more than 4 suspect peaks was considered unreliable and was discarded.

The sequence fragments from BAC subclones isolated as described above were assembled using Gap4 software from R. Staden (Bonfield et al. 1995). This software allows the reconstruction of a single sequence from sequence fragments. The sequence deduced from the alignment of different fragments is called the consensus sequence. Directed sequencing techniques (primer walking) were used to complete sequences and link contigs.

Potential functional sequences were then identified as described in Example 17.

### Example 17

### Identification of Functional Sequences

Potential exons in BAC-derived human genomic sequences were located by homology searches on protein, nucleic acid and EST (Expressed Sequence Tags) public databases. Main public databases were locally reconstructed as mentioned in Example 9. The protein database, NRPU (Non-redundant Protein Unique) is formed by a non-redundant fusion of the Genpept (Benson et al., Nucleic Acids Res. 24:1-5 (1996), the disclosure of which is incorporated herein by reference in its entirety), Swissprot (Bairoch, A. and Apweiler, R., Nucleic Acids Res. 24:21-25 (1996), the disclosure of which is incorporated herein by reference in its entirety) and PIR/NBRF (George et al., Nucleic Acids Res. 24:17-20 (1996), the disclosure of which is incorporated herein by reference in its entirety) databases. Redundant data were eliminated by using the NRDB software (Benson et al. (1996), supra) and internal repeats were masked with the XNU software (Benson et al., supra). Homologies found using the NRPU database allowed the identification of sequences corresponding to potential coding exons related to known proteins.

The EST local database is composed by the gbest section (1-9) of GenBank (Benson et al. (1996), supra), and thus contains all publicly available transcript fragments. Homologies found with this database allowed the localization of potentially transcribed regions.

The local nucleic acid database contained all sections of GenBank and EMBL (Rodriguez-Tome et al., Nucleic Acids Res. 24:6-12 (1996), the disclosure of which is incorporated herein by reference in its entirety) except the EST sections. Redundant data were eliminated as previously described.

Similarity searches in protein or nucleic acid databases were performed using the BLAST software (Altschul et al., J. Mol. Biol. 215:403410 (1990), the disclosure of which is incorporated herein by reference in its entirety). Alignments were refined using the Fasta software, and multiple alignments used Clustal W. Homology thresholds were adjusted for each analysis based on the length- and the complexity of the tested region, as well as on the size of the reference database.

Potential exon sequences identified as above were used as probes to screen cDNA libraries. Extremities of positive clones were sequenced and the sequence stretches were positioned on the genomic sequence determined above. Primers were then designed using the results from these alignments in order to enable the cloning of cDNAs derived from the gene associated with prostate cancer that was identified using the above procedures.

The obtained cDNA molecules were then sequenced and results of Northern blot analysis of prostate mRNAs supported the existence of a major cDNA having a 5-6 kb length. The structure of the gene associated with prostate cancer was evaluated as described in Example 18.

### Example 18

### Analysis of Gene Structure

The intron/exon structure of the gene was finally completely deduced by aligning the mRNA sequence from the cDNA obtained as described above and the genomic DNA sequence obtained as described above. This alignment permitted the determination of the positions of the introns and exons, the positions of the start and end nucleotides defining each of the at least 8 exons, the locations and phases of the 5′ and 3′ splice sites, the position of the stop codon, and the position of the polyadenylation site to be determined in the genomic sequence. This analysis also yielded the positions of the coding region in the mRNA, and the locations of the polyadenylation signal and polyA stretch in the mRNA.

The gene identified as described above comprises at least 8 exons and spans more than 52 kb. A G/C rich putative promoter region was identified upstream of the coding sequence. A CCAAT in the putative promoter was also identified. The promoter region was identified as described in Prestridge, D. S., Predicting Pol II Promoter Sequences Using Transcription Factor Binding Sites, J. Mol. Biol. 249:923-932 (1995), the disclosure of which is incorporated herein by reference in its entirety.

Additional analysis using conventional techniques, such as a 5 ′RACE reaction using the Marathon-Ready human prostate cDNA kit from Clontech (Catalog. No. PT1156-1), may be performed to confirm that the 5′ of the cDNA obtained above is the authentic 5′ end in the mRNA.

Alternatively, the 5 ′sequence of the transcript can be determined by conducting a PCR amplification with a series of primers extending from the 5 ′end of the identified coding region.

### Example 19

### Detection of Biallelic Markers in the Candidate Gene: DNA Extraction

Donors were unrelated and healthy. They presented a sufficient diversity for being representative of a French heterogeneous population. The DNA from 100 individuals was extracted and tested for the detection of the biallelic markers.

30 ml of peripheral venous blood were taken from each donor in the presence of EDTA. Cells (pellet) were collected after centrifugation for 10 minutes at 2000 rpm. Red cells were lysed by a lysis solution (50 ml final volume: 10 mM Tris pH 7.6; 5 mM MgCl2; 10 mM NaCl). The solution was centrifuged (10 minutes, 2000 rpm) as many times as necessary to eliminate the residual red cells present in the supernatant, after resuspension of the pellet in the lysis solution.

The pellet of white cells was lysed overnight at 42° C. with 3.7 ml of lysis solution composed: of:

3 ml TE 10-2 (Tris-HCl 10 mM, EDTA 2 mM)/NaCl 0.4 M

200 μl SDS 10%

500 μl K-proteinase (2 mg K-proteinase in TE 10-2/NaCl 0.4 M).

For the extraction of proteins, 1 ml saturated NaCl (6M) (1/3.5 v/v) was added. After vigorous agitation, the solution was centrifuged for 20 minutes at 10000 rpm. For the precipitation of DNA, 2 to 3 volumes of 100% ethanol were added to the previous supernatant, and the solution was centrifuged for 30 minutes at 2000 rpm. The DNA solution was rinsed three times with 70% ethanol to eliminate salts, and centrifuged for 20 minutes at 2000 rpm. The pellet was dried at 37° C., and resuspended in 1 ml TE 10-1 or 1 ml water. The DNA concentration was evaluated by measuring the OD at 260 nm (1 unit OD=50 μg/ml DNA).

To determine the presence of proteins in the DNA solution, the OD 260/OD 280 ratio was determined. Only DNA preparations having a OD 260/OD 280 ratio between 1.8 and 2 were used in the subsequent examples described below.

The pool was constituted by mixing equivalent quantities of DNA from each individual.

### Example 20

### Detection of the Biallelic Markers: Ampification of Genomic DNA by PCR

The amplification of specific genomic sequences of the DNA samples of Example 19 was carried out on the pool of DNA obtained previously using the amplification primers of SEQ ID Nos: 542 to 553 and 564 to 575. In addition, 50 individual samples were similarly amplified.

PCR assays were performed using the following protocol:

| | | |
|---|---|---|
| Final volume | 25 | μl |
| DNA | 2 | ng/μl |
| MgCl2 | 2 | mM |
| dNTP (each) | 200 | μM |
| primer (each) | 2.9 | ng/μl |
| Ampli Taq Gold DNA polymerase | 0.05 | unit/μl |
| PCR buffer (10× = 0.1 M TrisHCl pH 8.3 0.5 M KCl) | 1× | |

Pairs of first primers were designed to amplify the promoter region, exons, and 3′ end of the candidate asthma-associated gene using the sequence information of the candidate gene and the OSP software (Hillier & Green, 1991). These first primers were about 20 nucleotides in length and contained a common oligonucleotide tail upstream of the specific bases targeted for amplification which was useful for sequencing. The synthesis of these primers was performed following the phosphoramidite method, on a GENSET UFPS 24.1 synthesizer.

DNA amplification was performed on a Genius II thermocycler. After heating at 94° C. for 10 min, 40 cycles were performed. Each cycle comprised: 30 sec at 94° C., 55° C. for 1 min, and 30 sec at 72° C. For final elongation, 7 min at 72° C. ended the amplification. The quantities of the amplification products obtained were determined on 96-well microtiter plates, using a fluorometer and Picogreen as intercalant agent (Molecular Probes).

### Example 21

### Detection of the Biallelic Markers Sequencing of Amplified Genomic DNA and Identification of Polymorphisms

The sequencing of the amplified DNA obtained in Example 20 was carried out on ABI 377 sequencers. The sequences of the amplification products were determined using automated dideoxy terminator sequencing reactions with a dye terminator cycle sequencing protocol. The products of the sequencing reactions were run on sequencing gels and the sequences were analyzed as formerly described.

The sequence data were further evaluated using the above mentioned polymorphism analysis software designed to detect the presence of biallelic markers among the pooled amplified fragments. The polymorphism search was based on the presence of superimposed peaks in the electrophoresis pattern resulting from different bases occurring at the same position as described previously.

Allelic frequencies were determined in a population of random blood donors from French Caucasian origin. Their wide range is due to the fact that, besides screening a pool of 100 individuals to generate biallelic markers as described above, polymorphism searches were also conducted in an individual testing format for 50 samples. This strategy was chosen here to provide a potential shortcut towards the identification of putative causal mutations in the association studies using them. Biallelic markers found in only one individual were not considered in the association studies.

### Example 22

### Validation of the Polymorphisms through Microsequencing

The biallelic markers identified in Example 21 were further confirmed and their respective frequencies were determined through microsequencing. Microsequencing was carried out for each individual DNA sample described in Example 18.

Amplification from genomic DNA of individuals was performed by PCR as described above for the detection of the biallelic markers with the same set of PCR primers described above.

The preferred primers used in microsequencing had about 19 nucleotides in length and hybridized just upstream of the considered polymorphic base.

Five primers hybridized with the non-coding strand of the gene. For the biallelic markers 10-204-326, 10-35-358 and 10-36-164, primers hybridized with the coding strand of the gene.

The microsequencing reaction was performed as described in Example 13.

### Example 23

### Association of a Frequent LSR Polymorphism with Elevated Plasma TG in Obese Adolescents

The association of selected SNPs with clinical values related to metabolic disorders was determined. This example and the following are exemplary only and do not indicate that there are not other significant associations between markers, clinical values, and metabolic disease. However, they do provide examples of methods useful for identifying significant associations useful in diagnostics, predictive medicine, and pharmacogenomics.

Marker Selection

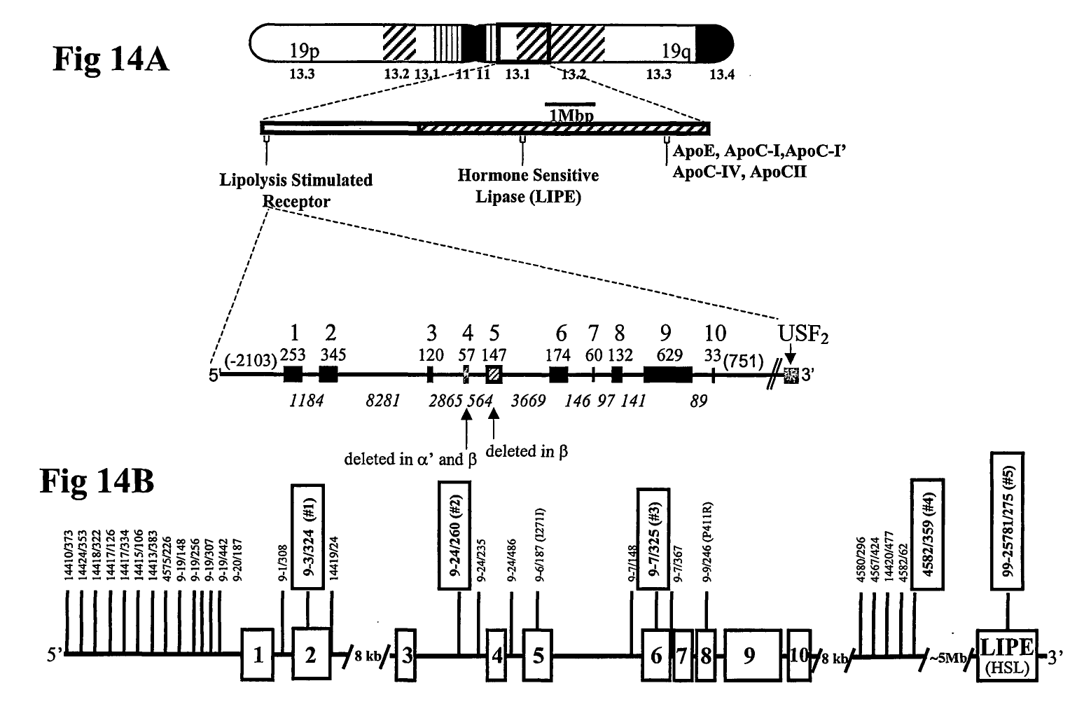

Five markers were selected based on the following three criteria: 1) equidistant coverage of the LSR gene; 2) within the USF2 and LIPE genes; and 3) allele frequency >10%. That the SNPs result in an amino acid change in the LSR protein was not a criteria; many intronic markers can also modulate gene function by affecting the stability of mRNA, the rate of splicing or the production of splice variants. The positions of the five markers are indicated by open boxes in FIG. 14B. Markers 1, 2, and 3 are listed in SEQ ID Nos 532, 533 and 534, respectively. Three of the markers are located within the LSR gene (markers 1-3). Markers #1 and #3 are within coding regions. Polymorphism at the site of marker #1 does not translate into variation at the protein level. (Val →Val). Marker #3 causes a Ser →Asn substitution in the extracellular domain of the receptor that contains the putative lipoprotein binding site. Marker #2 is located in intron 3, 137 bp upstream of the splice site that generates the different LSR isoforms. Markers #4 (SEQ ID No. 535) and #5 are found in introns of the USF2 gene and LIPE gene, respectively. The relative locations of USF2 and LIPE to LSR are shown in FIG. 14A.

As a control, 18 random markers distributed in various genomic regions were selected. Chromosomal localization, allele frequency, and Hardy-Weinberg equilibrium testing of those markers is provided in Table 5. All markers used in these studies were in Hardy-Weinberg equilibrium (Table 5). Quality control using known polymorphic sites inserted within each genotyping plate was performed systematically; results indicated an accuracy >98%. Automatic genotype calling on the 23 different SNP's used for this study led to unambiguous genotyping in 96.7% of cases. Ambiguous genotypes were not considered for the analysis. The percentage of ambiguous genotyping occurring for each marker is given in Table 5 below, which begins on the next page.

| | | | | Hardy- | AMBIGUOUS |
|---|---|---|---|---|---|
| | CHROMOSOMAL | ALLELIC | ALLELE | Weinberg | GENOTYPE |
| SNPS* | LOCALIZATION | VARIATION | FREQUENCY (%) | Equilibrium χ2 | (%) |
| SNP# | | | | | |
| 1 | 19Q13.1 | C → T | 73 | 0.365 | 1.3 |
| 2 | 19Q13.1 | A → G | 62 | 1.660 | 3.1 |
| 3 | 19Q13.1 | G → A | 89 | 0.735 | 0.6 |
| 4 | 19Q13.1 | G → T | 70 | 0.681 | 0.0 |
| 5 | 19Q13.2 | T → C | 65 | 0.091 | 8.2 |
| RANDOM | | | | | |
| SNP | | | | | |
| A | 7P12-P14 | T → C | 65 | 0.252 | 3.1 |
| B | 12P13 | G → A | 52 | 0.049 | 1.3 |
| C | 12P13 | C → A | 63 | 2.172 | 2.5 |
| D | 13Q22 | T → C | 74 | 1.194 | 0.6 |
| E | 14Q24.1 | A → G | 54 | 0.027 | 1.3 |
| F | 14Q31 | T → C | 62 | 0.322 | 1.3 |
| G | 14Q31 | G → G | 64 | 0.092 | 6.3 |
| H | 14Q22-Q23 | T → A | 79 | 0.594 | 6.3 |
| I | 16Q22-Q24 | G → A | 54 | 1.166 | 5.7 |
| J | 16Q24 | A → G | 62 | 0.656 | 3.8 |
| K | 17P13.3 | T → C | 72 | 1.790 | 3.8 |
| L | 17P13.3 | A → G | 78 | 0.562 | 4.4 |
| M | 18P11-P31 | A → G | 51 | 0.319 | 2.5 |
| N | 21Q22.8 | A → G | 56 | 0.054 | 7.5 |
| O | 21Q22 | C → T | 59 | 1.475 | 3.1 |
| P | 21Q22.3 | A → G | 70 | 2.070 | 3.1 |
| Q | 21Q22.3 | T → C | 60 | 1.709 | 4.4 |
| R | 21Q22.1 | A → G | 56 | 1.060 | 0.0 |

*SNPs were identified using a pool of 100 DNA clones, as described herein. The allele frequency and Hardy-Weinberg equilibrium were measured for each marker. The % of each ambiguous genotype that was not considered in the analysis is provided in the last column.

Subject Selection

The subjects participating in the study were 161 unrelated Caucasian girls that lived in the region of Paris. Obese girls attended weight reduction program at the Margency clinic or Saint Vincent de Paul hospital. All subjects developed severe obesity in early childhood as defined by a BMI exceeding the 98^{th} percentile of the population.

At the time of admission weights and heights were recorded, blood samples were collected, the buffy coat was isolated for DNA preparation and the plasma was separated for biochemical analysis. Plasma TG, total cholesterol and FFA, were determined using commercially available enzymatic kits and following manufacturer instructions. Blood sampling and testing of these subjects were performed prior to any weight reduction treatment.

Clinical Characteristics

The subjects clinical characteristics are described in Table 6. These values are for plasma samples collected after an overnight fast without standardization of the meal taken the night prior to admission in the clinical laboratory. It has been shown that under these conditions, plasma TG concentrations can vary considerably from day to day in the same individual (21).

TABLE 6
CHARACTERISTICS OF OBESE CHILDREN

| Parameter | Value* |
|---|---|
| n | 161 |
| Age (yrs) | 12 ± 0.2 |
| Body mass index (kg/m²) | 30.4 ± 0.5 |
| Plasma triglycerides (mg/dl) | 104 ± 4.0 |
| Plasma total cholesterol (mg/dl) | 172 ± 3.0 |
| Plasma FFA (mM) | 0.612 ± 0.022 |

*Mean ± SEM

SNP Identification

The amplicon of interest included the exons and introns of the LSR, USF₂ and LIPE genes. Random markers were generated from amplicons derived from BAC sequences of the indicated genomic regions. PCR primers were used to amplify the corresponding genomic sequence in a pool of DNA from 100 unrelated individuals (blood donors of French origin).

Amplification products from pooled DNA samples were sequenced on both strands by fluorescent automated sequencing on ABI 377 sequences (Perkin Elmer), using a dye-primer cycle analysis and DNA sequence extraction with ABI Prism DNA sequencing Analysis software. Sequence data analysis were automatically processed with AnaPolys (Genset, Paris, France), a software program designed to detect the presence of SNPs among pooled amplified fragments.

Genotyping Amplification products containing the SNPs were obtained by performing PCR reactions similar to those described for SNP identification (and supra). Genotyping of individual DNA samples was performed using a microsequencing procedure.

Statistical Analysis

Allelic frequencies and χ² test of Hardy Weinberg proportions were performed as data were collected (Hill, W. G. (1974) in Heredity, (Edinburgh), pp. 229-239; Terwilliger, J. O. (1994) Handbookfor Human Genetic Linkage (John Hopkins University Press, Baltimore); Schneider et al. (1997) Arlequin: A software for population genetic data analysis, 1.1 edition (Genetics and Biometry Laboratory, Department of Anthropology, University of Geneva, Geneva)). Differences in genotype frequencies within obese subjects separated according to the secondary phenotype were analyzed using 3×2χ² analysis. Two locus linkage disequilibrium (D) values were calculated from unphased genotypic data for pairs of SNPs located within the 19q13 locus (Hill, W. G. (1974) in Heredity, (Edinburgh), pp. 229-239; Terwilliger, J. O. (1994) Handbook for Human Genetic Linkage (John Hopkins University Press, Baltimore)) and were tested for significance from estimates of the four haplotypes frequencies which were obtained from the output of EH computer program (Schneider et al. (1997) Arlequin: A software for population genetic data analysis, 1.1 edition (Genetics and Biometry Laboratory, Department of Anthropology, University of Geneva, Geneva)). D′ was calculated as D/Dₘₐₓ using Dₘₐₓ positive and negative obtained from allele frequency products. The SAS programming language was used to construct, analyze and format databases for input into other genetic linkage computer programs.

Comparison of Genotypic Frequencies

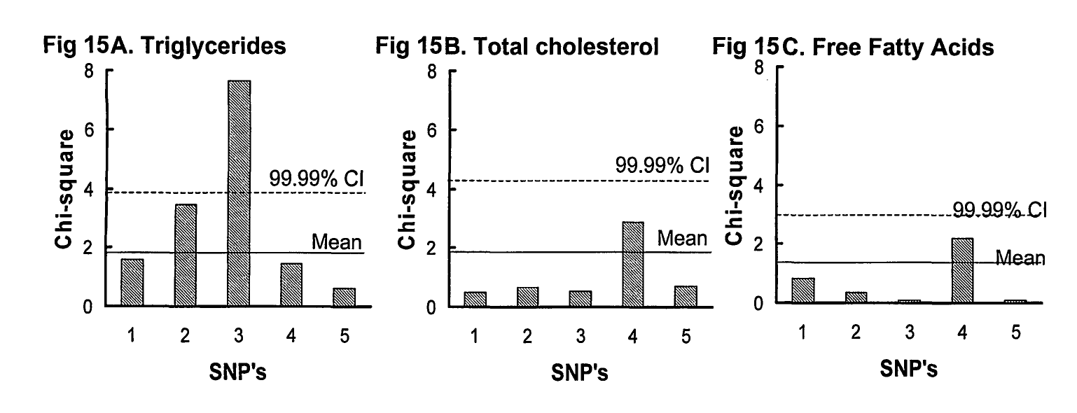

The genotype frequencies (for test and control markers) of subjects that had plasma TG, total cholesterol, and FFA values above the mean value of the population were compared with the genotype frequencies of subjects with values below the mean. The χ² value obtained for each of the 5 candidate markers is shown in FIG. 15. Only the genotype frequency of LSR SNP #3 shows a significant difference between the two groups of obese subjects, and only for those subjects with plasma TG above or below the mean of the population (FIG. 15A). This χ² value exceeded the 99.99% confidence interval of the mean χ² obtained with the random markers and that of any χ² obtained with the 18 random markers. The random marker mean and 99.99% confidence interval are shown as a solid and dotted line, respectively. No significant changes in genotype frequency of LSR markers were observed when the obese population was separated according to the total cholesterol or FFA levels. These data suggest that the mutation G+A at base 19739, causing a Ser →Asn substitution (amino acid residue 363), selectively influences plasma TG levels in obese adolescent girls.

In adolescent girls, normal plasma TG values range between 37 and 131 mg/dl (20); hypertriglycerdemia is >130 mg/dl TG. A comparison of the genotype frequency of hypertriglyceridemic individuals relative to those with normal TG showed that 33% of the hypertriglyceridemic individuals (n=35) had at least one A allele, while only 16% of the normotriglyceridemic individuals (n=125) had the A allele (χ²=4.5, p<0.04). Calculation of the odds ratio of being hypertriglyceridemic for obese girls as a direct consequence of LSR mutation returned a value of 2.5.

The LSR SNP #3 polymorphism that causes an asparagine to serine mutation in the external domain of the LSR protein, is in close proximity with the LSR putative lipoprotein binding domain. Thus, this polymorphism of the LSR gene appears to cause a mutation in the LSR protein that decreases the activity of LSR as lipoprotein receptor. Since LSR serves primarily for the removal of TG rich lipoprotein, impairment of this function due to genetic polymorphism is therefore likely to cause hyperlipidemia in obese adolescent girls. Although this result was found in studies with adolescent girls, there is no a priori reason to suspect that a similar result will not be found with adolescent boys, or that a similar effect is not also present in adults of both sexes.

### Example 24

### Association of a Frequent LSR Polymorphism with Postprandial Lipemia in Obese Adolescents

In this study, both fasting and postprandial plasma TG were determined for 34 obese adolescent girls admitted to clinical research centers. The plasma TG values were measured in a research laboratory. Except as otherwise indicated, the materials and methods were the same as those described for Example 23, above.

Subject Selection and Testing

A subset of the subjects described in Example 23 (n=34) were admitted to the clinic on the evening prior to the test. They consumed a normal standard test meal, and were not allowed anything except water for 12 h. At 8:00 AM, plasma was collected and the individuals consumed a standardized high fat test meal within 15 min. The high fat test meal provided 1000 kcal, contained 62% fat (29% saturated, 27% monounsaturated and 44% polyunsaturated fat), 29% carbohydrate and 9% protein, and consisted of bread and butter, eggs with mayonnaise, cheese, salad with sunflower oil, and applesauce. Blood samples were collected before, and 2 and 4 hour after this meal.

Comparison of Genotypic Frequencies

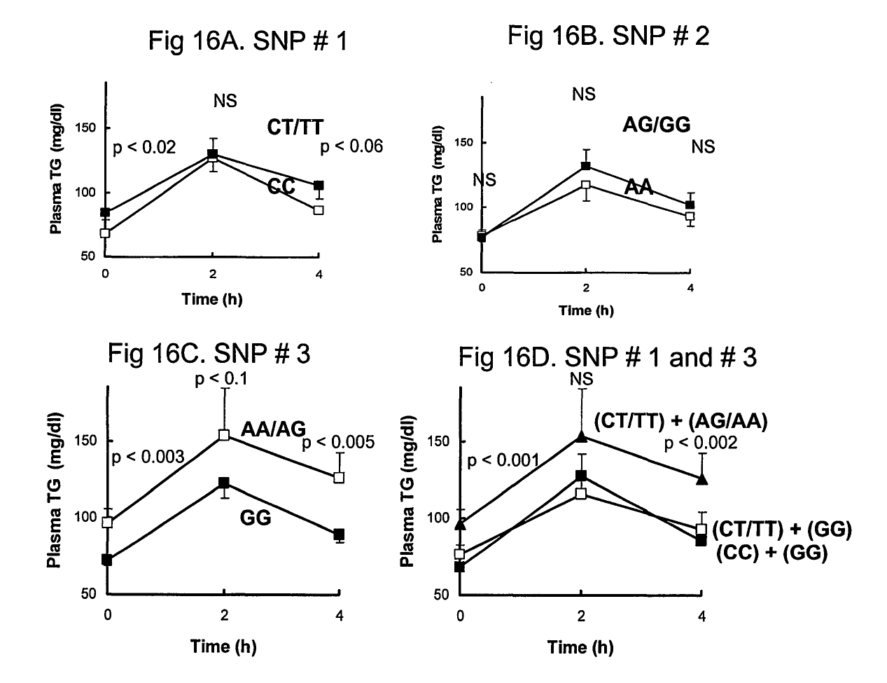

The effect of LSR genotype (Markers #1, 2 and 3) on the postprandial triglyceride response to the test meal is shown in FIGS. 16A-C. Subjects that were homozygous GG (Ser) for marker #3 had a significantly lower plasma TG level both before and 4 h after the meal (FIG. 16C). Genotype differences at LSR marker #2 had no detectable effect on fasting and postprandial lipemia (FIG. 1613). Interestingly, LSR marker #1 appeared to exert significant influence on fasting plasma TG levels (FIG. 16A).

To determine whether the apparent fasting effect of genotype at marker #1 was independent of LSR marker #3, we plotted the plasma TG response, taking into account the genotype of both marker #1 and #3 (FIG. 16D). LSR marker #1 polymorphisms had no influence on the postprandial response of individuals that had the normal GG genotype at marker #3. However, no individual was found to combine the frequent allele at marker #1 and the rare allele at marker #3. Thus, it is not possible to determine whether such associations would aggravate or reduce the abnormal lipid response seen in subjects with the Asn mutation.

The simplest explanation for the influence of SNP marker #1 on fasting plasma lipid values is that this marker is in linkage disequilibrium with marker #3 and simply translates, although to a lower degree, the abnormality of function caused by amino acid substitution. To test for this possibility, the degree of linkage disequilibrium among all 5 test markers was determined. The data show that all 3 markers within the LSR gene are in linkage disequilibrium (data not shown). It is therefore not surprising that although silent at the protein level, marker #1 influences significantly plasma TG by virtue of linkage with marker #3. This also explains why none of the 161 subjects had both CC and AG or AA genotype for marker #1 and #3, respectively.

Sequence Analysis

Genomic DNA of subjects homozygous for either the Ser (n=12) or Asn (n=3) substitution was amplified by PCR and all LSR exons were sequenced in both directions. No other coding mutation besides the Ser→Asn substitution were detected. Thus, the influence of marker #3 on plasma TG appears to result directly from the mutation it causes in the LSR protein. SNP#3 appears to directly influence both fasting and postprandial plasma TG per se, not simply signaling the presence of another unidentified mutation.

Significance and Hypotheses

Although not intending to be limited in any way, the inventors hypothesize that the mutation of LSR exon 6 that removes an alcohol function group and introduces a basic amino acid reduces the efficiency of the receptor and decreases the rate of removal of dietary TG. The fact that this mutation is associated with lower levels of fasting and 4 h postprandial plasma TG, but does not significantly affect plasma TG measured 2 h after the meal, is in keeping with this interpretation. The inventors further hypothesize that at the time of the postprandial peak (2 h), plasma TG levels are mostly determined by the rate of release of chylomicrons by the intestine and the rate of TG hydrolysis by lipoprotein lipase and possibly also hepatic lipase. After 4 h, however, alternate mechanisms relying on cellular uptake of chylomicron remnants appear to play a significant role (Karpe, et al. (1997) J. Lipid Res. 38, 2335-2343).

Thus, at least in obese adolescent girls, polymorphisms of the LSR gene significantly influence the metabolism of TG-rich lipoproteins. Genetic evidence supports the notion that the LDL-receptor and the LSR contribute to the removal of lipoproteins. Defects of the LDL-receptor cause primarily hypercholesterolemia, while defects of the LSR influence in obese adolescent girls hypertriglyceridemia without hypercholesterolemia. Although functional mutation of the LDL-receptor causes massive hypercholesterolemia in most affected individuals, mutation of the LSR gene only increased by 2.5 fold the odds of being hypertriglyceridemic for obese adolescent girls. In addition, a number of individuals with the mutation have low levels of TG and conversely about two-thirds of obese subjects with hypertriglyceridemia show no abnormalities at the level of the LSR gene. Clearly, environmental factors and other genes also influence plasma TG levels. It will be possible to simultaneously analyze the influence of those genes and thereby to determine their relative importance with respect to each other.

In women, hypertriglyceridemia, which is the most common lipid abnormality observed in survivors of myocardial infarction (Goldstein et al. (1973) J. Clin. Invest. 52, 1533-1543), is considered an independent risk factor of cardiovascular disease (Austin, et al. (1998) Am. J. Cardiol. 81, 7B-12B). Thus, genotyping LSR marker #3 may provide a diagnostic tool to predict the risk of cardiovascular complication in obese subjects (and potentially even in non-obese subjects).

It is also postulated that it is possible that LSR polymorphisms contribute to hypertriglyceridemia only in subjects with excess body weight. Indeed, decreased LSR expression may reveal the functional effect of small mutations in the LSR protein that would otherwise remain silent. In this perspective, it is interesting to note that defective clearance of chylomicrons that occurs in type III hyperlipidemia is often rapidly corrected by weight reduction (Mahley, R. W., and Rall, Jr., S. C. (1995) in The Molecular Basis of Inherited Disease, eds. Scriver, et al. (McGraw Hill Inc., New York), pp. 1953-1980). Since LSR does not bind β-VLDL isolated from a subject with type III hyperlipidemia and with the apoE2/2 phenotype (Yen, et al. (1994) Biochemistry 33, 1172-1180.), this suggests that reduced LSR expression due to excess body weight causes, in conjunction with abnormal ApoE isoforms, the appearance of type m hyperlipidemia.

### Example 25

### Association of a Frequent LSR Polymorphism with Insulin and Glucose Levels in Obese Adolescents

### Insulin:

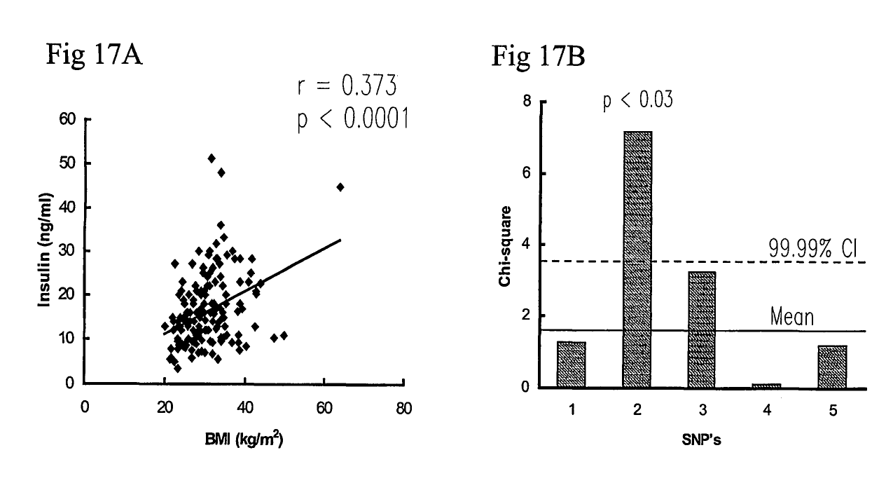

In obese children, insulin is strongly and positively correlated with BMI, in agreement with previous studies (FIG. 17A). The association of LSR polymorphism with these variables was determined using an analysis similar to that described above (Example 23).

The obese population was divided into separate populations based on whether the individual fell above or below the insulin-BMI regression line; genotype frequencies in each group were compared (FIG. 17B). The results show that LSR polymorphism shows an association with insulin levels relative to BMI. Genotype frequencies of marker #2 were significantly different in subjects with high insulin to BMI ratios (p<0.03). The χ² value largely exceeded that defined by the distribution of random markers. Subjects homozygous for the A allele, had significantly higher Insulin to BMI ratios than subjects that were either heterozygous or homozygous for the G allele: 0.571+/−0.058 and 0.505+/−0.058 (p<0.05) respectively.

Thus, the data indicate that in individuals homozygous for the A allele, the level of circulating insulin normalized to BMI is higher than in those with the G allele. This suggests that LSR plays a previously unsuspected role in determining plasma insulin levels, and may also influence the level of insulin resistance in obese adolescent girls. Again, there is no reason to suspect that similar results would not be found for adolescent boys, or adults of both sexes.

Glucose Response

To further validate the association of LSR marker #2 with insulin sensitivity, a subset of 120 overnight fasted obese children received 50 g of glucose per os. Both plasma glucose and insulin concentrations were measured on samples collected prior and 2 hours after this test.

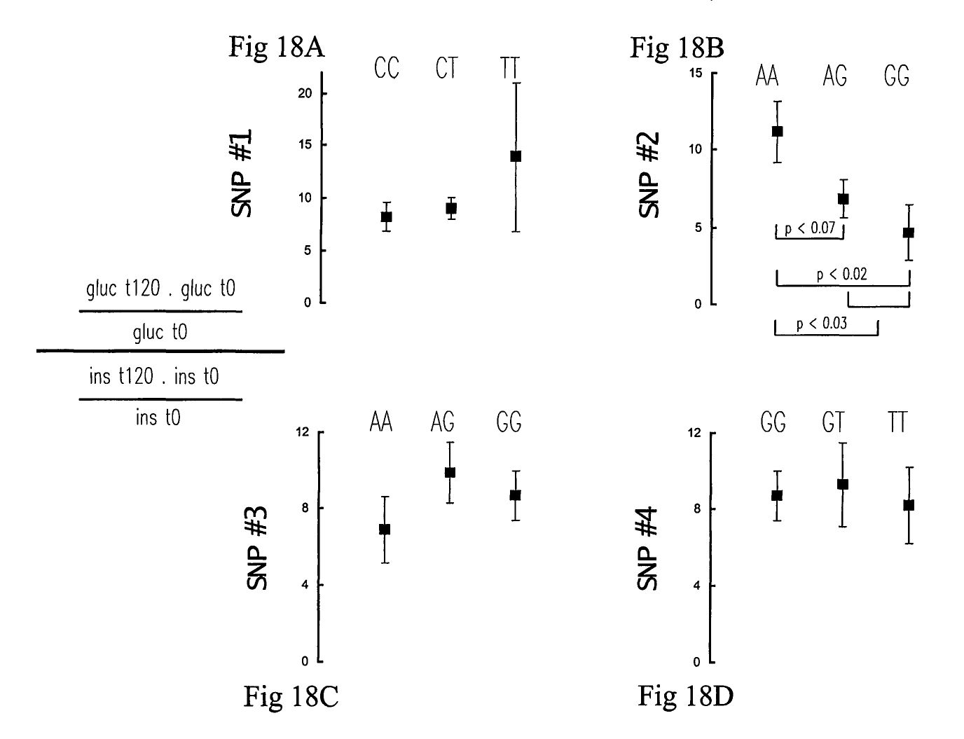

Subjects with AA genotype for marker #2 showed a significantly higher increase in plasma glucose relative to insulin than those that were GG (FIG. 18B). Subjects heterozygous for marker #2 had an intermediate response. In the group that were AA at marker #2, 7 individuals out of 54 had plasma glucose levels at 2 h greater than 120 mg/dl. In the AG/GG group only 2 out of 66 had values greater than 120 mg/dl (p<0.05). Genotype differences at the site of markers #1, 3, or 4 did not significantly influence the glucose to insulin changes after glucose load (FIGS. 18A, 18C, and 18D).

Consistent with the association of marker #2 with insulin to BMI ratios (FIG. 17), subjects homozygous for the A allele at the level of LSR marker 2 had a significantly higher increase in glucose relative to insulin than those heterozygous or homozygous for the G allele (FIG. 18). This indicates that individuals with relatively higher insulin to body weight ratio (FIG. 17), are also those with a relatively higher degree of glucose intolerance. Thus, genotyping LSR marker 2 allows prediction not only of the insulin to BMI ratio, but also of the level of glucose tolerance. This indicates that this marker is a significant predictor of the risk in developing type II diabetes at a later age and thus useful for predictive medicine and diagnostics.

The putative molecular mechanisms through which the products of the LSR gene influence insulin sensitivity are two-fold (although the inventors do not wish to be limited by the following hypotheses). First, LSR is a receptor that undergoes conformational changes upon binding of FFA. The LSR primary sequence is compatible with a function of receptor signalling through phosphorylation. FFA concentration in the portal system have been shown to significantly influence the risk of development of type II diabetes. It is therefore speculated that FFA binding to LSR causes signalling to the cell that decreases the efficiency of insulin signalling to the insulin receptor. Second, the LSR α′ subunit binds with leptin with high affinity and causes mobilization of LSR from intracellular vesicles to the cell surface. Leptin has been previously shown to modulate insulin sensitivity. Thus, it is possible that the polymorphism at the level of LSR marker #2, indicates a dysfunction of the receptor in either its ability to bind leptin, to bind FFA or to signal to the cell.

### Example 26

### Association of Frequent LSR Polymorhisms with Obesity in Adolescents

All subjects (case and control) were female Caucasians. Subjects in the case group developed severe excess body weight during childhood (BMI>than 98th percentile (n=138)), while control subjects remained lean throughout adulthood (BMI 18-23 (n=78)). All subjects participating to this study lived in the regions of Paris or Brussels. Some clinical characteristics of case and controls are summarized in Table 6 (above).

The genotype of markers 1, 2, and 3 of LSR were determined for the populations of lean and obese subjects. Analysis of the genotype association showed that obese subjects had a much greater frequency of CT/TT, AA, GG genotypes at markers #1, #2 and #3, respectively. This genotype association was found with a frequency of 23% in the obese group, and with a frequency of 2.5% in the lean group.

Estimation of the probability that this difference in frequency occurred randomly was determined by Chi square analysis. The calculated Chi square was 15.98 (p<0.00008). Thus, it unlikely that the genotype association defined above occurred with a greater frequency in the obese population by chance. It is more probable that this polymorphism indicates the presence of a dysfunction of the receptor that directly increases the chance that an individual will become obese.

Estimation of the probability that the SNPs correlate with obesity and indicate a receptor dysfunction was determined by calculation of the odds ratio. This calculation returned an estimate of 11.5. Thus, individuals who are CT/TT+AA+GG for LSR markers 1, 2 and 3 are 11.5 times more likely to become obese than those individuals with a different genotype for those markers. Thus, genotyping LSR Markers #1, #2 and #3 allows a prediction of the probability that an individual will become obese. The molecular mechanisms through which LSR could cause obesity have been described previously, and include 1) binding of plasma FFA, 2) processing of dietary lipids, 2) processing of leptin, 3) leptin signaling, 4) modulation of insulin sensitivity, and 5) leptin transport across the blood brain barrier.

### Example 27

### Forensic Matching by Microseguencing

DNA samples are isolated from forensic specimens of, for example, hair, semen, blood or skin cells by conventional methods. A panel of PCR primers based on a number of the sequences of SEQ ID Nos 1 to 1132 is then utilized according to the methods described herein to amplify DNA of approximately 500 bases in length from the forensic specimen. The alleles present at each of-the selected biallelic markers site according to biallelic markers SEQ ID Nos 1 to 1132 are then identified according Example 13. A simple database comparison of the analysis results determines the differences, if any, between the sequences from a subject individual or from a database and those from the forensic sample. In a preferred method, statistically significant differences between the suspect's DNA sequences and those from the sample conclusively prove a lack of identity. This lack of identity can be proven, for example, with only one sequence. Identity, on the other hand, should be demonstrated with a large number of sequences, all matching. Preferably, a minimum of 13, 17, 20, 25, 30, 40, 50, 66, 70, 85, 88, 100, 187, 200 or 500 biallelic markers are used to test identity between the suspect and the sample.

Although this invention has been described in terms of certain preferred embodiments, other embodiments which will be apparent to those of ordinary skill in the art of view of the disclosure herein are also within the scope of this invention. Accordingly, the scope of the invention is intended to be defined only by reference to the appended claims.

TABLE 1a

| | | | | | | | SEQ ID No. of | |
|---|---|---|---|---|---|---|---|---|
| | | | Position | | | | Amplification Primers | |
| SEQ | | | of marker | | | Preferred | | Down- |
| ID | | | in | Allele | | microseq. | Upstream | stream |
| No. | Marker Name | Length | SEQ ID | 1^{st} | 2^{nd} | primer | (PU) | (RP) |
| 1 | 99-55095-221 | 47 | 23 | A | G | S | 172 | 343 |
| 2 | 99-55102-112 | 47 | 23 | G | T | S | 173 | 344 |
| 3 | 99-26407-73 | 47 | 23 | A | G | S | 174 | 345 |
| 4 | 99-26412-162 | 47 | 23 | G | C | S | 175 | 346 |
| 5 | 99-26413-307 | 47 | 23 | A | T | S | 176 | 347 |
| 6 | 99-26424-423 | 47 | 23 | C | T | A | 177 | 348 |
| 7 | 99-44320-377 | 47 | 23 | A | T | SA | 178 | 349 |
| 8 | 99-41889-287 | 47 | 23 | C | T | S | 179 | 350 |
| 9 | 99-45453-183 | 47 | 23 | A | T | S | 180 | 351 |
| 10 | 99-43427-381 | 47 | 23 | G | T | A | 181 | 352 |
| 11 | 99-55124-180 | 47 | 23 | C | T | S | 182 | 353 |
| 12 | 99-46316-65 | 47 | 23 | A | T | SA | 183 | 354 |
| 13 | 99-46325-327 | 47 | 23 | C | T | SA | 184 | 355 |
| 14 | 99-46332-107 | 47 | 23 | A | G | S | 185 | 356 |
| 15 | 99-47064-85 | 47 | 23 | C | T | | 186 | 357 |
| 16 | 99-47065-42 | 47 | 23 | C | T | A | 187 | 358 |
| 17 | 99-47066-258 | 47 | 23 | A | G | S | 188 | 359 |
| 18 | 99-38883-331 | 47 | 23 | C | T | S | 189 | 360 |
| 19 | 99-38888-357 | 47 | 23 | C | T | | 190 | 361 |
| 20 | 99-38889-169 | 47 | 23 | C | T | S | 191 | 362 |
| 21 | 99-26458-340 | 47 | 23 | G | C | S | 192 | 363 |
| 22 | 99-47321-421 | 47 | 23 | G | T | SA | 193 | 364 |
| 23 | 99-36198-261 | 47 | 23 | C | T | A | 194 | 365 |
| 24 | 99-36200-309 | 47 | 23 | A | G | | 195 | 366 |
| 25 | 99-33135-357 | 47 | 23 | C | T | S | 196 | 367 |
| 26 | 99-34451-198 | 47 | 23 | C | T | S | 197 | 368 |
| 27 | 99-34452-238 | 47 | 23 | A | C | SA | 198 | 369 |
| 28 | 99-48928-392 | 47 | 23 | G | T | | 199 | 370 |
| 29 | 99-55720-283 | 47 | 23 | A | G | S | 200 | 371 |
| 30 | 99-55721-111 | 47 | 23 | C | T | A | 201 | 372 |
| 31 | 99-501-57 | 47 | 23 | A | G | A | 202 | 373 |
| 32 | 99-34556-66 | 47 | 23 | C | T | S | 203 | 374 |
| 33 | 99-34561-414 | 47 | 23 | G | C | S | 204 | 375 |
| 34 | 99-28061-254 | 47 | 23 | G | C | S | 205 | 376 |
| 35 | 99-28062-68 | 47 | 23 | A | C | SA | 206 | 377 |
| 36 | 99-28063-331 | 47 | 23 | C | T | S | 207 | 378 |
| 37 | 99-28067-380 | 47 | 23 | C | T | SA | 208 | 379 |
| 38 | 99-28080-254 | 47 | 23 | G | C | A | 209 | 380 |
| 39 | 99-459-347 | 47 | 23 | A | G | A | 210 | 381 |
| 40 | 99-32104-363 | 47 | 23 | A | G | S | 211 | 382 |
| 41 | 99-26970-119 | 47 | 23 | G | C | S | 212 | 383 |
| 42 | 99-27502-165 | 47 | 23 | A | T | S | 213 | 384 |
| 43 | 99-26994-129 | 47 | 23 | C | T | A | 214 | 385 |
| 44 | 99-26995-256 | 47 | 23 | A | C | SA | 215 | 386 |
| 45 | 99-26997-298 | 47 | 23 | A | G | S | 216 | 387 |
| 46 | 99-27001-115 | 47 | 23 | G | T | S | 217 | 388 |
| 47 | 99-32161-353 | 47 | 23 | A | G | | 218 | 389 |
| 48 | 99-32165-233 | 47 | 23 | G | T | | 219 | 390 |
| 49 | 99-32168-440 | 47 | 23 | C | T | | 220 | 391 |
| 50 | 99-32172-210 | 47 | 23 | A | C | | 221 | 392 |
| 51 | 99-32174-130 | 47 | 23 | A | G | | 222 | 393 |
| 52 | 99-57310-483 | 47 | 23 | A | G | | 223 | 394 |
| 53 | 99-57311-339 | 47 | 23 | G | C | | 224 | 395 |
| 54 | 99-57314-120 | 47 | 23 | C | T | | 225 | 396 |
| 55 | 99-26359-395 | 47 | 23 | C | T | A | 226 | 397 |
| 56 | 99-27232-379 | 47 | 23 | A | G | S | 227 | 398 |
| 57 | 99-27595-373 | 47 | 23 | G | C | SA | 228 | 399 |
| 58 | 99-26492-146 | 47 | 23 | C | T | A | 229 | 400 |
| 59 | 99-26501-314 | 47 | 23 | C | T | S | 230 | 401 |
| 60 | 99-28350-202 | 47 | 23 | A | G | A | 231 | 402 |
| 61 | 99-28355-255 | 47 | 23 | G | C | A | 232 | 403 |
| 62 | 99-28360-156 | 47 | 23 | A | G | S | 233 | 404 |
| 63 | 99-28363-114 | 47 | 23 | A | G | | 234 | 405 |
| 64 | 99-28364-288 | 47 | 23 | A | G | | 235 | 406 |
| 65 | 99-27049-396 | 47 | 23 | C | T | S | 236 | 407 |
| 66 | 99-27054-298 | 47 | 23 | A | C | S | 237 | 408 |
| 67 | 99-27056-327 | 47 | 23 | C | T | A | 238 | 409 |
| 68 | 99-27586-122 | 47 | 23 | A | G | S | 239 | 410 |
| 69 | 99-29193-332 | 47 | 23 | A | C | | 240 | 411 |
| 70 | 99-56867-257 | 47 | 23 | A | T | | 241 | 412 |
| 71 | 99-26399-320 | 47 | 23 | C | T | S | 242 | 413 |
| 72 | 99-41887-343 | 47 | 23 | A | C | S | 243 | 414 |
| 73 | 99-27851-30 | 47 | 23 | A | G | A | 244 | 415 |
| 74 | 99-38884-129 | 47 | 23 | A | G | S | 245 | 416 |
| 75 | 99-38897-96 | 47 | 23 | A | T | | 246 | 417 |
| 76 | 99-36178-395 | 47 | 23 | A | T | S | 247 | 418 |
| 77 | 99-36201-377 | 47 | 23 | C | T | | 248 | 419 |
| 78 | 99-32207-353 | 47 | 23 | A | G | S | 249 | 420 |
| 79 | 99-55707-362 | 47 | 23 | C | T | | 250 | 421 |
| 80 | 99-48925-395 | 47 | 23 | A | G | | 251 | 422 |
| 81 | 99-55725-246 | 47 | 23 | C | T | S | 252 | 423 |
| 82 | 99-28082-267 | 47 | 23 | C | T | | 253 | 424 |
| 83 | 99-28083-222 | 47 | 23 | A | G | | 254 | 425 |
| 84 | 99-27498-237 | 47 | 23 | C | T | S | 255 | 426 |
| 85 | 99-27503-68 | 47 | 23 | G | C | | 256 | 427 |
| 86 | 99-26511-185 | 47 | 23 | A | G | SA | 257 | 428 |
| 87 | 99-26989-152 | 47 | 23 | C | T | | 258 | 429 |
| 88 | 99-32158-71 | 47 | 23 | C | T | | 259 | 430 |
| 89 | 99-32162-317 | 47 | 23 | A | G | | 260 | 431 |
| 90 | 99-32166-332 | 47 | 23 | G | T | | 261 | 432 |
| 91 | 99-32169-219 | 47 | 23 | G | C | | 262 | 433 |
| 92 | 99-57305-320 | 47 | 23 | A | G | S | 263 | 434 |
| 93 | 99-57308-151 | 47 | 23 | A | G | | 264 | 435 |
| 94 | 99-26477-419 | 47 | 23 | C | T | A | 265 | 436 |
| 95 | 99-26479-79 | 47 | 23 | C | T | A | 266 | 437 |
| 96 | 99-26480-284 | 47 | 23 | C | T | A | 267 | 438 |
| 97 | 99-26503-298 | 47 | 23 | A | G | S | 268 | 439 |
| 98 | 99-27042-125 | 47 | 23 | A | C | S | 269 | 440 |
| 99 | 99-27050-269 | 47 | 23 | A | T | A | 270 | 441 |
| 100 | 99-27583-97 | 47 | 23 | C | T | A | 271 | 442 |
| 101 | 99-55097-137 | 47 | 23 | C | T | S | 272 | 443 |
| 102 | 99-41727-169 | 47 | 23 | A | G | S | 273 | 444 |
| 103 | 99-44259-417 | 47 | 23 | A | G | | 274 | 445 |
| 104 | 99-44260-155 | 47 | 23 | C | T | S | 275 | 446 |
| 105 | 99-41009-244 | 47 | 23 | C | T | A | 276 | 447 |
| 106 | 99-41010-102 | 47 | 23 | G | C | A | 277 | 448 |
| 107 | 99-41828-310 | 47 | 23 | C | T | S | 278 | 449 |
| 108 | 99-45821-385 | 47 | 23 | A | G | S | 279 | 450 |
| 109 | 99-26385-78 | 47 | 23 | C | T | SA | 280 | 451 |
| 110 | 99-48212-159 | 47 | 23 | A | G | S | 281 | 452 |
| 111 | 99-36203-346 | 47 | 23 | A | G | S | 282 | 453 |
| 112 | 99-441-155 | 47 | 23 | C | T | SA | 283 | 454 |
| 113 | 99-444-226 | 47 | 23 | C | T | S | 284 | 455 |
| 114 | 99-55706-54 | 47 | 23 | C | T | A | 285 | 456 |
| 115 | 99-55708-159 | 47 | 23 | A | T | S | 286 | 457 |
| 116 | 99-55709-240 | 47 | 23 | A | C | A | 287 | 458 |
| 117 | 99-36422-176 | 47 | 23 | A | T | SA | 288 | 459 |
| 118 | 99-36426-50 | 47 | 23 | A | T | SA | 289 | 460 |
| 119 | 99-54269-455 | 47 | 23 | A | G | | 290 | 461 |
| 120 | 99-54270-270 | 47 | 23 | G | T | A | 291 | 462 |
| 121 | 99-54272-97 | 47 | 23 | G | C | S | 292 | 463 |
| 122 | 99-54276-127 | 47 | 23 | C | T | A | 293 | 464 |
| 123 | 99-54277-41 | 47 | 23 | C | T | | 294 | 465 |
| 124 | 99-49542-187 | 47 | 23 | G | C | S | 295 | 466 |
| 125 | 99-49544-423 | 47 | 23 | G | C | A | 296 | 467 |
| 126 | 99-49545-396 | 47 | 23 | G | C | | 297 | 468 |
| 127 | 99-49547-344 | 47 | 23 | A | G | S | 298 | 469 |
| 128 | 99-457-400 | 47 | 23 | A | G | A | 299 | 470 |
| 129 | 99-463-90 | 47 | 23 | C | T | S | 300 | 471 |
| 130 | 99-32097-347 | 47 | 23 | A | G | S | 301 | 472 |
| 131 | 99-32110-64 | 47 | 23 | A | G | | 302 | 473 |
| 132 | 99-32117-226 | 47 | 23 | C | T | SA | 303 | 474 |
| 133 | 99-26972-44 | 47 | 23 | A | G | | 304 | 475 |
| 134 | 99-26973-239 | 47 | 23 | G | C | | 305 | 476 |
| 135 | 99-26974-450 | 47 | 23 | C | T | | 306 | 477 |
| 136 | 99-26975-40 | 47 | 23 | C | T | | 307 | 478 |
| 137 | 99-26978-122 | 47 | 23 | A | T | | 308 | 479 |
| 138 | 99-26981-171 | 47 | 23 | C | T | SA | 309 | 480 |
| 139 | 99-26983-292 | 47 | 23 | C | T | | 310 | 481 |
| 140 | 99-26987-196 | 47 | 23 | A | C | SA | 311 | 482 |
| 141 | 99-32153-348 | 47 | 23 | C | T | | 312 | 483 |
| 142 | 99-32156-127 | 47 | 23 | C | T | | 313 | 484 |
| 143 | 99-57302-162 | 47 | 23 | G | T | A | 314 | 485 |
| 144 | 99-26349-339 | 47 | 23 | C | T | S | 315 | 486 |
| 145 | 99-26352-71 | 47 | 23 | A | G | S | 316 | 487 |
| 146 | 99-26370-60 | 47 | 23 | G | C | S | 317 | 488 |
| 147 | 99-28332-445 | 47 | 23 | A | C | A | 318 | 489 |
| 148 | 99-28339-362 | 47 | 23 | A | G | S | 319 | 490 |
| 149 | 99-28342-137 | 47 | 23 | A | G | | 320 | 491 |
| 150 | 99-28343-487 | 47 | 23 | A | G | S | 321 | 492 |
| 151 | 99-28352-101 | 47 | 23 | A | C | S | 322 | 493 |
| 152 | 99-28371-233 | 47 | 23 | G | C | A | 323 | 494 |
| 153 | 99-27041-274 | 47 | 23 | A | G | A | 324 | 495 |
| 154 | 99-29191-241 | 47 | 23 | A | G | | 325 | 496 |
| 155 | 99-27074-429 | 47 | 23 | A | G | S | 326 | 497 |
| 156 | 99-27076-313 | 47 | 23 | C | T | S | 327 | 498 |
| 157 | 99-27079-302 | 47 | 23 | A | T | SA | 328 | 499 |
| 158 | 99-27082-303 | 47 | 23 | A | G | S | 329 | 500 |
| 159 | 99-55890-43 | 47 | 23 | A | T | S | 330 | 501 |
| 160 | 99-55901-112 | 47 | 23 | G | C | | 331 | 502 |
| 161 | 99-55903-324 | 47 | 23 | A | T | | 332 | 503 |
| 162 | 9-42-169 | 47 | 23 | A | G | A | 333 | 504 |
| 163 | 99-41725-266 | 47 | 23 | A | G | S | 334 | 505 |
| 164 | 99-45440-313 | 47 | 23 | C | T | S | 335 | 506 |
| 165 | 99-438-82 | 47 | 23 | C | T | S | 336 | 507 |
| 166 | 99-36428-363 | 47 | 23 | A | G | A | 337 | 508 |
| 167 | 99-36433-318 | 47 | 23 | A | G | | 338 | 509 |
| 168 | 99-26968-300 | 47 | 23 | A | G | SA | 339 | 510 |
| 169 | 99-26345-57 | 47 | 23 | A | C | A | 340 | 511 |
| 170 | 99-27070-379 | 47 | 23 | C | T | S | 341 | 512 |
| 171 | 99-27581-206 | 47 | 23 | G | T | S | 342 | 513 |

TABLE 7

| SEQ | | | |
|---|---|---|---|
| ID | Marker | Chromosomal | |
| No. | Name | Localization | Adjacent STS (including aliases) |
| 1 | 99-55095-221 | 10p12.1-p11.2 | I-8535; 11392; 8535; D10S1568; EST91372; |
| | | | RH50926; G00-588-494; G05895; T40568; SHGC- |
| | | | 13375; T40568; SHGC-33797; R94235; stSG4085; |
| | | | RH44419; |
| 2 | 99-55102-112 | 10p12.1-p11.2 | I-8535; 11392; 8535; D10S1568; EST91372; |
| | | | RH50926; G00-588-494; G05895; T40568; SHGC- |
| | | | 13375; T40568; SHGC-33797; R94235; stSG4085; |
| | | | RH44419; |
| 3 | 99-26407-73 | 10p12.1-p11.2 | 238we5; AFM238we5; D10S550; |
| 4 | 99-26412-162 | 10p12.1-p11.2 | 238we5; AFM238we5; D10S550; |
| 5 | 99-26413-307 | 10p12.1-p11.2 | 238we5; AFM238we5; D10S550; |
| 6 | 99-26424-423 | 10p12.1-p11.2 | 238we5; AFM238we5; D10S550; |
| 7 | 99-44320-377 | 10p12 | |
| 8 | 99-41889-287 | 3q26 | |
| 9 | 99-45453-183 | 10p12 | |
| 10 | 99-43427-381 | 3q26.2 | WI-3044; 8169; 3044; D3S3122; G02683; |
| | | | MR3406; HHAd19e7; RH58631; R15842; |
| | | | SGC32223; |
| 11 | 99-55124-180 | 10p12.1-p11.2 | WI-8535; 11392; 8535; D10S1568; EST91372; |
| | | | RH50926; G00-588-494; G05895; T40568; 240xd6; |
| | | | AFM240xd6; 10S553; SHGC-13375; T40568; |
| | | | TIGR-A006T43; RH26037; stSG4085; RH44419; |
| | | | AFM; a204yb9; D10S1662; a204yb9; RH42685; ; |
| 12 | 99-46316-65 | 3q26.1-q26.2 | RH58533; G04564; WI-4113; |
| 13 | 99-46325-327 | 3q26.1-q26.2 | RH858533; G04564; WI-4113; |
| 14 | 99-46332-107 | 3q26.1-q26.2 | CHLC.GATA67A03; D3S2458; G08267; GATA- |
| | | | D3S2458; |
| 15 | 99-47064-85 | 3q26.2-q26.3 | RH58588; SGC44328; |
| 16 | 99-47065-42 | 3q26.2-q26.3 | RH58588; SGC44328; |
| 17 | 99-47066-258 | 3q26.2-q26.3 | RH58588; SGC44328; |
| 18 | 99-38883-331 | 3q26.1-q26.2 | |
| 19 | 99-38888-357 | 3q26.1-q26.2 | |
| 20 | 99-38889-169 | 3q26.1-q26.2 | |
| 21 | 99-26458-340 | 10p12.1-p11.2 | RH51062; R49361; WI-21812; RH51573; T83671; |
| | | | SGC33575; STS37757; RH44689; AFMb351zc1; |
| | | | D10S1734; b351zc1; |
| 22 | 99-47321-421 | 10p12 | WI-13843; 36589; 13843; EST239214; RH51513; |
| | | | R62459; WI-17514; 17514; EST250445; RH51289; |
| | | | R73587; AFMa175yd1; D10S1653; a175yd1; |
| 23 | 99-36198-261 | 3q26.3-q27 | WI-12636; 36269; 12636; EST327155; RH58715; |
| | | | R88719; |
| 24 | 99-36200-309 | 3q26.3-q27 | WI-12636; 36269; 12636; EST327155; RH58715; |
| | | | R88719; |
| 25 | 99-33135-357 | 3q26.2-q26.3 | 031yc5; AFM031yc5; D3S1258; RH5531; |
| 26 | 99-34451-198 | 3q26.3-q27 | WI-4441; 8190; 4441; D3S3065; G02704; |
| | | | MR7335; |
| 27 | 99-34452-238 | 3q26.3-q27 | WI-4441; 8190; 4441; D3S3065; G02704; |
| | | | MR7335; |
| 28 | 99-48928-392 | 10p12-p11.2 | |
| 29 | 99-55720-283 | 10p12.1-p11.2 | HLC.GGAA7H02; CHLC.31921; |
| | | | CHLC.GGAA7H02.31921; D10S1215; GGAA- |
| | | | D10S1215; G08825; WI-10806; 18662; 10806; |
| | | | D10S2164; G00-677-758; MR14911; G11884; |
| 30 | 99-55721-111 | 10p12.1-p11.2 | HLG.GGAA7H02; CHLC.31921; |
| | | | CHLC.GGAA7H02.31921; D10S1215; GGAA- |
| | | | D10S1215; G08825; WI-10806; 18662; 10806; |
| | | | D10S2164; G00-677-758; MR14911; G11884; |
| 31 | 99-501-57 | 10p12.1-p11.2 | AFMa106vf5; D10S1639; a106vf5; |
| 32 | 99-34556-66 | 10p12.1-p11.2 | I-17512; 17512; EST249648; RH51204; R72790; |
| | | | 295th1; AFM295th1; D10S586; RH2890; |
| | | | AFMa204yb9; D10S1662; a204yb9; RH42685; |
| | | | AFM295th1; |
| 33 | 99-34561-414 | 10p12.1-p11.2 | I-17512; 17512; EST249648; RH51204; R72790; |
| | | | 295th1; AFM295th1; D10S586; RH2890; |
| | | | AFMa204yb9; D10S1662; a204yb9; RH42685; |
| | | | AFM295th1; |
| 34 | 99-28061-254 | 10p12.1-p11.2 | WI-3231; 9716; 3231; D10S1578; G04230; G00- |
| | | | 588-756; MR3873; HALd26c11; ; |
| 35 | 99-28062-68 | 10p12.1-p11.2 | WI-3231; 9716; 3231; D10S1578; G04230; G00- |
| | | | 588-756; MR3873; HALd26c11; ; |
| 36 | 99-28063-331 | 10p12.1-p11.2 | WI-3231; 9716; 3231; D10S1578; G04230; G00- |
| | | | 588-756; MR3873; HALd26c11; ; |
| 37 | 99-28067-380 | 10p12.1-p11.2 | WI-3231; 9716; 3231; D10S1578; G04230; G00- |
| | | | 588-756; MR3873; HALd26c11; ; |
| 38 | 99-28080-254 | 10p12.1-p11.2 | WI-3231; 9716; 3231; D10S1578; G04230; G00- |
| | | | 588-756; MR3873; HALd26c11; ; |
| 39 | 99-459-347 | 10p12.1-p11.2 | 164tg9; AFM164tg9; D10S204; RH51372; |
| | | | SGC38221; |
| 40 | 99-32104-363 | 10p12.1-p11.2 | WI-18103; H56129; 18103; EST357452; RH51196; |
| | | | RH51356; D12273; SGC32671; SHGC-36745; |
| | | | R43751; |
| 41 | 99-26970-119 | 10p12 | 220xd4; AFM220xd4; D10S548; SHGC-2012; |
| | | | Z23664; |
| 42 | 99-27502-165 | 10p12 | 220xd4; AFM220xd4; D10S548; SHGC-2012; |
| | | | Z23664; |
| 43 | 99-26994-129 | 10p12.3-p12.2 | CHLC.GATA70E11; CHLC.GATA70E11.P18111; |
| | | | G08807; GATA-P18111; SHGC-8756; D10S2336; |
| 44 | 99-26995-256 | 10p12.3-p12.2 | CHLC.GATA70E11; CHLC.GATA70E11.P18111; |
| | | | G08807; GATA-P18111; SGGC-8756; D10S2336; |
| 45 | 99-26997-298 | 10p12.3-p12.2 | CHLC.GATA70E11; CHLC.GATA70E11.P18111; |
| | | | G08807; GATA-P18111; SHGC-8756; D10S2336; |
| 46 | 99-27001-115 | 10p12.3-p12.2 | CHLC.GATA70E11; CHLC.GATA70E11.P18111; |
| | | | G08807; GATA-P18111; SHGC-8756; D10S2336; |
| 47 | 99-32161-353 | 10p12.3-p12.2 | CHLC.GATA70E11; CHLC.GATA70E11.P18111; |
| | | | G08807; GATA-P18111; SHGC-8756; D10S2336; |
| 48 | 99-32165-233 | 10p12.3-p12.2 | CHLC.GATA70E11; CHLC.GATA70E11.P18111; |
| | | | G08807; GATA-P18111; SHGC-8756; D10S2336; |
| 49 | 99-32168-440 | 10p12.3-p12.2 | CHLC.GATA70E11; CHLC.GATA70E11.P18111; |
| | | | G08807; GATA-P18111; SHGC-8756; D10S2336; |
| 50 | 99-32172-210 | 10p12.3-p12.2 | CHLC.GATA70E11; CHLC.GATA70E11.P18111; |
| | | | G08807; GATA-P18111; SHGC-8756; D10S2336; |
| 51 | 99-32174-130 | 10p12.3-p12.2 | CHLC.GATA70E11; CHLC.GATA70E11.P18111; |
| | | | G08807; GATA-P18111; SHGC-8756; D10S2336; |
| 52 | 99-57310-483 | 10p12 | WI-12318; 35600; 12318; EST126093; RH51251; |
| | | | R16259; |
| 53 | 99-57314-120 | 10p12 | WI-12318; 35600; 12318; EST126093; RH51251; |
| | | | R16259; |
| 54 | 99-57314-120 | 10p12 | WI-12318; 35600; 12318; ST126093; H51251; |
| | | | R16259 |
| 55 | 99-26359-395 | 10p12.1-p11.2 | AFMc012xd5; D10S1747; c012xd5; AFMc012yc5; |
| | | | D10S1749; c012yc5; |
| 56 | 99-27232-379 | 10p12.1-p11.2 | WI-11237; 11237; MR14913; |
| 57 | 99-27595-373 | 10p12.1-p11.2 | WI-11237; 11237; MR14913; |
| 58 | 99-26492-146 | 10p12.1-p11.2 | WI-15162; H41127; 15162; EST302178; RH51077; |
| | | | SHGC-37336; 324xc1; AFM324xc1; D10S595; |
| | | | RH2647; |
| 59 | 99-26501-314 | 10p12.1-p11.2 | WI-15162; H41127; 15162; EST302178; RH51077; |
| | | | SHGC-37336; 324xc1; FM324xc1; D10S595; |
| | | | RH2647; |
| 60 | 99-28350-202 | 10p12.2-p12.1 | TIGR-A004U34; 40095; A004U34; RH51118; |
| | | | R43555; WI-3231; 9716; 3231; D10S1578; |
| | | | G04230; G00-588-756; MR3873; HALd26c11; WI- |
| | | | 31071; RH51413; RH51135; T62561; WI-8502; |
| 61 | 99-28355-255 | 10p12.2-p12.1 | TIGR-A004U34; 40095; A004U34; RH51118; |
| | | | R43555; WI-3231; 9716; 3231; D10S1578; |
| | | | G04230; G00-588-756; MR3873; HALd26c11; WI- |
| | | | 31071; RH51413; RH51135; T62561; WI-8502; |
| 62 | 99-28360-156 | 10p12.2-p12.1 | TIGR-A004U34; 40095; A004U34; RH51118; |
| | | | R43555; WI-3231; 9716; 3231; D10S1578; |
| | | | G04230; G00-588-756; MR3873; HALd26c11; WI- |
| | | | 31071; RH51413; RH51135; T62561; WI-8502; |
| 63 | 99-28363-114 | 10p12.2-p12.1 | TIGR-A004U34; 40095; A004U34; RH51118; |
| | | | R43555; WI-3231; 9716; 3231; D10S1578; |
| | | | G04230; G00-588-756; MR3873; HALd26c11; WI- |
| | | | 31071; RH51413; RH51135; T62561; WI-8502; |
| 64 | 99-28364-288 | 10p12.2-p12.1 | TIGR-A004U34; 40095; A004U34; RH51118; |
| | | | R43555; WI-3231; 9716; 3231; D10S1578; |
| | | | G04230; G00-588-756; MR3873; HALd26c11; WI- |
| | | | 31071; RH51413; RH51135; T62561; WI-8502; |
| 65 | 99-27049-396 | 10p12.1-p11.2 | RH51110; SGC31510; GAD2; |
| 66 | 99-27054-298 | 10p12.1-p11.2 | RH51110; SGC31510; GAD2; |
| 67 | 99-27056-327 | 10p12.1-p11.2 | RH51110; SGC31510; GAD2; |
| 68 | 99-27586-122 | 10p12.1-p11.2 | RH51110; SGC31510; GAD2; |
| 69 | 99-29193-332 | 10p12.1-p11.2 | RH51110; SGC31510; GAD2; |
| 70 | 99-56867-257 | 3q26 | |
| 71 | 99-26399-320 | 10p12.1-p11.2 | 238we5; AFM238we5; D10S550; |
| 72 | 99-41887-343 | 3q26 | |
| 73 | 99-27851-30 | 3q26 | |
| 74 | 99-38884-129 | 3q26.1-q26.2 | |
| 75 | 99-38897-96 | 3q26.1-q26.2 | |
| 76 | 99-36178-395 | 3q26.3-q27 | WI-12636; 36269; 12636; EST327155; RH58715; |
| | | | R88719; |
| 77 | 99-36201-377 | 3q26.3-q27 | WI-12636; 36269; 12636; EST327155; RH58715; |
| | | | R88719; |
| 78 | 99-32207-353 | 10p12.1-p11.2 | 338ta5; AFM338ta5; D10S600; |
| 79 | 99-55707-362 | 10p12.1-p11.2 | 199zb6; AFM199zb6; D10S213; |
| 80 | 99-48925-395 | 10p12-p11.2 | |
| 81 | 99-55725-246 | 10p12.1-p11.2 | B0952G04.T7.sl; ; |
| 82 | 99-28082-267 | 10p12.1-p11.2 | WI-3231; 9716; 3231; D10S1578; G04230; G00- |
| | | | 588-756; MR3873; HALd26c11; ; |
| 83 | 99-28083-222 | 10p12.1-p11.2 | WI-3231; 9716; 3231; D10S1578; G04230; G00- |
| | | | 588-756; MR3873; HALd26c11; ; |
| 84 | 99-27498-237 | 10p12 | 220xd4; AFM220xd4; D10S548; SHGC-2012; |
| | | | Z23664; |
| 85 | 99-27503-68 | 10p12 | 220xd4; AFM220xd4; D10S548; SHGC-2012; |
| | | | Z23664; |
| 86 | 99-26511-185 | 10p12.1-p11.2 | 198wf8; AFM198wf8; D10S211; RH15188; |
| | | | RH51521; D59489; SGC34521; STS38720; |
| | | | RH45652; |
| 87 | 99-26989-152 | 10p12.3-p12.2 | CHLC.GATA70E11; CHLC.GATA70E11.P18111; |
| | | | G08807; GATA-P18111; SHGC-8756; D10S2336; |
| 88 | 99-32158-71 | 10p12.3-p12.2 | CHLC.GATA70E11; CHLC.GATA70E11.P18111; |
| | | | G08807; GATA-P18111; SHGC-8756; D10S2336; |
| 89 | 99-32162-317 | 10p12.3-p12.2 | CHLC.GATA70E11; CHLC.GATA70E11.P18111; |
| | | | G08807; GATA-P18111; SHGC-8756; D10S2336; |
| 90 | 99-32166-332 | 10p12.3-p12.2 | CHLC.GATA70E11; CHLC.GATA70E11.P18111; |
| | | | G08807; GATA-P18111; SHGC-8756; D10S2336; ; |
| 91 | 99-32169-219 | 10p12.3-p12.2 | CHLC.GATA70E11; CHLC.GATA70E11.P18111; |
| | | | G08807; GATA-P18111; SHGC-8756; D10S2336; |
| 92 | 99-57305-320 | 10p12 | WI-12318; 35600; 12318; EST126093; RH51251; |
| | | | R16259; |
| 93 | 99-57308-151 | 10p12 | WI-12318; 35600; 12318; EST126093; RH51251; |
| | | | R16259 |
| 94 | 99-26477-419 | 10p12.1-p11.2 | WI-15162; H41127; 15162; EST302178; RH51077; |
| | | | SHGC-37336; 324xc1; AFM324xc1; D10S595; |
| | | | RH2647; |
| 95 | 99-26479-79 | 10p12.1-p11.2 | WI-15162; H41127; 15162; EST302178; RH51077; |
| | | | SHGC-37336; 324xc1; AFM324xc1; D10S595; |
| | | | RH2647; |
| 96 | 99-26480-284 | 10p12.1-p11.2 | WI-15162; H41127; 15162; EST302178; RH51077; |
| | | | SHGC-37336; 324xc1; AFM324xc1; D10S595; |
| | | | RH2647; |
| 97 | 99-26503-298 | 10p12.1-p11.2 | WI-15162; H41127; 15162; EST302178; RH51077; |
| | | | SHGC-37336; 324xc1; AFM324xc1; D10S595; |
| | | | RH2647; |
| 98 | 99-27042-125 | 10p12.1-p11.2 | RH51110; SGC31510; GAD2; |
| 99 | 99-27050-269 | 10p12.1-p11.2 | RH51110; SGC31510; GAD2; |
| 100 | 99-27583-97 | 10p12.3-p12.2 | WI-13343; 35381; 13343; EST22841; RH51180; |
| | | | T03813; |
| 101 | 99-55097-137 | 10p12.1-p11.2 | WI-8535; 11392; 8535; D10S1568; EST91372; |
| | | | RH50926; G00-588-494; G05895; T40568; SHGC- |
| | | | 13375; T40568; SHGC-33797; R94235; stSG4085; |
| | | | RH44419; |
| 102 | 99-41727-169 | 3q26.2 | WI-12842; 37488; 12842; RH58207; Z41421; |
| | | | HSCZTH032; RH58706; D20091; SGC32810; |
| 103 | 99-44259-417 | 10p12 | |
| 104 | 99-44260-155 | 10p12 | |
| 105 | 99-41009-244 | 3q26.3-q27 | RH58067; T36048; WI-16592; |
| 106 | 99-41010-102 | 3q26.3-q27 | RH58067; T36048; WI-16592; |
| 107 | 99-41828-310 | 3q26.2 | WI-21907; 21907; Hsa.27778; STS-R51246; |
| | | | RH58312; R51246; R58904; R59093; WI-14138; |
| 108 | 99-45821-385 | 10p12 | |
| 109 | 99-26385-78 | 10p12 | WI-11394; 34941; 11394; EST157636; BMI1; |
| | | | RH51068; T87515; SHGC-11041; L13689; BMI1; |
| | | | STS55217; RH78476; |
| 110 | 99-48212-159 | 3q26.3-q27 | CHLC.GATA28E03; D3S2433; G08242; GATA- |
| | | | D3S2433; |
| 111 | 99-36203-346 | 3q26.3-q27 | WI-12636; 36269; 12636; EST327155; RH58715; |
| | | | R88719; ; |
| 112 | 99-441-155 | 10p12.1-p11.2 | 338ta5; AFM338ta5; D10S600; |
| 113 | 99-444-226 | 10p12.1-p11.2 | 338ta5; AFM338ta5; D10S600; |
| 114 | 99-55706-54 | 10p12.1-p11.2 | 199zb6; AFM199zb6; D10S213; |
| 115 | 99-55708-159 | 10p12.1-p11.2 | 199zb6; AFM199zb6; D10S213; |
| 116 | 99-55709-240 | 10p12.1-p11.2 | 199zb6; AFM199zb6; D10S213; |
| 117 | 99-36422-176 | 3q26.2-q26.3 | TIGR-A004V10; 40295; A004V10; RH58181; |
| | | | Z41342; |
| 118 | 99-36426-50 | 3q26.2-q26.3 | TIGR-A004V10; 40295; A004V10; RH58181; |
| | | | Z41342; |
| 119 | 99-54269-455 | 3q26.1 | |
| 120 | 99-54270-270 | 3q26.1 | |
| 121 | 99-54272-97 | 3q26.1 | |
| 122 | 99-54276-127 | 3q26.1 | |
| 123 | 99-54277-41 | 3q26.1 | |
| 124 | 99-49542-187 | 3q26.1 | |
| 125 | 99-49544-423 | 3q26.1 | |
| 126 | 99-49545-396 | 3q26.1 | |
| 127 | 99-49547-344 | 3q26.1 | |
| 128 | 99-457-400 | 10p12.1-p11.2 | 164tg9; AFM164tg9; D10S204; RH51372; |
| | | | SGC38221; |
| 129 | 99-463-90 | 10p12.1-p11.2 | 164tg9; AFM164tg9; D10S204; RH51372; |
| | | | SGC38221; |
| 130 | 99-32097-347 | 10p12.1-p11.2 | WI-18103; H56129; 18103; EST357452; RH51196; |
| | | | RH51356; D12273; SGC32671; SHGC-36745; |
| | | | R43751; |
| 131 | 99-32110-64 | 10p12.1-p11.2 | WI-18103; H56129; 18103; EST357452; RH51196; |
| | | | RH51356; D12273; SGC32671; SHGC-36745; |
| | | | R43751; |
| 132 | 99-32117-226 | 10p12.1-p11.2 | WI-18103; H56129; 18103; EST357452; RH51196; |
| | | | RH51356; D12273; SGC32671; SHGC-36745; |
| | | | R43751; |
| 133 | 99-26972-44 | 10p12 | 220xd4; AFM220xd4; D10S548; SHGC-2012; |
| | | | Z23664; |
| 134 | 99-26973-239 | 10p12 | 220xd4; AFM220xd4; D10S548; SHGC-2012; |
| | | | Z23664; |
| 135 | 99-26974-450 | 10p12 | 220xd4; AFM220xd4; D10S548; SHGC-2012; |
| | | | Z23664; |
| 136 | 99-26975-40 | 10p12 | 220xd4; AFM220xd4; D10S548; SHGC-2012; |
| | | | Z23664; |
| 137 | 99-26978-122 | 10p12 | 220xd4; AFM220xd4; D10S548; SHGC-2012; |
| | | | Z23664; |
| 138 | 99-26981-171 | 10p12 | 220xd4; AFM220xd4; D10S548; SHGC-2012; |
| | | | Z23664; |
| 139 | 99-26983-292 | 10p12 | 220xd4; AFM220xd4; D10S548; SHGC-2012; |
| | | | Z23664; |
| 140 | 99-26987-196 | 10p12.3-p12.2 | CHLC.GATA70E11; CHLC.GATA70E11.P18111; |
| | | | G08807; GATA-P18111; SHGC-8756; D10S2336; |
| 141 | 99-32153-348 | 10p12.3-p12.2 | CHLC.GATA70E11; CHLC.GATA70E11.P18111; |
| | | | G08807; GATA-P18111; SHGC-8756; D10S2336; |
| 142 | 99-32156-127 | 10p12.3-p12.2 | CHLC.GATA70E11; CHLC.GATA70E11.P18111; |
| | | | G08807; GATA-P18111; SHGC-8756; D10S2336; |
| 143 | 99-57302-162 | 10p12 | WI-12318; 35600; 12318; EST126093; RH51251; |
| | | | R16259; |
| 144 | 99-26349-339 | 10p12.1-p11.2 | AFMc012xd5; D10S1747; c012xd5; AFMc012yc5; |
| | | | D10S1749; c012yc5; |
| 145 | 99-26352-71 | 10p12.1-p11.2 | AFMc012xd5; D10S1747; c012xd5; AFMc012yc5; |
| | | | D10S1749; c012yc5; |
| 146 | 99-26370-60 | 10p12.2-p12.1 | RH51100; H52704; SGG35686; SHGC-3893; |
| 147 | 99-28332-445 | 10p12.1-p11.2 | SHGC-2052; Z24559; 342xe9; AFM342xe9; |
| | | | D10S601; |
| 148 | 99-28339-362 | 10p12.1-p11.2 | SHGC-2052; Z24559; 342xe9; AFM342xe9; |
| | | | D10S601; |
| 149 | 99-28342-137 | 10p12.1-p11.2 | SHGC-2052; Z24559; 342xe9; AFM342xe9; |
| | | | D10S601; |
| 150 | 99-28343-487 | 10p12.1-p11.2 | SHGC-2052; Z24559; 342xe9; AFM342xe9; |
| | | | D10S601; |
| 151 | 99-28352-101 | 10p12.2-p12.1 | TIGR-A004U34; 40095; A004U34; RH51118; |
| | | | R43555; WI-3231; 9716; 3231; D10S1578; |
| | | | G04230; G00-588-756; MR3873; HALd26c11; WI- |
| | | | 31071; RH51413; RH51135; T62561; WI-8502; |
| 152 | 99-28371-233 | 10p12.2-p12.1 | TIGR-A004U34; 40095; A004U34; RH51118; |
| | | | R43555; WI-3231; 9716; 3231; D10S1578; |
| | | | G04230; G00-588-756; MR3873; HALd26c11; WI- |
| | | | 31071; RH51413; RH51135; T62561; WI-8502; |
| 153 | 99-27041-274 | 10p12.1-p11.2 | RH51110; SGC31510; GAD2; |
| 154 | 99-29191-241 | 10p12.1-p11.2 | RH51110; SGG31510; GAD2; |
| 155 | 99-27074-429 | 10p12.3-p12.2 | WI-13343; 35381; 13343; EST22841; RH51180; |
| | | | T03813; |
| 156 | 99-27076-313 | 10p12.3-p12.2 | WI-13343; 35381; 13343; EST22841; RH51180; |
| | | | T03813; |
| 157 | 99-27079-302 | 10p12.3-p12.2 | WI-13343; 35381; 13343; EST22841; RH51180; |
| | | | T03813; |
| 158 | 99-27082-303 | 10p12.3-p12.2 | WI-13343; 35381; 13343; EST22841; RH51180; |
| | | | T03813; |
| 159 | 99-55890-43 | 3q26.1-q26.2 | |
| 160 | 99-55901-112 | 3q26.1-q26.2 | |
| 161 | 99-55903-324 | 3q26.1-q26.2 | |
| 162 | 9-42-169 | 19q13.2 | WI-12417; H16169; 34750; 12417; EST276107; |
| | | | RH55689 |
| 163 | 99-41725-266 | 3q26.2 | WI-12842; 37488; 12842; RH58207; Z41421; |
| | | | HSCZTH032; RH58706; D20091; SGC32810; |
| 164 | 99-45440-313 | 10p12.1-p11.2 | AFMa204xe1; D10S1660; a204xe1; STS34233; |
| | | | RH40225; |
| 165 | 99-438-82 | 10p12.1-p11.2 | 338ta5; AFM338ta5; D10S600; |
| 166 | 99-36428-363 | 3q26.2-q26.3 | TIGR-A004V10; 40295; A004V10; RH58181; |
| | | | Z41342; |
| 167 | 99-36433-318 | 3q26.2-q26.3 | TIGR-A004V10; 40295; A004V10; RH58181; |
| | | | Z41342; |
| 168 | 99-26968-300 | 10p12 | 220xd4; AFM220xd4; D10S548; SHGC-2012; |
| | | | Z23664; |
| 169 | 99-26345-57 | 10p12.1-p11.2 | AFMc012xd5; D10S1747; c012xd5; AFMc012yc5; |
| | | | D10S1749; c012yc5; |
| 170 | 99-27070-379 | 10p12.3-p12.2 | WI-13343; 35381; 13343; EST22841; RH51180; |
| | | | T03813; |
| 171 | 99-27581-206 | 10p12.3-p12.2 | WI-13343; 35381; 13343; EST22841; RH51180; |
| | | | T03813; |

What is claimed is:

1. A method of genotyping comprising determining the identity of a nucleotide at a map-related biallelic marker in a biological sample, wherein said map-related biallelic marker is selected from the group consisting of the biallelic markers of SEQ ID Nos. 1 to 171, and the complements thereto.

2. A method according to claim 1, wherein said map-related biallelic marker is selected from the group consisting of the biallelic markers of SEQ ID Nos. 1 to 100 and 101 to 162, and the complements thereto.

3. A method according to claims 1 or 2, wherein the identity of a nucleotide at 5 biallelic markers selected from the group consisting of the biallelic markers of SEQ ID Nos. 1 to 100, and the complements thereto is determined.

4. A method according to claims 1 or 2, wherein the identity of a nucleotide at 10 biallelic markers selected from the group consisting of the biallelic markers of SEQ ID Nos. 1 to 171, and the complements thereto is determined.

5. A method according to claims 1 or 2, wherein the identity of a nucleotide at 20 biallelic markers selected from the group consisting of the biallelic markers of SEQ ID Nos. 1 to 171, and the complements thereto is determined.

6. A method according to claim 1 or 2, wherein the identity of a nucleotide at 50 biallelic markers selected from the group consisting of the biallelic markers of SEQ ID Nos. 1 to 171, and the complements thereto is determined.

7. A method according to claim 1 or 2, wherein the identity of a nucleotide at 100 biallelic markers selected from the group consisting of the biallelic markers of SEQ ID Nos. 1 to 171, and the complements thereto is determined.

8. A method of genotyping comprising determining the identity of a nucleotide at a set of biallelic markers in a biological sample, and the complements thereto, wherein said set comprises 10 map-related biallelic markers selected from the group consisting of the biallelic markers of SEQ ID Nos. 1 to 171, wherein said biallelic markers are selected to have a heterozygosity rate of at least about 0.18, and are separated from one another by an average distance of 10 kb to 200 kb.

9. A method according to claim 8 wherein said set of biallelic markers comprises 20 map-related biallelic markers selected from the group consisting of the biallelic markers of SEQ ID Nos. 1 to 171, wherein said biallelic markers are selected to have a heterozygosity rate of at least about 0.18, and are separated from one another by an average distance of 10 kb to 200 kb.

10. A method according to claim 8 wherein said set of biallelic markers comprises 100 map-related biallelic markers selected from the group consisting of the biallelic markers of SEQ ID Nos. 1 to 171, wherein said biallelic markers are selected to have a heterozygosity rate of at least about 0.18, and are separated from one another by an average distance of 10 kb to 200 kb.

11. A method according to claim 8, 9 or 10 wherein said map-related biallelic markers are selected to have a heterozygosity rate of at least about 0.32.

12. A method according to claim 8, 9 or 10, wherein said map-related biallelic markers are separated from one another by an average distance of 25 kb to 50 kb.

13. A method according to claim 1, wherein said biological sample is derived from a single subject.

14. A method according to claim 13, wherein the identity of the nucleotides at said biallelic marker is determined for both copies of said biallelic marker present in said subject's genome.

15. A method according claim 1, wherein said biological sample is derived from multiple subjects.

16. A method according to claim 1, further comprising amplifying a portion of said sequence comprising the biallelic marker prior to said determining step.

17. A method according to claim 16, wherein said amplifying is performed by PCR.

18. A method according to claim 1, wherein said determining is performed by a hybridization assay, a sequencing assay, a microsequencing assay, or an enzyme-based mismatch detection assay.

19. A method of determining the frequency in a population of an allele of a map-related biallelic marker, comprising:
a) genotyping individuals from said population for said biallelic marker according to the method of claim 1; and
b) determining the proportional representation of said biallelic marker in said population.

20. A method according to claim 19, wherein said map-related biallelic marker is selected from the group consisting of the biallelic markers of SEQ ID Nos. 1 to 171, and the complements thereto.

21. A method according to claim 19, wherein said map-related biallelic marker is selected from the group consisting of the biallelic markers of SEQ ID Nos. 1 to 100 and 101 to 162, and the complements thereto.

22. A method according to claim 19, wherein said genotyping of step a) is performed on each individual of said population.

23. A method according to claim 19, wherein said genotyping is performed on a single biological sample derived from said population.

24. A method of detecting an association between an allele and a phenotype, comprising the steps of:
a) determining the frequency of at least one map-related biallelic marker allele in a trait positive population according to the method of claim 19;
b) determining the frequency of said map-related biallelic marker allele in a control population according to the method of claim 19; and
c) determining whether a statistically significant association exists between said allele and said phenotype.

25. A method of estimating the frequency of a haplotype for a set of biallelic markers in a population, comprising:
a) genotyping each individual in said population for at least one map-related biallelic marker according to claim 13;
b) genotyping each individual in said population for a second biallelic marker by determining the identity of the nucleotides at said second biallelic marker for both copies of said second biallelic marker present in the genome; and
c) applying a haplotype determination method to the identities of the nucleotides determined in steps a) and b) to obtain an estimate of said frequency.

26. A method according to claim 25, wherein said haplotype determination method is selected from the group consisting of asymmetric PCR amplification, double PCR amplification of specific alleles, the Clark method, or an expectation maximization algorithm.

27. A method according to claim 25, wherein said map-related biallelic marker is selected from the group consisting of the biallelic markers of SEQ ID Nos. 1 to 171, and the complements thereto.

28. A method according to claim 25, wherein said map-related biallelic marker is selected from the group consisting of the biallelic markers of SEQ ID Nos. 1 to 100, 101 to 162, and the complements thereto.

29. A method of detecting an association between a haplotype and a phenotype, comprising the steps of:
a) estimating the frequency of at least one haplotype in a trait positive population according to the method of claim 25;
b) estimating the frequency of said haplotype in a control population according to the method of claim 25; and
c) determining whether a statistically significant association exists between said haplotype and said phenotype.

30. A method according to either claim 24 or 29, wherein said control population is a trait negative population.

31. A method according to either claim 24 or 29, wherein said case control population is a random population.

32. A method according to claim 24, wherein each of said genotyping of steps a) and b) is performed on a single pooled biological sample derived from each of said populations.

33. A method according to claim 42, wherein said genotyping of steps a) and b) is performed separately on biological samples derived from each individual in said populations.

34. A method according to either claim 24 or 29, wherein said phenotype is selected from the group consisting of disease, drug response, drug efficacy, treatment response, treatment efficacy, and drug toxicity.

35. A method according to claim 24, wherein the identity of the nucleotides at all of the biallelic markers of SEQ ID Nos. 1 to 171 is determined in steps a) and b).

36. A method according to claim 24, wherein the identity of the nucleotides at 10 of the biallelic markers of SEQ ID Nos. 1 to 171 is determined in steps a) and b).

37. A method of identifying a gene associated with a detectable trait comprising the steps of:
a) determining the frequency of each allele of at least one map-related biallelic marker in individuals having said detectable trait and individuals lacking said detectable trait according to the method of claim 23;
b) identifying at least one allele of said biallelic marker having a statistically significant association with said detectable trait; and
c) identifying a gene in linkage disequilibrium with said allele.

38. The method according to claim 37, further comprising the step of: d) identifying a mutation in gene which is associated with said detectable trait.

39. A method of identifying biallelic markers associated with a detectable trait comprising the steps of:
a) determining the frequencies of a set of biallelic markers comprising at least one map-related biallelic marker selected from the group consisting of the biallelic markers of SEQ ID Nos. 1 to 171 in individuals who express said detectable trait and individuals who do not express said detectable trait; and
b) identifying at least one biallelic marker in said set which are statistically associated with the expression of said detectable trait.

40. A method for determining whether an individual is at risk of developing a detectable trait or suffers from a detectable trait associated with said trait comprising the steps of:
a) obtaining a nucleic acid sample from said individual;
b) screening said nucleic acid sample with at least one map-related biallelic marker selected from the group consisting of the biallelic markers of SEQ ID Nos. 1 to 171; and
c) determining whether said nucleic acid sample contains at least one biallelic marker statistically associated with said detectable trait.

41. The method according to any one of claims 37, 39 and 40, wherein said detectable trait is selected from the group consisting of disease, drug response, drug efficacy, treatment response, treatment efficacy, and drug toxicity.

42. A method of administering a drug or treatment comprising:
a) obtaining a nucleic acid sample from an individual;
b) determining the identity of the polymorphic base of at least one map-related biallelic marker according to the method of claim 13 which is associated with a positive response to said drug or treatment, or at least one map-related biallelic marker which is associated with a negative response to said drug or treatment; and
c) administering said drug or treatment to said individual if said nucleic acid sample contains at least one biallelic marker associated with a positive response to said drug or treatment, or if said nucleic acid sample lacks at least one biallelic markers associated with a negative response to said drug or treatment.

43. A method of selecting an individual for inclusion in a clinical trial of a drug or treatment comprising:
a) obtaining a nucleic acid sample from an individual;
b) determining the identity of the polymorphic base of at least one map-related biallelic marker according to the method of claim 13 which is associated with a positive response to said drug or treatment, or at least one biallelic marker associated with a negative response to said drug or treatment in said nucleic acid sample; and
c) including said individual in said clinical trial if said nucleic acid sample contains at least one biallelic marker which is associated with a positive response to said drug or treatment, or if said nucleic acid sample lacks at least one biallelic markers associated with a negative response to said drug or treatment.

44. The method according to claim 43, wherein said administering step comprises administering said drug or treatment to said individual if said nucleic acid sample contains at least one biallelic marker associated with a positive response to said drug or treatment, and said nucleic acid sample lacks at least one biallelic marker associated with a negative response to said drug or treatment.

45. Use of a polynucleotide for use in determining the identity of nucleotides at a map-related biallelic marker selected from the group consisting of the biallelic markers of SEQ ID Nos. 1 to 171.

46. Use of a polynucleotide according to claim 45, wherein said determining is performed in a hybridization assay, sequencing assay, microsequencing assay, or an enzyme-based mismatch detection assay.

47. Use of a polynucleotide for use in amplifying a segment of nucleotides comprising a map-related biallelic marker selected from the group consisting of the biallelic markers of SEQ ID Nos. 1 to 171.

48. Use of a polynucleotide according to either of claims 45 and 47, wherein said map-related biallelic marker is selected from the group consisting of the biallelic markers of SEQ IID Nos. 1 to 100, 101 to 162 and the complements thereto.

49. Use of a polynucleotide according to any one of claims 45 to 47, wherein said polynucleotide is attached to a solid support.

50. Use of a polynucleotide according to claim 49, wherein said polynucleotide is attached to an array.

51. Use of a polynucleotide according to claim 50, wherein said array is addressable.

52. Use of a polynucleotide according to any one of claims 45 to 47, wherein said polynucleotide further comprises a label.

53. Use of a computer readable medium having stored thereon the sequence of a polynucleotide comprising a contiguous span of 12 nucleotides selected from the group consisting of SEQ ID Nos. 1 to 171 comprising a map-related biallelic marker, to analyze a nucleotide sequence.

54. Use of a computer system comprising a processor and a data storage device wherein said data storage device has stored thereon the sequence of a polynucleotide comprising a contiguous span of 12 nucleotides selected from the group consisting of SEQ ID Nos. 1 to 171 comprising a map-related biallelic marker, to analyze a nucleotide sequence.

55. The use of a computer system according to claim 54, wherein said computer system further comprises a sequence comparer and a data storage device having reference sequences stored thereon.

56. A method for comparing a first sequence to a reference sequence, comprising the steps of:
a) reading said first sequence and said reference sequence through use of a computer program which compares sequences; and
b) determining differences between said first sequence and said reference sequence with said computer program;
wherein said first sequence is the sequence of a polynucleotide comprising a contiguous span of 12 nucleotides selected from the group consisting of SEQ ID Nos. 1 to 171 comprising a map-related biallelic marker.

57. Use of a computer system according to claim 54 wherein said data storage device has stored thereon the sequence of 10 polynucleotides comprising a map-related biallelic marker selected from the group consisting of SEQ ID Nos. 1 to 171.


SEQUENCE LISTING (579 sequences)

SEQ ID NO: 1 - DNA, 47 bp, Homo Sapiens
  allele 24: 99-55095-221  polymorphic base A or G
tgcaactttgattttgcactcaaragatttctgtagtagaaaaaata

SEQ ID NO: 2 - DNA, 47 bp, Homo Sapiens
  allele 24: 99-55102-112  polymorphic base G or T
attgaattacaggttgcagctttkaactcttcaccttttaaactgtc

SEQ ID NO: 3 - DNA, 47 bp, Homo Sapiens
  allele 24: 99-26407-73  polymorphic base A or G
ttcttttatggtgttacagagtcrgaaaagtaattttaccagaagag

SEQ ID NO: 4 - DNA, 47 bp, Homo Sapiens
  allele 24: 99-26412-162  polymorphic base G or C
gagatatatgcacatttgtaaaastatgaaagaacataaaaggatca

SEQ ID NO: 5 - DNA, 47 bp, Homo Sapiens
  allele 24: 99-26413-307  polymorphic base A or T
ctccctctgtgctgaagatgtgtwgctgtgggttattttaaaaatcc

SEQ ID NO: 6 - DNA, 47 bp, Homo Sapiens
  allele 24: 99-26424-423  polymorphic base C or T
atgggaaagagaagtaaagatgayaacagattgagctgaacggaatt

SEQ ID NO: 7 - DNA, 47 bp, Homo Sapiens
  allele 24: 99-44320-377  polymorphic base A or T
ttagagttttaggaggaagcaaawataaacacagggttccatctgcc

SEQ ID NO: 8 - DNA, 47 bp, Homo Sapiens
  allele 24: 99-41889-287  polymorphic base C or T
atggcagcctgagcagactaatayggtgtgtatcaatgtctttctct

SEQ ID NO: 9 - DNA, 47 bp, Homo Sapiens
  allele 24: 99-45453-183  polymorphic base A or T
ttaacaagtgcaattagcatgtawcaatgaaataattgcttgagttc

SEQ ID NO: 10 - DNA, 47 bp, Homo Sapiens
  allele 24: 99-43427-381  polymorphic base G or T
gttgtgctgcgtcacagtacaaakgctgtgttatgttcaggactaaa

SEQ ID NO: 11 - DNA, 47 bp, Homo Sapiens
  allele 24: 99-55124-180  polymorphic base C or T
gtcaacaatgattattgccaatayagttacacaaatgccaattgcag

SEQ ID NO: 12 - DNA, 47 bp, Homo Sapiens
  allele 24: 99-46316-65  polymorphic base A or T
agcacgcgcgtgcccttccaggawggttggctgcttctcgttgctca

SEQ ID NO: 13 - DNA, 47 bp, Homo Sapiens
  allele 24: 99-46325-327  polymorphic base C or T
tggcacataacagataatgaattycagctgaatttaaaagaagacaa

SEQ ID NO: 14 - DNA, 47 bp, Homo Sapiens
  allele 24: 99-46332-107  polymorphic base A or G
tgtataatcttaccttttgtgctrtatttgttagattgtattaacaa

SEQ ID NO: 15 - DNA, 47 bp, Homo Sapiens
  allele 24: 99-47064-85  polymorphic base C or T
tgaagtatctagaggtagatgggytagatccaaaggtaatatatctc

SEQ ID NO: 16 - DNA, 47 bp, Homo Sapiens
  allele 24: 99-47065-42  polymorphic base C or T
atgctcaaaccccaaattgggagytcttttcttataaaatgttatga

SEQ ID NO: 17 - DNA, 47 bp, Homo Sapiens
  allele 24: 99-47066-258  polymorphic base A or G
tcacttgctcctttggtgggctcragtatgcccctggtggcactgag

SEQ ID NO: 18 - DNA, 47 bp, Homo Sapiens
  allele 24: 99-38883-331  polymorphic base C or T
agagatgtggctcacagtacagcygtaagtactacagattaaagggc

SEQ ID NO: 19 - DNA, 47 bp, Homo Sapiens
  allele 24: 99-38888-357  polymorphic base C or T
ggatgaggagccagggaaaggacytggacagaactaagaagctgcag

SEQ ID NO: 20 - DNA, 47 bp, Homo Sapiens
  allele 24: 99-38889-169  polymorphic base C or T
aggaaggcttgggtaggagatggyaagggtccaactaaggggaaatt

SEQ ID NO: 21 - DNA, 47 bp, Homo Sapiens
  allele 24: 99-26458-340  polymorphic base G or C
cgtgataggcagagtaaaattacsccatattctactgcatagaaagc

SEQ ID NO: 22 - DNA, 47 bp, Homo Sapiens
  allele 24: 99-47321-421  polymorphic base G or T
gacatgagctgagcccagactttkctcgaagccaatctcgtttcact

SEQ ID NO: 23 - DNA, 47 bp, Homo Sapiens
  allele 24: 99-36198-261  polymorphic base C or T
atatatgaccttcacattgcaagyctgcctgtgctgaaactttcttg

SEQ ID NO: 24 - DNA, 47 bp, Homo Sapiens
  allele 24: 99-36200-309  polymorphic base A or G
ctcttggagtcatccttgatcctrtatataatcagaaaatccttttg

SEQ ID NO: 25 - DNA, 47 bp, Homo Sapiens
  allele 24: 99-33135-357  polymorphic base C or T
aactttctcctttgtctctcaagycttaaagaactttgctttcccaa

SEQ ID NO: 26 - DNA, 47 bp, Homo Sapiens
  allele 24: 99-34451-198  polymorphic base C or T
ccatgattggcacctgtatcttcyctcctggcttcactggggctgac

SEQ ID NO: 27 - DNA, 47 bp, Homo Sapiens
  allele 24: 99-34452-238  polymorphic base A or C
aaaaatatagtcttattatttggmatttaaacagtcatggaaaaaat

SEQ ID NO: 28 - DNA, 47 bp, Homo Sapiens
  allele 24: 99-48928-392  polymorphic base G or T
tagagataccaagattttattctktctccatgggaatgttaaacata

SEQ ID NO: 29 - DNA, 47 bp, Homo Sapiens
  allele 24: 99-55720-283  polymorphic base A or G
aagattaaaacacaaacacacacrcacacacacacacaagcaacttc

SEQ ID NO: 30 - DNA, 47 bp, Homo Sapiens
  allele 24: 99-55721-111  polymorphic base C or T
ataacatggctttcgaagttgctygtgtgtgtgtgtgygtgtgtgtt

SEQ ID NO: 31 - DNA, 47 bp, Homo Sapiens
  allele 24: 99-501-57  polymorphic base A or G
acagattaatccaacgtagcaacraattactaggcacccaattttca

SEQ ID NO: 32 - DNA, 47 bp, Homo Sapiens
  allele 24: 99-34556-66  polymorphic base C or T
ttgagtaacctttgagatgaaaaygaggactcaactgatgttgctac

SEQ ID NO: 33 - DNA, 47 bp, Homo Sapiens
  allele 24: 99-34561-414  polymorphic base G or C
atggtgaacagattttagatagasttttcatggcaaagggaaacaaa

SEQ ID NO: 34 - DNA, 47 bp, Homo Sapiens
  allele 24: 99-28061-254  polymorphic base G or C
ttcgataacttaactgctgtagastgcaattttttttactaattgcc

SEQ ID NO: 35 - DNA, 47 bp, Homo Sapiens
  allele 24: 99-28062-68  polymorphic base A or C
gttgcttatttataaaataaagamcatgatttctgtcttagtacttt

SEQ ID NO: 36 - DNA, 47 bp, Homo Sapiens
  allele 24: 99-28063-331  polymorphic base C or T
tatattagcagcctcactccttgyggaattctggaacagagcaaagt

SEQ ID NO: 37 - DNA, 47 bp, Homo Sapiens
  allele 24: 99-28067-380  polymorphic base C or T
taggagagaaagcagagaggaaaytagctaagtaggtaaaaacagcc

SEQ ID NO: 38 - DNA, 47 bp, Homo Sapiens
  allele 24: 99-28080-254  polymorphic base G or C
attaaaaaaaaaaattaacagcasgttgtgaattgtgttggcaatta

SEQ ID NO: 39 - DNA, 47 bp, Homo Sapiens
  allele 24: 99-459-347  polymorphic base A or G
atgggaggtgctcctgaatgtgcrggtactaacagggagaagatgca

SEQ ID NO: 40 - DNA, 47 bp, Homo Sapiens
  allele 24: 99-32104-363  polymorphic base A or G
ggcatattattaaaaggctttggrcatgacccataactcatgatatg

SEQ ID NO: 41 - DNA, 47 bp, Homo Sapiens
  allele 24: 99-26970-119  polymorphic base G or C
aaacagtaatcacacaatgtaaasaattagaaaaaactagtcaaatt

SEQ ID NO: 42 - DNA, 47 bp, Homo Sapiens
  allele 24: 99-27502-165  polymorphic base A or T
ggactgcaacctacgtatttagtwctagaggtgtttagtacccgagt

SEQ ID NO: 43 - DNA, 47 bp, Homo Sapiens
  allele 24: 99-26994-129  polymorphic base C or T
ttaacagttttgtaaaggaatttyatatattatctatctgattctct

SEQ ID NO: 44 - DNA, 47 bp, Homo Sapiens
  allele 24: 99-26995-256  polymorphic base A or C
tggttttcacctcgggtataggtmatcaataaaatgggatgcttctc

SEQ ID NO: 45 - DNA, 47 bp, Homo Sapiens
  allele 24: 99-26997-298  polymorphic base A or G
gattgttaccttggcactgatgcrtagggactctaggatctgggacg

SEQ ID NO: 46 - DNA, 47 bp, Homo Sapiens
  allele 24: 99-27001-115  polymorphic base G or T
taatcctgttaaaatctaatataktctcattaaatgaaatattcccc

SEQ ID NO: 47 - DNA, 47 bp, Homo Sapiens
  allele 24: 99-32161-353  polymorphic base A or G
tcatcggatgtcattccccatatrcgttagcatcgtcttaactttac

SEQ ID NO: 48 - DNA, 47 bp, Homo Sapiens
  allele 24: 99-32165-233  polymorphic base G or T
cagcaatgttcttcattcagatakctgctgaaggcaccattctaatt

SEQ ID NO: 49 - DNA, 47 bp, Homo Sapiens
  allele 24: 99-32168-440  polymorphic base C or T
gtaaagttaagacgatgctaacgyatatggggaatgacatccgatga

SEQ ID NO: 50 - DNA, 47 bp, Homo Sapiens
  allele 24: 99-32172-210  polymorphic base A or C
ccatcaacattttctaaacacacmatacactttctcatctctgtgtt

SEQ ID NO: 51 - DNA, 47 bp, Homo Sapiens
  allele 24: 99-32174-130  polymorphic base A or G
aggtctgatactgccctttgatartcacagtgataggcaatttggaa

SEQ ID NO: 52 - DNA, 47 bp, Homo Sapiens
  allele 24: 99-57310-483  polymorphic base A or G
agggacaggagtaagactgagagrccagaaaatctagttaggaggtt

SEQ ID NO: 53 - DNA, 47 bp, Homo Sapiens
  allele 24: 99-57311-339  polymorphic base G or C
caagttaaggtctaatcagcagtsaccctaggaatggttaatggtga

SEQ ID NO: 54 - DNA, 47 bp, Homo Sapiens
  allele 24: 99-57314-120  polymorphic base C or T
aagagaagtatttactaatgaagyagaagctacatgagctaatattt

SEQ ID NO: 55 - DNA, 47 bp, Homo Sapiens
  allele 24: 99-26359-395  polymorphic base C or T
tttcacacacattatttcaaatayagttctcccaaaaaaacctgcgg

SEQ ID NO: 56 - DNA, 47 bp, Homo Sapiens
  allele 24: 99-27232-379  polymorphic base A or G
cctcagccccccaaacagctgggrctgtaggcatgcactaccccgtc

SEQ ID NO: 57 - DNA, 47 bp, Homo Sapiens
  allele 24: 99-27595-373  polymorphic base G or C
tctctttaagtgaattttgaagtsaccaaaaagtcatggatatacta

SEQ ID NO: 58 - DNA, 47 bp, Homo Sapiens
  allele 24: 99-26492-146  polymorphic base C or T
attacttttctttatttttttacyttattttattttcttttagagac

SEQ ID NO: 59 - DNA, 47 bp, Homo Sapiens
  allele 24: 99-26501-314  polymorphic base C or T
tcacagaaaagatcgtggaaccayggaagtatacataatacaggtag

SEQ ID NO: 60 - DNA, 47 bp, Homo Sapiens
  allele 24: 99-28350-202  polymorphic base A or G
tttccctctcttcttcccattatrgccatattaaatgtggctttttt

SEQ ID NO: 61 - DNA, 47 bp, Homo Sapiens; identical to SEQ ID NO: 38
  allele 24: 99-28355-255  polymorphic base G or C

SEQ ID NO: 62 - DNA, 47 bp, Homo Sapiens
  allele 24: 99-28360-156  polymorphic base A or G
aagaattgtgccaatgggatagcrcattcatgatactaatgatattg

SEQ ID NO: 63 - DNA, 47 bp, Homo Sapiens
  allele 24: 99-28363-114  polymorphic base A or G
tgtctgagctaaggggcctggaarctgcagtgattttgcctaagttg

SEQ ID NO: 64 - DNA, 47 bp, Homo Sapiens
  allele 24: 99-28364-288  polymorphic base A or G
ggggtaaaaaaactccacaactcrtctccctgcatatcttcccctcc

SEQ ID NO: 65 - DNA, 47 bp, Homo Sapiens
  allele 24: 99-27049-396  polymorphic base C or T
agtcttgtctgaatttggaagcayatttacttggaatcaggatacat

SEQ ID NO: 66 - DNA, 47 bp, Homo Sapiens
  allele 24: 99-27054-298  polymorphic base A or C
gcaggacccatttattcactgacmaggaagaagccctgggttgggca

SEQ ID NO: 67 - DNA, 47 bp, Homo Sapiens
  allele 24: 99-27056-327  polymorphic base C or T
aaaggaaagcaagcaaatgactayctgaggttgttttgttactagag

SEQ ID NO: 68 - DNA, 47 bp, Homo Sapiens
  allele 24: 99-27586-122  polymorphic base A or G
agaggaggaggagggagggagggrgattgttttgaagtgactgagta

SEQ ID NO: 69 - DNA, 47 bp, Homo Sapiens
  allele 24: 99-29193-332  polymorphic base A or C
ctgctgttgtagacactacgtaamcaaacaagcatggctagtgccaa

SEQ ID NO: 70 - DNA, 47 bp, Homo Sapiens
  allele 24: 99-56867-257  polymorphic base A or T
gcaaagagtaattcattaatactwacacagatgagtacataggaaat

SEQ ID NO: 71 - DNA, 47 bp, Homo Sapiens
  allele 24: 99-26399-320  polymorphic base C or T
cactgggaaggtggacaatcccaygggggagtctctttgggggttgg

SEQ ID NO: 72 - DNA, 47 bp, Homo Sapiens
  allele 24: 99-41887-343  polymorphic base A or C
tatttggtgttttgaaagactacmgtctcttaagagacaaagatact

SEQ ID NO: 73 - DNA, 47 bp, Homo Sapiens
  allele 24: 99-27851-30  polymorphic base A or G
tcaaatcacctccttgctgcggcrgacaagagacttcattcagttcc

SEQ ID NO: 74 - DNA, 47 bp, Homo Sapiens
  allele 24: 99-38884-129  polymorphic base A or G
aatacagcagattccagaacctgragcatgaagatgagtatctggaa

SEQ ID NO: 75 - DNA, 47 bp, Homo Sapiens
  allele 24: 99-38897-96  polymorphic base A or T
ggactaaaggtaaaataaactgtwgtcttagaaaaaggacaattagc

SEQ ID NO: 76 - DNA, 47 bp, Homo Sapiens
  allele 24: 99-36178-395  polymorphic base A or T
cctaaaacaacttcatgtagtaawaggcttttgtaaagtggtcaact

SEQ ID NO: 77 - DNA, 47 bp, Homo Sapiens
  allele 24: 99-36201-377  polymorphic base C or T
tctcctagtaaagcccatctcacyttgttatgacaaaatgatttatg

SEQ ID NO: 78 - DNA, 47 bp, Homo Sapiens
  allele 24: 99-32207-353  polymorphic base A or G
cacacattacctaataaatgctcraatgcataatattaatacatatt

SEQ ID NO: 79 - DNA, 47 bp, Homo Sapiens
  allele 24: 99-55707-362  polymorphic base C or T
tgnagcccacaactgagtctctgytttcttcctataaaggagctttc

SEQ ID NO: 80 - DNA, 47 bp, Homo Sapiens
  allele 24: 99-48925-395  polymorphic base A or G
gcaacaaaaatgtatctgctgcartggtggggatacgagagtatgat

SEQ ID NO: 81 - DNA, 47 bp, Homo Sapiens
  allele 24: 99-55725-246  polymorphic base C or T
gagataagttgtaacacagtaagygtactggaaagtgtgcttggaca

SEQ ID NO: 82 - DNA, 47 bp, Homo Sapiens
  allele 24: 99-28082-267  polymorphic base C or T
catttggattgaagtcttagctcyaatgcctactggtgatttgaatt

SEQ ID NO: 83 - DNA, 47 bp, Homo Sapiens
  allele 24: 99-28083-222  polymorphic base A or G
tgatagtaatagcattctgatttrtaaccataataatgaaatggtca

SEQ ID NO: 84 - DNA, 47 bp, Homo Sapiens
  allele 24: 99-27498-237  polymorphic base C or T
actttgagaaataatgagctaagyaactgacttaaatgaaaaagaaa

SEQ ID NO: 85 - DNA, 47 bp, Homo Sapiens
  allele 24: 99-27503-68  polymorphic base G or C
ggcccaacagccaatgggcggtastgcagcctcgcggtccctcacca

SEQ ID NO: 86 - DNA, 47 bp, Homo Sapiens
  allele 24: 99-26511-185  polymorphic base A or G
ttgttcaagtacccactggaccartggttttgcaggaaatgtgaatc

SEQ ID NO: 87 - DNA, 47 bp, Homo Sapiens
  allele 24: 99-26989-152  polymorphic base C or T
ttttatcaatgtctacttaattgygaccacctgatttaaaaattctg

SEQ ID NO: 88 - DNA, 47 bp, Homo Sapiens
  allele 24: 99-32158-71  polymorphic base C or T
aaggtcacagcactcttcttcatygctcctatcaaagctaccatttc

SEQ ID NO: 89 - DNA, 47 bp, Homo Sapiens
  allele 24: 99-32162-317  polymorphic base A or G
aagggcagggactctgtagttgtrtttatcattgtatctctagtatt

SEQ ID NO: 90 - DNA, 47 bp, Homo Sapiens
  allele 24: 99-32166-332  polymorphic base G or T
tcctaaaagcaagaaattattttkttcatagaaggaataatataatt

SEQ ID NO: 91 - DNA, 47 bp, Homo Sapiens
  allele 24: 99-32169-219  polymorphic base G or C
ctccattctcctcatctcagacasaacagccctttagagcaaatttg

SEQ ID NO: 92 - DNA, 47 bp, Homo Sapiens
  allele 24: 99-57305-320  polymorphic base A or G
tattatagcctcatttgatttttrttggcaatgtggtcattctaata

SEQ ID NO: 93 - DNA, 47 bp, Homo Sapiens
  allele 24: 99-57308-151  polymorphic base A or G
atggcttctccatttttcagttcragctttctgatttcacacacatg

SEQ ID NO: 94 - DNA, 47 bp, Homo Sapiens
  allele 24: 99-26477-419  polymorphic base C or T
aattcagtgtttatgtttttttayagaattatcaattgttttgaatc

SEQ ID NO: 95 - DNA, 47 bp, Homo Sapiens
  allele 24: 99-26479-79  polymorphic base C or T
caatgggtttgactcaattaaagyttcttttattcgcataagaattt

SEQ ID NO: 96 - DNA, 47 bp, Homo Sapiens
  allele 24: 99-26480-284  polymorphic base C or T
tgaaaaatggagcactgtaccttytggctacacctacctgtactggg

SEQ ID NO: 97 - DNA, 47 bp, Homo Sapiens
  allele 24: 99-26503-298  polymorphic base A or G
tgtctctgtcttatatcagttccrtttcacacttctgcctctgagtt

SEQ ID NO: 98 - DNA, 47 bp, Homo Sapiens
  allele 24: 99-27042-125  polymorphic base A or C
tatattctgcacatttagccagcmtctttggagtcctcattttggga

SEQ ID NO: 99 - DNA, 47 bp, Homo Sapiens
  allele 24: 99-27050-269  polymorphic base A or T
gccttctgtaggcgaaactgtggwttaccaagtgttattaactaaat

SEQ ID NO: 100 - DNA, 47 bp, Homo Sapiens
  allele 24: 99-27583-97  polymorphic base C or T
tgctccattccgaagagctgaaaytccgcgtgatggattatctttgt

SEQ ID NO: 101 - DNA, 47 bp, Homo Sapiens
  allele 24: 99-55097-137  polymorphic base C or T
tcccttgggtctcagcctcagtgygtctctgagcagggtggagtttc

SEQ ID NO: 102 - DNA, 47 bp, Homo Sapiens
  allele 24: 99-41727-169  polymorphic base A or G
gttataaaagtagtcaatgaactraaatctgaagtgcctagaagagg

SEQ ID NO: 103 - DNA, 47 bp, Homo Sapiens
  allele 24: 99-44259-417  polymorphic base A or G
cacttccttcaatggaaatggccrtttctgagtggtgacaacactgt

SEQ ID NO: 104 - DNA, 47 bp, Homo Sapiens
  allele 24: 99-44260-155  polymorphic base C or T
aagcatggactcctgcagtcataygcattttctctgtgttagcatga

SEQ ID NO: 105 - DNA, 47 bp, Homo Sapiens
  allele 24: 99-41009-244  polymorphic base C or T
atggaagggggaacagagaaacayatagaaatagccaaggtctgtgt

SEQ ID NO: 106 - DNA, 47 bp, Homo Sapiens
  allele 24: 99-41010-102  polymorphic base G or C
tctctctttctctctttctctctstttctatctatgtcacataaaag

SEQ ID NO: 107 - DNA, 47 bp, Homo Sapiens
  allele 24: 99-41828-310  polymorphic base C or T
attgagatggtactatttaaacaygtaacaaacactttaaagacaaa

SEQ ID NO: 108 - DNA, 47 bp, Homo Sapiens
  allele 24: 99-45821-385  polymorphic base A or G
aantttacatgggaaatacagaarcagattcaaggaagtaaaagaca

SEQ ID NO: 109 - DNA, 47 bp, Homo Sapiens
  allele 24: 99-26385-78  polymorphic base C or T
cgtattcatccttgtccttcagayaggtaagtacaactggaaaaatc

SEQ ID NO: 110 - DNA, 47 bp, Homo Sapiens
  allele 24: 99-48212-159  polymorphic base A or G
aaaggaggtaaaagtaggcaaggraagtttggaggacaaagttattt

SEQ ID NO: 111 - DNA, 47 bp, Homo Sapiens
  allele 24: 99-36203-346  polymorphic base A or G
aaataccggtaactagaagaaatrcctagaatttgatcaccaacaat

SEQ ID NO: 112 - DNA, 47 bp, Homo Sapiens
  allele 24: 99-441-155  polymorphic base C or T
ttttttcaaactcaattaaggaayttatttttaaaccacagctaaca

SEQ ID NO: 113 - DNA, 47 bp, Homo Sapiens
  allele 24: 99-444-226  polymorphic base C or T
tgttctacatcacaagtagggtaygggtgtgttacctgtggttcctc

SEQ ID NO: 114 - DNA, 47 bp, Homo Sapiens
  allele 24: 99-55706-54  polymorphic base C or T
gccatcctcaaaaagcttaaaatycttgttccaatcattgttccaat

SEQ ID NO: 115 - DNA, 47 bp, Homo Sapiens
  allele 24: 99-55708-159  polymorphic base A or T
gcatctcatgtgcatttcctgttwattccctgtacatctcatgttca

SEQ ID NO: 116 - DNA, 47 bp, Homo Sapiens
  allele 24: 99-55709-240  polymorphic base A or C
tggggttgacagccctggcattgmagacacactcactagctctgtca

SEQ ID NO: 117 - DNA, 47 bp, Homo Sapiens
  allele 24: 99-36422-176  polymorphic base A or T
ttaaaaccctaaggcagttttttwaaaaataccattgtttttaatat

SEQ ID NO: 118 - DNA, 47 bp, Homo Sapiens
  allele 24: 99-36426-50  polymorphic base A or T
ccacctgccaacgttttctgaccwagttctaccccagctccaccccc

SEQ ID NO: 119 - DNA, 47 bp, Homo Sapiens
  allele 24: 99-54269-455  polymorphic base A or G
caaaggctaaggcagaactgccarttgctagagggtttgcttttttc

SEQ ID NO: 120 - DNA, 47 bp, Homo Sapiens
  allele 24: 99-54270-270  polymorphic base G or T
aactctcacttgctttcctggcakgtccagatatggatttggaagag

SEQ ID NO: 121 - DNA, 47 bp, Homo Sapiens
  allele 24: 99-54272-97  polymorphic base G or C
gcacttttctgcaatattttatcsgatcagcatagattgcatgcctc

SEQ ID NO: 122 - DNA, 47 bp, Homo Sapiens
  allele 24: 99-54276-127  polymorphic base C or T
tactcaaagcataatcttaattgyttatgatttagataaataaaatt

SEQ ID NO: 123 - DNA, 47 bp, Homo Sapiens
  allele 24: 99-54277-41  polymorphic base C or T
tggaaaatgctaatttggctccgyatgcagcccattgggcaacatct

SEQ ID NO: 124 - DNA, 47 bp, Homo Sapiens
  allele 24: 99-49542-187  polymorphic base G or C
ggtggagtcagagccttgtttttstgaatcttttaagagtcagcaat

SEQ ID NO: 125 - DNA, 47 bp, Homo Sapiens
  allele 24: 99-49544-423  polymorphic base G or C
gaaaaagtttaagaagaaataatstcttcatcaggatataaggaaaa

SEQ ID NO: 126 - DNA, 47 bp, Homo Sapiens
  allele 24: 99-49545-396  polymorphic base G or C
ctgaatttttattgcattttgttsagttatataatgtaaaataaagt

SEQ ID NO: 127 - DNA, 47 bp, Homo Sapiens
  allele 24: 99-49547-344  polymorphic base A or G
tttatgagacttagagttactttrtaagataatctgtgattaaatga

SEQ ID NO: 128 - DNA, 47 bp, Homo Sapiens
  allele 24: 99-457-400  polymorphic base A or G
tagtgctgcagtcccaaagacacrgtctggccagggtgtatgtgggg

SEQ ID NO: 129 - DNA, 47 bp, Homo Sapiens
  allele 24: 99-463-90  polymorphic base C or T
acttgggtatgagattgttaaaayagtttgactaaatcaaacagctc

SEQ ID NO: 130 - DNA, 47 bp, Homo Sapiens
  allele 24: 99-32097-347  polymorphic base A or G
ggggtgtgaattgggcatttgatratagtgacttttctgttttctgg

SEQ ID NO: 131 - DNA, 47 bp, Homo Sapiens
  allele 24: 99-32110-64  polymorphic base A or G
tgccaagacatccccacatggggrctggcaataaagagatgtactta

SEQ ID NO: 132 - DNA, 47 bp, Homo Sapiens
  allele 24: 99-32117-226  polymorphic base C or T
agaatttcatcttaatttttttgyagtgcttatgatttgagtgcatt

SEQ ID NO: 133 - DNA, 47 bp, Homo Sapiens
  allele 24: 99-26972-44  polymorphic base A or G
tggaaggctataaaagcctatacrggtaatgcagtatccttaaagca

SEQ ID NO: 134 - DNA, 47 bp, Homo Sapiens
  allele 24: 99-26973-239  polymorphic base G or C
gagatcagcggaagaggacgttastaacaaaggagacttagaaggaa

SEQ ID NO: 135 - DNA, 47 bp, Homo Sapiens
  allele 24: 99-26974-450  polymorphic base C or T
gttgaattacagggacacgcaacygataattataataaacttctccc

SEQ ID NO: 136 - DNA, 47 bp, Homo Sapiens
  allele 24: 99-26975-40  polymorphic base C or T
gtggtctctgccaggagaaagaaytccccctgtaggaagccaagttc

SEQ ID NO: 137 - DNA, 47 bp, Homo Sapiens
  allele 24: 99-26978-122  polymorphic base A or T
ataaatttgagtgtgtttattccwtaaaatgaggtctacataactgt

SEQ ID NO: 138 - DNA, 47 bp, Homo Sapiens
  allele 24: 99-26981-171  polymorphic base C or T
aatgttttaaaagataaaagttgytggctattaccttgaaaatctaa

SEQ ID NO: 139 - DNA, 47 bp, Homo Sapiens
  allele 24: 99-26983-292  polymorphic base C or T
tccaccatatatcatttataacayatttaattttgcttaagatgacg

SEQ ID NO: 140 - DNA, 47 bp, Homo Sapiens
  allele 24: 99-26987-196  polymorphic base A or C
aatttgtctgagccacagttagamagtggcaaaagagacttgaacca

SEQ ID NO: 141 - DNA, 47 bp, Homo Sapiens
  allele 24: 99-32153-348  polymorphic base C or T
agcttggattttattaagagcaaygcggagggacacaggagagagga

SEQ ID NO: 142 - DNA, 47 bp, Homo Sapiens
  allele 24: 99-32156-127  polymorphic base C or T
tggcctctaacgttcctaagacaygtaaggtgttacttctaggccag

SEQ ID NO: 143 - DNA, 47 bp, Homo Sapiens
  allele 24: 99-57302-162  polymorphic base G or T
acaatatttttcaaattgtgtgckgcagttcagcattttcacccagt

SEQ ID NO: 144 - DNA, 47 bp, Homo Sapiens
  allele 24: 99-26349-339  polymorphic base C or T
cagcttctaggtttccgtgacctyagttagcaaaggcaggaaaagat

SEQ ID NO: 145 - DNA, 47 bp, Homo Sapiens
  allele 24: 99-26352-71  polymorphic base A or G
ccgaaagccattaaggaaaatgartcactttctggatgcattgaaca

SEQ ID NO: 146 - DNA, 47 bp, Homo Sapiens
  allele 24: 99-26370-60  polymorphic base G or C
aggtagaagtcagggccagagacstaggttaatcatttcttttctaa

SEQ ID NO: 147 - DNA, 47 bp, Homo Sapiens
  allele 24: 99-28332-445  polymorphic base A or C
taataatttttaaaagccagcatmtgtggattcccattttgaccttg

SEQ ID NO: 148 - DNA, 47 bp, Homo Sapiens
  allele 24: 99-28339-362  polymorphic base A or G
cattttgccattgacaggagcccrcagttgtaagcctgctgggtttc

SEQ ID NO: 149 - DNA, 47 bp, Homo Sapiens
  allele 24: 99-28342-137  polymorphic base A or G
ccaccacccttacccagtcttccrccttcccacacctgaactccaaa

SEQ ID NO: 150 - DNA, 47 bp, Homo Sapiens
  allele 24: 99-28343-487  polymorphic base A or G
gtctgaccctacttattttttgtrtaaagtgggaccgcactggtgat

SEQ ID NO: 151 - DNA, 47 bp, Homo Sapiens
  allele 24: 99-28352-101  polymorphic base A or C
taagggaaagtacccaaggaaggmtcaaactcttagatagggatgtc

SEQ ID NO: 152 - DNA, 47 bp, Homo Sapiens
  allele 24: 99-28371-233  polymorphic base G or C
acgtaattccttcaacaaatattsagctggacactgtctaattcatg

SEQ ID NO: 153 - DNA, 47 bp, Homo Sapiens
  allele 24: 99-27041-274  polymorphic base A or G
ttgttgatttaatcaacctagccrgctgtcatgtgggattagaataa

SEQ ID NO: 154 - DNA, 47 bp, Homo Sapiens
  allele 24: 99-29191-241  polymorphic base A or G
tgaagacataaaacataaaatcartacacagtggatcagaataaata

SEQ ID NO: 155 - DNA, 47 bp, Homo Sapiens
  allele 24: 99-27074-429  polymorphic base A or G
acagggcaaccaagaagaattgtrctcagagcaccgtatgatccatc

SEQ ID NO: 156 - DNA, 47 bp, Homo Sapiens
  allele 24: 99-27076-313  polymorphic base C or T
catgccaggctaacaaaaagaaaygcattacagttcttgaaactctg

SEQ ID NO: 157 - DNA, 47 bp, Homo Sapiens
  allele 24: 99-27079-302  polymorphic base A or T
ggatggtttgttgcaatttttttwaattaccaaacaattttttaagg

SEQ ID NO: 158 - DNA, 47 bp, Homo Sapiens
  allele 24: 99-27082-303  polymorphic base A or G
tagcaaaatattcaaaagaattartaaatagaggcaattcatttgac

SEQ ID NO: 159 - DNA, 47 bp, Homo Sapiens
  allele 24: 99-55890-43  polymorphic base A or T
ctgagtgagatgaggcggcttttwttctgtgtcgttgactttcttga

SEQ ID NO: 160 - DNA, 47 bp, Homo Sapiens
  allele 24: 99-55901-112  polymorphic base G or C
aaaagtgaagaaaaaaggttcagsgcttctgctcctaagatgaaaaa

SEQ ID NO: 161 - DNA, 47 bp, Homo Sapiens
  allele 24: 99-55903-324  polymorphic base A or T
tcaagaaagtcaacgacacagaawaaaagccgcctcatctcactcag

SEQ ID NO: 162 - DNA, 47 bp, Homo Sapiens
  allele 24: 9-42-169  polymorphic base A or G
ggtggtgtgggtcccagagggttrgcacgctaaagcagcaaacacac

SEQ ID NO: 163 - DNA, 47 bp, Homo Sapiens
  allele 24: 99-41725-266  polymorphic base A or G
ctaaccactcccttgagattagcrctaaatctttccaccagcctaca

SEQ ID NO: 164 - DNA, 47 bp, Homo Sapiens
  allele 24: 99-45440-313  polymorphic base C or T
tatacctggctatgtatattctayggaaataattcatcctaagtgcg

SEQ ID NO: 165 - DNA, 47 bp, Homo Sapiens
  allele 24: 99-438-82  polymorphic base C or T
acagtcatagggtttttgtttagygtgagtcctttcatgtcttccaa

SEQ ID NO: 166 - DNA, 47 bp, Homo Sapiens
  allele 24: 99-36428-363  polymorphic base A or G
ctcctagatgactaaacctgctcrgctgaaatcaaatgctctcatgc

SEQ ID NO: 167 - DNA, 47 bp, Homo Sapiens
  allele 24: 99-36433-318  polymorphic base A or G
agccaataagaacagtgagtgacrtaccctggcacatagaaatatca

SEQ ID NO: 168 - DNA, 47 bp, Homo Sapiens
  allele 24: 99-26968-300  polymorphic base A or G
gagaataagaccctcccacttgcrtttgttaattgactttgatgagc

SEQ ID NO: 169 - DNA, 47 bp, Homo Sapiens
  allele 24: 99-26345-57  polymorphic base A or C
tcaagcaagtaagaacagaaatamcagcatttggcttttgagttaat

SEQ ID NO: 170 - DNA, 47 bp, Homo Sapiens
  allele 24: 99-27070-379  polymorphic base C or T
gaataccattttccaaaagaagayaccagatcatctctgtatggaat

SEQ ID NO: 171 - DNA, 47 bp, Homo Sapiens
  allele 24: 99-27581-206  polymorphic base G or T
gcctcttccacaagacagcttctktggggtggagacaggagtaggat

SEQ ID NO: 172 - DNA, 18 bp, Homo Sapiens
  primer_bind 1..18: upstream amplification primer 99-55095 for SEQ 1,
gaatctcatctctgccac

SEQ ID NO: 173 - DNA, 18 bp, Homo Sapiens
  primer_bind 1..18: upstream amplification primer 99-55102 for SEQ 2,
tgctactgcctcttattc

SEQ ID NO: 174 - DNA, 18 bp, Homo Sapiens
  primer_bind 1..18: upstream amplification primer 99-26407 for SEQ 3,
gcaaaatccaaagcactc

SEQ ID NO: 175 - DNA, 21 bp, Homo Sapiens
  primer_bind 1..21: upstream amplification primer 99-26412 for SEQ 4,
gcatatcagatacatacatac

SEQ ID NO: 176 - DNA, 18 bp, Homo Sapiens
  primer_bind 1..18: upstream amplification primer 99-26413 for SEQ 5,
caatgttggcaatatggg

SEQ ID NO: 177 - DNA, 21 bp, Homo Sapiens
  primer_bind 1..21: upstream amplification primer 99-26424 for SEQ 6,
cttacaatttcagaaagtacc

SEQ ID NO: 178 - DNA, 20 bp, Homo Sapiens
  primer_bind 1..20: upstream amplification primer 99-44320 for SEQ 7,
ccttattgtccagatatatg

SEQ ID NO: 179 - DNA, 19 bp, Homo Sapiens
  primer_bind 1..19: upstream amplification primer 99-41889 for SEQ 8,
cttttttgttaggctactg

SEQ ID NO: 180 - DNA, 19 bp, Homo Sapiens
  primer_bind 1..19: upstream amplification primer 99-45453 for SEQ 9,
tcaaagcattagtttacgg

SEQ ID NO: 181 - DNA, 20 bp, Homo Sapiens
  primer_bind 1..20: upstream amplification primer 99-43427 for SEQ 10,
ccatttgtgtcctcaaaaag

SEQ ID NO: 182 - DNA, 19 bp, Homo Sapiens
  primer_bind 1..19: upstream amplification primer 99-55124 for SEQ 11,
gcatttttagacgtaaggg

SEQ ID NO: 183 - DNA, 18 bp, Homo Sapiens
  primer_bind 1..18: upstream amplification primer 99-46316 for SEQ 12,
atcccagtacaccatacc

SEQ ID NO: 184 - DNA, 19 bp, Homo Sapiens
  primer_bind 1..19: upstream amplification primer 99-46325 for SEQ 13,
actggggcacactgattac

SEQ ID NO: 185 - DNA, 18 bp, Homo Sapiens
  primer_bind 1..18: upstream amplification primer 99-46332 for SEQ 14,
gccattgataaacctgtc

SEQ ID NO: 186 - DNA, 21 bp, Homo Sapiens
  primer_bind 1..21: upstream amplification primer 99-47064 for SEQ 15,
ctaccaaatctcaaatacttc

SEQ ID NO: 187 - DNA, 18 bp, Homo Sapiens
  primer_bind 1..18: upstream amplification primer 99-47065 for SEQ 16,
cgatgcttttagaggaac

SEQ ID NO: 188 - DNA, 19 bp, Homo Sapiens
  primer_bind 1..19: upstream amplification primer 99-47066 for SEQ 17,
gctctccaacaaagttacc

SEQ ID NO: 189 - DNA, 20 bp, Homo Sapiens
  primer_bind 1..20: upstream amplification primer 99-38883 for SEQ 18,
catacatcccaaacattatc

SEQ ID NO: 190 - DNA, 18 bp, Homo Sapiens
  primer_bind 1..18: upstream amplification primer 99-38888 for SEQ 19,
ctccagagatgaatttcc

SEQ ID NO: 191 - DNA, 18 bp, Homo Sapiens
  primer_bind 1..18: upstream amplification primer 99-38889 for SEQ 20,
ggctgagatggtgatatg

SEQ ID NO: 192 - DNA, 18 bp, Homo Sapiens
  primer_bind 1..18: upstream amplification primer 99-26458 for SEQ 21,
gcctctttacctattcag

SEQ ID NO: 193 - DNA, 21 bp, Homo Sapiens
  primer_bind 1..21: upstream amplification primer 99-47321 for SEQ 22,
taaggaatagagagtgtgaag

SEQ ID NO: 194 - DNA, 18 bp, Homo Sapiens
  primer_bind 1..18: upstream amplification primer 99-36198 for SEQ 23,
aggcaatggaatgaggag

SEQ ID NO: 195 - DNA, 18 bp, Homo Sapiens
  primer_bind 1..18: upstream amplification primer 99-36200 for SEQ 24,
acgttgcagtggagtaag

SEQ ID NO: 196 - DNA, 19 bp, Homo Sapiens
  primer_bind 1..19: upstream amplification primer 99-33135 for SEQ 25,
agtcttgaaaataccacag

SEQ ID NO: 197 - DNA, 20 bp, Homo Sapiens
  primer_bind 1..20: upstream amplification primer 99-34451 for SEQ 26,
gtagaaattggaatgttgag

SEQ ID NO: 198 - DNA, 20 bp, Homo Sapiens
  primer_bind 1..20: upstream amplification primer 99-34452 for SEQ 27,
ctatttcatgtaaatggctg

SEQ ID NO: 199 - DNA, 19 bp, Homo Sapiens
  primer_bind 1..19: upstream amplification primer 99-48928 for SEQ 28,
gagattgccatagttccag

SEQ ID NO: 200 - DNA, 20 bp, Homo Sapiens
  primer_bind 1..20: upstream amplification primer 99-55720 for SEQ 29,
gctataattactgtgagatg

SEQ ID NO: 201 - DNA, 18 bp, Homo Sapiens
  primer_bind 1..18: upstream amplification primer 99-55721 for SEQ 30,
cagggattctctcatcac

SEQ ID NO: 202 - DNA, 19 bp, Homo Sapiens
  primer_bind 1..19: upstream amplification primer 99-501 for SEQ 31,
cctgcaagatagatcaatg

SEQ ID NO: 203 - DNA, 19 bp, Homo Sapiens
  primer_bind 1..19: upstream amplification primer 99-34556 for SEQ 32,
tgttcctcacagcattttc

SEQ ID NO: 204 - DNA, 19 bp, Homo Sapiens
  primer_bind 1..19: upstream amplification primer 99-34561 for SEQ 33,
aagacataatctaccctcc

SEQ ID NO: 205 - DNA, 19 bp, Homo Sapiens
  primer_bind 1..19: upstream amplification primer 99-28061 for SEQ 34,
ttttaatcaaaccgccaac

SEQ ID NO: 206 - DNA, 19 bp, Homo Sapiens
  primer_bind 1..19: upstream amplification primer 99-28062 for SEQ 35,
atgactttttgaactggac

SEQ ID NO: 207 - DNA, 20 bp, Homo Sapiens
  primer_bind 1..20: upstream amplification primer 99-28063 for SEQ 36,
ctgagtaccattttcttctg

SEQ ID NO: 208 - DNA, 18 bp, Homo Sapiens
  primer_bind 1..18: upstream amplification primer 99-28067 for SEQ 37,
tactgcatgaagagaacc

SEQ ID NO: 209 - DNA, 18 bp, Homo Sapiens
  primer_bind 1..18: upstream amplification primer 99-28080 for SEQ 38,
acagcttagcaacacagg

SEQ ID NO: 210 - DNA, 19 bp, Homo Sapiens
  primer_bind 1..19: upstream amplification primer 99-459 for SEQ 39,
acatcatcgcagcctcttg

SEQ ID NO: 211 - DNA, 20 bp, Homo Sapiens
  primer_bind 1..20: upstream amplification primer 99-32104 for SEQ 40,
gtggcaattccgtgctgatg

SEQ ID NO: 212 - DNA, 19 bp, Homo Sapiens
  primer_bind 1..19: upstream amplification primer 99-26970 for SEQ 41,
taagtcaattcacctatgg

SEQ ID NO: 213 - DNA, 19 bp, Homo Sapiens
  primer_bind 1..19: upstream amplification primer 99-27502 for SEQ 42,
ccttcacagatgataattc

SEQ ID NO: 214 - DNA, 19 bp, Homo Sapiens
  primer_bind 1..19: upstream amplification primer 99-26994 for SEQ 43,
atatagttctccttactcc

SEQ ID NO: 215 - DNA, 19 bp, Homo Sapiens
  primer_bind 1..19: upstream amplification primer 99-26995 for SEQ 44,
atcacactttcttcttacc

SEQ ID NO: 216 - DNA, 19 bp, Homo Sapiens
  primer_bind 1..19: upstream amplification primer 99-26997 for SEQ 45,
gagaagaaaagacccatac

SEQ ID NO: 217 - DNA, 19 bp, Homo Sapiens
  primer_bind 1..19: upstream amplification primer 99-27001 for SEQ 46,
atgaagggaaaggaaatac

SEQ ID NO: 218 - DNA, 20 bp, Homo Sapiens
  primer_bind 1..20: upstream amplification primer 99-32161 for SEQ 47,
gcttgcccttctttttagag

SEQ ID NO: 219 - DNA, 18 bp, Homo Sapiens
  primer_bind 1..18: upstream amplification primer 99-32165 for SEQ 48,
cacacttccctggaatac

SEQ ID NO: 220 - DNA, 21 bp, Homo Sapiens
  primer_bind 1..21: upstream amplification primer 99-32168 for SEQ 49,
ggcacaaaagcaaaaattaag

SEQ ID NO: 221 - DNA, 19 bp, Homo Sapiens
  primer_bind 1..19: upstream amplification primer 99-32172 for SEQ 50,
ctttgagtgtctctctagg

SEQ ID NO: 222 - DNA, 19 bp, Homo Sapiens
  primer_bind 1..19: upstream amplification primer 99-32174 for SEQ 51,
gataacatttctcagcagc

SEQ ID NO: 223 - DNA, 18 bp, Homo Sapiens
  primer_bind 1..18: upstream amplification primer 99-57310 for SEQ 52,
ataagaacagagaagggg

SEQ ID NO: 224 - DNA, 18 bp, Homo Sapiens
  primer_bind 1..18: upstream amplification primer 99-57311 for SEQ 53,
tttgagccacattgccac

SEQ ID NO: 225 - DNA, 19 bp, Homo Sapiens
  primer_bind 1..19: upstream amplification primer 99-57314 for SEQ 54,
tctagttaggaggttatac

SEQ ID NO: 226 - DNA, 18 bp, Homo Sapiens
  primer_bind 1..18: upstream amplification primer 99-26359 for SEQ 55,
ttaggggaaagggggtag

SEQ ID NO: 227 - DNA, 20 bp, Homo Sapiens
  primer_bind 1..20: upstream amplification primer 99-27232 for SEQ 56,
gctagaagtaataatgtgac

SEQ ID NO: 228 - DNA, 18 bp, Homo Sapiens
  primer_bind 1..18: upstream amplification primer 99-27595 for SEQ 57,
aattaaggcagacccaag

SEQ ID NO: 229 - DNA, 18 bp, Homo Sapiens
  primer_bind 1..18: upstream amplification primer 99-26492 for SEQ 58,
cgtagagttttcctaacc

SEQ ID NO: 230 - DNA, 19 bp, Homo Sapiens
  primer_bind 1..19: upstream amplification primer 99-26501 for SEQ 59,
cttatagtgagaaaaaccc

SEQ ID NO: 231 - DNA, 21 bp, Homo Sapiens
  primer_bind 1..21: upstream amplification primer 99-28350 for SEQ 60,
caattctaaagttcatgttcc

SEQ ID NO: 232 - DNA, 19 bp, Homo Sapiens
  primer_bind 1..19: upstream amplification primer 99-28355 for SEQ 61,
gataacagcttagcaacac

SEQ ID NO: 233 - DNA, 19 bp, Homo Sapiens
  primer_bind 1..19: upstream amplification primer 99-28360 for SEQ 62,
tcacaaagctacattcatc

SEQ ID NO: 234 - DNA, 18 bp, Homo Sapiens
  primer_bind 1..18: upstream amplification primer 99-28363 for SEQ 63,
aagtgatcccaggatttg

SEQ ID NO: 235 - DNA, 21 bp, Homo Sapiens
  primer_bind 1..21: upstream amplification primer 99-28364 for SEQ 64,
gtcaaaaatattgtgcaagtc

SEQ ID NO: 236 - DNA, 18 bp, Homo Sapiens
  primer_bind 1..18: upstream amplification primer 99-27049 for SEQ 65,
tctggtttcctccctttg

SEQ ID NO: 237 - DNA, 19 bp, Homo Sapiens
  primer_bind 1..19: upstream amplification primer 99-27054 for SEQ 66,
gcagaaatgcaaagttacc

SEQ ID NO: 238 - DNA, 19 bp, Homo Sapiens
  primer_bind 1..19: upstream amplification primer 99-27056 for SEQ 67,
ttccaaatcttagcactac

SEQ ID NO: 239 - DNA, 18 bp, Homo Sapiens
  primer_bind 1..18: upstream amplification primer 99-27586 for SEQ 68,
cgtcattctagtgatctg

SEQ ID NO: 240 - DNA, 19 bp, Homo Sapiens
  primer_bind 1..19: upstream amplification primer 99-29193 for SEQ 69,
gtaaggttgaaatggttag

SEQ ID NO: 241 - DNA, 19 bp, Homo Sapiens
  primer_bind 1..19: upstream amplification primer 99-56867 for SEQ 70,
aggtgtcttgtcgtcttac

SEQ ID NO: 242 - DNA, 18 bp, Homo Sapiens
  primer_bind 1..18: upstream amplification primer 99-26399 for SEQ 71,
ggtctgtggattttttgg

SEQ ID NO: 243 - DNA, 18 bp, Homo Sapiens
  primer_bind 1..18: upstream amplification primer 99-41887 for SEQ 72,
ttcctaatgccccaaaac

SEQ ID NO: 244 - DNA, 18 bp, Homo Sapiens
  primer_bind 1..18: upstream amplification primer 99-27851 for SEQ 73,
gtggtttcaaatcacctc

SEQ ID NO: 245 - DNA, 18 bp, Homo Sapiens
  primer_bind 1..18: upstream amplification primer 99-38884 for SEQ 74,
ttcccaaaccactcaaag

SEQ ID NO: 246 - DNA, 18 bp, Homo Sapiens
  primer_bind 1..18: upstream amplification primer 99-38897 for SEQ 75,
agacttcagagacacagg

SEQ ID NO: 247 - DNA, 18 bp, Homo Sapiens
  primer_bind 1..18: upstream amplification primer 99-36178 for SEQ 76,
ccaactaggttagaagtc

SEQ ID NO: 248 - DNA, 19 bp, Homo Sapiens
  primer_bind 1..19: upstream amplification primer 99-36201 for SEQ 77,
gctctggaggcaaaaaaac

SEQ ID NO: 249 - DNA, 20 bp, Homo Sapiens
  primer_bind 1..20: upstream amplification primer 99-32207 for SEQ 78,
ctgtcttgcttattttcttc

SEQ ID NO: 250 - DNA, 21 bp, Homo Sapiens
  primer_bind 1..21: upstream amplification primer 99-55707 for SEQ 79,
gatgagaggtattacaggaag

SEQ ID NO: 251 - DNA, 20 bp, Homo Sapiens
  primer_bind 1..20: upstream amplification primer 99-48925 for SEQ 80,
gcaagaatctccaaataccc

SEQ ID NO: 252 - DNA, 21 bp, Homo Sapiens
  primer_bind 1..21: upstream amplification primer 99-55725 for SEQ 81,
gatagacatgagcctatatcc

SEQ ID NO: 253 - DNA, 18 bp, Homo Sapiens
  primer_bind 1..18: upstream amplification primer 99-28082 for SEQ 82,
tctcttttgtgtctctgg

SEQ ID NO: 254 - DNA, 19 bp, Homo Sapiens
  primer_bind 1..19: upstream amplification primer 99-28083 for SEQ 83,
gtttgaggaaaataatggg

SEQ ID NO: 255 - DNA, 19 bp, Homo Sapiens
  primer_bind 1..19: upstream amplification primer 99-27498 for SEQ 84,
cagagacagaaatgacaac

SEQ ID NO: 256 - DNA, 19 bp, Homo Sapiens
  primer_bind 1..19: upstream amplification primer 99-27503 for SEQ 85,
gtgatggagagaacttatg

SEQ ID NO: 257 - DNA, 20 bp, Homo Sapiens
  primer_bind 1..20: upstream amplification primer 99-26511 for SEQ 86,
gataaataggatgtgaactg

SEQ ID NO: 258 - DNA, 18 bp, Homo Sapiens
  primer_bind 1..18: upstream amplification primer 99-26989 for SEQ 87,
aggcaagccatgtatttc

SEQ ID NO: 259 - DNA, 21 bp, Homo Sapiens
  primer_bind 1..21: upstream amplification primer 99-32158 for SEQ 88,
gatctgatctaagtcccaatg

SEQ ID NO: 260 - DNA, 18 bp, Homo Sapiens
  primer_bind 1..18: upstream amplification primer 99-32162 for SEQ 89,
cttccccacaccttcatc

SEQ ID NO: 261 - DNA, 18 bp, Homo Sapiens
  primer_bind 1..18: upstream amplification primer 99-32166 for SEQ 90,
tagcatcatcttcacagg

SEQ ID NO: 262 - DNA, 18 bp, Homo Sapiens
  primer_bind 1..18: upstream amplification primer 99-32169 for SEQ 91,
gtgtcaacatcatgtcgg

SEQ ID NO: 263 - DNA, 19 bp, Homo Sapiens
  primer_bind 1..19: upstream amplification primer 99-57305 for SEQ 92,
agcatacataagaatgagg

SEQ ID NO: 264 - DNA, 18 bp, Homo Sapiens
  primer_bind 1..18: upstream amplification primer 99-57308 for SEQ 93,
atcaccacccaacatcag

SEQ ID NO: 265 - DNA, 18 bp, Homo Sapiens
  primer_bind 1..18: upstream amplification primer 99-26477 for SEQ 94,
aaattcctctgtagcacc

SEQ ID NO: 266 - DNA, 18 bp, Homo Sapiens
  primer_bind 1..18: upstream amplification primer 99-26479 for SEQ 95,
ggaactaccctctttctg

SEQ ID NO: 267 - DNA, 20 bp, Homo Sapiens
  primer_bind 1..20: upstream amplification primer 99-26480 for SEQ 96,
gattggatctgataattagc

SEQ ID NO: 268 - DNA, 19 bp, Homo Sapiens
  primer_bind 1..19: upstream amplification primer 99-26503 for SEQ 97,
tcaggcagaagacaaacag

SEQ ID NO: 269 - DNA, 20 bp, Homo Sapiens
  primer_bind 1..20: upstream amplification primer 99-27042 for SEQ 98,
tgtcacctctctgcttgctc

SEQ ID NO: 270 - DNA, 19 bp, Homo Sapiens
  primer_bind 1..19: upstream amplification primer 99-27050 for SEQ 99,
ggttattaggagtgtgatg

SEQ ID NO: 271 - DNA, 20 bp, Homo Sapiens
  primer_bind 1..20: upstream amplification primer 99-27583 for SEQ 100,
ctcagttgacagaaaattgg

SEQ ID NO: 272 - DNA, 19 bp, Homo Sapiens
  primer_bind 1..19: upstream amplification primer 99-55097 for SEQ 101,
tgctcagttcataaacagg

SEQ ID NO: 273 - DNA, 18 bp, Homo Sapiens
  primer_bind 1..18: upstream amplification primer 99-41727 for SEQ 102,
ctacttgctggttagttc

SEQ ID NO: 274 - DNA, 21 bp, Homo Sapiens
  primer_bind 1..21: upstream amplification primer 99-44259 for SEQ 103,
ctgcttttttatctgaaactg

SEQ ID NO: 275 - DNA, 19 bp, Homo Sapiens
  primer_bind 1..19: upstream amplification primer 99-44260 for SEQ 104,
ggttgtgcatgtatttgtg

SEQ ID NO: 276 - DNA, 19 bp, Homo Sapiens
  primer_bind 1..19: upstream amplification primer 99-41009 for SEQ 105,
tgcagacagaatgactccc

SEQ ID NO: 277 - DNA, 18 bp, Homo Sapiens
  primer_bind 1..18: upstream amplification primer 99-41010 for SEQ 106,
ataaatccttccacccac

SEQ ID NO: 278 - DNA, 19 bp, Homo Sapiens
  primer_bind 1..19: upstream amplification primer 99-41828 for SEQ 107,
actcagatttctgactttc

SEQ ID NO: 279 - DNA, 19 bp, Homo Sapiens
  primer_bind 1..19: upstream amplification primer 99-45821 for SEQ 108,
cagtgaaattagcaaaccc

SEQ ID NO: 280 - DNA, 18 bp, Homo Sapiens
  primer_bind 1..18: upstream amplification primer 99-26385 for SEQ 109,
cctcgggtgtatgttttg

SEQ ID NO: 281 - DNA, 19 bp, Homo Sapiens
  primer_bind 1..19: upstream amplification primer 99-48212 for SEQ 110,
ctgctttgaatataagacc

SEQ ID NO: 282 - DNA, 18 bp, Homo Sapiens
  primer_bind 1..18: upstream amplification primer 99-36203 for SEQ 111,
tgttatgttgagaggtgg

SEQ ID NO: 283 - DNA, 19 bp, Homo Sapiens
  primer_bind 1..19: upstream amplification primer 99-441 for SEQ 112,
aaacaagacaagcacaatc

SEQ ID NO: 284 - DNA, 18 bp, Homo Sapiens
  primer_bind 1..18: upstream amplification primer 99-444 for SEQ 113,
tgtatttccaacagcctc

SEQ ID NO: 285 - DNA, 18 bp, Homo Sapiens
  primer_bind 1..18: upstream amplification primer 99-55706 for SEQ 114,
tcgtttgttcatttgggg

SEQ ID NO: 286 - DNA, 18 bp, Homo Sapiens
  primer_bind 1..18: upstream amplification primer 99-55708 for SEQ 115,
ctctctccattctcaaac

SEQ ID NO: 287 - DNA, 18 bp, Homo Sapiens
  primer_bind 1..18: upstream amplification primer 99-55709 for SEQ 116,
aggtgataaaagaggagg

SEQ ID NO: 288 - DNA, 18 bp, Homo Sapiens
  primer_bind 1..18: upstream amplification primer 99-36422 for SEQ 117,
agatgactttactaggcg

SEQ ID NO: 289 - DNA, 18 bp, Homo Sapiens
  primer_bind 1..18: upstream amplification primer 99-36426 for SEQ 118,
caatcacctcatcaaacc

SEQ ID NO: 290 - DNA, 18 bp, Homo Sapiens
  primer_bind 1..18: upstream amplification primer 99-54269 for SEQ 119,
tcatctgtcctgtgttac

SEQ ID NO: 291 - DNA, 18 bp, Homo Sapiens
  primer_bind 1..18: upstream amplification primer 99-54270 for SEQ 120,
tacccatgtagggaagag

SEQ ID NO: 292 - DNA, 19 bp, Homo Sapiens
  primer_bind 1..19: upstream amplification primer 99-54272 for SEQ 121,
aagttgaccaattctcaag

SEQ ID NO: 293 - DNA, 19 bp, Homo Sapiens
  primer_bind 1..19: upstream amplification primer 99-54276 for SEQ 122,
gggaatataacatttcagc

SEQ ID NO: 294 - DNA, 19 bp, Homo Sapiens
  primer_bind 1..19: upstream amplification primer 99-54277 for SEQ 123,
ttttgctttgtctgagatg

SEQ ID NO: 295 - DNA, 19 bp, Homo Sapiens
  primer_bind 1..19: upstream amplification primer 99-49542 for SEQ 124,
gaggtaagagataataagg

SEQ ID NO: 296 - DNA, 18 bp, Homo Sapiens
  primer_bind 1..18: upstream amplification primer 99-49544 for SEQ 125,
aacaatgtgaactggagg

SEQ ID NO: 297 - DNA, 18 bp, Homo Sapiens
  primer_bind 1..18: upstream amplification primer 99-49545 for SEQ 126,
agttggtagatttggcac

SEQ ID NO: 298 - DNA, 21 bp, Homo Sapiens
  primer_bind 1..21: upstream amplification primer 99-49547 for SEQ 127,
gtaaattgctcttgttacttc

SEQ ID NO: 299 - DNA, 18 bp, Homo Sapiens
  primer_bind 1..18: upstream amplification primer 99-457 for SEQ 128,
ccctagattccttaaacc

SEQ ID NO: 300 - DNA, 20 bp, Homo Sapiens
  primer_bind 1..20: upstream amplification primer 99-463 for SEQ 129,
tccatgtgacacctgtagac

SEQ ID NO: 301 - DNA, 18 bp, Homo Sapiens
  primer_bind 1..18: upstream amplification primer 99-32097 for SEQ 130,
atctgagttagtggttgg

SEQ ID NO: 302 - DNA, 18 bp, Homo Sapiens
  primer_bind 1..18: upstream amplification primer 99-32110 for SEQ 131,
gctgagatttttctcctg

SEQ ID NO: 303 - DNA, 18 bp, Homo Sapiens
  primer_bind 1..18: upstream amplification primer 99-32117 for SEQ 132,
ccacatccttttggcttc

SEQ ID NO: 304 - DNA, 18 bp, Homo Sapiens
  primer_bind 1..18: upstream amplification primer 99-26972 for SEQ 133,
tatctctctactgctctg

SEQ ID NO: 305 - DNA, 18 bp, Homo Sapiens
  primer_bind 1..18: upstream amplification primer 99-26973 for SEQ 134,
ataccgccgcaatcaatg

SEQ ID NO: 306 - DNA, 20 bp, Homo Sapiens
  primer_bind 1..20: upstream amplification primer 99-26974 for SEQ 135,
agtctgtgtggttagagcgg

SEQ ID NO: 307 - DNA, 20 bp, Homo Sapiens
  primer_bind 1..20: upstream amplification primer 99-26975 for SEQ 136,
gaagatatagtaggaggtgg

SEQ ID NO: 308 - DNA, 20 bp, Homo Sapiens
  primer_bind 1..20: upstream amplification primer 99-26978 for SEQ 137,
ttaggtcattctggttattg

SEQ ID NO: 309 - DNA, 19 bp, Homo Sapiens
  primer_bind 1..19: upstream amplification primer 99-26981 for SEQ 138,
ccagaacacagcggttttg

SEQ ID NO: 310 - DNA, 20 bp, Homo Sapiens
  primer_bind 1..20: upstream amplification primer 99-26983 for SEQ 139,
gctaactcaccgaagaatac

SEQ ID NO: 311 - DNA, 21 bp, Homo Sapiens
  primer_bind 1..21: upstream amplification primer 99-26987 for SEQ 140,
attgtttacctactatgtacc

SEQ ID NO: 312 - DNA, 19 bp, Homo Sapiens
  primer_bind 1..19: upstream amplification primer 99-32153 for SEQ 141,
gacaaagagatcccattac

SEQ ID NO: 313 - DNA, 18 bp, Homo Sapiens
  primer_bind 1..18: upstream amplification primer 99-32156 for SEQ 142,
tcctgaaccaaacctaac

SEQ ID NO: 314 - DNA, 18 bp, Homo Sapiens
  primer_bind 1..18: upstream amplification primer 99-57302 for SEQ 143,
ctaacaccatcttagcac

SEQ ID NO: 315 - DNA, 19 bp, Homo Sapiens
  primer_bind 1..19: upstream amplification primer 99-26349 for SEQ 144,
gtcaaagccgtaaatcaag

SEQ ID NO: 316 - DNA, 20 bp, Homo Sapiens
  primer_bind 1..20: upstream amplification primer 99-26352 for SEQ 145,
atggtgtgctggtgggattg

SEQ ID NO: 317 - DNA, 18 bp, Homo Sapiens
  primer_bind 1..18: upstream amplification primer 99-26370 for SEQ 146,
tgttgcgtggtcagtttc

SEQ ID NO: 318 - DNA, 18 bp, Homo Sapiens
  primer_bind 1..18: upstream amplification primer 99-28332 for SEQ 147,
tagagtcaacaatgggag

SEQ ID NO: 319 - DNA, 18 bp, Homo Sapiens
  primer_bind 1..18: upstream amplification primer 99-28339 for SEQ 148,
tcattattgcggcttctc

SEQ ID NO: 320 - DNA, 19 bp, Homo Sapiens
  primer_bind 1..19: upstream amplification primer 99-28342 for SEQ 149,
gcaaaatattaagacagcc

SEQ ID NO: 321 - DNA, 20 bp, Homo Sapiens
  primer_bind 1..20: upstream amplification primer 99-28343 for SEQ 150,
ggacatattagaaaaacgag

SEQ ID NO: 322 - DNA, 18 bp, Homo Sapiens
  primer_bind 1..18: upstream amplification primer 99-28352 for SEQ 151,
tcaccctttgtatcagtc

SEQ ID NO: 323 - DNA, 18 bp, Homo Sapiens
  primer_bind 1..18: upstream amplification primer 99-28371 for SEQ 152,
ttagtcacccctgcaatc

SEQ ID NO: 324 - DNA, 20 bp, Homo Sapiens
  primer_bind 1..20: upstream amplification primer 99-27041 for SEQ 153,
catgaaggaagaaatgaatc

SEQ ID NO: 325 - DNA, 18 bp, Homo Sapiens
  primer_bind 1..18: upstream amplification primer 99-29191 for SEQ 154,
gttcttgggatggttctg

SEQ ID NO: 326 - DNA, 18 bp, Homo Sapiens
  primer_bind 1..18: upstream amplification primer 99-27074 for SEQ 155,
tttgtgggagaatggaag

SEQ ID NO: 327 - DNA, 18 bp, Homo Sapiens
  primer_bind 1..18: upstream amplification primer 99-27076 for SEQ 156,
atgaaggtgaaagggaag

SEQ ID NO: 328 - DNA, 18 bp, Homo Sapiens
  primer_bind 1..18: upstream amplification primer 99-27079 for SEQ 157,
gttatgcttctcagagtg

SEQ ID NO: 329 - DNA, 21 bp, Homo Sapiens
  primer_bind 1..21: upstream amplification primer 99-27082 for SEQ 158,
gagttttgaagatattagctg

SEQ ID NO: 330 - DNA, 18 bp, Homo Sapiens
  primer_bind 1..18: upstream amplification primer 99-55890 for SEQ 159,
cctggaatcagatgagac

SEQ ID NO: 331 - DNA, 19 bp, Homo Sapiens
  primer_bind 1..19: upstream amplification primer 99-55901 for SEQ 160,
acatttcgtcttagcaatc

SEQ ID NO: 332 - DNA, 21 bp, Homo Sapiens
  primer_bind 1..21: upstream amplification primer 99-55903 for SEQ 161,
gtactttaaaaatgggctttg

SEQ ID NO: 333 - DNA, 20 bp, Homo Sapiens
  primer_bind 1..20: upstream amplification primer 9-42 for SEQ 162,
ataagtgtggctgtggaccc

SEQ ID NO: 334 - DNA, 18 bp, Homo Sapiens
  primer_bind 1..18: upstream amplification primer 99-41725 for SEQ 163,
atatgctgacaaggagac

SEQ ID NO: 335 - DNA, 19 bp, Homo Sapiens
  primer_bind 1..19: upstream amplification primer 99-45440 for SEQ 164,
cttattttttcccttaccc

SEQ ID NO: 336 - DNA, 18 bp, Homo Sapiens
  primer_bind 1..18: upstream amplification primer 99-438 for SEQ 165,
gagttctttcctgtagtc

SEQ ID NO: 337 - DNA, 18 bp, Homo Sapiens
  primer_bind 1..18: upstream amplification primer 99-36428 for SEQ 166,
atgagggtaagcagcaac

SEQ ID NO: 338 - DNA, 20 bp, Homo Sapiens
  primer_bind 1..20: upstream amplification primer 99-36433 for SEQ 167,
gaaatcaaaagtcttgagtc

SEQ ID NO: 339 - DNA, 20 bp, Homo Sapiens
  primer_bind 1..20: upstream amplification primer 99-26968 for SEQ 168,
ctttcttgtgtgcaaaagag

SEQ ID NO: 340 - DNA, 19 bp, Homo Sapiens
  primer_bind 1..19: upstream amplification primer 99-26345 for SEQ 169,
cactgaagatgttgagaac

SEQ ID NO: 341 - DNA, 18 bp, Homo Sapiens
  primer_bind 1..18: upstream amplification primer 99-27070 for SEQ 170,
agaccctaagtgaatgcc

SEQ ID NO: 342 - DNA, 18 bp, Homo Sapiens
  primer_bind 1..18: upstream amplification primer 99-27581 for SEQ 171,
catttctctcttcccttc

SEQ ID NO: 343 - DNA, 21 bp, Homo Sapiens
  primer_bind 1..21: downstream amplification primer 99-55095 for SEQ 1, in complement
gaacatctcttctttttacac

SEQ ID NO: 344 - DNA, 21 bp, Homo Sapiens
  primer_bind 1..21: downstream amplification primer 99-55102 for SEQ 2, in complement
tttatactgttaaagcccctg

SEQ ID NO: 345 - DNA, 20 bp, Homo Sapiens
  primer_bind 1..20: downstream amplification primer 99-26407 for SEQ 3, in complement
cagtatatcacaaccatgac

SEQ ID NO: 346 - DNA, 20 bp, Homo Sapiens
  primer_bind 1..20: downstream amplification primer 99-26412 for SEQ 4, in complement
cagaagcataaaactattgg

SEQ ID NO: 347 - DNA, 18 bp, Homo Sapiens
  primer_bind 1..18: downstream amplification primer 99-26413 for SEQ 5, in complement
catctcaaggtgcttatc

SEQ ID NO: 348 - DNA, 19 bp, Homo Sapiens
  primer_bind 1..19: downstream amplification primer 99-26424 for SEQ 6, in complement
tcacaattccgttcagctc

SEQ ID NO: 349 - DNA, 21 bp, Homo Sapiens
  primer_bind 1..21: downstream amplification primer 99-44320 for SEQ 7, in complement
ggaaaaagaaataggaagtag

SEQ ID NO: 350 - DNA, 18 bp, Homo Sapiens
  primer_bind 1..18: downstream amplification primer 99-41889 for SEQ 8, in complement
tggcacttagaccttcac

SEQ ID NO: 351 - DNA, 21 bp, Homo Sapiens
  primer_bind 1..21: downstream amplification primer 99-45453 for SEQ 9, in complement
cactttagttaaggttgtgtc

SEQ ID NO: 352 - DNA, 18 bp, Homo Sapiens
  primer_bind 1..18: downstream amplification primer 99-43427 for SEQ 10, in complement
acaaaacctcaggagttc

SEQ ID NO: 353 - DNA, 21 bp, Homo Sapiens
  primer_bind 1..21: downstream amplification primer 99-55124 for SEQ 11, in complement
tggactcacttgatttctggg

SEQ ID NO: 354 - DNA, 18 bp, Homo Sapiens
  primer_bind 1..18: downstream amplification primer 99-46316 for SEQ 12, in complement
ttcttctgttgtagcccc

SEQ ID NO: 355 - DNA, 21 bp, Homo Sapiens
  primer_bind 1..21: downstream amplification primer 99-46325 for SEQ 13, in complement
tttccacctatcatcaagaac

SEQ ID NO: 356 - DNA, 18 bp, Homo Sapiens
  primer_bind 1..18: downstream amplification primer 99-46332 for SEQ 14, in complement
aagttgtgcaaccttgcc

SEQ ID NO: 357 - DNA, 18 bp, Homo Sapiens
  primer_bind 1..18: downstream amplification primer 99-47064 for SEQ 15, in complement
cctggaggacttacatac

SEQ ID NO: 358 - DNA, 18 bp, Homo Sapiens
  primer_bind 1..18: downstream amplification primer 99-47065 for SEQ 16, in complement
tgatttaggggaccacac

SEQ ID NO: 359 - DNA, 21 bp, Homo Sapiens
  primer_bind 1..21: downstream amplification primer 99-47066 for SEQ 17, in complement
agtgtttttccctctcctacc

SEQ ID NO: 360 - DNA, 20 bp, Homo Sapiens
  primer_bind 1..20: downstream amplification primer 99-38883 for SEQ 18, in complement
gtctgttatgctaataatcc

SEQ ID NO: 361 - DNA, 18 bp, Homo Sapiens
  primer_bind 1..18: downstream amplification primer 99-38888 for SEQ 19, in complement
ccttgttcagatccttag

SEQ ID NO: 362 - DNA, 19 bp, Homo Sapiens
  primer_bind 1..19: downstream amplification primer 99-38889 for SEQ 20, in complement
gaaaatgggtgggaatatg

SEQ ID NO: 363 - DNA, 19 bp, Homo Sapiens
  primer_bind 1..19: downstream amplification primer 99-26458 for SEQ 21, in complement
ctccttctcatacaacttc

SEQ ID NO: 364 - DNA, 21 bp, Homo Sapiens
  primer_bind 1..21: downstream amplification primer 99-47321 for SEQ 22, in complement
tttgatagtgaaacgagattg

SEQ ID NO: 365 - DNA, 20 bp, Homo Sapiens
  primer_bind 1..20: downstream amplification primer 99-36198 for SEQ 23, in complement
ccattacaaaggagattgac

SEQ ID NO: 366 - DNA, 21 bp, Homo Sapiens
  primer_bind 1..21: downstream amplification primer 99-36200 for SEQ 24, in complement
gaagttaggaatattaagagg

SEQ ID NO: 367 - DNA, 18 bp, Homo Sapiens
  primer_bind 1..18: downstream amplification primer 99-33135 for SEQ 25, in complement
gtgactttcctatcaagc

SEQ ID NO: 368 - DNA, 18 bp, Homo Sapiens
  primer_bind 1..18: downstream amplification primer 99-34451 for SEQ 26, in complement
ggggttatcaccaaaaag

SEQ ID NO: 369 - DNA, 21 bp, Homo Sapiens
  primer_bind 1..21: downstream amplification primer 99-34452 for SEQ 27, in complement
tgttttgttttcttcaacctg

SEQ ID NO: 370 - DNA, 21 bp, Homo Sapiens
  primer_bind 1..21: downstream amplification primer 99-48928 for SEQ 28, in complement
accccaagaaaatagagcgag

SEQ ID NO: 371 - DNA, 21 bp, Homo Sapiens
  primer_bind 1..21: downstream amplification primer 99-55720 for SEQ 29, in complement
tgaaaataagagtgaaaaggg

SEQ ID NO: 372 - DNA, 21 bp, Homo Sapiens
  primer_bind 1..21: downstream amplification primer 99-55721 for SEQ 30, in complement
gcaggtattctacagaaatgg

SEQ ID NO: 373 - DNA, 19 bp, Homo Sapiens
  primer_bind 1..19: downstream amplification primer 99-501 for SEQ 31, in complement
gtctcttgttaatgtaggg

SEQ ID NO: 374 - DNA, 20 bp, Homo Sapiens
  primer_bind 1..20: downstream amplification primer 99-34556 for SEQ 32, in complement
tatgagagagtgtcttgccc

SEQ ID NO: 375 - DNA, 21 bp, Homo Sapiens
  primer_bind 1..21: downstream amplification primer 99-34561 for SEQ 33, in complement
ttatcagcctcttttgtttcc

SEQ ID NO: 376 - DNA, 18 bp, Homo Sapiens
  primer_bind 1..18: downstream amplification primer 99-28061 for SEQ 34, in complement
cactatatcttcctcctc

SEQ ID NO: 377 - DNA, 21 bp, Homo Sapiens
  primer_bind 1..21: downstream amplification primer 99-28062 for SEQ 35, in complement
gcactaaaatgagatagaaac

SEQ ID NO: 378 - DNA, 18 bp, Homo Sapiens
  primer_bind 1..18: downstream amplification primer 99-28063 for SEQ 36, in complement
cctcggaagcaaaaagac

SEQ ID NO: 379 - DNA, 19 bp, Homo Sapiens
  primer_bind 1..19: downstream amplification primer 99-28067 for SEQ 37, in complement
agtttccaaagacaatgag

SEQ ID NO: 380 - DNA, 20 bp, Homo Sapiens
  primer_bind 1..20: downstream amplification primer 99-28080 for SEQ 38, in complement
catcaatacttatctctgtc

SEQ ID NO: 381 - DNA, 18 bp, Homo Sapiens
  primer_bind 1..18: downstream amplification primer 99-459 for SEQ 39, in complement
actcctacctcctttgtc

SEQ ID NO: 382 - DNA, 21 bp, Homo Sapiens
  primer_bind 1..21: downstream amplification primer 99-32104 for SEQ 40, in complement
ggagggagtatgaaaagaatg

SEQ ID NO: 383 - DNA, 20 bp, Homo Sapiens
  primer_bind 1..20: downstream amplification primer 99-26970 for SEQ 41, in complement
caatgggtagcagatataag

SEQ ID NO: 384 - DNA, 21 bp, Homo Sapiens
  primer_bind 1..21: downstream amplification primer 99-27502 for SEQ 42, in complement
ttagtacaactgcaaaagtcc

SEQ ID NO: 385 - DNA, 19 bp, Homo Sapiens
  primer_bind 1..19: downstream amplification primer 99-26994 for SEQ 43, in complement
gcaaaaaaagtttagccag

SEQ ID NO: 386 - DNA, 19 bp, Homo Sapiens
  primer_bind 1..19: downstream amplification primer 99-26995 for SEQ 44, in complement
cattctttctgctttctac

SEQ ID NO: 387 - DNA, 19 bp, Homo Sapiens
  primer_bind 1..19: downstream amplification primer 99-26997 for SEQ 45, in complement
gctgacttcatagcaaaac

SEQ ID NO: 388 - DNA, 21 bp, Homo Sapiens
  primer_bind 1..21: downstream amplification primer 99-27001 for SEQ 46, in complement
gattttgattacttagttgcc

SEQ ID NO: 389 - DNA, 19 bp, Homo Sapiens
  primer_bind 1..19: downstream amplification primer 99-32161 for SEQ 47, in complement
tcaatgaggttgaaagatg

SEQ ID NO: 390 - DNA, 21 bp, Homo Sapiens
  primer_bind 1..21: downstream amplification primer 99-32165 for SEQ 48, in complement
ggtaatgaaagaaatggtttg

SEQ ID NO: 391 - DNA, 21 bp, Homo Sapiens
  primer_bind 1..21: downstream amplification primer 99-32168 for SEQ 49, in complement
tttgttcacatttgtcatcgg

SEQ ID NO: 392 - DNA, 21 bp, Homo Sapiens
  primer_bind 1..21: downstream amplification primer 99-32172 for SEQ 50, in complement
ttgtgatgagtgaggaaacgg

SEQ ID NO: 393 - DNA, 21 bp, Homo Sapiens
  primer_bind 1..21: downstream amplification primer 99-32174 for SEQ 51, in complement
caccttttcccattcttttcc

SEQ ID NO: 394 - DNA, 19 bp, Homo Sapiens
  primer_bind 1..19: downstream amplification primer 99-57310 for SEQ 52, in complement
cattgcttctatggattac

SEQ ID NO: 395 - DNA, 21 bp, Homo Sapiens
  primer_bind 1..21: downstream amplification primer 99-57311 for SEQ 53, in complement
tccactcttggatttgatgac

SEQ ID NO: 396 - DNA, 21 bp, Homo Sapiens
  primer_bind 1..21: downstream amplification primer 99-57314 for SEQ 54, in complement
ctttatactatcttcccatac

SEQ ID NO: 397 - DNA, 21 bp, Homo Sapiens
  primer_bind 1..21: downstream amplification primer 99-26359 for SEQ 55, in complement
gcttgagtcacaatagctggg

SEQ ID NO: 398 - DNA, 18 bp, Homo Sapiens
  primer_bind 1..18: downstream amplification primer 99-27232 for SEQ 56, in complement
tataagaccctgctgatg

SEQ ID NO: 399 - DNA, 18 bp, Homo Sapiens
  primer_bind 1..18: downstream amplification primer 99-27595 for SEQ 57, in complement
caattcaggatgatgcag

SEQ ID NO: 400 - DNA, 19 bp, Homo Sapiens
  primer_bind 1..19: downstream amplification primer 99-26492 for SEQ 58, in complement
ccacagtatttacttcttg

SEQ ID NO: 401 - DNA, 21 bp, Homo Sapiens
  primer_bind 1..21: downstream amplification primer 99-26501 for SEQ 59, in complement
tcttcacacctttaagaaatg

SEQ ID NO: 402 - DNA, 21 bp, Homo Sapiens
  primer_bind 1..21: downstream amplification primer 99-28350 for SEQ 60, in complement
aaacaacactgctatatttcc

SEQ ID NO: 403 - DNA, 19 bp, Homo Sapiens
  primer_bind 1..19: downstream amplification primer 99-28355 for SEQ 61, in complement
tctctgtcacaatttttcc

SEQ ID NO: 404 - DNA, 21 bp, Homo Sapiens
  primer_bind 1..21: downstream amplification primer 99-28360 for SEQ 62, in complement
taagggtacatttaatgcctc

SEQ ID NO: 405 - DNA, 21 bp, Homo Sapiens
  primer_bind 1..21: downstream amplification primer 99-28363 for SEQ 63, in complement
cattgtaagctaaaaacaacc

SEQ ID NO: 406 - DNA, 21 bp, Homo Sapiens
  primer_bind 1..21: downstream amplification primer 99-28364 for SEQ 64, in complement
cccaccaaaatcaagtaacag

SEQ ID NO: 407 - DNA, 19 bp, Homo Sapiens
  primer_bind 1..19: downstream amplification primer 99-27049 for SEQ 65, in complement
ctgtgagctaaccatattg

SEQ ID NO: 408 - DNA, 20 bp, Homo Sapiens
  primer_bind 1..20: downstream amplification primer 99-27054 for SEQ 66, in complement
gtttagcaaacccaaacatc

SEQ ID NO: 409 - DNA, 21 bp, Homo Sapiens
  primer_bind 1..21: downstream amplification primer 99-27056 for SEQ 67, in complement
cagaattttttagggtaaagg

SEQ ID NO: 410 - DNA, 18 bp, Homo Sapiens
  primer_bind 1..18: downstream amplification primer 99-27586 for SEQ 68, in complement
agggtttggaagagttgg

SEQ ID NO: 411 - DNA, 21 bp, Homo Sapiens
  primer_bind 1..21: downstream amplification primer 99-29193 for SEQ 69, in complement
actattcacactacaattctc

SEQ ID NO: 412 - DNA, 21 bp, Homo Sapiens
  primer_bind 1..21: downstream amplification primer 99-56867 for SEQ 70, in complement
tcacttaggattacactgagg

SEQ ID NO: 413 - DNA, 18 bp, Homo Sapiens
  primer_bind 1..18: downstream amplification primer 99-26399 for SEQ 71, in complement
aggtacatgctggttacg

SEQ ID NO: 414 - DNA, 21 bp, Homo Sapiens
  primer_bind 1..21: downstream amplification primer 99-41887 for SEQ 72, in complement
aacatgattaagagaaccaac

SEQ ID NO: 415 - DNA, 18 bp, Homo Sapiens
  primer_bind 1..18: downstream amplification primer 99-27851 for SEQ 73, in complement
tagtgtcacataggcaag

SEQ ID NO: 416 - DNA, 20 bp, Homo Sapiens
  primer_bind 1..20: downstream amplification primer 99-38884 for SEQ 74, in complement
ttacctctcaaacacatacc

SEQ ID NO: 417 - DNA, 19 bp, Homo Sapiens
  primer_bind 1..19: downstream amplification primer 99-38897 for SEQ 75, in complement
tagttggtggtggaactgg

SEQ ID NO: 418 - DNA, 18 bp, Homo Sapiens
  primer_bind 1..18: downstream amplification primer 99-36178 for SEQ 76, in complement
ctgttttattccccactc

SEQ ID NO: 419 - DNA, 18 bp, Homo Sapiens
  primer_bind 1..18: downstream amplification primer 99-36201 for SEQ 77, in complement
tcagactgagctggcagg

SEQ ID NO: 420 - DNA, 19 bp, Homo Sapiens
  primer_bind 1..19: downstream amplification primer 99-32207 for SEQ 78, in complement
ctcactctcctctttattc

SEQ ID NO: 421 - DNA, 20 bp, Homo Sapiens
  primer_bind 1..20: downstream amplification primer 99-55707 for SEQ 79, in complement
taaaagagccctggcgagac

SEQ ID NO: 422 - DNA, 20 bp, Homo Sapiens
  primer_bind 1..20: downstream amplification primer 99-48925 for SEQ 80, in complement
agatccacaccaaagtcccc

SEQ ID NO: 423 - DNA, 20 bp, Homo Sapiens
  primer_bind 1..20: downstream amplification primer 99-55725 for SEQ 81, in complement
ccacaaactcccacttagcc

SEQ ID NO: 424 - DNA, 21 bp, Homo Sapiens
  primer_bind 1..21: downstream amplification primer 99-28082 for SEQ 82, in complement
cgtgaatttgttgatattgtc

SEQ ID NO: 425 - DNA, 21 bp, Homo Sapiens
  primer_bind 1..21: downstream amplification primer 99-28083 for SEQ 83, in complement
caaatatacagagaatatgcc

SEQ ID NO: 426 - DNA, 19 bp, Homo Sapiens
  primer_bind 1..19: downstream amplification primer 99-27498 for SEQ 84, in complement
tgggaagatttgactattg

SEQ ID NO: 427 - DNA, 18 bp, Homo Sapiens
  primer_bind 1..18: downstream amplification primer 99-27503 for SEQ 85, in complement
attctattccgtgccctg

SEQ ID NO: 428 - DNA, 19 bp, Homo Sapiens
  primer_bind 1..19: downstream amplification primer 99-26511 for SEQ 86, in complement
taacccttcaaacttcttc

SEQ ID NO: 429 - DNA, 18 bp, Homo Sapiens
  primer_bind 1..18: downstream amplification primer 99-26989 for SEQ 87, in complement
agctcttatcccattgac

SEQ ID NO: 430 - DNA, 21 bp, Homo Sapiens
  primer_bind 1..21: downstream amplification primer 99-32158 for SEQ 88, in complement
cttcggttatgtgatctggtg

SEQ ID NO: 431 - DNA, 21 bp, Homo Sapiens
  primer_bind 1..21: downstream amplification primer 99-32162 for SEQ 89, in complement
gtggtaagttctttttggttg

SEQ ID NO: 432 - DNA, 21 bp, Homo Sapiens
  primer_bind 1..21: downstream amplification primer 99-32166 for SEQ 90, in complement
aatcagtaagtgagaaagagg

SEQ ID NO: 433 - DNA, 21 bp, Homo Sapiens
  primer_bind 1..21: downstream amplification primer 99-32169 for SEQ 91, in complement
ggaattactgagagaagacgg

SEQ ID NO: 434 - DNA, 18 bp, Homo Sapiens
  primer_bind 1..18: downstream amplification primer 99-57305 for SEQ 92, in complement
gggtcttaggtcagtatc

SEQ ID NO: 435 - DNA, 19 bp, Homo Sapiens
  primer_bind 1..19: downstream amplification primer 99-57308 for SEQ 93, in complement
cccatattttttccaaacg

SEQ ID NO: 436 - DNA, 21 bp, Homo Sapiens
  primer_bind 1..21: downstream amplification primer 99-26477 for SEQ 94, in complement
ttcttatacccttcagcgttc

SEQ ID NO: 437 - DNA, 18 bp, Homo Sapiens
  primer_bind 1..18: downstream amplification primer 99-26479 for SEQ 95, in complement
aattctggttctggagtg

SEQ ID NO: 438 - DNA, 21 bp, Homo Sapiens
  primer_bind 1..21: downstream amplification primer 99-26480 for SEQ 96, in complement
tctgagttaaactaatttggg

SEQ ID NO: 439 - DNA, 21 bp, Homo Sapiens
  primer_bind 1..21: downstream amplification primer 99-26503 for SEQ 97, in complement
tgagttgatgcagaccatgac

SEQ ID NO: 440 - DNA, 21 bp, Homo Sapiens
  primer_bind 1..21: downstream amplification primer 99-27042 for SEQ 98, in complement
gtgttttatgtcactgctgtc

SEQ ID NO: 441 - DNA, 19 bp, Homo Sapiens
  primer_bind 1..19: downstream amplification primer 99-27050 for SEQ 99, in complement
tgaatccttggaaggggag

SEQ ID NO: 442 - DNA, 20 bp, Homo Sapiens
  primer_bind 1..20: downstream amplification primer 99-27583 for SEQ 100, in complement
actgtaaaagaagaagctgg

SEQ ID NO: 443 - DNA, 21 bp, Homo Sapiens
  primer_bind 1..21: downstream amplification primer 99-55097 for SEQ 101, in complement
ggtggatagaaaggaagatgg

SEQ ID NO: 444 - DNA, 18 bp, Homo Sapiens
  primer_bind 1..18: downstream amplification primer 99-41727 for SEQ 102, in complement
aggttctcctcctttgtg

SEQ ID NO: 445 - DNA, 19 bp, Homo Sapiens
  primer_bind 1..19: downstream amplification primer 99-44259 for SEQ 103, in complement
agatttgttgaagaaggtc

SEQ ID NO: 446 - DNA, 20 bp, Homo Sapiens
  primer_bind 1..20: downstream amplification primer 99-44260 for SEQ 104, in complement
agtaaagaaacaggaagcag

SEQ ID NO: 447 - DNA, 20 bp, Homo Sapiens
  primer_bind 1..20: downstream amplification primer 99-41009 for SEQ 105, in complement
actcttgttctcccctcacc

SEQ ID NO: 448 - DNA, 21 bp, Homo Sapiens
  primer_bind 1..21: downstream amplification primer 99-41010 for SEQ 106, in complement
tcatctaccttttcaaaatcc

SEQ ID NO: 449 - DNA, 21 bp, Homo Sapiens
  primer_bind 1..21: downstream amplification primer 99-41828 for SEQ 107, in complement
tagttgttgatagcttttcgg

SEQ ID NO: 450 - DNA, 20 bp, Homo Sapiens
  primer_bind 1..20: downstream amplification primer 99-45821 for SEQ 108, in complement
cagaatctctcttatgatac

SEQ ID NO: 451 - DNA, 19 bp, Homo Sapiens
  primer_bind 1..19: downstream amplification primer 99-26385 for SEQ 109, in complement
gatattgttctgcttgatg

SEQ ID NO: 452 - DNA, 19 bp, Homo Sapiens
  primer_bind 1..19: downstream amplification primer 99-48212 for SEQ 110, in complement
ttcagagaactttatgccc

SEQ ID NO: 453 - DNA, 21 bp, Homo Sapiens
  primer_bind 1..21: downstream amplification primer 99-36203 for SEQ 111, in complement
gctgatttttgacaaaacagg

SEQ ID NO: 454 - DNA, 21 bp, Homo Sapiens
  primer_bind 1..21: downstream amplification primer 99-441 for SEQ 112, in complement
ctaacccagtatgtatattcc

SEQ ID NO: 455 - DNA, 20 bp, Homo Sapiens
  primer_bind 1..20: downstream amplification primer 99-444 for SEQ 113, in complement
gacagactcaatagatagac

SEQ ID NO: 456 - DNA, 20 bp, Homo Sapiens
  primer_bind 1..20: downstream amplification primer 99-55706 for SEQ 114, in complement
ctatactttaaggagcaagg

SEQ ID NO: 457 - DNA, 19 bp, Homo Sapiens
  primer_bind 1..19: downstream amplification primer 99-55708 for SEQ 115, in complement
aaatgacgtgcaagtgggg

SEQ ID NO: 458 - DNA, 19 bp, Homo Sapiens
  primer_bind 1..19: downstream amplification primer 99-55709 for SEQ 116, in complement
agcatttactaagaactgg

SEQ ID NO: 459 - DNA, 21 bp, Homo Sapiens
  primer_bind 1..21: downstream amplification primer 99-36422 for SEQ 117, in complement
gacataaagtaccaagagcag

SEQ ID NO: 460 - DNA, 18 bp, Homo Sapiens
  primer_bind 1..18: downstream amplification primer 99-36426 for SEQ 118, in complement
cacatacctctgacaaac

SEQ ID NO: 461 - DNA, 21 bp, Homo Sapiens
  primer_bind 1..21: downstream amplification primer 99-54269 for SEQ 119, in complement
ttttaaggtgagaagaaatgg

SEQ ID NO: 462 - DNA, 21 bp, Homo Sapiens
  primer_bind 1..21: downstream amplification primer 99-54270 for SEQ 120, in complement
cattgcgtgtttttcacgtag

SEQ ID NO: 463 - DNA, 18 bp, Homo Sapiens
  primer_bind 1..18: downstream amplification primer 99-54272 for SEQ 121, in complement
tagggtaaagagacacag

SEQ ID NO: 464 - DNA, 19 bp, Homo Sapiens
  primer_bind 1..19: downstream amplification primer 99-54276 for SEQ 122, in complement
ggaacctgtttctcaaaag

SEQ ID NO: 465 - DNA, 18 bp, Homo Sapiens
  primer_bind 1..18: downstream amplification primer 99-54277 for SEQ 123, in complement
gcctaatatgctggcttg

SEQ ID NO: 466 - DNA, 21 bp, Homo Sapiens
  primer_bind 1..21: downstream amplification primer 99-49542 for SEQ 124, in complement
ttaggcaacactttcagcaac

SEQ ID NO: 467 - DNA, 18 bp, Homo Sapiens
  primer_bind 1..18: downstream amplification primer 99-49544 for SEQ 125, in complement
ctctactggctaacaaac

SEQ ID NO: 468 - DNA, 21 bp, Homo Sapiens
  primer_bind 1..21: downstream amplification primer 99-49545 for SEQ 126, in complement
tctcttttatatacatgaggg

SEQ ID NO: 469 - DNA, 21 bp, Homo Sapiens
  primer_bind 1..21: downstream amplification primer 99-49547 for SEQ 127, in complement
caaaaccacacttacttcacc

SEQ ID NO: 470 - DNA, 20 bp, Homo Sapiens
  primer_bind 1..20: downstream amplification primer 99-457 for SEQ 128, in complement
ctttcttaccttccattctc

SEQ ID NO: 471 - DNA, 21 bp, Homo Sapiens
  primer_bind 1..21: downstream amplification primer 99-463 for SEQ 129, in complement
gaagaaagagagccagaagac

SEQ ID NO: 472 - DNA, 19 bp, Homo Sapiens
  primer_bind 1..19: downstream amplification primer 99-32097 for SEQ 130, in complement
gcaagtttagccttatttc

SEQ ID NO: 473 - DNA, 19 bp, Homo Sapiens
  primer_bind 1..19: downstream amplification primer 99-32110 for SEQ 131, in complement
aaggatttggtgtgttttc

SEQ ID NO: 474 - DNA, 21 bp, Homo Sapiens
  primer_bind 1..21: downstream amplification primer 99-32117 for SEQ 132, in complement
ctcctccacttattccaattc

SEQ ID NO: 475 - DNA, 19 bp, Homo Sapiens
  primer_bind 1..19: downstream amplification primer 99-26972 for SEQ 133, in complement
atgaggctcttcttttttc

SEQ ID NO: 476 - DNA, 21 bp, Homo Sapiens
  primer_bind 1..21: downstream amplification primer 99-26973 for SEQ 134, in complement
ctccctctaactcttctcttc

SEQ ID NO: 477 - DNA, 20 bp, Homo Sapiens
  primer_bind 1..20: downstream amplification primer 99-26974 for SEQ 135, in complement
ctgtgcattcaggtctaggg

SEQ ID NO: 478 - DNA, 18 bp, Homo Sapiens
  primer_bind 1..18: downstream amplification primer 99-26975 for SEQ 136, in complement
catgaggtcatgtcattg

SEQ ID NO: 479 - DNA, 21 bp, Homo Sapiens
  primer_bind 1..21: downstream amplification primer 99-26978 for SEQ 137, in complement
taatgatttttaccttcccac

SEQ ID NO: 480 - DNA, 21 bp, Homo Sapiens
  primer_bind 1..21: downstream amplification primer 99-26981 for SEQ 138, in complement
ttgctatcattctctgctttg

SEQ ID NO: 481 - DNA, 20 bp, Homo Sapiens
  primer_bind 1..20: downstream amplification primer 99-26983 for SEQ 139, in complement
tcacccagagcaccaaaacc

SEQ ID NO: 482 - DNA, 21 bp, Homo Sapiens
  primer_bind 1..21: downstream amplification primer 99-26987 for SEQ 140, in complement
atctaaagctgtggatacgtg

SEQ ID NO: 483 - DNA, 21 bp, Homo Sapiens
  primer_bind 1..21: downstream amplification primer 99-32153 for SEQ 141, in complement
gaacaaaagaagagagcagcc

SEQ ID NO: 484 - DNA, 18 bp, Homo Sapiens
  primer_bind 1..18: downstream amplification primer 99-32156 for SEQ 142, in complement
ggtaaggggtatagagag

SEQ ID NO: 485 - DNA, 19 bp, Homo Sapiens
  primer_bind 1..19: downstream amplification primer 99-57302 for SEQ 143, in complement
tggcaacataaagtatgag

SEQ ID NO: 486 - DNA, 21 bp, Homo Sapiens
  primer_bind 1..21: downstream amplification primer 99-26349 for SEQ 144, in complement
aattgctcaaagcctccgaac

SEQ ID NO: 487 - DNA, 21 bp, Homo Sapiens
  primer_bind 1..21: downstream amplification primer 99-26352 for SEQ 145, in complement
aaaaatgatttgggggacggg

SEQ ID NO: 488 - DNA, 21 bp, Homo Sapiens
  primer_bind 1..21: downstream amplification primer 99-26370 for SEQ 146, in complement
ccaccatatatccattccttc

SEQ ID NO: 489 - DNA, 21 bp, Homo Sapiens
  primer_bind 1..21: downstream amplification primer 99-28332 for SEQ 147, in complement
aagtgtcaaggtcaaaatggg

SEQ ID NO: 490 - DNA, 18 bp, Homo Sapiens
  primer_bind 1..18: downstream amplification primer 99-28339 for SEQ 148, in complement
ggctgaactctccttaac

SEQ ID NO: 491 - DNA, 18 bp, Homo Sapiens
  primer_bind 1..18: downstream amplification primer 99-28342 for SEQ 149, in complement
tgttacagatccccagag

SEQ ID NO: 492 - DNA, 18 bp, Homo Sapiens
  primer_bind 1..18: downstream amplification primer 99-28343 for SEQ 150, in complement
ccaggtaagaagatggag

SEQ ID NO: 493 - DNA, 19 bp, Homo Sapiens
  primer_bind 1..19: downstream amplification primer 99-28352 for SEQ 151, in complement
tcacacatagcctggagag

SEQ ID NO: 494 - DNA, 19 bp, Homo Sapiens
  primer_bind 1..19: downstream amplification primer 99-28371 for SEQ 152, in complement
ggtcttcaaatctgtgttc

SEQ ID NO: 495 - DNA, 21 bp, Homo Sapiens
  primer_bind 1..21: downstream amplification primer 99-27041 for SEQ 153, in complement
ggagaaggttagaatagaagg

SEQ ID NO: 496 - DNA, 20 bp, Homo Sapiens
  primer_bind 1..20: downstream amplification primer 99-29191 for SEQ 154, in complement
cttacgaatcaaaaggggac

SEQ ID NO: 497 - DNA, 18 bp, Homo Sapiens
  primer_bind 1..18: downstream amplification primer 99-27074 for SEQ 155, in complement
atgatggatcatacggtg

SEQ ID NO: 498 - DNA, 21 bp, Homo Sapiens
  primer_bind 1..21: downstream amplification primer 99-27076 for SEQ 156, in complement
agaacaaaaataaggaagagg

SEQ ID NO: 499 - DNA, 18 bp, Homo Sapiens
  primer_bind 1..18: downstream amplification primer 99-27079 for SEQ 157, in complement
cagtcagttggtaatggg

SEQ ID NO: 500 - DNA, 21 bp, Homo Sapiens
  primer_bind 1..21: downstream amplification primer 99-27082 for SEQ 158, in complement
aaagtgctaaagtacattggg

SEQ ID NO: 501 - DNA, 21 bp, Homo Sapiens
  primer_bind 1..21: downstream amplification primer 99-55890 for SEQ 159, in complement
caaaaatctttagcaactgac

SEQ ID NO: 502 - DNA, 21 bp, Homo Sapiens
  primer_bind 1..21: downstream amplification primer 99-55901 for SEQ 160, in complement
gacatcaagaattttcattcc

SEQ ID NO: 503 - DNA, 19 bp, Homo Sapiens
  primer_bind 1..19: downstream amplification primer 99-55903 for SEQ 161, in complement
agtgttcctaagtattctg

SEQ ID NO: 504 - DNA, 20 bp, Homo Sapiens
  primer_bind 1..20: downstream amplification primer 9-42 for SEQ 162, in complement
tcaagtctggctgtggaacc

SEQ ID NO: 505 - DNA, 18 bp, Homo Sapiens
  primer_bind 1..18: downstream amplification primer 99-41725 for SEQ 163, in complement
cttgacaaacccaggaac

SEQ ID NO: 506 - DNA, 21 bp, Homo Sapiens
  primer_bind 1..21: downstream amplification primer 99-45440 for SEQ 164, in complement
atcagcacatacacgaaacac

SEQ ID NO: 507 - DNA, 21 bp, Homo Sapiens
  primer_bind 1..21: downstream amplification primer 99-438 for SEQ 165, in complement
cattcatttcttcaacttacc

SEQ ID NO: 508 - DNA, 18 bp, Homo Sapiens
  primer_bind 1..18: downstream amplification primer 99-36428 for SEQ 166, in complement
tctcttaggtaagaaccg

SEQ ID NO: 509 - DNA, 21 bp, Homo Sapiens
  primer_bind 1..21: downstream amplification primer 99-36433 for SEQ 167, in complement
cttattaattctcactcatcc

SEQ ID NO: 510 - DNA, 21 bp, Homo Sapiens
  primer_bind 1..21: downstream amplification primer 99-26968 for SEQ 168, in complement
acagcatagcaactgctttcc

SEQ ID NO: 511 - DNA, 18 bp, Homo Sapiens
  primer_bind 1..18: downstream amplification primer 99-26345 for SEQ 169, in complement
aagtgctgagacccagag

SEQ ID NO: 512 - DNA, 20 bp, Homo Sapiens
  primer_bind 1..20: downstream amplification primer 99-27070 for SEQ 170, in complement
aaatgtaacagaagccagtg

SEQ ID NO: 513 - DNA, 21 bp, Homo Sapiens
  primer_bind 1..21: downstream amplification primer 99-27581 for SEQ 171, in complement
ccatgttcaagtatctgtgtc

SEQ ID NO: 514 - DNA, 47 bp, Homo Sapiens
  allele 24: 99-344-439  polymorphic base G or A
tgctgccaaggatccatgtcagcrtgctcctctctgagccctggtct

SEQ ID NO: 515 - DNA, 47 bp, Homo Sapiens
  allele 24: 99-366-274  polymorphic base C or T
agggcctggcttcagggacagctyaggaaatgtttgttgagttagtg

SEQ ID NO: 516 - DNA, 47 bp, Homo Sapiens
  allele 24: 99-359-308  polymorphic base A or G
ctacagagtcatcgcctccatccrgtctcaacaaatcctggcagctc

SEQ ID NO: 517 - DNA, 47 bp, Homo Sapiens
  allele 24: 99-355-219  polymorphic base A or G
ggagtttcggggagtttcgggagrgttcctgggaagaagctcctccc

SEQ ID NO: 518 - DNA, 47 bp, Homo Sapiens
  allele 24: 99-365-344  polymorphic base C or T
cctaccaagcaagcagccccagcytagggtcagacagggtgagcctc

SEQ ID NO: 519 - DNA, 47 bp, Homo Sapiens
  allele 24: 99-2452-54  polymorphic base C or T
tgggcgcggacatggaggacgtgygcggccgcctggtgcagtaccgc

SEQ ID NO: 520 - DNA, 47 bp, Homo Sapiens
  allele 24: 99-123-381  polymorphic base C or T
tttctcatcctcacacctcactgygcccctcctgaacccactccttt

SEQ ID NO: 521 - DNA, 47 bp, Homo Sapiens
  allele 24: 4-26-29  polymorphic base A or G
ccctgtnagacacgtcctgtatcrttgttgagatgggaaagtgcatc

SEQ ID NO: 522 - DNA, 47 bp, Homo Sapiens
  allele 24: 4-14-240  polymorphic base C or T
gcagggagcagaccagacatgatytgttctagtctagctgattcata

SEQ ID NO: 523 - DNA, 47 bp, Homo Sapiens
  allele 24: 4-77-151  polymorphic base G or C
gctgttcagactaaacttggagastacagtcagtcagagaacttgct

SEQ ID NO: 524 - DNA, 47 bp, Homo Sapiens
  allele 24: 99-217-277  polymorphic base C or T
atatagttcacgttatgttcatayttaattgttgcattttgtttgcc

SEQ ID NO: 525 - DNA, 47 bp, Homo Sapiens
  allele 24: 4-67-40  polymorphic base C or T
gccagtgaaatacagacttaattygtcatgactgaacgaatttgttt

SEQ ID NO: 526 - DNA, 47 bp, Homo Sapiens
  allele 24: 99-213-164  polymorphic base A or G
cccccaggggtggacaacaccagrgctcaggggcttgaatgctaagg

SEQ ID NO: 527 - DNA, 47 bp, Homo Sapiens
  allele 24: 99-221-377  polymorphic base A or C
agcttgagaaaccagaaaagccamaaggaggctcctaccacatgggt

SEQ ID NO: 528 - DNA, 47 bp, Homo Sapiens
  allele 24: 99-135-196  polymorphic base A or G
agtcactatatctatgtttaatgragatagaaagagatgcagaaatg

SEQ ID NO: 529 - DNA, 47 bp, Homo Sapiens
  allele 24: 99-1482-32  polymorphic base A or C
agtgaagtctgagggggaaaaatmaaccctatagagggaaggatctg

SEQ ID NO: 530 - DNA, 47 bp, Homo Sapiens
  allele 24: 4-73-134  polymorphic base G or C
gttttccttatgatgttacatggsttatttttaaaggtaatgaaaac

SEQ ID NO: 531 - DNA, 47 bp, Homo Sapiens
  allele 24: 4-65-324  polymorphic base C or T
ggtgctgctcagcggcttgcacgyagacttgctaggaagaaatgcag

SEQ ID NO: 532 - DNA, 47 bp, Homo Sapiens
  allele 24: 9-3-324  polymorphic base C or T
aacccaggctacaacccctacgtygagtgccaggacagcgtgcgcac

SEQ ID NO: 533 - DNA, 47 bp, Homo Sapiens
  allele 24: 9-24-260  polymorphic base A or G
cgggtggagggtaggagccatgcrctagggcttcagcccccagcccc

SEQ ID NO: 534 - DNA, 47 bp, Homo Sapiens
  allele 24: 9-7-325  polymorphic base A or G
ataccctggagacgttgacaggartagctcaggtgaggccgggggaa

SEQ ID NO: 535 - DNA, 47 bp, Homo Sapiens
  allele 24: 99-4582-359  polymorphic base G or T
acagggtttcaccgtgttagccakgatggtctcgatctcctgacctc

SEQ ID NO: 536 - DNA, 20 bp, Homo Sapiens
  primer_bind 1..20: upstream amplification primer 99-344 for SEQ 514,
gctctcatattcattgggtg

SEQ ID NO: 537 - DNA, 18 bp, Homo Sapiens
  primer_bind 1..18: upstream amplification primer 99-366 for SEQ 515,
tctctcccgtgttaaatg

SEQ ID NO: 538 - DNA, 18 bp, Homo Sapiens
  primer_bind 1..18: upstream amplification primer 99-359 for SEQ 516,
aatcttcttgctcctgtc

SEQ ID NO: 539 - DNA, 18 bp, Homo Sapiens
  primer_bind 1..18: upstream amplification primer 99-355 for SEQ 517,
aggttaggggtgtatttc

SEQ ID NO: 540 - DNA, 18 bp, Homo Sapiens
  primer_bind 1..18: upstream amplification primer 99-365 for SEQ 518,
agactgtgaccttagacc

SEQ ID NO: 541 - DNA, 18 bp, Homo Sapiens
  primer_bind 1..18: upstream amplification primer 99-2452 for SEQ 519,
gacgagaccatgaaggag

SEQ ID NO: 542 - DNA, 18 bp, Homo Sapiens
  primer_bind 1..18: upstream amplification primer 99-123 for SEQ 520,
aaagccaggactagaagg

SEQ ID NO: 543 - DNA, 18 bp, Homo Sapiens
  primer_bind 1..18: upstream amplification primer 4-26 for SEQ 521,
tacagccctgtaagacac

SEQ ID NO: 544 - DNA, 18 bp, Homo Sapiens
  primer_bind 1..18: upstream amplification primer 4-14 for SEQ 522,
tctaacctctcatccaac

SEQ ID NO: 545 - DNA, 18 bp, Homo Sapiens
  primer_bind 1..18: upstream amplification primer 4-77 for SEQ 523,
tgttgatttacaggcggc

SEQ ID NO: 546 - DNA, 19 bp, Homo Sapiens
  primer_bind 1..19: upstream amplification primer 99-217 for SEQ 524,
ggtgggaatttactatatg

SEQ ID NO: 547 - DNA, 18 bp, Homo Sapiens
  primer_bind 1..18: upstream amplification primer 4-67 for SEQ 525,
aagttcaccttctcaagc

SEQ ID NO: 548 - DNA, 20 bp, Homo Sapiens
  primer_bind 1..20: upstream amplification primer 99-213 for SEQ 526,
atactggcagcgtgtgcttc

SEQ ID NO: 549 - DNA, 19 bp, Homo Sapiens
  primer_bind 1..19: upstream amplification primer 99-221 for SEQ 527,
ccctttttcttcactgttc

SEQ ID NO: 550 - DNA, 18 bp, Homo Sapiens
  primer_bind 1..18: upstream amplification primer 99-135 for SEQ 528,
tggaagttgttattgccc

SEQ ID NO: 551 - DNA, 21 bp, Homo Sapiens
  primer_bind 1..21: upstream amplification primer 99-1482 for SEQ 529,
atcaaatcagtgaagtctgag

SEQ ID NO: 552 - DNA, 18 bp, Homo Sapiens
  primer_bind 1..18: upstream amplification primer 4-73 for SEQ 530,
atcgctggaacattctgg

SEQ ID NO: 553 - DNA, 20 bp, Homo Sapiens
  primer_bind 1..20: upstream amplification primer 4-65 for SEQ 531,
gatttaagctacgctattag

SEQ ID NO: 554 - DNA, 20 bp, Homo Sapiens
  primer_bind 1..20: upstream amplification primer 9-3 for SEQ 532,
tgtcaagctggtgacaggtg

SEQ ID NO: 555 - DNA, 20 bp, Homo Sapiens
  primer_bind 1..20: upstream amplification primer 9-24 for SEQ 533,
agagctgagatcctatttcg

SEQ ID NO: 556 - DNA, 19 bp, Homo Sapiens
  primer_bind 1..19: upstream amplification primer 9-7 for SEQ 534,
aatgaggaccaacagggac

SEQ ID NO: 557 - DNA, 19 bp, Homo Sapiens
  primer_bind 1..19: upstream amplification primer 99-4582 for SEQ 535,
gggggaagggaatgtgaag

SEQ ID NO: 558 - DNA, 19 bp, Homo Sapiens
  primer_bind 1..19: downstream amplification primer 99-344 for SEQ 514, in complement
tggctgcggttagatgctc

SEQ ID NO: 559 - DNA, 18 bp, Homo Sapiens
  primer_bind 1..18: downstream amplification primer 99-366 for SEQ 515, in complement
aggggtaactcttgattg

SEQ ID NO: 560 - DNA, 18 bp, Homo Sapiens
  primer_bind 1..18: downstream amplification primer 99-359 for SEQ 516, in complement
accaaggcatagcttctc

SEQ ID NO: 561 - DNA, 18 bp, Homo Sapiens
  primer_bind 1..18: downstream amplification primer 99-355 for SEQ 517, in complement
atacagccagggagatag

SEQ ID NO: 562 - DNA, 18 bp, Homo Sapiens
  primer_bind 1..18: downstream amplification primer 99-365 for SEQ 518, in complement
aattgctacccccaattc

SEQ ID NO: 563 - DNA, 18 bp, Homo Sapiens
  primer_bind 1..18: downstream amplification primer 99-2452 for SEQ 519, in complement
tcgaaccagctcttgagg

SEQ ID NO: 564 - DNA, 18 bp, Homo Sapiens
  primer_bind 1..18: downstream amplification primer 99-123 for SEQ 520, in complement
tattcagaaaggagtggg

SEQ ID NO: 565 - DNA, 18 bp, Homo Sapiens
  primer_bind 1..18: downstream amplification primer 4-26 for SEQ 521, in complement
tgaggactgctaggaaag

SEQ ID NO: 566 - DNA, 20 bp, Homo Sapiens
  primer_bind 1..20: downstream amplification primer 4-14 for SEQ 522, in complement
gactgtatcctttgatgcac

SEQ ID NO: 567 - DNA, 20 bp, Homo Sapiens
  primer_bind 1..20: downstream amplification primer 4-77 for SEQ 523, in complement
ggaaaggtactcattcatag

SEQ ID NO: 568 - DNA, 21 bp, Homo Sapiens
  primer_bind 1..21: downstream amplification primer 99-217 for SEQ 524, in complement
gtttattttgtgtgagctttg

SEQ ID NO: 569 - DNA, 20 bp, Homo Sapiens
  primer_bind 1..20: downstream amplification primer 4-67 for SEQ 525, in complement
tgaaagagtttattctctgg

SEQ ID NO: 570 - DNA, 21 bp, Homo Sapiens
  primer_bind 1..21: downstream amplification primer 99-213 for SEQ 526, in complement
ttattgccccacatgcttgag

SEQ ID NO: 571 - DNA, 19 bp, Homo Sapiens
  primer_bind 1..19: downstream amplification primer 99-221 for SEQ 527, in complement
tcattcgtctggctaggtc

SEQ ID NO: 572 - DNA, 18 bp, Homo Sapiens
  primer_bind 1..18: downstream amplification primer 99-135 for SEQ 528, in complement
aaacacctcccattgtgc

SEQ ID NO: 573 - DNA, 20 bp, Homo Sapiens
  primer_bind 1..20: downstream amplification primer 99-1482 for SEQ 529, in complement
acaaatctatataaggctgg

SEQ ID NO: 574 - DNA, 20 bp, Homo Sapiens
  primer_bind 1..20: downstream amplification primer 4-73 for SEQ 530, in complement
ctcttggttaaacagcagtg

SEQ ID NO: 575 - DNA, 18 bp, Homo Sapiens
  primer_bind 1..18: downstream amplification primer 4-65 for SEQ 531, in complement
tggctctgcatttcttcc

SEQ ID NO: 576 - DNA, 20 bp, Homo Sapiens
  primer_bind 1..20: downstream amplification primer 9-3 for SEQ 532, in complement
tgccctgcccaacatacttc

SEQ ID NO: 577 - DNA, 19 bp, Homo Sapiens
  primer_bind 1..19: downstream amplification primer 9-24 for SEQ 533, in complement
ctagatgcctcagagccac

SEQ ID NO: 578 - DNA, 18 bp, Homo Sapiens
  primer_bind 1..18: downstream amplification primer 9-7 for SEQ 534, in complement
ccaagacactcttccttc

SEQ ID NO: 579 - DNA, 18 bp, Homo Sapiens
  primer_bind 1..18: downstream amplification primer 99-4582 for SEQ 535, in complement
gcaggtgccctgtctaag